SEQUENCE LISTING (115 sequences)

SEQ ID NO: 1 - protein, 1051 aa, Streptococcus suis
MKKKAVVGSVAALVLGMSLCSYQLGRFQAMEEQKNRVSYIEDSQSVQTTVAEQLTPDQVSAKENIDAEQIVVKITDQGYVTSHGDHFHYYNGKVPFDAIFSEELVMKDPNYVLQDSHIINEVQDGYVIKVDGKYYLYLKDPSKHKNVRSKEEVERQKGISSADSKNQAAGNSKDGRYRTDDGYVFNPTDVIEDTGDGFIVPHGDHFHFIPKKDLSAAELKAAQDYWNQKGSVSSASGSQYGDRNNRAQQTTISAGQGQDLASLLAQLDATPLSQRHVEADGLVFDPRTITKKTAAGVIVPHGDHYHFIPYSQMSPLEEKISRMIGVNGAGVSSGAQASHSQHTLTQPNRPVTPIGTVTTQPVSPTQPVLPTQPKQSTGKVVSYKGRQIPAYGKGLDGKAYFTSDGYTFSKDSITSVDDQGLIASHGDHFHYVGFGELEDFEIKQVEEWVNEKAGKQVPPKTSEQVGNDAKPTTPSQGNDSKPVKPIQEENRPAFEYKQVTAKRKLAGKVVYEMEVGGKTYTYGRDELDLMKISFAELTLAEKDKQYIFDIAPLAEGDLKPAMLVGMDQIPMKGANATYDTGQSFIIPHIDHIHVLPYTWLSKEQIATIRYIMQHPEIRPSAWTTSGHGDGEATDLVPPILNATPKANRLGLKNWQIIHTAEEVMDARAKGKFATNDGYIFSAEDVLDPASFVFSQAFSLPRATGGSLRSISKKDLSKEELEAVQTLLDKRDAEELAKNVTPIEKRAGLKNWQIVHSAEELAEAKAAGKYTTKDGYIFDPADLLDPKVKIGTDNYRIPRVITDGYRRINKSDLNYLSELIPAEAMVAQREKSNSSLPSTPAPTETGASAGETTTPEQPQVAKETAEEIYNRVEAKKVVPFEALTYNAGYATEVRNGTLVIPHQDHYHYVSFKWFDQGSARSPEGYSLEDFLATVKYYMTNPQERPVSDDGWGVFTPNTPSESTEETETEESDEEIISEETEEIDEFTEELKRRAEEFGMDFKTFEQSLVTLSDRYKVSFEAFEYDAASKVVRLVDKDGVKRTISLPSLEEQV

SEQ ID NO: 2 - protein, 843 aa, Streptococcus suis
MVNKERNIDMKQGKTLLYVAGASLICAVALVTYQASNYREQSNHQKTTSIETSQSKQSKEIPSDSKKEVPGIDKATDDGFLLTDESQIEEKTDLGIIVNHGDHKHFFFYSDLKNTKWAYLIPENYQEKNHSSQPKRSQLSSGSKQIEDEYVFDPKDIVAEDANGYTVRHGDHYHYILKSSLGGTTHRQLTSDSRQSPTLPIVHQQEGIPGIDFQTSDGFLFDGQNISGVTETGILVRHGKHLHLIHFETLKRSKWSYLVNQYKPNVESDKKAQPEVENTEYQTKLDYLAKELNLEPSRFKKVIVDGQIGLEYPHGDHTHIVLLKEIDTTKPFESPEDRILKQKDGETLEQRKERLIKQYMERFKVKREDITIDGNYMSVRHGDHAHVYKIDPSLPDDPERDVKTETVNLAIEKQLVYGPFYTEGSTENLTRNGVHQKYRPEGLQDIKNFILVTFSTNSDFGNFVVNGKKTKRVYYLVRKDLNWEDLNIAYPQTLQQKGRVFNGWNATLPKSGKMAREHQSFYADFDNVYRKPTKNIYTPSDDVSNIDLSDYVPVKYSAIANGRLKLNGEIRAGFIYFVKSDLTWKQAKEQGLVVPEPVSSKDYEFIEFRTVITGGEKDTDYVSATTSLAAFGTTAPRIGPYIANNTENPTDINDPSRHPNYYWHDPKNYVALAFRAGEGGQLQTHLGTSKTVVYLVRKGLTLNQAGIFPPSLKSDTGYKRDYTKPVIPNVAWDTPILEDTVYDIHFDKVESDSKDGTVEPGDTWLPGNPLANTPDVPNGEEDADSWLDDLLTPSPEATDIETVTEAETTTEDTKATESSTAAPIDIPKKSSTEEEPSEDFIFP

SEQ ID NO: 3 - protein, 858 aa, Streptococcus suis
MKKKTTLFALTGIILSCHLFLAGCQKTENTAINHQDVQSSNISKVDENTIVVSEIKSNGYMRLYGEESYFFEGPIPYAAHFLKDTLPDSDYKLNKEDIQYQLEKGYIIKVNEQYFYYPKDKDANVISISEAKKLTKNQKKPTSQAKKGVAGVDYPTDDGFLFENEGQILSKTDEGLVLEHNGHSHFIFYKDLKESKWSYLVPKEYIENSQSSSHSNAKQAKLSQTFDDGYVFDPKDVVAEDANGYTVRHGDHYHYIWKSSLQYGQGNTGETIGKKGQITVLDNHSLHTPTTRNNFVPLKSTTEKGHTSTQSNSKKAFPGIDYPTSDGFLFDGSGVQGQTALGLLIGHGTHTHLLPYSHLIGSPWESYIPTQYLETARAEYHSSTSTQVNVEIPLSPQEGGKPNSSVEEEREAKKSYLAENLHVSKAKIKVIETDAGPAFVYPHGDHSHTILIEKVEVGKPIEDPHGDPHAHDKIGMATLKQLGFDDEIIEDILHATADTPFPSNETDSEKMREWLKTVKYLNIGQRKDPLKRAGLDLMPNVEVLGIGFTPIDDVKPILQFKKLKQLWMTSTGVKDYQFLKEIPTLEGIDLSQNGVSDLGFLEEFPNLKVVSAAGNDIEDITILAKLKALESLNLDHNKVTDLSPLADLSQLTAVSLDNNRITDLSALQNKKKLTRLYISQNPQLDISTLKTENLEELTANESNVKDLQFVKNNPNLTSLTLKNNKITELQGIEENEKLVNLDVEGNQIKTLEIEGKQESVVRLNVADNQLKNLEGVNDYKALEDLNASKNDIETLAITEPNKTLKTIDVSENHIPKEELNLNDQKIPSAIAEHFPAVEGGSIENNQPKEVDKEAKVSE

SEQ ID NO: 4 - protein, 858 aa, Streptococcus suis
MKKKTTLFALTGIILSCHLFLAGCQKTENTAINHQDVQSSNISKVDENTIVVSEIKSNGYMRLYGEESYFFEGPIPYAAHFLKDTLPDSDYKLNKEDIQYQLEKGYIIKVNEQYFYYPKDKDANVISISEAKKLTKNQKKPTSQAKKGVAGVDYPTDDGFLFENEGQILSKTDEGLVLEHNGHSHFIFYKDLKESKWSYLVPKEYIENSQSSSHSNAKQAKLSQTFDDGYVFDPKDIVAEDANGYTVRHGDHYHYIWKSSLQYGQGNTGETIGKKGQITVLDNHSLHTPTTRNNFVPLKSTTEKGHTSTQSNSKKAFPGIDYPTSDGFIFDGSGVQGQTALGLLIGHGTHTHLLPYSHLIGSPWESYIPTQYLETARAEYHSSTSTQVDVEIPLSPQEGVKPNSSVEEEREAKKSYLAENMHVSKEKIKVIETDAGPAFVYPHGDHSHTILIEKVEVGKPIEDPHGDPHAHDKIGMATLKQLGFDDEIIEDILHATADTPFPSNETDSEKMREWLKTVKYLNIGQRKDPLKRAGLDLMPNVEVLGIGFTPIDDVKPILQFKKLKQLWMTSTGVKDYQFLKEIPTLEGIDLSQNGVSDLGFLEEFPNLKVVSAAGNDIEDITILAKLKALESLNLDHNKVTDLSPLADLSQLTAVSLDNNRITDLSALQNKKKLTRLYISQNPQLDISTLKTENLEELTANESNVKDLQFVKNNPNLTSLTLKNNKITELQGIEENEKLVNLDVEGNQIKTLEIEGKQESVVRLNVADNQLKNLEGINDYKALEDLNASKNDIETLAITEPNKTLKTIDVSENHIPKEELNLNDQKIPSAIAEHFPAVEGGSIENNQPKEVDKEAKVGE

SEQ ID NO: 5 - protein, 503 aa, Streptococcus suis
MKKVGLLFLSVSALLLGACGNSTASEDGKLNIVTTFYPVYEFTKQVAGDEANVDLLVKAGTEVHGYEPSAKDIARIQEADAFVYENENMETWVHDVEKSLDTTKVNVISATEGMLLLPGGEEEHEGHDHSEEGHSHAYDPHVWLSPERAITLVENIRDSLVAKYPEKKDAFETNAAAYIEKLDALDAKYSETLSAAKQKYFVTQHTAFAYLALDYGLKQVSITGVAADEDPTPSRLAELTEYINKYGIKYIYFEENASKSVAETLAKETGVQLDVLNPLESLTDEDMKNGKDYISVMEDNLIALEKTTSQEGSEILPEEGAETAQTVYNGYFEDSAVKDRTLSDYAGEWQSVYPYLLDGTLDQVWDYKAKIKGGMTAEEYKTYYDTGYKTDVDQINITDNTMEFVVGDKKEKFTYKYVGYKILTYKKGNRGVRFLFEATDANAGNYKYVQFSDHNIAPVKTGHFHIYFGGESQEKLLEELENWPTYYPVGLTGLEIGQEMLAH

SEQ ID NO: 6 - protein, 1585 aa, Streptococcus suis
MKKKETFSLRKYKIGTVSVLLGAVFLFAGAPSVAADELTSLVETKVEATVPDVIVSESASESPVVEELVDTSVEATPTDVTTTDNVEETLGSEALENITNTEVEATQPAVETPAISEKKVEEEEKLAVADETTAITNQEEAKPQNIDSNTIITVPKVWDSGYKGEGTVVAIIDSGLDVDHDVLHISDLSTAKYKSEKEIEAAKEAAGITYGEWFNDKVVFGYNYVDVNTVLKEEDKRSHGMHVTSIATGNPTQPVAGQLMYGVAPEAQVMFMRVFSDLKATTGAALYVKAIEDAVKLGADSINLSLGGANGSVVNMNENVTAAIEAARRAGVSVVIAAGNDGTFGSGHSNPSADYPDYGLVGAPSTARDAISVASYNNTTVGSKVINIIGLENNADLNYGKSSFDNPEKSPVSFEIGKEYEYVYAGIGQASDFDGLNLIGKLALIKRGTISFSEKIANATAAGAVGVVIFNSRPGEANVSMQLDDTAIAIPSIFIPLEFGEALASNSYKIAFNNETDIRPNPEAGLLSDFSSWGLSADGELKPDLAAPGGAIYAAINDNDYANMQGTSMASPHVAGAAVLVKQYLQATYPTKSPQEIEALVKHLLMSTAKAHVNKETTAYTSPRQQGAGIIDTAAAISTGLYLTGEDGYGSITLGNVEDIFSFTVTLHNITNEDKTLNYSTQLTTDTVQNGLITLAPCLLAEIPGGKVTVKANSSTTVTINVDAASFAEELTGLMKNGYYLEGFVRFTDVADVGDIVSIPYVGFRGEFQNLAVLEEPIYNLIADGKGGFYFEPVTAQPDTVDISHHYTGLVTGSTELIYSTDKRSDFAIKTLGTFKNEAGYFVLELDESGKPHLAISPNGDDNQDSLALKGVFLRNYTDLVASVYAADDTERTNPLWESQPQSGNKNFYSGDPKNPKSSIIYPTEWNGTDSEGNALADGKYQYVLTYSSEVPGAAVQTMIFDVIIDRESPVITTATYDETNFTFNPRPAIEKGESGLYREQVFYLVADASGVTTIPSLLENGDVTVSDNKVFVAQNDDGSFTLPLDLADISKFYYTVEDYAGNISYEKVENLISIGNEKGLVTVNILDKDTNSPVPILFSYSVTDETGKIVAELPRDAGDTSVLKLPFGTYTFDLFLYDTEWSSLAGETKAVVTISEENSTAEVNFYVTLKDKANLLVDIDALLPSGSTIQLVTADGQTIQLPNAKYSKTDYGKFVPVGTYTILPTLPEGYEFLEELDVAVLANQSNVKKLTLINKVALKELIAELAGLEETARYYNASPELQTAYAKALEDANAVYANKHNQVQVDSALANLVAAREQLNGQATDKEKLIAEVSNYTPTQANFIYYNAENTKQIAYDTAVRSAQLVLNQENVTQAVVNQALADLLAAKANLDGQKTDISALRSAVSVSSVLKATDAKYLNASENVKQAYDQAVEAAKAILADESASQASVDQALAVLTSAQAELNGIATSTNDAKEPANTATDKKDEGTVTPPPIDSEKVDVQAPPVKDTGNSGHVSIGQKPNPQPTLPRPVTLQASLSSPNQEKQVTQLPNTGDNDTRYYLVLGVIIGLGTLLVSKRRHKEEV

SEQ ID NO: 7 - protein, 1692 aa, Streptococcus suis
MKQKWSQIENKQRFSIKKLSVGVASVSIGFFITGVPMVQADTSGEGLESTVAVATDMDSRQNSAVEKKEDGPLSDDPVKTEQVDEPVAEEGVVEEVVDTEAGEESGLLTDQAATEIETTAGKTTDESKEKEDISGKEASAPQTIPQESQLEPEEVTTGRYILQFSEENRNLVLDKLKKIDGVKIVHEYKEVLTGASVEVGKESLSDVKAITELTSLEESRRIRPTLHTAKQLVGALKASSKYQTDGRGMVIAVIDSGLDIKHKDMRLDDGVIPKIKDITPSTTGTYTLKVPHGYNYVSGNDNLYDDTHEPHGMHIAGTLAGNATDEEVASKKGVDGIAPNAQLLVYKIFSNDPKNYKAETEDAAYAAIEDAIKHGADVISLSVGYYDSGLPGNAYYTIAKRAAEKGIIITAAIGNAGASSSDTSFDLHTNNALGAVDTATTVGVAATPAVIAVGSARNTHLVQREFMLNGQSFGYYPIGYTTLTEGKYEFVDAGNGHWEEVQGLDLAGKVAVIKKDKFDLKDAVRNLKFKDVAGIIVINTDQGWNKDYYRTHQLLVDDKTLLSYSSIWGISLSGEDGRRLLEVANQSQGNTGLVLKPTIGMKKLIEVPTVSGFSSWGPTVNLELKPEIVAPGEDVYATLNDNRYGSMSGTSMASPIVAGASALLLPRIRQMTPPEGMTRMDLLRIILMNTATPLVDVLDSSGHALENSPRQQGAGLLQIDRAFETDVILHHRLKGGVELKEIGRETEFEVTLENLGNQQRSFAISAGKVLTSQDVPVDRIGRSGKVVKEIHATEIKGSSIHLSEQSIQLGPKEKRTIRLKLDAGEAKDQFAEGYIYFKSLTEGQSDISIPYFGFVGDWSKERIVDAPAWETSSKLKLTSVLSSYKHNKSGRYIELGREKIQDNQSPLNPDNIAIQNQHSDSQIGNAFVRFALLRDITNYDLDIVKEATEDAPVLRRIDTGTMLSRVRYVDYFESLSEYSKLRTPIELHRWDGKVYDASNDENIPAPEGQYFFRLRVKNKENGAYQYTYLPVKIDNQKPEIVAIDTNRLSSHRELVVTAKDNNKVWEVRANLNGEDLLVEKVVDDAGQLHYHLKEVELPLDAKNHLRVEVMDIAGNVVAVEKDLMAPVIQFKNLEDLMAIRSKKTVEIKANVSAQVSDVQANLDAQAVNYSLENGQLSLQIPEQSDGRHSFELILKDKDGNLIYTKTLNYLVDNEKPTIDLDIEKDEEDEEVIQIGKNGRFTLKGKVSDNVSLPKDIKLYYSNLDIGKGERKIIDVKEDGSFEQDFFKSDFPRAIMLTAVDEKGNKLKDLRINTSPESLDEEEETEVPITVNNWLIDPIRFNKESLGRELDSGLVDFKKQEDGTYLFTFEIEAETEQAHSVRINGGEKRYFEDGKLTYPVTLIEEGNVVDISVYNEADELTYTKKYQMLVDTENPVLQLENEVLPLERQVVDSEEDEDEENQYAGVLLADADGHLTLTGSAKDNGIYWSLKINEDFVARGGFWRQYGNNEKAFRYELHSLKDGDTVKLDLSDSFGNAVVKKYKVRLNDKEVSEQVPEKDLHVERSDKDQTPSIPILKSEAHIPMPKEENSLAPQTGSTEIALLTGDTREDGVEHLGKLTKHEEPLGISDERIEVSVPHREFFERSGIGETGALAADTSGKLPQTGDSLGSVFISTLLGLFGGAMALGNLKRKE

SEQ ID NO: 8 - protein, 1061 aa, Streptococcus suis
MKLSYKKRLLNQVLLASTVLLAASLAQGTVFANTEEIPPTTSETVTPLPEETPITKTSTSEATDNLVEGKETEKQTEEIADTSPTPVSTEEDTTSSEPNAEETTLRTANNDNQDTTEEKSAVPTIDTVTLETKTVYLSEAVTITESITLPDTTEKIEWTLDGKSISEWKTWNLKEGDFTGDTFITVEESRQDNQLHLNIQLAALFGEDLSKRTPSNIRRTYRHFIKNMLLEGTSADGNLIISKTLHFRPYEAYRTHEEMLTEIEETKNNAATDRLVRIESIGQSAEGRDIKMAVVAKDQASIDKYLTETTPLMLTQPDQMLKQLQAGTFDYKLPILINNTHADEQPGIDVVTSLFKEFAQKDTITFPSTDADGNPVTLHLKVTDLLDKFIFLFNFTENPDGDVKNLRSLVNGLDPNRDTGFQVNPETQAIVRQIHKWNPISVLDIHGFVSGFLIEPATPPHDPNFEYDLLADIMLEKAHEMGRAGIANSKYERYTIPKVHWGDGWDDSFSGYTAVYAMYHGILGHTIEIPEGNQESFKAGFFAVLGGVHNMATKPDSLMEMRLKYYSRGVNKVEDPKAESELVGPDGAVVGRVKKDQPKFFPDYYVIPMTLDKHNDMQEAFKMIEYFNRNGVVVKELTEDVGNFRKGDLVVDMAQAKRGFANHVLYAGSDESAWGAMYAELVVNFPDMKGFSAKAVFEENTFSGKLGSITWTKAPRTTEIDFKAPYYVVANTSESAVQAINQAIKSGAKVYLTDDGYIMETNQFSHLLDTYALYGEPLYKKPLGQELKAMKVYAPSHSYSWAGDFAILANAALAVERMGFEIVNSADEADAIILESDQFDASVFGKKPIIIVGGVAMQKLEELGILAGFNAEQFTDGGDYEGLMRAIIDDKDPLTSGYAMNGLFYSNSGNWIEGIPEGFKTLVKIADKDYYIAGWWPGHDKLANKIVAIAGNYQDQPVFIYAGNPTNKVHPVHFFRWVSNALFGSQLASLEDLPAVEILVPQPEMPKNILDEAPKTTTVVHTTKSTEAKQLPQTGEKTNYIAIALGSLLLGSIALRRKERS

SEQ ID NO: 9 - protein, 1141 aa, Streptococcus suis
MNIQERFSLRKSAVGLVSVSLLCAIYTSTVAADTVVTGVNEIIEESQVKDEVSIESEKNESLDGSNIEIVEEIADNIPSPVIAEGEVAVEMKVDRGTENVVSRNDTEVTTSEQNQIEVTETKEILNQTSYQTESGEQRQIIWAHGITPPAMEQSGGFVKEKYGDYLNYTAPFEAGKGYYDTNKSLNASFIDLNLCFAAVSSNMVHWWLEQNSSYVERYLKEKKGTVNVEENYAITDLRRYINSFQNQQNSRVFDMFKTYYGYRTNGFVSDALVDLFINGYKPKAQGGVNLEDSQLVPDSRGGFFYDVFKEKKLTNRIFSGSYERFGEDVRTVLESKGLLGLTYRTLGYATHIVTVWGAEYDNQGKIKAVYITDSDDQQEQIGLKRMGITRDASGNPRLNNHMKNNSAGALLDYVHTIRLGQDLWEEYFNPLAKAKETASQTLADTKKALDLSIQGQSELPESMRLIYLEKLNNLYNQGILSIQKAESSEMLSGALENGLNSLKSLDFPISEVGNALAPDLPVGDRSTVSDVDSLSSQETSSTNLEADTENAGIIADGTNQLHFPVEAQTTSSVEAEGDNVFEQEADTLPIIIENKDEFGSELSRNMQTSETDSLVVAVEEDVKNDEVAQVEELLESEKVENQSSELLSDTLIVESANDKEEDRVEAVVSEQPDSIPHQNVEISLVEPTNVETETVVTPINDAATPHGSPTYIDNSVTESVATPLEKDSIQAGETEIAEPTSSESTNVETETVVTPVNDVATPHGSPTYIDNSVTESVATPLEKDSIQAGETEIAEPTSSESTNVETETVVTPVNDVATPHGSPTYIDNSVTESVATPLEKDSIQAGETEIAEPTSSESTSVEAELVDNSEIHAATSSVTPCGSSAYADGSTTESVATPLEKDSIQTGNTEIAEPTSSKSTNVEAASVDNSEIHADASLTAVSSVNLDNPVIEPVAISLIGSKRDTNAEVEVSSLSKREVRKTNTDGLISVQSKVIKKELLESSLAEAGSPLLEATIAQSSNSNSTEIGMSYQNTVLLESNNTERQVSKAEIVMEHKETELVETVSSASEPVVLVENISQTSNNTIESGKNMGVQSQAGAKQILGVEQSSKVSTPTSRQIMGVGLLTLVLGSALGLLKKRRK

SEQ ID NO: 10 - protein, 1089 aa, Streptococcus suis
MIPIQQHGKRWLILTLTLSTSVLALASIGTSQPVVFADENSHLQSPKNTNKIEVLNWEAFSKKLKDYSSDQRQFHVLKLGFENRLGTLSTREELEEFGKNNNFLVINGKVTQNIHDFPHILVMNKGDVIAHNEEDYHNQMRELRFSGNGDLHNSMEPKRIHALFKIELDSNKRQLLNAAGLGTAENSLKNINGMTIYSHGLTVDNKYYEDYSKYTHNSVKNINVTKERFIANDDLIHKLIESSEAMKQSSERDKVKAFVQYVANHTTYDWEAANKAVQNYADINYYLGSDLFAVTERQKAMCVGFSTTAARAFNMLGLPAYVVVGKNAEGVPHATARVYYDKKWHTIDGTGFITGNKHQRSAKYSEKHFSTIGEDSYDVVEAGQEPKAERNYMIIDSNYESWAMKQKTADLLLFNKEKSLVGLDYIAYVEPTYITENKKNHLLDIYKALKRKVEETKATDKDDKDDKQEGYDRVLQFVNSDIDKLSALSKITEEEFKALENSMDLARVFLGQMNAKAGKEFSEGETYQSYLKNRQKNNTNSDDDRNRNELQENQANSDEVSQNSKDASAPSVNSAQQSEELEGTPSTQETISAAPSQQTPAAPKALQAKTELEDKTETSSLGNTEMVSPSSETAQNTVDSKEESDAKLPYVEPSSKESSEAQVNTVSSPQVSSASPTSSETISTDTVTASQEKAESRVSAPQVISASQTSPETNSAEVVTTSQEVAKAPVSAPQVSSEIQTLSETAPTEVATEAPELSALESNPAPQTSLETTPTEEVTETPEPSVVESNSASPTSPETNSAEAVTTSQEMAEPSVSVPQVSSEIPTSSETAPAEVATEVSEESSSASQPSPETTPTETVTTSQEVAEAPVSAPQVSSEIPTSSETAPAEVATEVTEPSVVASSSASPTSPEANSTEVVVAKQEVADPLVSAPQTSSASLTLEVPKNEHLDEKADATTPNGVEANTHEAVSVPTSDIRVQDAGSDTVPQPYSLSATLFEEAISTVEPVGVATSSQERSAVAGKVKVPTSLERSNNSVEEEKVVDSNATIENREPEKKEILTSENVLNSLVTIWVALSTSFFMRYFSRGK

SEQ ID NO: 11 - protein, 721 aa, Streptococcus suis
MPKKGLFMKKKKILLPVMSTLLLAPFVLAQQVQAAETTTAATTTNQPATTDATATVPATTDATATVPATSVENVATEETVVPAAEETVEAVIIHTNDVHGRILEEKNVIGDAKAAAVIEEERAKVENTIVVDAGDAFQGLPISNSTKGEDRANIMNQVGYDAMAVGNHEFDFGMDQAIKYKETLNFPLLSANTYVNGARVFEASTIVDKTPTVVGDEFVVIGVTTPETATKTHPKNVEGVTFTDPVTEVNKVIDEVEARALADNRVYKNYIILAHLGVDSTTPVEWRGSTLAEALSKNSKLAGKRVIVIDGHSHTVEATTYGDNVTYNQTGSYLNNIGKVTLKSDKLLGEASLISAADTKNVTPNAKIAALVDEIKAKYEAENAQVVIENNPVELNGDRSNVRVRETNLGNAVTDAIYAYGQTGFSNKTSLAVTNGGGLRATIAKDQPVTKGDIIAVLPFGNIVSQITVTGQQIYDMFTKSLSSTLQVNPETGEMLLDENGMPLFEASGGFLHISGANVFYDPTLPVEERVLLIGILNPETGEYDALDLEKTYYLATNDFLAAGGDGYTMLGGAREEGPSMDSVFAEYLKTADLSAYEVVNPYSRIIPVNSSIDTDEDGYPDFIEILLDTDPENPASNPETVPAENTDSPSNQVQNTSATDKKAPVDSPKVGDKKTEVASPAKTTKAGVLPNTGDQMNLTLSLFGLGLAGLAVAVGRRKEN

SEQ ID NO: 12 - protein, 674 aa, Streptococcus suis
MKKNIRLKSSILALVAGFSVIATQAVLADELAVQIMGVNDFHGALDMTGTARLEGETVRNAGTAALLDAYMDDSQAEFEETAAETETPAESIRVQAGDMVGASPSNSGLLQDEPTVKVFNKMDVEYGTLGNHEFDEGLDEYNRIMTGEAPKKGQFNEIVDNYTREAAKQEIVIANVIDKETGEIPYGWKPYAIKTIPVNDKEAKIGFIGVVTTEIPNLVLKKNYEQYTFLNEAETIAKYARELAEKGVNAIVVLAHVPATSKDGVAAGEAADMIAKLNEIYPEHSVDLVFAGHNHVYTNGTTGKTLIVQATSQGKAYADVRAVYDTDIADFKAVPTAKIIAVAPGQKTPSPEIQAIVDEANTIVKKVTEQKIATASQATDISREVNEFKESAVGNLVTSAQLAIAKKSGYDVDFAMTNDGGIRADLKVQEDGTVTWGAAQAVQPFGNILQVVQMTGEQIYTALNQQYDEGEKYFLQMSGIKYIYTKADNPTEENPYKVVKAFKEDGTEIVPTETYTLVINDFLFGGGDGFSIFKEAKLIGAINPDTEVFVEYLTDLEKAGQTISATIPGRKAFVEKYVEEPKAEEKEDNAGTTTDVKTPEKANDGGDSVTNQKATEQPAPSGSMAPISNKKTEKASGNQTLPNTGQEALGSLLISLGGLVSLGMAVSVRRKEGE

SEQ ID NO: 13 - protein, 813 aa, Streptococcus suis
MNFRFSKCAVALTLALLAASNPKLAQAEEILNTTPASSTEASQAVPVESDTTEEADNTESPVPATTEAENPSSSETAETSDPTSETTDTTTSEARTVTPAATETSQPVEGQTVDVRILATTDLHTNLVNYDYYQDKPVETLGLAKTAVLIEEAKKENPNVVLVDNGDTIQGTPLGNYKSIVDPIEEGEQHPMYAALETLGFDVGTLGNHEFNYGLAYLEKVIRTANMPLVNANVLDPTTKDFLYTPYTIVKKTFTDTEGKKVTLNVGVTGIVPPQILNWDKAYLEGKVIVRDAVEAVRDIIPTMRENGADIVLVLSHSGIGDDQYEVGEENVGYQIASLSGVDAVITGHSHAEFPGTAEKPSFYAKYSGVDDTNGKINGTPVTMAGKYGDHLGVIDLNLVFKDGKWTTTSSKAAIRKIDTKSSVADGRIIDLAKEAHNETIKYVRQQVGETTAPINSFFALVQDDPSVQIVNNAQIWYAKQQLAGTSEANLPILSAAAPFKAGTRGDASAYTDIPAGPIAIKNVADLYLYDNVVAILKVNGAQLKEWLEMSAGQFNQVDLSSTEPQNLVNTDFRTYNFDVIDGVTYQYDITQPNKYDRDGKIVNETASRVRNLQYNGQDVTADQEFIVVTNNYRANGTFPGVREASINRLLNLENRQAIINYIIAEKVINPTADNNWTFTDSIKGLDLRFLTADRAKSLVTDQECIVYLQASTASEGFGEFKFVYTESKVVTPDEQQSDQGNTGQDIVLESGQRITLPAVNPPAPAPQHKLASPHSQASTKTLPATGEATSMLSLLGLTLIGFVGAWTKKKEH

SEQ ID NO: 14 - protein, 1035 aa, Streptococcus suis
MFLHVKYIKEMFVKKRTLLFLSLCASALNAQLVQADMVNAPSSQVSSSQVAETGGQLSVVDEAGQIKAILSNIQGEIIGVTAQFSSETNSGITVTFSMDEQGRYVAVLDRSAFAEEDQVFSLKLTAQLTDGSFQTLSDYSFEWKKDVVSAAEKPTALDNSDATPSKIEEEATSTTTSSTVNSSLTAGATSSTSNSLTRSSSNTIAGSSNGSTSVPSARVSTASSSVNVTQPTGTITIENRNDAQGTFDVRVTNVSSPKDISSVILPTWSQTDDLRWYEATRQSDGSYKLTVNKKDHKYRTGTYTVHLYYKDSSGGLTGAGGTTTHLSEAKPTGTITIENRNDAQGTFDVRVTNVSSPKDISSVILPTWSQTDDLRWYEATRQSDGSYKLTVNKKDHKYRTGTYTVHLYYKDSSGGLTGAGGTTTHLSEVKPTGTITIENRNDAQGTFDVRVTNVSSPKDIASVLLPTWSQSDDIRWYEATRQSDGSYKLTVNKKDHKYRTGTYTVHLYYKDSNGGLTGAGGTTTHLSEAKPTGTITIENRNDAQGTFDVRVTNVSSPKDIASVLLPTWSQSDDIRWYEATRQSDGSYKLTVNKKDHKYRTGTYTVHLYYKDSNGGLTGAGGTTTHLSEAKPTGTITIENRNDAQGTFDVRVTNVSSPKDIASVLLPTWSQSDDIRWYEATRQSDGSYKLTVNKKDHKYRTGTYTVHLYYKDSNGGLTGAGGTTTHLSEAKPTGTITIENRNDAQGTFDVRVTNVSSPKDIASVLLPTWSQSDDIRWYEATRQSDGSYKLTVNKKDHKYRTGTYTVHLYYKDSNGGLTGAGGTTTHLSNPSAQRSYTVYIDPGHGGRDSGASYGGVHEKNLALSVSNKLRENLLQYGINVLMTRTGDYDVDFKTERSRMTNASNADLFISIHFNATGAGVSNATGIETYWYQYNPEYQPKINKEMHNNPTRLAESEILANKVQESLIKETGAVNRGVRRETFAVLRETAIPAILVELGFMDNPSELQVIKQDSYHTRLAKALAQGVMNWYGAVEGK

SEQ ID NO: 15 - protein, 418 aa, Streptococcus suis
MKKKILATIMLSTVVLSNANYVAVISANDVDSQIATKNQQISELTAQQAEAQQQVDAIQGQVDAIVSEQAKLTEENTRLEAESQTLAADIERLSADIVSRDGALKEQARSAQVDGSASSYINTILDSKSIIDAVSRVNAMREIISANNRMLEQQKADKEAIVEKQKANQEAITTLAANRQKLEDDAQVLQVRQAELEAAKLNLAVQKATAEDEKNSLLVQKAAAEEAARQAAARQAEYQAQQAALAQQQVASVSAPVVSTLVETTVTETVAAPTQTVSQSTPTVSTPTTSTSSGSGSSAAANNARYDASSYPVGECTWGVKSQLSWVGPYWGDAKQWLASARAEGFSTGSTPQVGAIAVWTGGYYGHVAVVTAVQSSTSIQVVESNYMGRRYIGNHRGGYFNPTTTSEGAVYYIYPPY

SEQ ID NO: 16 - protein, 725 aa, Streptococcus suis
MNSKIFSLRKSKMGLVSVAIAFLWIGTGMNMETAMAEETDATALETQLESTESSLTNTVSENAEAEEVTDEVPSEEKKSEEMEDMEEELSFIENHLEVAPIQAGDQTISGNTTPGGYVAITIDGEAITSIENILEADDKGDFSYRLSKPLAHSQTVEISALPKQFWTLEADSEERKVVVRTNRHPEAYEIPAKRLEKTSNGMHQVFIEPVFEHTSKVIGHTSVKGSVYLSINGSFVSDKTLIDPKDGRFEVTFSESLAGSKFKADDRLVLSFVSEDGQPVITNTIVKPLVKEKVSSQMTVKPLSSATSVLEGTTFPLGRVHLYNADTSEFIMEAIADETGHYKIALPALQSEDKYYRLTHNQQEDLVSVHLDTVDGSSILLDKSVMASLATYLQDADMDEATDEDPIIVPKLHNKKDYIVGRTIHLNAYVRMVSSIKGKQYPPVQVDELGFFGFQIQDLQLPFEKGERIRFEIIDPVTNNIIASKEEVVGQYLEDEDVMDLPFQVEKVTTDHGYISGKTAPDVMIELVSTQNGEEIIGKTSTDSTGRFEFDLGSRVLKNGETLSFRAFDKEGEQVAWEVVTVQKGNGHRINKPDKKDEKEEQPSKEITKNIEQSNTLEQTTLPPVRQTLTDKKVEQNAEPSKEETVSIFDDSKKDMPTKQEKMARTVRDKGTKGNVSVHDSGENTQVQSLPKTGEKTSLVANIMLSIILFLFALFIGKKKITESE

SEQ ID NO: 17 - protein, 347 aa, Streptococcus suis
MKKKAIVKPLLMSGIFLALLGGATLPSGAVPVVAAETSQSTTYHLTDDEKVAVREYIQAKMTIDMQEYRLAFLEGMMEEMASGSAEAAWDEEIADLKANLTAEQVVVLDELEANLIGSIAQHYHYLFETLTVAGKSGREEAAAIVSKYESEDDASTPEAELAALKYAREVIVELLNKESAAIDNYIAYAEATGQELAGLLESGNSNLESITSATIGYGQALATASQPKFPYDFSEMDRQIAELTASLQSKVEDKSTAKTENTGVQTSQSATNGSNDLQTVPDQGGGQISDVATGKGNISEAGQKKVIPNDNAKVLPKTSGKSSLPLTVLGLITIFAGWLLTNKQEEK

SEQ ID NO: 18 - protein, 494 aa, Streptococcus suis
MEATVPDVIVSESASESPVVEELVDTSVEATPTDVTTTDNVEETLGSEALENITNTEVEATQPAVETPAISEKKVEEEEKLAVADETTAITNQEEAKPQNIDSNTIITVPKVWDSGYKGEGTVVAIIDSGLDVDHDVLHISDLSTAKYKSEKEIEAAKEAAGITYGEWFNDKVVFGYNYVDVNTVLKEEDKRSHGMHVTSIATGNPTQPVAGQLMYGVAPEAQVMFMRVFSDLKATTGAALYVKAIEDAVKLGADSINLSLGGANGSVVNMNENVTAAIEAARRAGVSVVIAAGNDGTFGSGHSNPSADYPDYGLVGAPSTARDAISVASYNNTTVGSKVINIIGLENNADLNYGKSSFDNPEKSPVSFEIGKEYEYVYAGIGQASDFDGLNLIGKLALIKRGTISFSEKIANATAAGAVGVVIFNSRPGEANVSMQLDDTAIAIPSIFIPLEFGEALASNSYKIAFNNETDIRPNPEAGLLSDFSSWGLSFER

SEQ ID NO: 19 - protein, 580 aa, Streptococcus suis
MNTKKWRTSLLIPGIVLFGTVALVNNVSAQEVKNTIISAKQPDGGQATSKAVNVKIPAVVRLFGRELLENEFKFELREANGEELPVLDTAQNTKEGQVRFKNLSFDKPGKYWYTISEVKDELGGIEYDSKYIVAKITVEDRNGQLQAMIEFIDNDNVFNNFYTPAPAAASLSIKKVLEGRTLNTGEFEFVLKNEKGDEIEKVSNQADGSVNFSALTFTKEGTYTYTVSEVDGGLGDIIYDKSDIKATVTVKDNNHGQLVSTVTYENSDQIFENILNPGKLIAPTTDSVITDNEVSKEAMTGKEKGNIEPPKEQIANEEKDNIEASEKQMPSIVNDMVVTPEKQMTNKENDKVVISEKQMPSVVNENAVTPEKQMTNKENDNIETSEKQMPSVVNENAVTPEKQMTNKEKDNIETSEKQMPSIVNDMVVTPQEQMANKENDKVVISEKQMPSIVNDMVVTPQEQMANKENDKVETSEKQMPVNEKDNAVTPEKQMANKEKENIETSKKQIPVNENNQNGTVEENSNTKPTTEKTDKQETSTFKTETAKQILPVTGEKGSLWLLTSGIIGLAIALFTRKRKL

SEQ ID NO: 20 - protein, 219 aa, Streptococcus suis
SNSSLPSTPAPTETGASAGETTTPEQPQVAKETAEEIYNRVEAKKVVPFEALTYNAGYATEVRNGTLVIPHQDHYHYVSFKWFDQGSARSPEGYSLEDFLATVKYYMTNPQERPVSDDGWGVFTPNTPSESTEETETEESDEEIISEETEEIDEFTEELKRRAEEFGMDFKTFEQSLVTLSDRYKVSFEAFEYDAASKVVRLVDKDGVKRTISLPSLEE

SEQ ID NO: 21 - protein, 133 aa, Streptococcus suis
SLKSDTGYKRDYTKPVIPNVAWDTPILEDTVYDIHFDKVESDSKDGTVEPGDTWLPGNPLANTPDVPNGEEDADSWLDDLLTPSPEATDIETVTEAETTTEDTKATESSTAAPIDIPKKSSTEEEPSEDFIFP

SEQ ID NO: 22 - protein, 169 aa, Streptococcus suis
KTENLEELTANESNVKDLQFVKNNPNLTSLTLKNNKITELQGIEENEKLVNLDVEGNQIKTLEIEGKQESVVRLNVADNQLKNLEGVNDYKALEDLNASKNDIETLAITEPNKTLKTIDVSENHIPKEELNLNDQKIPSAIAEHFPAVEGGSIENNQPKEVDKEAKVSE

SEQ ID NO: 23 - protein, 54 aa, Streptococcus suis
GVNGAGVSSGAQASHSQHTLTQPNRPVTPIGTVTTQPVSPTQPVLPTQPKQSTG

SEQ ID NO: 24 - protein, 28 aa, Streptococcus suis
DTTKPFESPEDRILKQKDGETLEQRKER

SEQ ID NO: 25 - protein, 34 aa, Streptococcus suis
SSTSTQVDVEIPLSPQEGVKPNSSVEEEREAKKS

SEQ ID NO: 26 - protein, 34 aa, Streptococcus suis
SSTSTQVNVEIPLSPQEGGKPNSSVEEEREAKKS

SEQ ID NO: 27 - protein, 24 aa, Streptococcus suis
PGGEEEHEGHDHSEEGHSHAYDPH

SEQ ID NO: 28 - protein, 84 aa, Streptococcus suis
SESPVVEELVDTSVEATPTDVTTTDNVEETLGSEALENITNTEVEATQPAVETPAISEKKVEEEEKLAVADETTAITNQEEAKP

SEQ ID NO: 29 - protein, 112 aa, Streptococcus suis
DTSGEGLESTVAVATDMDSRQNSAVEKKEDGPLSDDPVKTEQVDEPVAEEGVVEEVVDTEAGEESGLLTDQAATEIETTAGKTTDESKEKEDISGKEASAPQTIPQESQLEP

SEQ ID NO: 30 - protein, 91 aa, Streptococcus suis
NTEEIPPTTSETVTPLPEETPITKTSTSEATDNLVEGKETEKQTEEIADTSPTPVSTEEDTTSSEPNAEETTLRTANNDNQDTTEEKSAVP

SEQ ID NO: 31 - protein, 38 aa, Streptococcus suis
SRNDTEVTTSEQNQIEVTETKEILNQTSYQTESGEQRQ

SEQ ID NO: 32 - protein, 36 aa, Streptococcus suis
TPHGSPTYIDNSVTESVATPLEKDSIQAGETEIAEP

SEQ ID NO: 33 - protein, 89 aa, Streptococcus suis
KNRQKNNTNSDDDRNRNELQENQANSDEVSQNSKDASAPSVNSAQQSEELEGTPSTQETISAAPSQQTPAAPKALQAKTELEDKTETSS

SEQ ID NO: 34 - protein, 316 aa, Streptococcus suis
DEIKAKYEAENAQVVIENNPVELNGDRSNVRVRETNLGNAVTDAIYAYGQTGFSNKTSLAVTNGGGLRATIAKDQPVTKGDIIAVLPFGNIVSQITVTGQQIYDMFTKSLSSTLQVNPETGEMLLDENGMPLFEASGGFLHISGANVFYDPTLPVEERVLLIGILNPETGEYDALDLEKTYYLATNDFLAAGGDGYTMLGGAREEGPSMDSVFAEYLKTADLSAYEVVNPYSRIIPVNSSIDTDEDGYPDFIEILLDTDPENPASNPETVPAENTDSPSNQVQNTSATDKKAPVDSPKVGDKKTEVASPAKTTKAG

SEQ ID NO: 35 - protein, 289 aa, Streptococcus suis
EIQAIVDEANTIVKKVTEQKIATASQATDISREVNEFKESAVGNLVTSAQLAIAKKSGYDVDFAMTNDGGIRADLKVQEDGTVTWGAAQAVQPFGNILQVVQMTGEQIYTALNQQYDEGEKYFLQMSGIKYIYTKADNPTEENPYKVVKAFKEDGTEIVPTETYTLVINDFLFGGGDGFSIFKEAKLIGAINPDTEVFVEYLTDLEKAGQTISATIPGRKAFVEKYVEEPKAEEKEDNAGTTTDVKTPEKANDGGDSVTNQKATEQPAPSGSMAPISNKKTEKASGNQT

SEQ ID NO: 36 - protein, 334 aa, Streptococcus suis
GETTAPINSFFALVQDDPSVQIVNNAQIWYAKQQLAGTSEANLPILSAAAPFKAGTRGDASAYTDIPAGPIAIKNVADLYLYDNVVAILKVNGAQLKEWLEMSAGQFNQVDLSSTEPQNLVNTDFRTYNFDVIDGVTYQYDITQPNKYDRDGKIVNETASRVRNLQYNGQDVTADQEFIVVTNNYRANGTFPGVREASINRLLNLENRQAIINYIIAEKVINPTADNNWTFTDSIKGLDLRFLTADRAKSLVTDQECIVYLQASTASEGFGEFKFVYTESKVVTPDEQQSDQGNTGQDIVLESGQRITLPAVNPPAPAPQHKLASPHSQASTKT

SEQ ID NO: 37 - protein, 78 aa, Streptococcus suis
PTALDNSDATPSKIEEEATSTTTSSTVNSSLTAGATSSTSNSLTRSSSNTIAGSSNGSTSVPSARVSTASSSVNVTQP

SEQ ID NO: 38 - protein, 26 aa, Streptococcus suis
PTQTVSQSTPTVSTPTTSTSSGSGSS

SEQ ID NO: 39 - protein, 45 aa, Streptococcus suis
EETDATALETQLESTESSLTNTVSENAEAEEVTDEVPSEEKKSEE

SEQ ID NO: 40 - protein, 229 aa, Streptococcus suis
AETSQSTTYHLTDDEKVAVREYIQAKMTIDMQEYRLAFLEGMMEEMASGSAEAAWDEEIADLKANLTAEQVVVLDELEANLIGSIAQHYHYLFETLTVAGKSGREEAAAIVSKYESEDDASTPEAELAALKYAREVIVELLNKESAAIDNYIAYAEATGQELAGLLESGNSNLESITSATIGYGQALATASQPKFPYDFSEMDRQIAELTASLQSKVEDKSTAKTENTG

SEQ ID NO: 41 - protein, 139 aa, Streptococcus suis
GSKVINIIGLENNADLNYGKSSFDNPEKSPVPFEIGKEYEYVYAGIGQASDFDGLNLTGKLALIKRGTISFSEKIANATAAGAVGVVIFNSRPGEANVSMQLDDTAIAIPSIFIPLEFGEALAANSYKIAFNNETDIRP

SEQ ID NO: 42 - protein, 254 aa, Streptococcus suis
QEVKNTIISAKQPDGGQATSKAVNVKIPAVVRLFGRELLENEFKFELREANGEELPVLDTAQNTKEGQVRFKNLSFDKPGKYWYTISEVKDELGGIEYDSKYIVAKITVEDRNGQLQAMIEFIDNDNVFNNFYTPAPAAASLSIKKVLEGRTLNTGEFEFVLKNEKGDEIEKVSNQADGSVNFSALTFTKEGTYTYTVSEVDGGLGDIIYDKSDIKATVTVKDNNHGQLVSTVTYENSDQIFENILNPGKLIAP

SEQ ID NO: 43 - protein, 523 aa, synthetic construct
  REGION 1..523: note = Fusion polypeptide of fragments of S. suis proteins.
SLKSDTGYKRDYTKPVIPNVAWDTPILEDTVYDIHFDKVESDSKDGTVEPGDTWLPGNPLANTPDVPNGEEDADSWLDDLLTPSPEATDIETVTEAETTTEDTKATESSTAAPIDIPKKSSTEEEPSEDFIFPKTENLEELTANESNVKDLQFVKNNPNLTSLTLKNNKITELQGIEENEKLVNLDVEGNQIKTLEIEGKQESVVRLNVADNQLKNLEGVNDYKALEDLNASKNDIETLAITEPNKTLKTIDVSENHIPKEELNLNDQKIPSAIAEHFPAVEGGSIENNQPKEVDKEAKVSEGSSNSSLPSTPAPTETGASAGETTTPEQPQVAKETAEEIYNRVEAKKVVPFEALTYNAGYATEVRNGTLVIPHQDHYHYVSFKWFDQGSARSPEGYSLEDFLATVKYYMTNPQERPVSDDGWGVFTPNTPSESTEETETEESDEEIISEETEEIDEFTEELKRRAEEFGMDFKTFEQSLVTLSDRYKVSFEAFEYDAASKVVRLVDKDGVKRTISLPSLEE

SEQ ID NO: 44 - protein, 323 aa, synthetic construct
  REGION 1..323: note = Fusion polypeptide of fragments of S. suis proteins.
EETDATALETQLESTESSLTNTVSENAEAEEVTDEVPSEEKKSEEGVNGAGVSSGAQASHSQHTLTQPNRPVTPIGTVTTQPVSPTQPVLPTQPKQSTGPTALDNSDATPSKIEEEATSTTTSSTVNSSLTAGATSSTSNSLTRSSSNTIAGSSNGSTSVPSARVSTASSSVNVTQPDTTKPFESPEDRILKQKDGETLEQRKERSSTSTQVDVEIPLSPQEGVKPNSSVEEEREAKKSPTQTVSQSTPTVSTPTTSTSSGSGSSPGGEEEHEGHDHSEEGHSHAYDPHSSTSTQVNVEIPLSPQEGGKPNSSVEEEREAKKS

SEQ ID NO: 45 - protein, 450 aa, synthetic construct
  REGION 1..450: note = Fusion polypeptide of fragments of S. suis proteins.
SESPVVEELVDTSVEATPTDVTTTDNVEETLGSEALENITNTEVEATQPAVETPAISEKKVEEEEKLAVADETTAITNQEEAKPDTSGEGLESTVAVATDMDSRQNSAVEKKEDGPLSDDPVKTEQVDEPVAEEGVVEEVVDTEAGEESGLLTDQAATEIETTAGKTTDESKEKEDISGKEASAPQTIPQESQLEPNTEEIPPTTSETVTPLPEETPITKTSTSEATDNLVEGKETEKQTEEIADTSPTPVSTEEDTTSSEPNAEETTLRTANNDNQDTTEEKSAVPSRNDTEVTTSEQNQIEVTETKEILNQTSYQTESGEQRQTPHGSPTYIDNSVTESVATPLEKDSIQAGETEIAEPKNRQKNNTNSDDDRNRNELQENQANSDEVSQNSKDASAPSVNSAQQSEELEGTPSTQETISAAPSQQTPAAPKALQAKTELEDKTETSS

SEQ ID NO: 46 - protein, 943 aa, synthetic construct
  REGION 1..943: note = Fusion polypeptide of fragments of S. suis proteins.
DEIKAKYEAENAQVVIENNPVELNGDRSNVRVRETNLGNAVTDAIYAYGQTGFSNKTSLAVTNGGGLRATIAKDQPVTKGDIIAVLPFGNIVSQITVTGQQIYDMFTKSLSSTLQVNPETGEMLLDENGMPLFEASGGFLHISGANVFYDPTLPVEERVLLIGILNPETGEYDALDLEKTYYLATNDFLAAGGDGYTMLGGAREEGPSMDSVFAEYLKTADLSAYEVVNPYSRIIPVNSSIDTDEDGYPDFIEILLDTDPENPASNPETVPAENTDSPSNQVQNTSATDKKAPVDSPKVGDKKTEVASPAKTTKAGEFEIQAIVDEANTIVKKVTEQKIATASQATDISREVNEFKESAVGNLVTSAQLAIAKKSGYDVDFAMTNDGGIRADLKVQEDGTVTWGAAQAVQPFGNILQVVQMTGEQIYTALNQQYDEGEKYFLQMSGIKYIYTKADNPTEENPYKVVKAFKEDGTEIVPTETYTLVINDFLFGGGDGFSIFKEAKLIGAINPDTEVFVEYLTDLEKAGQTISATIPGRKAFVEKYVEEPKAEEKEDNAGTTTDVKTPEKANDGGDSVTNQKATEQPAPSGSMAPISNKKTEKASGNQTGTGETTAPINSFFALVQDDPSVQIVNNAQIWYAKQQLAGTSEANLPILSAAAPFKAGTRGDASAYTDIPAGPIAIKNVADLYLYDNVVAILKVNGAQLKEWLEMSAGQFNQVDLSSTEPQNLVNTDFRTYNFDVIDGVTYQYDITQPNKYDRDGKIVNETASRVRNLQYNGQDVTADQEFIVVTNNYRANGTFPGVREASINRLLNLENRQAIINYIIAEKVINPTADNNWTFTDSIKGLDLRFLTADRAKSLVTDQECIVYLQASTASEGFGEFKFVYTESKVVTPDEQQSDQGNTGQDIVLESGQRITLPAVNPPAPAPQHKLASPHSQASTKT

SEQ ID NO: 47 - protein, 967 aa, synthetic construct
  REGION 1..967: note = Fusion polypeptide of fragments of S. suis proteins.
DEIKAKYEAENAQVVIENNPVELNGDRSNVRVRETNLGNAVTDAIYAYGQTGFSNKTSLAVTNGGGLRATIAKDQPVTKGDIIAVLPFGNIVSQITVTGQQIYDMFTKSLSSTLQVNPETGEMLLDENGMPLFEASGGFLHISGANVFYDPTLPVEERVLLIGILNPETGEYDALDLEKTYYLATNDFLAAGGDGYTMLGGAREEGPSMDSVFAEYLKTADLSAYEVVNPYSRIIPVNSSIDTDEDGYPDFIEILLDTDPENPASNPETVPAENTDSPSNQVQNTSATDKKAPVDSPKVGDKKTEVASPAKTTKAGEFEIQAIVDEANTIVKKVTEQKIATASQATDISREVNEFKESAVGNLVTSAQLAIAKKSGYDVDFAMTNDGGIRADLKVQEDGTVTWGAAQAVQPFGNILQVVQMTGEQIYTALNQQYDEGEKYFLQMSGIKYIYTKADNPTEENPYKVVKAFKEDGTEIVPTETYTLVINDFLFGGGDGFSIFKEAKLIGAINPDTEVFVEYLTDLEKAGQTISATIPGRKAFVEKYVEEPKAEEKEDNAGTTTDVKTPEKANDGGDSVTNQKATEQPAPSGSMAPISNKKTEKASGNQTGTGETTAPINSFFALVQDDPSVQIVNNAQIWYAKQQLAGTSEANLPILSAAAPFKAGTRGDASAYTDIPAGPIAIKNVADLYLYDNVVAILKVNGAQLKEWLEMSAGQFNQVDLSSTEPQNLVNTDFRTYNFDVIDGVTYQYDITQPNKYDRDGKIVNETASRVRNLQYNGQDVTADQEFIVVTNNYRANGTFPGVREASINRLLNLENRQAIINYIIAEKVINPTADNNWTFTDSIKGLDLRFLTADRAKSLVTDQECIVYLQASTASEGFGEFKFVYTESKVVTPDEQQSDQGNTGQDIVLESGQRITLPAVNPPAPAPQHKLASPHSQASTKTLEGLKTRNKKAKSDKLIVRRRNQK

SEQ ID NO: 48 - protein, 622 aa, synthetic construct
  REGION 1..622: note = Fusion polypeptide of fragments of S. suis proteins.
AETSQSTTYHLTDDEKVAVREYIQAKMTIDMQEYRLAFLEGMMEEMASGSAEAAWDEEIADLKANLTAEQVVVLDELEANLIGSIAQHYHYLFETLTVAGKSGREEAAAIVSKYESEDDASTPEAELAALKYAREVIVELLNKESAAIDNYIAYAEATGQELAGLLESGNSNLESITSATIGYGQALATASQPKFPYDFSEMDRQIAELTASLQSKVEDKSTAKTENTGGSKVINIIGLENNADLNYGKSSFDNPEKSPVPFEIGKEYEYVYAGIGQASDFDGLNLTGKLALIKRGTISFSEKIANATAAGAVGVVIFNSRPGEANVSMQLDDTAIAIPSIFIPLEFGEALAANSYKIAFNNETDIRPQEVKNTIISAKQPDGGQATSKAVNVKIPAVVRLFGRELLENEFKFELREANGEELPVLDTAQNTKEGQVRFKNLSFDKPGKYWYTISEVKDELGGIEYDSKYIVAKITVEDRNGQLQAMIEFIDNDNVFNNFYTPAPAAASLSIKKVLEGRTLNTGEFEFVLKNEKGDEIEKVSNQADGSVNFSALTFTKEGTYTYTVSEVDGGLGDIIYDKSDIKATVTVKDNNHGQLVSTVTYENSDQIFENILNPGKLIAP

SEQ ID NO: 49 - protein, 530 aa, synthetic construct
  REGION 1..530: note = Fusion polypeptide of fragments of S. suis proteins.
MSLKSDTGYKRDYTKPVIPNVAWDTPILEDTVYDIHFDKVESDSKDGTVEPGDTWLPGNPLANTPDVPNGEEDADSWLDDLLTPSPEATDIETVTEAETTTEDTKATESSTAAPIDIPKKSSTEEEPSEDFIFPKTENLEELTANESNVKDLQFVKNNPNLTSLTLKNNKITELQGIEENEKLVNLDVEGNQIKTLEIEGKQESVVRLNVADNQLKNLEGVNDYKALEDLNASKNDIETLAITEPNKTLKTIDVSENHIPKEELNLNDQKIPSAIAEHFPAVEGGSIENNQPKEVDKEAKVSEGSSNSSLPSTPAPTETGASAGETTTPEQPQVAKETAEEIYNRVEAKKVVPFEALTYNAGYATEVRNGTLVIPHQDHYHYVSFKWFDQGSARSPEGYSLEDFLATVKYYMTNPQERPVSDDGWGVFTPNTPSESTEETETEESDEEIISEETEEIDEFTEELKRRAEEFGMDFKTFEQSLVTLSDRYKVSFEAFEYDAASKVVRLVDKDGVKRTISLPSLEEHHHHHH

SEQ ID NO: 50 - protein, 327 aa, synthetic construct
  REGION 1..327: note = Fusion polypeptide of fragments of S. suis proteins.
MTGSEETDATALETQLESTESSLTNTVSENAEAEEVTDEVPSEEKKSEEGVNGAGVSSGAQASHSQHTLTQPNRPVTPIGTVTTQPVSPTQPVLPTQPKQSTGPTALDNSDATPSKIEEEATSTTTSSTVNSSLTAGATSSTSNSLTRSSSNTIAGSSNGSTSVPSARVSTASSSVNVTQPDTTKPFESPEDRILKQKDGETLEQRKERSSTSTQVDVEIPLSPQEGVKPNSSVEEEREAKKSPTQTVSQSTPTVSTPTTSTSSGSGSSPGGEEEHEGHDHSEEGHSHAYDPHSSTSTQVNVEIPLSPQEGGKPNSSVEEEREAKKS

SEQ ID NO: 51 - protein, 454 aa, synthetic construct
  REGION 1..454: note = Fusion polypeptide of fragments of S. suis proteins.
MTGSSESPVVEELVDTSVEATPTDVTTTDNVEETLGSEALENITNTEVEATQPAVETPAISEKKVEEEEKLAVADETTAITNQEEAKPDTSGEGLESTVAVATDMDSRQNSAVEKKEDGPLSDDPVKTEQVDEPVAEEGVVEEVVDTEAGEESGLLTDQAATEIETTAGKTTDESKEKEDISGKEASAPQTIPQESQLEPNTEEIPPTTSETVTPLPEETPITKTSTSEATDNLVEGKETEKQTEEIADTSPTPVSTEEDTTSSEPNAEETTLRTANNDNQDTTEEKSAVPSRNDTEVTTSEQNQIEVTETKEILNQTSYQTESGEQRQTPHGSPTYIDNSVTESVATPLEKDSIQAGETEIAEPKNRQKNNTNSDDDRNRNELQENQANSDEVSQNSKDASAPSVNSAQQSEELEGTPSTQETISAAPSQQTPAAPKALQAKTELEDKTETSS

SEQ ID NO: 52 - protein, 971 aa, synthetic construct
  REGION 1..971: note = Fusion polypeptide of fragments of S. suis proteins.
MTGSDEIKAKYEAENAQVVIENNPVELNGDRSNVRVRETNLGNAVTDAIYAYGQTGFSNKTSLAVTNGGGLRATIAKDQPVTKGDIIAVLPFGNIVSQITVTGQQIYDMFTKSLSSTLQVNPETGEMLLDENGMPLFEASGGFLHISGANVFYDPTLPVEERVLLIGILNPETGEYDALDLEKTYYLATNDFLAAGGDGYTMLGGAREEGPSMDSVFAEYLKTADLSAYEVVNPYSRIIPVNSSIDTDEDGYPDFIEILLDTDPENPASNPETVPAENTDSPSNQVQNTSATDKKAPVDSPKVGDKKTEVASPAKTTKAGEFEIQAIVDEANTIVKKVTEQKIATASQATDISREVNEFKESAVGNLVTSAQLAIAKKSGYDVDFAMTNDGGIRADLKVQEDGTVTWGAAQAVQPFGNILQVVQMTGEQIYTALNQQYDEGEKYFLQMSGIKYIYTKADNPTEENPYKVVKAFKEDGTEIVPTETYTLVINDFLFGGGDGFSIFKEAKLIGAINPDTEVFVEYLTDLEKAGQTISATIPGRKAFVEKYVEEPKAEEKEDNAGTTTDVKTPEKANDGGDSVTNQKATEQPAPSGSMAPISNKKTEKASGNQTGTGETTAPINSFFALVQDDPSVQIVNNAQIWYAKQQLAGTSEANLPILSAAAPFKAGTRGDASAYTDIPAGPIAIKNVADLYLYDNVVAILKVNGAQLKEWLEMSAGQFNQVDLSSTEPQNLVNTDFRTYNFDVIDGVTYQYDITQPNKYDRDGKIVNETASRVRNLQYNGQDVTADQEFIVVTNNYRANGTFPGVREASINRLLNLENRQAIINYIIAEKVINPTADNNWTFTDSIKGLDLRFLTADRAKSLVTDQECIVYLQASTASEGFGEFKFVYTESKVVTPDEQQSDQGNTGQDIVLESGQRITLPAVNPPAPAPQHKLASPHSQASTKTLEGLKTRNKKAKSDKLIVRRRNQK

SEQ ID NO: 53 - protein, 949 aa, synthetic construct
  REGION 1..949: note = Fusion polypeptide of fragments of S. suis proteins.
MTGSDEIKAKYEAENAQVVIENNPVELNGDRSNVRVRETNLGNAVTDAIYAYGQTGFSNKTSLAVTNGGGLRATIAKDQPVTKGDIIAVLPFGNIVSQITVTGQQIYDMFTKSLSSTLQVNPETGEMLLDENGMPLFEASGGFLHISGANVFYDPTLPVEERVLLIGILNPETGEYDALDLEKTYYLATNDFLAAGGDGYTMLGGAREEGPSMDSVFAEYLKTADLSAYEVVNPYSRIIPVNSSIDTDEDGYPDFIEILLDTDPENPASNPETVPAENTDSPSNQVQNTSATDKKAPVDSPKVGDKKTEVASPAKTTKAGEFEIQAIVDEANTIVKKVTEQKIATASQATDISREVNEFKESAVGNLVTSAQLAIAKKSGYDVDFAMTNDGGIRADLKVQEDGTVTWGAAQAVQPFGNILQVVQMTGEQIYTALNQQYDEGEKYFLQMSGIKYIYTKADNPTEENPYKVVKAFKEDGTEIVPTETYTLVINDFLFGGGDGFSIFKEAKLIGAINPDTEVFVEYLTDLEKAGQTISATIPGRKAFVEKYVEEPKAEEKEDNAGTTTDVKTPEKANDGGDSVTNQKATEQPAPSGSMAPISNKKTEKASGNQTGTGETTAPINSFFALVQDDPSVQIVNNAQIWYAKQQLAGTSEANLPILSAAAPFKAGTRGDASAYTDIPAGPIAIKNVADLYLYDNVVAILKVNGAQLKEWLEMSAGQFNQVDLSSTEPQNLVNTDFRTYNFDVIDGVTYQYDITQPNKYDRDGKIVNETASRVRNLQYNGQDVTADQEFIVVTNNYRANGTFPGVREASINRLLNLENRQAIINYIIAEKVINPTADNNWTFTDSIKGLDLRFLTADRAKSLVTDQECIVYLQASTASEGFGEFKFVYTESKVVTPDEQQSDQGNTGQDIVLESGQRITLPAVNPPAPAPQHKLASPHSQASTKTLE

SEQ ID NO: 54 - protein, 627 aa, synthetic construct
  REGION 1..627: note = Fusion polypeptide of fragments of S. suis proteins.
GPLGSAETSQSTTYHLTDDEKVAVREYIQAKMTIDMQEYRLAFLEGMMEEMASGSAEAAWDEEIADLKANLTAEQVVVLDELEANLIGSIAQHYHYLFETLTVAGKSGREEAAAIVSKYESEDDASTPEAELAALKYAREVIVELLNKESAAIDNYIAYAEATGQELAGLLESGNSNLESITSATIGYGQALATASQPKFPYDFSEMDRQIAELTASLQSKVEDKSTAKTENTGGSKVINIIGLENNADLNYGKSSFDNPEKSPVPFEIGKEYEYVYAGIGQASDFDGLNLTGKLALIKRGTISFSEKIANATAAGAVGVVIFNSRPGEANVSMQLDDTAIAIPSIFIPLEFGEALAANSYKIAFNNETDIRPQEVKNTIISAKQPDGGQATSKAVNVKIPAVVRLFGRELLENEFKFELREANGEELPVLDTAQNTKEGQVRFKNLSFDKPGKYWYTISEVKDELGGIEYDSKYIVAKITVEDRNGQLQAMIEFIDNDNVFNNFYTPAPAAASLSIKKVLEGRTLNTGEFEFVLKNEKGDEIEKVSNQADGSVNFSALTFTKEGTYTYTVSEVDGGLGDIIYDKSDIKATVTVKDNNHGQLVSTVTYENSDQIFENILNPGKLIAP

SEQ ID NO: 55 - protein, 626 aa, synthetic construct
  REGION 1..626: note = Fusion polypeptide of fragments of S. suis proteins.
MTGSAETSQSTTYHLTDDEKVAVREYIQAKMTIDMQEYRLAFLEGMMEEMASGSAEAAWDEEIADLKANLTAEQVVVLDELEANLIGSIAQHYHYLFETLTVAGKSGREEAAAIVSKYESEDDASTPEAELAALKYAREVIVELLNKESAAIDNYIAYAEATGQELAGLLESGNSNLESITSATIGYGQALATASQPKFPYDFSEMDRQIAELTASLQSKVEDKSTAKTENTGGSKVINIIGLENNADLNYGKSSFDNPEKSPVPFEIGKEYEYVYAGIGQASDFDGLNLTGKLALIKRGTISFSEKIANATAAGAVGVVIFNSRPGEANVSMQLDDTAIAIPSIFIPLEFGEALAANSYKIAFNNETDIRPQEVKNTIISAKQPDGGQATSKAVNVKIPAVVRLFGRELLENEFKFELREANGEELPVLDTAQNTKEGQVRFKNLSFDKPGKYWYTISEVKDELGGIEYDSKYIVAKITVEDRNGQLQAMIEFIDNDNVFNNFYTPAPAAASLSIKKVLEGRTLNTGEFEFVLKNEKGDEIEKVSNQADGSVNFSALTFTKEGTYTYTVSEVDGGLGDIIYDKSDIKATVTVKDNNHGQLVSTVTYENSDQIFENILNPGKLIAP

SEQ ID NO: 56 - protein, 229 aa, synthetic construct
  REGION 1..229: note = Recombinant polypeptide with affinty tag.
MTGSSNSSLPSTPAPTETGASAGETTTPEQPQVAKETAEEIYNRVEAKKVVPFEALTYNAGYATEVRNGTLVIPHQDHYHYVSFKWFDQGSARSPEGYSLEDFLATVKYYMTNPQERPVSDDGWGVFTPNTPSESTEETETEESDEEIISEETEEIDEFTEELKRRAEEFGMDFKTFEQSLVTLSDRYKVSFEAFEYDAASKVVRLVDKDGVKRTISLPSLEEHHHHHH

SEQ ID NO: 57 - protein, 307 aa, synthetic construct
  REGION 1..307: note = Recombinant fusion polypeptide.
MSLKSDTGYKRDYTKPVIPNVAWDTPILEDTVYDIHFDKVESDSKDGTVEPGDTWLPGNPLANTPDVPNGEEDADSWLDDLLTPSPEATDIETVTEAETTTEDTKATESSTAAPIDIPKKSSTEEEPSEDFIFPKTENLEELTANESNVKDLQFVKNNPNLTSLTLKNNKITELQGIEENEKLVNLDVEGNQIKTLEIEGKQESVVRLNVADNQLKNLEGVNDYKALEDLNASKNDIETLAITEPNKTLKTIDVSENHIPKEELNLNDQKIPSAIAEHFPAVEGGSIENNQPKEVDKEAKVSEGSLE

SEQ ID NO: 58 - DNA, 1050 bp, synthetic construct
  misc_feature 1..1050: note = Codon optimized DNA sequence of recombinant polypeptide.
  source 1..1050: mol_type = other DNA
catatgacaggatccgacgaaatcaaagcaaaatacgaagccgaaaacgcccaggttgtcatcgaaaataatccggtggaactgaatggtgaccgcagcaatgtgcgtgtccgcgaaaccaacctgggtaatgcggtgacggatgcaatttatgcttacggtcagaccggctttagtaacaaaacctccctggccgttacgaatggcggtggcctgcgtgcgaccatcgccaaagaccagccggtgacgaagggtgatattatcgcggttctgccgtttggcaatattgtttctcaaatcaccgtcacgggtcagcaaatttatgacatgttcaccaaaagcctgagctctacgctgcaggttaacccggaaaccggtgaaatgctgctggatgaaaatggcatgccgctgtttgaagcgtcaggtggcttcctgcatatctcgggcgccaacgtgttctatgatccgaccctgccggtcgaagaacgcgtgctgctgattggtatcctgaatccggaaacgggcgaatacgacgcactggatctggaaaaaacctattacctggctacgaacgactttctggcggccggtggcgatggttataccatgctgggtggcgcccgtgaagaaggcccgagcatggactctgttttcgcagaatacctgaagaccgcagatctgagcgcttatgaagtggttaacccgtactctcgcattatcccggtcaatagttccattgataccgacgaagatggctatccggattttattgaaatcctgctggacaccgatccggaaaacccggcaagtaatccggaaaccgttccggctgaaaacacggattcaccgtcgaaccaggtccaaaataccagtgcgacggacaaaaaggccccggtggattccccgaaagtgggcgataagaaaaccgaagtggcatccccggcaaaaacgaccaaagcaggtctcgagggactgaagacccgcaataagaaagccaaaagcgacaaacttattgttcgccgtcgcaatcagaagtaatgattaactagt

SEQ ID NO: 59 - DNA, 1977 bp, synthetic construct
  misc_feature 1..1977: note = Codon optimized DNA sequence of recombinant fusion polypeptide.
  source 1..1977: mol_type = other DNA
catatgacaggatccgaaattcaagccattgtggacgaagcaaataccatcgtcaaaaaagtcacggaacagaaaatcgcaacggcaagccaagcaacggacattagtcgtgaagtgaacgaatttaaagaaagcgcggtgggtaatctggttacctctgcccagctggcaattgctaaaaaatccggctatgatgttgacttcgcaatgaccaacgatggcggtatccgcgctgacctgaaagttcaggaagatggtacggtcacctggggtgcagcacaggcagtgcaaccgtttggtaacattctgcaggtggttcaaatgaccggcgaacagatctacacggctctgaatcagcaatatgatgaaggtgaaaaatactttctgcaaatgagcggcattaaatatatctacaccaaagcggataacccgacggaagaaaatccgtataaagtcgtgaaagccttcaaagaagatggcaccgaaattgtgccgacggaaacctacacgctggttatcaacgactttctgttcggcggtggcgatggtttttcaatcttcaaagaagcgaaactgattggcgccatcaatccggacaccgaagtttttgtcgaatatctgacggatctggaaaaagcgggtcagaccatttcggccacgattccgggccgtaaagcattcgtcgaaaaatacgtggaagaaccgaaagcagaagaaaaagaagacaatgctggcaccacgaccgatgtcaaaaccccggaaaaagccaacgatggtggcgacagtgttaccaatcagaaagcaacggaacaaccggctccgagtggctcaatggccccgattagcaataagaagacggaaaaagcaagtggcaaccaaaccggtaccggcgaaacgacggccccgattaacagcttctttgccctggttcaggacgacccgagtgtccagattgtcaataacgctcagatttggtatgctaaacagcaactggcaggcaccagcgaagcaaacctgccgattctgtcggcagcagcaccgtttaaagcaggcacccgtggtgatgctagcgcatacacggacatcccggcaggtccgattgcaatcaaaaatgttgcagatctgtatctgtacgacaacgtggttgcaattctgaaagtcaatggcgctcagctgaaagaatggctggaaatgtctgcgggccagttcaaccaagtggatctgagctctaccgaaccgcagaacctggttaataccgattttcgtacgtataatttcgatgtgattgacggcgttacctatcagtacgatatcacgcaaccgaacaaatacgatcgcgacggtaaaatcgtcaatgaaaccgcatcacgtgtgcgcaacctgcagtataatggccaagatgtgacggcggaccaggaatttattgtcgtgaccaacaattaccgtgcaaacggcacgtttccgggcgtgcgtgaagcttcgatcaatcgcctgctgaacctggaaaatcgccaggcgattatcaactacatcatcgccgaaaaagtgatcaacccgaccgcggataacaattggacctttacggatagtatcaaaggtctggacctgcgtttcctgaccgccgatcgcgcaaaatccctggttacggaccaggaatgcattgtctatctgcaagctagtaccgcgtccgaaggctttggtgaatttaaattcgtttacaccgaaagcaaagttgtcacgccggatgaacagcaatctgaccagggcaacaccggtcaagatattgtcctggaaagcggtcagcgtatcacgctgccggcagtgaatccgccggcaccggctccgcagcacaaactggcgtccccgcattcgcaggcatccaccaaaaccctcgagggactgaagacccgcaataagaaagccaaaagcgacaaacttattgttcgccgtcgcaatcagaagtaatgattaactagt

SEQ ID NO: 60 - DNA, 2931 bp, synthetic construct
  misc_feature 1..2931: note = Codon optimized DNA sequence of recombinant fusion polypeptide.
  source 1..2931: mol_type = other DNA
catatgacaggatccgacgaaatcaaagcaaaatacgaagccgaaaacgcccaggttgtcatcgaaaataatccggtggaactgaatggtgaccgcagcaatgtgcgtgtccgcgaaaccaacctgggtaatgcggtgacggatgcaatttatgcttacggtcagaccggctttagtaacaaaacctccctggccgttacgaatggcggtggcctgcgtgcgaccatcgccaaagaccagccggtgacgaagggtgatattatcgcggttctgccgtttggcaatattgtttctcaaatcaccgtcacgggtcagcaaatttatgacatgttcaccaaaagcctgagctctacgctgcaggttaacccggaaaccggtgaaatgctgctggatgaaaatggcatgccgctgtttgaagcgtcaggtggcttcctgcatatctcgggcgccaacgtgttctatgatccgaccctgccggtcgaagaacgcgtgctgctgattggtatcctgaatccggaaacgggcgaatacgacgcactggatctggaaaaaacctattacctggctacgaacgactttctggcggccggtggcgatggttataccatgctgggtggcgcccgtgaagaaggcccgagcatggactctgttttcgcagaatacctgaagaccgcagatctgagcgcttatgaagtggttaacccgtactctcgcattatcccggtcaatagttccattgataccgacgaagatggctatccggattttattgaaatcctgctggacaccgatccggaaaacccggcaagtaatccggaaaccgttccggctgaaaacacggattcaccgtcgaaccaggtccaaaataccagtgcgacggacaaaaaggccccggtggattccccgaaagtgggcgataagaaaaccgaagtggcatccccggcaaaaacgaccaaagcaggtgaattcgaaattcaagccattgtggacgaagcaaataccatcgtcaaaaaagtcacggaacagaaaatcgcaacggcaagccaagcaacggacattagtcgtgaagtgaacgaatttaaagaaagcgcggtgggtaatctggttacctctgcccagctggcaattgctaaaaaatccggctatgatgttgacttcgcaatgaccaacgatggcggtatccgcgctgacctgaaagttcaggaagatggtacggtcacctggggtgcagcacaggcagtgcaaccgtttggtaacattctgcaggtggttcaaatgaccggcgaacagatctacacggctctgaatcagcaatatgatgaaggtgaaaaatactttctgcaaatgagcggcattaaatatatctacaccaaagcggataacccgacggaagaaaatccgtataaagtcgtgaaagccttcaaagaagatggcaccgaaattgtgccgacggaaacctacacgctggttatcaacgactttctgttcggcggtggcgatggtttttcaatcttcaaagaagcgaaactgattggcgccatcaatccggacaccgaagtttttgtcgaatatctgacggatctggaaaaagcgggtcagaccatttcggccacgattccgggccgtaaagcattcgtcgaaaaatacgtggaagaaccgaaagcagaagaaaaagaagacaatgctggcaccacgaccgatgtcaaaaccccggaaaaagccaacgatggtggcgacagtgttaccaatcagaaagcaacggaacaaccggctccgagtggctcaatggccccgattagcaataagaagacggaaaaagcaagtggcaaccaaaccggtaccggcgaaacgacggccccgattaacagcttctttgccctggttcaggacgacccgagtgtccagattgtcaataacgctcagatttggtatgctaaacagcaactggcaggcaccagcgaagcaaacctgccgattctgtcggcagcagcaccgtttaaagcaggcacccgtggtgatgctagcgcatacacggacatcccggcaggtccgattgcaatcaaaaatgttgcagatctgtatctgtacgacaacgtggttgcaattctgaaagtcaatggcgctcagctgaaagaatggctggaaatgtctgcgggccagttcaaccaagtggatctgagctctaccgaaccgcagaacctggttaataccgattttcgtacgtataatttcgatgtgattgacggcgttacctatcagtacgatatcacgcaaccgaacaaatacgatcgcgacggtaaaatcgtcaatgaaaccgcatcacgtgtgcgcaacctgcagtataatggccaagatgtgacggcggaccaggaatttattgtcgtgaccaacaattaccgtgcaaacggcacgtttccgggcgtgcgtgaagcttcgatcaatcgcctgctgaacctggaaaatcgccaggcgattatcaactacatcatcgccgaaaaagtgatcaacccgaccgcggataacaattggacctttacggatagtatcaaaggtctggacctgcgtttcctgaccgccgatcgcgcaaaatccctggttacggaccaggaatgcattgtctatctgcaagctagtaccgcgtccgaaggctttggtgaatttaaattcgtttacaccgaaagcaaagttgtcacgccggatgaacagcaatctgaccagggcaacaccggtcaagatattgtcctggaaagcggtcagcgtatcacgctgccggcagtgaatccgccggcaccggctccgcagcacaaactggcgtccccgcattcgcaggcatccaccaaaaccctcgagggactgaagacccgcaataagaaagccaaaagcgacaaacttattgttcgccgtcgcaatcagaagtaatgattaactagt

SEQ ID NO: 61 - DNA, 2850 bp, synthetic construct
  misc_feature 1..2850: note = Codon optimized DNA sequence of recombinant fusion polypeptide.
  source 1..2850: mol_type = other DNA
catatgacaggatccgacgaaatcaaagcaaaatacgaagccgaaaacgcccaggttgtcatcgaaaataatccggtggaactgaatggtgaccgcagcaatgtgcgtgtccgcgaaaccaacctgggtaatgcggtgacggatgcaatttatgcttacggtcagaccggctttagtaacaaaacctccctggccgttacgaatggcggtggcctgcgtgcgaccatcgccaaagaccagccggtgacgaagggtgatattatcgcggttctgccgtttggcaatattgtttctcaaatcaccgtcacgggtcagcaaatttatgacatgttcaccaaaagcctgagctctacgctgcaggttaacccggaaaccggtgaaatgctgctggatgaaaatggcatgccgctgtttgaagcgtcaggtggcttcctgcatatctcgggcgccaacgtgttctatgatccgaccctgccggtcgaagaacgcgtgctgctgattggtatcctgaatccggaaacgggcgaatacgacgcactggatctggaaaaaacctattacctggctacgaacgactttctggcggccggtggcgatggttataccatgctgggtggcgcccgtgaagaaggcccgagcatggactctgttttcgcagaatacctgaagaccgcagatctgagcgcttatgaagtggttaacccgtactctcgcattatcccggtcaatagttccattgataccgacgaagatggctatccggattttattgaaatcctgctggacaccgatccggaaaacccggcaagtaatccggaaaccgttccggctgaaaacacggattcaccgtcgaaccaggtccaaaataccagtgcgacggacaaaaaggccccggtggattccccgaaagtgggcgataagaaaaccgaagtggcatccccggcaaaaacgaccaaagcaggtgaattcgaaattcaagccattgtggacgaagcaaataccatcgtcaaaaaagtcacggaacagaaaatcgcaacggcaagccaagcaacggacattagtcgtgaagtgaacgaatttaaagaaagcgcggtgggtaatctggttacctctgcccagctggcaattgctaaaaaatccggctatgatgttgacttcgcaatgaccaacgatggcggtatccgcgctgacctgaaagttcaggaagatggtacggtcacctggggtgcagcacaggcagtgcaaccgtttggtaacattctgcaggtggttcaaatgaccggcgaacagatctacacggctctgaatcagcaatatgatgaaggtgaaaaatactttctgcaaatgagcggcattaaatatatctacaccaaagcggataacccgacggaagaaaatccgtataaagtcgtgaaagccttcaaagaagatggcaccgaaattgtgccgacggaaacctacacgctggttatcaacgactttctgttcggcggtggcgatggtttttcaatcttcaaagaagcgaaactgattggcgccatcaatccggacaccgaagtttttgtcgaatatctgacggatctggaaaaagcgggtcagaccatttcggccacgattccgggccgtaaagcattcgtcgaaaaatacgtggaagaaccgaaagcagaagaaaaagaagacaatgctggcaccacgaccgatgtcaaaaccccggaaaaagccaacgatggtggcgacagtgttaccaatcagaaagcaacggaacaaccggctccgagtggctcaatggccccgattagcaataagaagacggaaaaagcaagtggcaaccaaaccggtaccggcgaaacgacggccccgattaacagcttctttgccctggttcaggacgacccgagtgtccagattgtcaataacgctcagatttggtatgctaaacagcaactggcaggcaccagcgaagcaaacctgccgattctgtcggcagcagcaccgtttaaagcaggcacccgtggtgatgctagcgcatacacggacatcccggcaggtccgattgcaatcaaaaatgttgcagatctgtatctgtacgacaacgtggttgcaattctgaaagtcaatggcgctcagctgaaagaatggctggaaatgtctgcgggccagttcaaccaagtggatctgagctctaccgaaccgcagaacctggttaataccgattttcgtacgtataatttcgatgtgattgacggcgttacctatcagtacgatatcacgcaaccgaacaaatacgatcgcgacggtaaaatcgtcaatgaaaccgcatcacgtgtgcgcaacctgcagtataatggccaagatgtgacggcggaccaggaatttattgtcgtgaccaacaattaccgtgcaaacggcacgtttccgggcgtgcgtgaagcttcgatcaatcgcctgctgaacctggaaaatcgccaggcgattatcaactacatcatcgccgaaaaagtgatcaacccgaccgcggataacaattggacctttacggatagtatcaaaggtctggacctgcgtttcctgaccgccgatcgcgcaaaatccctggttacggaccaggaatgcatcgtctatctgcaagctagtaccgcgtccgaaggctttggtgaatttaaattcgtttacaccgaaagcaaagttgtcacgccggatgaacagcaatctgaccagggcaacaccggtcaagatattgtcctggaaagcggtcagcgtatcacgctgccggcagtgaatccgccggcaccggctccgcagcacaaactggcgtccccgcattcgcaggcatccaccaaaaccctcgag

SEQ ID NO: 62 - DNA, 699 bp, synthetic construct
  misc_feature 1..699: note = Codon optimized DNA sequence of recombinant polypeptide.
  source 1..699: mol_type = other DNA
catatgacaggatccagcaacagcagcctgccgagcaccccggcgccgaccgaaaccggtgcgagcgcgggtgaaaccaccaccccggaacagccgcaagtggcgaaggaaaccgcggaggaaatctacaaccgtgttgaggcgaagaaagtggttccgttcgaagcgctgacctacaacgcgggttatgcgaccgaggtgcgcaacggcaccctggttattccgcaccaggaccactaccactatgtgagcttcaagtggtttgatcaaggtagcgcgcgtagcccggagggttatagcctggaagacttcctggcgaccgttaaatactatatgaccaacccgcaggaacgtccggtgagcgacgatggttggggcgtttttaccccgaacaccccgagcgagagcaccgaggaaaccgaaaccgaggaaagcgacgaggaaatcattagcgaggaaaccgaggaaatcgatgagttcaccgaggaactgaagcgtcgtgcggaggaatttggtatggatttcaaaacctttgaacaaagcctggtgaccctgagcgaccgttacaaggttagcttcgaggcgtttgaatatgatgcggcgagcaaagtggttcgtctggtggacaaggatggcgttaaacgtaccattagcctgccgagcctggaggaacaccaccaccaccaccactaactcgag

SEQ ID NO: 63 - DNA, 924 bp, synthetic construct
  misc_feature 1..924: note = Codon optimized DNA sequence of recombinant polypeptide.
  source 1..924: mol_type = other DNA
catatgagcctgaaaagcgacaccggttacaaacgtgactacaccaagccggttatcccgaatgtggcgtgggacaccccgatcctggaagacaccgtgtacgacatccacttcgataaggttgagagcgacagcaaagatggtaccgtggaaccgggtgacacctggctgccgggtaacccgctggcgaacaccccggatgttccgaacggcgaggaagacgcggatagctggctggatgatctgctgaccccgagcccggaagcgaccgacatcgaaaccgtgaccgaagcggaaaccaccaccgaagataccaaagcgaccgagagcagcaccgcggcgccgatcgacattccgaagaaaagcagcaccgaggaagagccgagcgaagatttcatttttccgaagaccgagaacctggaagagctgaccgcgaacgagagcaacgtgaaggacctgcagtttgttaaaaacaacccgaacctgaccagcctgaccctgaagaacaacaaaatcaccgaactgcaaggtattgaagagaacgagaaactggtgaacctggatgttgaaggtaaccagatcaagaccctggaaattgagggcaaacaagagagcgtggttcgtctgaacgtggcggataaccagctgaagaacctggagggcgttaacgactataaagcgctggaagatctgaacgcgagcaagaacgacatcgaaaccctggcgattaccgagccgaacaagaccctgaaaaccatcgatgtgagcgaaaaccacattccgaaagaagagctgaacctgaacgaccaaaagatcccgagcgcgattgcggaacacttcccggcggttgagggcggtagcattgaaaacaaccagccgaaagaagtggacaaggaagcgaaagtgagcgaaggatccctcgag

SEQ ID NO: 64 - DNA, 1602 bp, synthetic construct
  misc_feature 1..1602: note = Codon optimized DNA sequence of recombinant fusion polypeptide.
  source 1..1602: mol_type = other DNA
catatgagcctgaaaagcgacaccggttacaaacgtgactacaccaagccggttatcccgaatgtggcgtgggacaccccgatcctggaagacaccgtgtacgacatccacttcgataaggttgagagcgacagcaaagatggtaccgtggaaccgggtgacacctggctgccgggtaacccgctggcgaacaccccggatgttccgaacggcgaggaagacgcggatagctggctggatgatctgctgaccccgagcccggaagcgaccgacatcgaaaccgtgaccgaagcggaaaccaccaccgaagataccaaagcgaccgagagcagcaccgcggcgccgatcgacattccgaagaaaagcagcaccgaggaagagccgagcgaagatttcatttttccgaagaccgagaacctggaagagctgaccgcgaacgagagcaacgtgaaggacctgcagtttgttaaaaacaacccgaacctgaccagcctgaccctgaagaacaacaaaatcaccgaactgcaaggtattgaagagaacgagaaactggtgaacctggatgttgaaggtaaccagatcaagaccctggaaattgagggcaaacaagagagcgtggttcgtctgaacgtggcggataaccagctgaagaacctggagggcgttaacgactataaagcgctggaagatctgaacgcgagcaagaacgacatcgaaaccctggcgattaccgagccgaacaagaccctgaaaaccatcgatgtgagcgaaaaccacattccgaaagaagagctgaacctgaacgaccaaaagatcccgagcgcgattgcggaacacttcccggcggttgagggcggtagcattgaaaacaaccagccgaaagaagtggacaaggaagcgaaagtgagcgaaggatccagcaacagcagcctgccgagcaccccggcgccgaccgaaaccggtgcgagcgcgggtgaaaccaccaccccggaacagccgcaagtggcgaaggaaaccgcggaggaaatctacaaccgtgttgaggcgaagaaagtggttccgttcgaagcgctgacctacaacgcgggttatgcgaccgaggtgcgcaacggcaccctggttattccgcaccaggaccactaccactatgtgagcttcaagtggtttgatcaaggtagcgcgcgtagcccggagggttatagcctggaagacttcctggcgaccgttaaatactatatgaccaacccgcaggaacgtccggtgagcgacgatggttggggcgtttttaccccgaacaccccgagcgagagcaccgaggaaaccgaaaccgaggaaagcgacgaggaaatcattagcgaggaaaccgaggaaatcgatgagttcaccgaggaactgaagcgtcgtgcggaggaatttggtatggatttcaaaacctttgaacaaagcctggtgaccctgagcgaccgttacaaggttagcttcgaggcgtttgaatatgatgcggcgagcaaagtggttcgtctggtggacaaggatggcgttaaacgtaccattagcctgccgagcctggaggaacaccaccaccaccaccactaactcgag

SEQ ID NO: 65 - DNA, 1374 bp, synthetic construct
  misc_feature 1..1374: note = Codon optimized DNA sequence of recombinant fusion polypeptide.
  source 1..1374: mol_type = other DNA
catatgacaggatccagcgaaagcccggtggttgaggaactggtggataccagcgttgaggcgaccccgaccgatgtgaccaccaccgacaacgttgaggaaaccctgggtagcgaggcgctggaaaacatcaccaacaccgaggtggaagcgacccagccggcggttgaaaccccggcgattagcgagaagaaagtggaggaagaggaaaagctggcggttgcggatgaaaccaccgcgatcaccaaccaagaggaagcgaaaccggacaccagcggcgagggtctggaaagcaccgtggcggttgcgaccgatatggacagccgtcagaacagcgcggtggagaagaaagaagatggtccgctgagcgacgatccggttaagaccgaacaagtggacgagccggttgcggaggaaggcgtggttgaggaagtggttgataccgaggcgggtgaagaaagcggtctgctgaccgaccaagcggcgaccgagattgaaaccaccgcgggcaagaccaccgatgaaagcaaggagaaagaagacatcagcggcaaagaggcgagcgcgccgcaaaccattccgcaggaaagccaactggagccgaacaccgaggaaatcccgccgaccaccagcgaaaccgtgaccccgctgccggaggaaaccccgattaccaaaaccagcaccagcgaagcgaccgataacctggtggagggcaaggagaccgaaaaacagaccgaggaaattgcggataccagcccgaccccggttagcaccgaggaagacaccaccagcagcgagccgaacgcggaggaaaccaccctgcgtaccgcgaacaacgataaccaggacaccaccgaggaaaagagcgcggtgccgagccgtaacgacaccgaagttaccaccagcgagcagaaccaaatcgaggttaccgaaaccaaagagattctgaaccagaccagctaccaaaccgaaagcggtgaacaacgtcaaaccccgcacggcagcccgacctatatcgataacagcgtgaccgaaagcgttgcgaccccgctggagaaggacagcatccaggcgggcgagaccgaaattgcggaaccgaagaaccgtcagaagaacaacaccaacagcgacgatgaccgtaaccgtaacgaactgcaggagaaccaagcgaacagcgatgaagttagccagaacagcaaagatgcgagcgcgccgagcgttaacagcgcgcagcaaagcgaggaactggaaggcaccccgagcacccaagagaccattagcgcggcgccgagccagcaaaccccggcggcgccgaaggcgctgcaagcgaaaaccgagctggaagacaagaccgagaccagcagctaactcgag

SEQ ID NO: 66 - DNA, 993 bp, synthetic construct
  misc_feature 1..993: note = Codon optimized DNA sequence of recombinant fusion polypeptide.
  source 1..993: mol_type = other DNA
catatgacaggatccgaggaaaccgatgcgaccgcgctggagacccaactggagagcaccgaaagcagcctgaccaacaccgtgagcgagaacgcggaagcggaggaagtgaccgatgaagttccgagcgaggaaaagaaaagcgaggaaggtgtgaacggtgcgggcgttagcagcggtgcgcaggcgagccacagccaacacaccctgacccaaccgaaccgtccggtgaccccgatcggtaccgtgaccacccaaccggttagcccgacccaaccggttctgccgacccaaccgaagcaaagcaccggtccgaccgcgctggacaacagcgatgcgaccccgagcaaaatcgaggaagaggcgaccagcaccaccaccagcagcaccgttaacagcagcctgaccgcgggtgcgaccagcagcaccagcaacagcctgacccgtagcagcagcaacaccattgcgggtagcagcaacggcagcaccagcgtgccgagcgcgcgtgttagcaccgcgagcagcagcgtgaacgttacccagccggacaccaccaagccgttcgagagcccggaagaccgtatcctgaagcaaaaagatggcgagaccctggaacagcgtaaagagcgtagcagcaccagcacccaagtggatgttgagattccgctgagcccgcaggaaggcgtgaagccgaacagcagcgttgaagaggaacgtgaagcgaagaaaagcccgacccagaccgtgagccaaagcaccccgaccgttagcaccccgaccaccagcaccagcagcggtagcggcagcagcccgggtggcgaggaagagcatgagggtcacgaccacagcgaagagggccacagccacgcgtacgatccgcacagcagcaccagcacccaggtgaacgtggaaatccctttaagcccgcaggaaggtggcaaaccgaacagcagcgtggaagaggaacgcgaggcgaagaaaagctaactcgag

SEQ ID NO: 67 - DNA, 1881 bp, synthetic construct
  misc_feature 1..1881: note = Codon optimized DNA sequence of recombinant fusion polypeptide.
  source 1..1881: mol_type = other DNA
ggatccgcggagaccagccagagcaccacctaccacctgaccgatgacgagaaagttgcggttcgtgagtacattcaagcgaagatgaccattgacatgcaggagtaccgtctggcgttcctggaaggcatgatggaagaaatggcgagcggtagcgcggaggcggcgtgggatgaggaaatcgcggatctgaaagcgaacctgaccgcggaacaggtggttgtgctggatgagctggaagcgaacctgatcggtagcattgcgcaacactaccattacctgttcgaaaccctgaccgttgcgggtaaaagcggtcgtgaggaagcggcggcgattgtgagcaagtatgagagcgaagatgatgcgagcaccccggaggcggaactggcggcgctgaaatacgcgcgcgaggttattgtggaactgctgaacaaggaaagcgcggcgatcgacaattatattgcgtacgcggaggcgaccggtcaggaactggcgggcctgctggagagcggtaacagcaatctggaaagcatcaccagcgcgaccattggttacggtcaagcgctggcgaccgcgagccaaccgaagttcccgtacgactttagcgagatggatcgtcagatcgcggaactgaccgcgagcctgcaaagcaaggttgaggataaaagcaccgcgaagaccgaaaacaccggtggcagcaaagtgattaatatcattggcctggagaacaatgcggacctgaactatggtaaaagcagcttcgataatccggagaagagcccggttccgtttgaaatcggcaaggagtacgaatatgtgtacgcgggtattggccaagcgagcgatttcgatggcctgaacctgaccggtaaactggcgctgatcaagcgtggtaccattagcttcagcgagaaaattgcgaacgcgaccgcggcgggtgcggttggcgttgtgatttttaacagccgcccgggcgaagcgaatgtgagcatgcagctggacgataccgcgatcgcgattccgagcatcttcattccgctggagtttggtgaagcgctggcggcgaacagctacaagatcgcgttcaacaatgaaaccgacattcgtccgcaagaagttaaaaacaccatcattagcgcgaagcagccggatggtggccaagcgaccagcaaagcggttaacgtgaagatcccggcggttgtgcgtctgtttggccgcgagctgctggagaacgaattcaaatttgagctgcgtgaagcgaatggcgaggaactgccggttctggacaccgcgcagaacaccaaagaaggccaagtgcgcttcaagaatctgagctttgataagccgggtaaatattggtacaccatcagcgaggttaaggacgaactgggtggcattgagtatgatagcaaatacatcgttgcgaagattaccgtggaggaccgtaacggtcagctgcaagcgatgatcgaattcattgacaacgataacgtgttcaacaacttctacaccccggcgccggcggcggcgagcctgagcatcaagaaagttctggagggccgcaccctgaacaccggcgagttcgaatttgtgctgaagaacgaaaaaggcgacgagattgaaaaagttagcaaccaggcggatggtagcgtgaatttcagcgcgctgacctttaccaaagagggtacctatacctacaccgttagcgaagtggacggtggcctgggtgatatcatttacgacaaaagcgatatcaaggcgaccgttaccgtgaaagacaacaatcacggtcaactggttagcaccgtgacctatgagaatagcgaccagatttttgagaacatcctgaacccgggcaagctgattgcgccgtaactcgag

SEQ ID NO: 68 - DNA, 22 bp, synthetic construct
  misc_feature 1..22: note = PCR primer.
  source 1..22: mol_type = other DNA
gccgacatcataacggttctgg

SEQ ID NO: 69 - DNA, 28 bp, synthetic construct
  misc_feature 1..28: note = PCR primer.
  source 1..28: mol_type = other DNA
tataggtaccggtttggttgccacttgc

SEQ ID NO: 70 - DNA, 24 bp, synthetic construct
  misc_feature 1..24: note = PCR primer.
  source 1..24: mol_type = other DNA
atatggtaccggcgaaacgacggc

SEQ ID NO: 71 - DNA, 19 bp, synthetic construct
  misc_feature 1..19: note = PCR primer.
  source 1..19: mol_type = other DNA
ggtggtcccacctgacgtc

SEQ ID NO: 72 - DNA, 31 bp, synthetic construct
  misc_feature 1..31: note = PCR primer.
  source 1..31: mol_type = other DNA
tatagaattcacctgctttggtcgtttttgc

SEQ ID NO: 73 - DNA, 32 bp, synthetic construct
  misc_feature 1..32: note = PCR primer.
  source 1..32: mol_type = other DNA
atatgaattcgaaattcaagccattgtggacg

SEQ ID NO: 74 - protein, 316 aa, Streptococcus suis; identical to SEQ ID NO: 34

SEQ ID NO: 75 - protein, 334 aa, Streptococcus suis; identical to SEQ ID NO: 36

SEQ ID NO: 76 - protein, 289 aa, Streptococcus suis; identical to SEQ ID NO: 35

SEQ ID NO: 77 - protein, 362 aa, synthetic construct
  REGION 1..362: note = Fragment of S. suis protein with affinity-tag.
MTGSGETTAPINSFFALVQDDPSVQIVNNAQIWYAKQQLAGTSEANLPILSAAAPFKAGTRGDASAYTDIPAGPIAIKNVADLYLYDNVVAILKVNGAQLKEWLEMSAGQFNQVDLSSTEPQNLVNTDFRTYNFDVIDGVTYQYDITQPNKYDRDGKIVNETASRVRNLQYNGQDVTADQEFIVVTNNYRANGTFPGVREASINRLLNLENRQAIINYIIAEKVINPTADNNWTFTDSIKGLDLRFLTADRAKSLVTDQECIVYLQASTASEGFGEFKFVYTESKVVTPDEQQSDQGNTGQDIVLESGQRITLPAVNPPAPAPQHKLASPHSQASTKTLEGLKTRNKKAKSDKLIVRRRNQK

SEQ ID NO: 78 - protein, 317 aa, synthetic construct
  REGION 1..317: note = Fragment of S. suis protein with affinity-tag.
MTGSEIQAIVDEANTIVKKVTEQKIATASQATDISREVNEFKESAVGNLVTSAQLAIAKKSGYDVDFAMTNDGGIRADLKVQEDGTVTWGAAQAVQPFGNILQVVQMTGEQIYTALNQQYDEGEKYFLQMSGIKYIYTKADNPTEENPYKVVKAFKEDGTEIVPTETYTLVINDFLFGGGDGFSIFKEAKLIGAINPDTEVFVEYLTDLEKAGQTISATIPGRKAFVEKYVEEPKAEEKEDNAGTTTDVKTPEKANDGGDSVTNQKATEQPAPSGSMAPISNKKTEKASGNQTLEGLKTRNKKAKSDKLIVRRRNQK

SEQ ID NO: 79 - protein, 653 aa, synthetic construct
  REGION 1..653: note = Fusion polypeptide of fragments of S. suis proteins withaffinity-tag.
MTGSEIQAIVDEANTIVKKVTEQKIATASQATDISREVNEFKESAVGNLVTSAQLAIAKKSGYDVDFAMTNDGGIRADLKVQEDGTVTWGAAQAVQPFGNILQVVQMTGEQIYTALNQQYDEGEKYFLQMSGIKYIYTKADNPTEENPYKVVKAFKEDGTEIVPTETYTLVINDFLFGGGDGFSIFKEAKLIGAINPDTEVFVEYLTDLEKAGQTISATIPGRKAFVEKYVEEPKAEEKEDNAGTTTDVKTPEKANDGGDSVTNQKATEQPAPSGSMAPISNKKTEKASGNQTGTGETTAPINSFFALVQDDPSVQIVNNAQIWYAKQQLAGTSEANLPILSAAAPFKAGTRGDASAYTDIPAGPIAIKNVADLYLYDNVVAILKVNGAQLKEWLEMSAGQFNQVDLSSTEPQNLVNTDFRTYNFDVIDGVTYQYDITQPNKYDRDGKIVNETASRVRNLQYNGQDVTADQEFIVVTNNYRANGTFPGVREASINRLLNLENRQAIINYIIAEKVINPTADNNWTFTDSIKGLDLRFLTADRAKSLVTDQECIVYLQASTASEGFGEFKFVYTESKVVTPDEQQSDQGNTGQDIVLESGQRITLPAVNPPAPAPQHKLASPHSQASTKTLEGLKTRNKKAKSDKLIVRRRNQK

SEQ ID NO: 80 - protein, 22 aa, synthetic construct
  REGION 1..22: note = Affinty tag.
GLKTRNKKAKSDKLIVRRRNQK

SEQ ID NO: 81 - protein, 6 aa, synthetic construct
  REGION 1..6: note = Affinity tag.
HHHHHH

SEQ ID NO: 82 - protein, 26 aa, synthetic construct
  REGION 1..26: note = Affinity tag.
KPALGLKTRNKKAKSDKLIVRRRNQK

SEQ ID NO: 83 - protein, 8 aa, synthetic construct
  REGION 1..8: note = Affinity tag.
DYKDDDDK

SEQ ID NO: 84 - protein, 10 aa, synthetic construct
  REGION 1..10: note = Affinity tag.
EQKLISEEDL

SEQ ID NO: 85 - protein, 16 aa, synthetic construct
  REGION 1..16: note = Linker sequence.
GGGGSLVPRGSGGGGS

SEQ ID NO: 86 - protein, 6 aa, synthetic construct
  REGION 1..6: note = Linker sequence.
GSGSGS

SEQ ID NO: 87 - protein, 8 aa, synthetic construct
  REGION 1..8: note = Linker sequence.
GSGSGSGS

SEQ ID NO: 88 - protein, 16 aa, synthetic construct
  REGION 1..16: note = Linker sequence.
GSGSGSGSGSGSGSGS

SEQ ID NO: 89 - protein, 11 aa, synthetic construct
  REGION 1..11: note = Linker sequence.
GGSGGHMGSGG

SEQ ID NO: 90 - protein, 11 aa, synthetic construct
  REGION 1..11: note = Linker sequence.
GGSGGSGGSGG

SEQ ID NO: 91 - protein, 5 aa, synthetic construct
  REGION 1..5: note = Linker sequence.
GGSGG

SEQ ID NO: 92 - protein, 8 aa, synthetic construct
  REGION 1..8: note = Linker sequence.
GGSGGGGG

SEQ ID NO: 93 - protein, 18 aa, synthetic construct
  REGION 1..18: note = Linker sequence.
GGGSEGGGSEGGGSEGGG

SEQ ID NO: 94 - protein, 8 aa, synthetic construct
  REGION 1..8: note = Linker sequence.
AAGAATAA

SEQ ID NO: 95 - protein, 5 aa, synthetic construct
  REGION 1..5: note = Linker sequence.
GGGGG

SEQ ID NO: 96 - protein, 5 aa, synthetic construct
  REGION 1..5: note = Linker sequence.
GGSSG

SEQ ID NO: 97 - protein, 11 aa, synthetic construct
  REGION 1..11: note = Linker sequence.
GSGGGTGGGSG

SEQ ID NO: 98 - protein, 11 aa, synthetic construct
  REGION 1..11: note = Linker sequence.
GSGGGTGGGSG

SEQ ID NO: 99 - protein, 12 aa, synthetic construct
  REGION 1..12: note = Linker sequence.
GSGSGSGSGGSG

SEQ ID NO: 100 - protein, 14 aa, synthetic construct
  REGION 1..14: note = Linker sequence.
GSGGSGGSGGSGGS

SEQ ID NO: 101 - protein, 14 aa, synthetic construct
  REGION 1..14: note = Linker sequence.
GSGGSGSGGSGGSG

SEQ ID NO: 102 - protein, 5 aa, synthetic construct
  REGION 1..5: note = Cell wall anchor motif.
  SITE 3: note = misc_feature - Xaa can be any naturally occurring amino acid
LPXTG

SEQ ID NO: 103 - protein, 4 aa, synthetic construct
  REGION 1..4: note = N-terminal sequence of recombinant polypeptide.
MTGS

SEQ ID NO: 104 - protein, 5 aa, synthetic construct
  REGION 1..5: note = Linker sequence.
GGGGS

SEQ ID NO: 105 - protein, 5 aa, synthetic construct
  REGION 1..5: note = Linker sequence.
SSSSG

SEQ ID NO: 106 - protein, 523 aa, synthetic construct
  VARIANT 303: note = X is any amino acid or absent
  VARIANT 304: note = X is any amino acid or absent
SLKSDTGYKRDYTKPVIPNVAWDTPILEDTVYDIHFDKVESDSKDGTVEPGDTWLPGNPLANTPDVPNGEEDADSWLDDLLTPSPEATDIETVTEAETTTEDTKATESSTAAPIDIPKKSSTEEEPSEDFIFPKTENLEELTANESNVKDLQFVKNNPNLTSLTLKNNKITELQGIEENEKLVNLDVEGNQIKTLEIEGKQESVVRLNVADNQLKNLEGVNDYKALEDLNASKNDIETLAITEPNKTLKTIDVSENHIPKEELNLNDQKIPSAIAEHFPAVEGGSIENNQPKEVDKEAKVSEXXSNSSLPSTPAPTETGASAGETTTPEQPQVAKETAEEIYNRVEAKKVVPFEALTYNAGYATEVRNGTLVIPHQDHYHYVSFKWFDQGSARSPEGYSLEDFLATVKYYMTNPQERPVSDDGWGVFTPNTPSESTEETETEESDEEIISEETEEIDEFTEELKRRAEEFGMDFKTFEQSLVTLSDRYKVSFEAFEYDAASKVVRLVDKDGVKRTISLPSLEE

SEQ ID NO: 107 - protein, 943 aa, synthetic construct
  VARIANT 317: note = X is any amino acid or absent
  VARIANT 318: note = X is any amino acid or absent
  VARIANT 608: note = X is any amino acid or absent
  VARIANT 609: note = X is any amino acid or absent
DEIKAKYEAENAQVVIENNPVELNGDRSNVRVRETNLGNAVTDAIYAYGQTGFSNKTSLAVTNGGGLRATIAKDQPVTKGDIIAVLPFGNIVSQITVTGQQIYDMFTKSLSSTLQVNPETGEMLLDENGMPLFEASGGFLHISGANVFYDPTLPVEERVLLIGILNPETGEYDALDLEKTYYLATNDFLAAGGDGYTMLGGAREEGPSMDSVFAEYLKTADLSAYEVVNPYSRIIPVNSSIDTDEDGYPDFIEILLDTDPENPASNPETVPAENTDSPSNQVQNTSATDKKAPVDSPKVGDKKTEVASPAKTTKAGXXEIQAIVDEANTIVKKVTEQKIATASQATDISREVNEFKESAVGNLVTSAQLAIAKKSGYDVDFAMTNDGGIRADLKVQEDGTVTWGAAQAVQPFGNILQVVQMTGEQIYTALNQQYDEGEKYFLQMSGIKYIYTKADNPTEENPYKVVKAFKEDGTEIVPTETYTLVINDFLFGGGDGFSIFKEAKLIGAINPDTEVFVEYLTDLEKAGQTISATIPGRKAFVEKYVEEPKAEEKEDNAGTTTDVKTPEKANDGGDSVTNQKATEQPAPSGSMAPISNKKTEKASGNQTXXGETTAPINSFFALVQDDPSVQIVNNAQIWYAKQQLAGTSEANLPILSAAAPFKAGTRGDASAYTDIPAGPIAIKNVADLYLYDNVVAILKVNGAQLKEWLEMSAGQFNQVDLSSTEPQNLVNTDFRTYNFDVIDGVTYQYDITQPNKYDRDGKIVNETASRVRNLQYNGQDVTADQEFIVVTNNYRANGTFPGVREASINRLLNLENRQAIINYIIAEKVINPTADNNWTFTDSIKGLDLRFLTADRAKSLVTDQECIVYLQASTASEGFGEFKFVYTESKVVTPDEQQSDQGNTGQDIVLESGQRITLPAVNPPAPAPQHKLASPHSQASTKT

SEQ ID NO: 108 - protein, 967 aa, synthetic construct
  VARIANT 317: note = X is any amino acid or absent
  VARIANT 318: note = X is any amino acid or absent
  VARIANT 608: note = X is any amino acid or absent
  VARIANT 608: note = X is any amino acid or absent
  VARIANT 944: note = X is any amino acid or absent
  VARIANT 945: note = X is any amino acid or absent
DEIKAKYEAENAQVVIENNPVELNGDRSNVRVRETNLGNAVTDAIYAYGQTGFSNKTSLAVTNGGGLRATIAKDQPVTKGDIIAVLPFGNIVSQITVTGQQIYDMFTKSLSSTLQVNPETGEMLLDENGMPLFEASGGFLHISGANVFYDPTLPVEERVLLIGILNPETGEYDALDLEKTYYLATNDFLAAGGDGYTMLGGAREEGPSMDSVFAEYLKTADLSAYEVVNPYSRIIPVNSSIDTDEDGYPDFIEILLDTDPENPASNPETVPAENTDSPSNQVQNTSATDKKAPVDSPKVGDKKTEVASPAKTTKAGXXEIQAIVDEANTIVKKVTEQKIATASQATDISREVNEFKESAVGNLVTSAQLAIAKKSGYDVDFAMTNDGGIRADLKVQEDGTVTWGAAQAVQPFGNILQVVQMTGEQIYTALNQQYDEGEKYFLQMSGIKYIYTKADNPTEENPYKVVKAFKEDGTEIVPTETYTLVINDFLFGGGDGFSIFKEAKLIGAINPDTEVFVEYLTDLEKAGQTISATIPGRKAFVEKYVEEPKAEEKEDNAGTTTDVKTPEKANDGGDSVTNQKATEQPAPSGSMAPISNKKTEKASGNQTXXGETTAPINSFFALVQDDPSVQIVNNAQIWYAKQQLAGTSEANLPILSAAAPFKAGTRGDASAYTDIPAGPIAIKNVADLYLYDNVVAILKVNGAQLKEWLEMSAGQFNQVDLSSTEPQNLVNTDFRTYNFDVIDGVTYQYDITQPNKYDRDGKIVNETASRVRNLQYNGQDVTADQEFIVVTNNYRANGTFPGVREASINRLLNLENRQAIINYIIAEKVINPTADNNWTFTDSIKGLDLRFLTADRAKSLVTDQECIVYLQASTASEGFGEFKFVYTESKVVTPDEQQSDQGNTGQDIVLESGQRITLPAVNPPAPAPQHKLASPHSQASTKTXXGLKTRNKKAKSDKLIVRRRNQK

SEQ ID NO: 109 - protein, 530 aa, synthetic construct
  VARIANT 1: note = X is any amino acid or absent
  VARIANT 304: note = X is any amino acid or absent
  VARIANT 305: note = X is any amino acid or absent
  VARIANT 525: note = X is any amino acid or absent
  VARIANT 526: note = X is any amino acid or absent
  VARIANT 527: note = X is any amino acid or absent
  VARIANT 528: note = X is any amino acid or absent
  VARIANT 529: note = X is any amino acid or absent
  VARIANT 530: note = X is any amino acid or absent
XSLKSDTGYKRDYTKPVIPNVAWDTPILEDTVYDIHFDKVESDSKDGTVEPGDTWLPGNPLANTPDVPNGEEDADSWLDDLLTPSPEATDIETVTEAETTTEDTKATESSTAAPIDIPKKSSTEEEPSEDFIFPKTENLEELTANESNVKDLQFVKNNPNLTSLTLKNNKITELQGIEENEKLVNLDVEGNQIKTLEIEGKQESVVRLNVADNQLKNLEGVNDYKALEDLNASKNDIETLAITEPNKTLKTIDVSENHIPKEELNLNDQKIPSAIAEHFPAVEGGSIENNQPKEVDKEAKVSEXXSNSSLPSTPAPTETGASAGETTTPEQPQVAKETAEEIYNRVEAKKVVPFEALTYNAGYATEVRNGTLVIPHQDHYHYVSFKWFDQGSARSPEGYSLEDFLATVKYYMTNPQERPVSDDGWGVFTPNTPSESTEETETEESDEEIISEETEEIDEFTEELKRRAEEFGMDFKTFEQSLVTLSDRYKVSFEAFEYDAASKVVRLVDKDGVKRTISLPSLEEXXXXXX

SEQ ID NO: 110 - protein, 327 aa, synthetic construct
  VARIANT 1: note = X is any amino acid or absent
  VARIANT 2: note = X is any amino acid or absent
  VARIANT 3: note = X is any amino acid or absent
  VARIANT 4: note = X is any amino acid or absent
XXXXEETDATALETQLESTESSLTNTVSENAEAEEVTDEVPSEEKKSEEGVNGAGVSSGAQASHSQHTLTQPNRPVTPIGTVTTQPVSPTQPVLPTQPKQSTGPTALDNSDATPSKIEEEATSTTTSSTVNSSLTAGATSSTSNSLTRSSSNTIAGSSNGSTSVPSARVSTASSSVNVTQPDTTKPFESPEDRILKQKDGETLEQRKERSSTSTQVDVEIPLSPQEGVKPNSSVEEEREAKKSPTQTVSQSTPTVSTPTTSTSSGSGSSPGGEEEHEGHDHSEEGHSHAYDPHSSTSTQVNVEIPLSPQEGGKPNSSVEEEREAKKS

SEQ ID NO: 111 - protein, 454 aa, synthetic construct
  VARIANT 1: note = X is any amino acid or absent
  VARIANT 2: note = X is any amino acid or absent
  VARIANT 3: note = X is any amino acid or absent
  VARIANT 4: note = X is any amino acid or absent
XXXXSESPVVEELVDTSVEATPTDVTTTDNVEETLGSEALENITNTEVEATQPAVETPAISEKKVEEEEKLAVADETTAITNQEEAKPDTSGEGLESTVAVATDMDSRQNSAVEKKEDGPLSDDPVKTEQVDEPVAEEGVVEEVVDTEAGEESGLLTDQAATEIETTAGKTTDESKEKEDISGKEASAPQTIPQESQLEPNTEEIPPTTSETVTPLPEETPITKTSTSEATDNLVEGKETEKQTEEIADTSPTPVSTEEDTTSSEPNAEETTLRTANNDNQDTTEEKSAVPSRNDTEVTTSEQNQIEVTETKEILNQTSYQTESGEQRQTPHGSPTYIDNSVTESVATPLEKDSIQAGETEIAEPKNRQKNNTNSDDDRNRNELQENQANSDEVSQNSKDASAPSVNSAQQSEELEGTPSTQETISAAPSQQTPAAPKALQAKTELEDKTETSS

SEQ ID NO: 112 - protein, 971 aa, synthetic construct
  VARIANT 1: note = X is any amino acid or absent
  VARIANT 2: note = X is any amino acid or absent
  VARIANT 3: note = X is any amino acid or absent
  VARIANT 4: note = X is any amino acid or absent
  VARIANT 321: note = X is any amino acid or absent
  VARIANT 322: note = X is any amino acid or absent
  VARIANT 612: note = X is any amino acid or absent
  VARIANT 613: note = X is any amino acid or absent
  VARIANT 948: note = X is any amino acid or absent
  VARIANT 949: note = X is any amino acid or absent
XXXXDEIKAKYEAENAQVVIENNPVELNGDRSNVRVRETNLGNAVTDAIYAYGQTGFSNKTSLAVTNGGGLRATIAKDQPVTKGDIIAVLPFGNIVSQITVTGQQIYDMFTKSLSSTLQVNPETGEMLLDENGMPLFEASGGFLHISGANVFYDPTLPVEERVLLIGILNPETGEYDALDLEKTYYLATNDFLAAGGDGYTMLGGAREEGPSMDSVFAEYLKTADLSAYEVVNPYSRIIPVNSSIDTDEDGYPDFIEILLDTDPENPASNPETVPAENTDSPSNQVQNTSATDKKAPVDSPKVGDKKTEVASPAKTTKAGXXEIQAIVDEANTIVKKVTEQKIATASQATDISREVNEFKESAVGNLVTSAQLAIAKKSGYDVDFAMTNDGGIRADLKVQEDGTVTWGAAQAVQPFGNILQVVQMTGEQIYTALNQQYDEGEKYFLQMSGIKYIYTKADNPTEENPYKVVKAFKEDGTEIVPTETYTLVINDFLFGGGDGFSIFKEAKLIGAINPDTEVFVEYLTDLEKAGQTISATIPGRKAFVEKYVEEPKAEEKEDNAGTTTDVKTPEKANDGGDSVTNQKATEQPAPSGSMAPISNKKTEKASGNQTXXGETTAPINSFFALVQDDPSVQIVNNAQIWYAKQQLAGTSEANLPILSAAAPFKAGTRGDASAYTDIPAGPIAIKNVADLYLYDNVVAILKVNGAQLKEWLEMSAGQFNQVDLSSTEPQNLVNTDFRTYNFDVIDGVTYQYDITQPNKYDRDGKIVNETASRVRNLQYNGQDVTADQEFIVVTNNYRANGTFPGVREASINRLLNLENRQAIINYIIAEKVINPTADNNWTFTDSIKGLDLRFLTADRAKSLVTDQECIVYLQASTASEGFGEFKFVYTESKVVTPDEQQSDQGNTGQDIVLESGQRITLPAVNPPAPAPQHKLASPHSQASTKTXXGLKTRNKKAKSDKLIVRRRNQK

SEQ ID NO: 113 - protein, 949 aa, synthetic construct
  VARIANT 1: note = X is any amino acid or absent
  VARIANT 2: note = X is any amino acid or absent
  VARIANT 3: note = X is any amino acid or absent
  VARIANT 4: note = X is any amino acid or absent
  VARIANT 321: note = X is any amino acid or absent
  VARIANT 322: note = X is any amino acid or absent
  VARIANT 612: note = X is any amino acid or absent
  VARIANT 613: note = X is any amino acid or absent
  VARIANT 948: note = X is any amino acid or absent
  VARIANT 949: note = X is any amino acid or absent
XXXXDEIKAKYEAENAQVVIENNPVELNGDRSNVRVRETNLGNAVTDAIYAYGQTGFSNKTSLAVTNGGGLRATIAKDQPVTKGDIIAVLPFGNIVSQITVTGQQIYDMFTKSLSSTLQVNPETGEMLLDENGMPLFEASGGFLHISGANVFYDPTLPVEERVLLIGILNPETGEYDALDLEKTYYLATNDFLAAGGDGYTMLGGAREEGPSMDSVFAEYLKTADLSAYEVVNPYSRIIPVNSSIDTDEDGYPDFIEILLDTDPENPASNPETVPAENTDSPSNQVQNTSATDKKAPVDSPKVGDKKTEVASPAKTTKAGXXEIQAIVDEANTIVKKVTEQKIATASQATDISREVNEFKESAVGNLVTSAQLAIAKKSGYDVDFAMTNDGGIRADLKVQEDGTVTWGAAQAVQPFGNILQVVQMTGEQIYTALNQQYDEGEKYFLQMSGIKYIYTKADNPTEENPYKVVKAFKEDGTEIVPTETYTLVINDFLFGGGDGFSIFKEAKLIGAINPDTEVFVEYLTDLEKAGQTISATIPGRKAFVEKYVEEPKAEEKEDNAGTTTDVKTPEKANDGGDSVTNQKATEQPAPSGSMAPISNKKTEKASGNQTXXGETTAPINSFFALVQDDPSVQIVNNAQIWYAKQQLAGTSEANLPILSAAAPFKAGTRGDASAYTDIPAGPIAIKNVADLYLYDNVVAILKVNGAQLKEWLEMSAGQFNQVDLSSTEPQNLVNTDFRTYNFDVIDGVTYQYDITQPNKYDRDGKIVNETASRVRNLQYNGQDVTADQEFIVVTNNYRANGTFPGVREASINRLLNLENRQAIINYIIAEKVINPTADNNWTFTDSIKGLDLRFLTADRAKSLVTDQECIVYLQASTASEGFGEFKFVYTESKVVTPDEQQSDQGNTGQDIVLESGQRITLPAVNPPAPAPQHKLASPHSQASTKTXX

SEQ ID NO: 114 - protein, 627 aa, synthetic construct
  VARIANT 1: note = X is any amino acid or absent
  VARIANT 2: note = X is any amino acid or absent
  VARIANT 3: note = X is any amino acid or absent
  VARIANT 4: note = X is any amino acid or absent
  VARIANT 5: note = X is any amino acid or absent
XXXXXAETSQSTTYHLTDDEKVAVREYIQAKMTIDMQEYRLAFLEGMMEEMASGSAEAAWDEEIADLKANLTAEQVVVLDELEANLIGSIAQHYHYLFETLTVAGKSGREEAAAIVSKYESEDDASTPEAELAALKYAREVIVELLNKESAAIDNYIAYAEATGQELAGLLESGNSNLESITSATIGYGQALATASQPKFPYDFSEMDRQIAELTASLQSKVEDKSTAKTENTGGSKVINIIGLENNADLNYGKSSFDNPEKSPVPFEIGKEYEYVYAGIGQASDFDGLNLTGKLALIKRGTISFSEKIANATAAGAVGVVIFNSRPGEANVSMQLDDTAIAIPSIFIPLEFGEALAANSYKIAFNNETDIRPQEVKNTIISAKQPDGGQATSKAVNVKIPAVVRLFGRELLENEFKFELREANGEELPVLDTAQNTKEGQVRFKNLSFDKPGKYWYTISEVKDELGGIEYDSKYIVAKITVEDRNGQLQAMIEFIDNDNVFNNFYTPAPAAASLSIKKVLEGRTLNTGEFEFVLKNEKGDEIEKVSNQADGSVNFSALTFTKEGTYTYTVSEVDGGLGDIIYDKSDIKATVTVKDNNHGQLVSTVTYENSDQIFENILNPGKLIAP

SEQ ID NO: 115 - protein, 626 aa, synthetic construct
  VARIANT 1: note = X is any amino acid or absent
  VARIANT 2: note = X is any amino acid or absent
  VARIANT 3: note = X is any amino acid or absent
  VARIANT 4: note = X is any amino acid or absent
XXXXAETSQSTTYHLTDDEKVAVREYIQAKMTIDMQEYRLAFLEGMMEEMASGSAEAAWDEEIADLKANLTAEQVVVLDELEANLIGSIAQHYHYLFETLTVAGKSGREEAAAIVSKYESEDDASTPEAELAALKYAREVIVELLNKESAAIDNYIAYAEATGQELAGLLESGNSNLESITSATIGYGQALATASQPKFPYDFSEMDRQIAELTASLQSKVEDKSTAKTENTGGSKVINIIGLENNADLNYGKSSFDNPEKSPVPFEIGKEYEYVYAGIGQASDFDGLNLTGKLALIKRGTISFSEKIANATAAGAVGVVIFNSRPGEANVSMQLDDTAIAIPSIFIPLEFGEALAANSYKIAFNNETDIRPQEVKNTIISAKQPDGGQATSKAVNVKIPAVVRLFGRELLENEFKFELREANGEELPVLDTAQNTKEGQVRFKNLSFDKPGKYWYTISEVKDELGGIEYDSKYIVAKITVEDRNGQLQAMIEFIDNDNVFNNFYTPAPAAASLSIKKVLEGRTLNTGEFEFVLKNEKGDEIEKVSNQADGSVNFSALTFTKEGTYTYTVSEVDGGLGDIIYDKSDIKATVTVKDNNHGQLVSTVTYENSDQIFENILNPGKLIAP

(19) United States
(12) Patent Application Publication
(10) Pub. No.: US 2025/0255949 A1
(43) Pub. Date: Aug. 14, 2025

(54) STREPTOCOCCUS SUIS VACCINE COMPOSITION COMPRISING IMMUNOGENIC FUSION POLYPEPTIDES

(71) Applicant: Intervacc AB, Hägersten (SE)

(72) Inventors: Sara Frosth, Hägersten (SE); Karin Jacobson, Hägersten (SE); Joakim Bjerketorp, Hägersten (SE); Lars FRYKBERG, Hägersten (SE); Bengt GUSS, Hägersten (SE); Jan-Ingmar FLOCK, Hägersten (SE); Andrew Stephen Waller, Hägersten (SE)

(21) Appl. No.: 18/857,553

(22) PCT Filed: Apr. 21, 2023
(86) PCT No.: PCT/EP2023/060533

§ 371 Date: Oct. 17, 2024

(30) Foreign Application Priority Data
Apr. 22, 2022 (EP) 22169492.0
May 24, 2022 (EP) 22175260.3

(51) Int. Cl. A61K 39/09; A61P 37/04; C07K 14/315

(52) U.S. Cl. CPC A61K 39/092; A61P 37/04; C07K 14/315; C07K 2319/40

(57) ABSTRACT

The present disclosure relates to immunogenic fusion polypeptides, immunogenic compositions and vaccine compositions comprising said fusion polypeptides and use thereof for immunization of mammals susceptible to Streptococcus suis infection. The disclosure also relates to methods for preparing, formulating and administrating such compositions.

### CROSS-REFERENCE TO RELATED APPLICATION

The present application is a § 371 national phase of International Application No. PCT/EP2023/060533, filed on Apr. 21, 2023, which claims the benefits of European Application No. 22169492.0, filed on Apr. 22, 2022, and European Application No. 22175260.3, filed on May 24, 2022, which applications are incorporated by reference herein.

### FIELD OF THE INVENTION

The present disclosure relates to immunogenic polypeptides, immunogenic compositions and vaccine compositions and use thereof for immunization of mammals susceptible to Streptococcus suis infection. The disclosure also relates to methods for preparing, formulating and administrating such compositions.

### BACKGROUND

Streptococcus suis is a major pathogen causing bacterial disease in pigs and is responsible for large economic losses in the swine industry. S. suis causes a variety of diseases in pigs, including meningitis, arthritis, pericarditis, polyserositis, septicaemia, pneumonia and sudden death. Additionally, it is also emerging as a zoonotic agent of meningitis and streptococcal toxic shock-like syndrome and there is a high prevalence of S. suis human disease in southeast and east Asia. However, human cases have also been reported in several European countries, Australia as well as North and South America. In western countries, S. suis infections are most often restricted to people who work in the swine industry, while in southeast and east Asia S. suis infections are a significant public health concern (Fittipaldi et al (2012), Future Microbiol. 7 (2); 259-279).

S. suis is a Gram-positive facultative anaerobic coccus, originally defined as Lancefield groups R, S, R/S or T. Later, a new typing system based on the type-specific capsular polysaccharide antigens located in the cell wall was proposed. This led to a system comprising 35 serotypes (Rasmussen and Andresen (1998), Int. J. Syst. Bacteriol. 48, 1063-1065) of which serotypes 2, 1, 9, 7 and ½ are the most prevalent. Especially, serotype 2 has been reported as a zoonotic agent.

Control of S. suis in pig herds is of a great interest to the pig industry. Pigs may acquire S. suis via both vertical and horizontal transmission and colonized animals typically harbor the bacteria in their tonsils. While the adult pigs usually serve as asymptomatic carries, some piglets will develop bacteremia, septicemia and/or meningitis due to dissemination of the bacteria from the tonsils and/or other mucosal surfaces. This usually occurs when maternal antibodies decline in the piglets at around 4 weeks of age. To cause disease, the bacterial must breach the epithelial barriers, reach and survive in the bloodstream, invade different organs and cause exaggerated inflammation. The actual early mechanisms used by S. suis to colonize the host are poorly understood, however several virulence factors have been proposed. The various S. suis virulence factors include; capsule, fibronectin/fibrinogen binding protein, serum opacity-like factor and modifications of the cell wall lipoteichoic acids and peptidoglycan (Fittipaldi supra). Furthermore, the virulence factors shared among various strains of the same serotype show a wide variation (Berthelot-Herault et al (2005) Can J Vet Res. July; 69(3):236-40; Quessy et al (1995) Infect Immun. May; 63(5):1975-9; Vecht et al (1992) Infect Immun. February; 60(2):550-6).

The development of vaccines has been focused on the above mentioned virulence factors. For example, vaccines compositions comprising a surface expressed protein, such as Sao and hp0245, are described in TW201412982, CN104248754 and CN102443053 and Li et al (2011) FEMS Micro Letters 316: 115-122. As of today, there is no well performing vaccine available against S. suis infection. In a recent study, the pan-surfome of S. suis was described identifying 113 surface expressed proteins (Gómez-Gascóna et al (2012), Journal of Proteomics October 22; 75(18):5654-66).

One commonly followed route to make a vaccine against a bacterial disease, is the production and testing of a whole cell vaccine preparation. Known in the art is Porcilis® Strepsuis, an attenuated bacterial vaccine for immunization of healthy pigs against disease caused by S. suis serotype 2. Additionally, vaccines comprising Streptococcus mutants deficient in capsular expression and the htpsA-gene have been described in WO0005378 and CN103352015, respectively. It seems likely however (Kebede et al (1990), Vet. Microbiol. 22: 249-257), that protection obtained with whole cell preparations is serotype-specific. Additionally, there are other disadvantages of whole cell vaccines. For example, unwanted reactions at and around the site of injection are common and it is often required to administer a large amount of non-specific protein, compared to the amount of material actually responsible for the induction of protection. The limited availability of efficient vaccines is likely caused by the large number of existing serotypes, the variation in virulence among strains and the still scarce knowledge about the factors that contribute to virulence and protection. A protein encoded by IgM-specific protease denoted Ide_{Ssuis} has been shown to elicit protective activity when immunized in pigs (Seele et al (2015) Vaccine, 33, 2207-2212). However, a vaccine based on one antigen alone may lose efficacy if mutations occur in the native protein of the infectious S. suis bacterial strain. WO2017005913 discloses vaccine compositions comprising said IgM specific protease as well as fragments of surface-anchored nucleotidases.

Lack of an effective vaccine against S. suis infections is a major problem in modern swine production. Thus, the provision of an effective and safe vaccine against S. suis infection is of great interest to the field.

### SUMMARY OF THE INVENTION

It is an object of the present disclosure to provide an effective S. suis vaccine, which vaccine overcomes the disadvantages of the prior art, for example in terms of elicited protection and cost efficiency.

It is an object of the present disclosure to provide an immunogenic polypeptide for the use as a medicament, such as in prophylactic treatment of S. suis infections.

Also provided are immunogenic polypeptide fragments and fusion polypeptides per se.

Another object of the present disclosure is to provide related immunogenic compositions and vaccine composition as well as related treatment methods.

Yet another object of the present invention is to provide an immunogenic mixture with adjuvant properties. Yet another object is to provide immunogenic compositions and vaccine compositions useful for treating piglets. In particular, immunogenic compositions and vaccine compositions for the protection of piglets against S. suis infection, wherein the composition is administered to the pregnant sow or gilt.

These and other objects which are evident to the skilled person from the present disclosure are met by different aspects of the invention as claimed in the appended claims and as generally disclosed herein.

Briefly, the pan-surfome of S. suis has been described (Gómez-Gascóna supra). Gómez-Gascóna and co-workers obtained 39 S. suis strains obtained from infected pigs, corresponding to 19 of the most prevalent serotypes. They identified a set of 113 proteins, corresponding to both common and unique surface proteins in these strains, and listed them as potential antigens for vaccine development. The present inventors identified a subset of these as particularly useful in the context of a vaccine against S. suis infection. Thus, the present disclosure is based on the finding made by the present inventors that recombinant variants in the form of fusion polypeptides comprising fragments of several different virulence factors are particularly useful in the context of prophylactic treatment of S. suis infection. In particular, the present inventors have identified recombinant variants in the form of fusion polypeptides comprising fragments which are unstructured (also referred to as disordered) and particularly useful in said context. Without being bound by theory, it is envisioned that an immunogenic composition and/or a vaccine comprising fragments of several antigens is likely to be efficacious, even if the bacterial strain acquires mutation in a subset of the native proteins which the immunogenic composition and/or vaccine is based on. Without being bound by theory, it is envisioned that in order to ensure protective effect of a vaccine composition, for example via inhibition of bacterial growth, it may be beneficial to induce antibodies against several native proteins, for example native proteins which may be involved in redundant systems important for bacterial function. As a non-limiting example, several zinc binding proteins are present at the surface of S. suis, thus it may be beneficial to include several such proteins in an immunogenic or vaccine composition. Additionally, it may be beneficial to include in an immunogenic or vaccine composition several different surface proteins in order to generate an antibody response. When an antibody binds to the surface of a bacterium, it labels the bacteria to be attacked by immune cells and complement system. It is envisioned certain surface proteins may be present in low amount or only under specific conditions and that the availability of some surface proteins may be restricted. Also, due to evolution many surface proteins exhibit mutations between different bacterial strains which may limit the effect of a specific antibody only to certain strains. The present inventors envision that including many proteins in an immunogenic or vaccine composition may overcome these potential obstacles.

Briefly, here follows a short description of the virulence factors whereof fragments have been utilized in the context of the present invention. Proteases have in many cases been shown to be important virulence factors but very few of them have been tested as vaccine components. There are several reasons for this; proteases are difficult or almost impossible to express in active form, the size of the proteases can cause problems and they are also probably unstable unless they are completely inactive. The IgM specific proteases denoted IdeSsuis (Seele et al (2013) Journal of Bacteriology, 195(5):930-940) is herein denoted M2 and corresponds to SEQ ID NO: 9. Additional proteases used in the context of the present invention are P1, P8, P11 and IgdE, corresponding to SEQ ID NO: 6, 7, 8 and 10, respectively.

The proteins denoted SP2, SP4 and SP7, and corresponding to SEQ ID NO:11, 13 and 12 respectively, show homology to the nucleotidase family of proteins. The importance of nucleotidases for virulence of bacteria has been shown for S. suis (Liu et al (2014) The Journal of Infectious Diseases, Vol 210, Issue 1, p. 35-45) and other bacteria.

Alterations in the concentrations of certain metal ions have a great impact in cell physiology and gene expression. Recent studies revealed that zinc has an effect on bacterial physiology during infection. Zinc-binding polypeptides utilized in the present invention are HtpS A (SEQ ID NO:1), HtpS B (SEQ ID NO:2), HtpS C (SEQ ID NO:3), HtpS C2 (SEQ ID NO:4) and AdcA (SEQ ID NO:5).

Surface proteins of Gram-positive bacteria often play a role in adherence of the bacteria to host tissue and are frequently required for virulence. A specific subgroup of extracellular proteins contains the cell wall-sorting motif LPXTG, which is the target for cleavage and covalent coupling to the peptidoglycan by enzymes called sortases. Proteins comprising an LPXTG-motif and utilized in the present invention are P6 (SEQ ID NO:17), C5a (SEQ ID NO:18) and Sao (SEQ ID NO:19).

Other virulence factors of interest are Amid1Sa (SEQ ID NO: 14), 15BSa (SEQ ID NO:15) and Hom17Sa (SEQ ID NO:16).

In a first aspect of the present disclosure, there is provided an immunogenic composition comprising at least one first and one second fusion polypeptide, which first fusion polypeptide comprises fragments of at least three native full length polypeptides of Streptococcus suis. By providing an immunogenic composition comprising fusion polypeptides each made up of several different fragments derived from different native full length polypeptides from S. suis, the present inventors have achieved successful vaccination of sows which exhibited significant antibody responses. Importantly, the inventors also show that the antibodies were transferred to piglets via the colostrum and lead to the reduction of the symptoms of S. suis challenge in the piglets following challenge at 4 weeks and 7 weeks of age as discussed in the Example section of the present application. As used herein, the term “piglets” refers to pigs of 0-12 weeks of age, such as 0-10 weeks of age. Hence, the term “piglets” used herein encompasses what is commonly referred to as piglets of 0-4 weeks of age, weaners of 4-10 weeks of age and early growers 10-12 weeks of age.

In one embodiment, an immunogenic composition is provided, wherein said second fusion polypeptide comprises fragments from at least three native full length polypeptides from S. suis.

As used herein, the term “fusion polypeptide” refers to a polypeptide comprising a least two units, such as at least three, which are derived from different native polypeptides or proteins. As used herein, the wording “fusion polypeptide comprises fragments of at least three native full length polypeptides of Streptococcus suis” is to be understood as each of the above mentioned units is a fragment of (in other words part of) a full length native polypeptide or protein. Thus, the units referred to above of the fusion polypeptide(s) as disclosed herein are not full length native polypeptides, but instead parts thereof. For the avoidance of any doubt, the term fragment thus refers to a part of the full length native polypeptide. Hence, the fusion polypeptides as disclosed herein are not fusions of several native full length polypeptides.

As an illustrative and non-limiting example, said fusion polypeptides may comprise a fragment of (in other words a part of) a full length native polypeptide A; a fragment of (in other words a part of) a full length native polypeptide B; and a fragment of (in other words a part of) a full length native polypeptide C, wherein A, B and C are different full length native polypeptides of S. suis.

Said immunogenic composition may comprise additional fusion polypeptides. Thus, in one embodiment, said immunogenic composition further comprises a third fusion polypeptide. In another embodiment, said immunogenic composition may further comprise a fourth fusion polypeptide. In another embodiment, said immunogenic composition may further comprise a fifth fusion polypeptide.

For the sake of clarity, it is to be understood that the designations first, second, third, fourth and fifth in relation to the fusion polypeptides is merely for distinguishing said fusion polypeptides from each other and is not limiting in terms of their presence in the composition or order of addition to the composition. Thus, a composition encompassed by the present disclosure, may comprise three fusion polypeptides, for example denoted a first, a second and a fourth fusion polypeptide or may comprise four fusion polypeptides, for example denoted a first, a second, a fourth and a fifth fusion polypeptide.

In one embodiment, said third fusion polypeptide and/or fourth fusion polypeptide and/or fifth comprise/comprises fragments of at least three native full length polypeptides from S. suis.

As discussed above, the present inventors have found that fusion polypeptides comprising fragments of virulence factors are particularly useful in the context of the present disclosure, for example fragments of zinc-binding proteins from S. suis; of proteases from S. suis, of nucleotidases from S. suis or of proteins from S. suis which comprise a LPXTG-motif. Also fragments of other virulence factors, such as Amid1 Sa, 15BSa and Hom17Sa are suitable. Thus, in one embodiment there is provided an immunogenic composition, wherein said at least three native full length polypeptides from S. suis are independently selected or selected from the group consisting of zinc-binding proteins from S. suis; proteases from S. suis; nucleotidases from S. suis; proteins from S. suis which comprise an LPXTG-motif; Amid1 Sa (SEQ ID NO:14) and any polypeptides with at least 80% identity to SEQ ID NO:14; 15BSa (SEQ ID NO:15) and polypeptides with at least 80% identity to SEQ ID NO:15; and Hom17Sa (SEQ ID NO:16) and any polypeptides with at least 80% identity to SEQ ID NO:16.

As used herein, the wording “independently selected” in reference to a group of specified items refers to that to the fact that when than more than one item is selected form the group of items, the decision of selecting a specific item is not influenced by decision of selecting any of the previous or following item(s).

As the skilled person will realize, the properties of a polypeptide, such as the immunogenicity of the polypeptides of the present disclosure, may be dependent on the sequence structure of the polypeptide and the presence and accessibility of immunogenic regions within said polypeptide. It is therefore possible to make minor changes to the sequence of amino acids in a polypeptide without affecting the function thereof. Thus, the disclosure encompasses modified variants of the immunogenic polypeptide as described herein, which are such that the immunogenic characteristics are retained. Thus, in some embodiments, the polypeptides may comprise a sequence which is at least 81%, such as at least 82% such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 93%, such as at least 95%, such as at least 96%, such as at least 97%, such as at least 98% or such as at least 99% identical to a sequence selected from any one of said SEQ ID NO:14, SEQ ID NO:15 and SEQ ID NO:16.

For example, it is possible that one or several amino acid residues belonging to a certain functional grouping of amino acid residues (e.g. hydrophobic, hydrophilic, polar etc.) could be exchanged for another amino acid residue from the same functional group. It is also possible, that one or several amino acid residues are exchanged for one or several amino acid residues that belong to a different functional group, provided that the resulting polypeptide retains its immunogenic properties.

The term “% identity”, as used throughout the specification, may for example be calculated as follows. The query sequence is aligned to the target sequence using the CLUSTAL W algorithm (Thompson et al, Nucleic Acids Research, 22: 4673-4680 (1994)). A comparison is made over the window corresponding to the shortest of the aligned sequences. The shortest of the aligned sequences may in some instances be the target sequence. In other instances, the query sequence may constitute the shortest of the aligned sequences. The amino acid residues at each position are compared, and the percentage of positions in the query sequence that have identical correspondences in the target sequence is reported as % identity. To clarify, the text above relating to %-identity is equally relevant to the second aspect of the present disclosure and will for the sake of brevity not be repeated.

In one embodiment, said at least three native full length polypeptides from S. suis are independently selected or selected from the group consisting of zinc-binding proteins from S. suis, proteases from S. suis; nucleotidases from S. suis and proteins from S. suis which comprise an LPXTG-motif (SEQ ID NO:102).

Thus, the immunogenic composition according to the present disclosure, may comprise a first fusion polypeptide, a second fusion polypeptide, a third fusion polypeptide, a fourth fusion polypeptide and/or a fifth fusion polypeptide which comprises at least three fragments of full length polypeptides. The fragments may be selected from fragments of zinc-binding proteins from S. suis; from fragments of proteases from S. suis; from fragments of nucleotidases from S. suis; from fragments of proteins from S. suis which comprise an LPXTG-motif; and/or from fragments of Amid1Sa (SEQ ID NO:14), 15BSa (SEQ ID NO:15) and Hom17Sa (SEQ ID NO:16). In one embodiment, said first fusion polypeptide comprises at least three fragments selected from fragments of zinc-binding proteins from S. suis; from fragments of proteases from S. suis; from fragments of nucleotidases from S. suis or from fragments of proteins from S. suis which comprise an LPXTG-motif. In one embodiment, said second fusion polypeptide comprises at least three fragments selected from fragments of zinc-binding proteins from S. suis; from fragments of proteases from S. suis; from fragments of nucleotidases from S. suis or from fragments of proteins from S. suis which comprise an LPXTG-motif. In one embodiment, said third fusion polypeptide comprises at least three fragments selected from fragments of zinc-binding proteins from S. suis; from fragments of proteases from S. suis; from fragments of nucleotidases from S. suis or from fragments of proteins from S. suis which comprise an LPXTG-motif. In one embodiment, said fourth fusion polypeptide comprises at least three fragments selected from fragments of zinc-binding proteins from S. suis; from fragments of proteases from S. suis; from fragments of nucleotidases from S. suis or from fragments of proteins from S. suis which comprise an LPXTG-motif. In one embodiment, said fifth fusion polypeptide comprises at least three fragments selected from fragments of zinc-binding proteins from S. suis; from fragments of proteases from S. suis; from fragments of nucleotidases from S. suis or from fragments of proteins from S. suis which comprise an LPXTG-motif. In particular, at least two of said first, second, third, fourth and fifth fusion polypeptides may comprise fragments of zinc-binding proteins from S. suis.

In particular embodiments, said zinc-binding proteins, proteases, nucleotidases, proteins which comprise an LPXTG-motif and other virulence factors may be selected from the group consisting of Htps A, Htps B, Htps C, Htps C2, AdcA, P1, P8, P11, M2, IdgE, SP2, SP7, SP4, P6, C5a, Sao, Amid1Sa, 15BSa and Hom17Sa, corresponding to SEQ ID NO:1-19, respectively. More specifically, in one embodiment, there is provided an immunogenic composition as disclosed herein, wherein said at least three native full length polypeptides from S. suis are independently selected or selected from the group consisting of polypeptides having an amino acid sequence according to any one of SEQ ID NO:1-19 and any polypeptides with at least 80% identity to any one of SEQ ID NO:1-19; such as the group consisting of polypeptides having an amino acid sequence according to any one of SEQ ID NO:1-19. In some embodiments, the polypeptides may comprise sequences which are at least 81%, such as at least 82% such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 93%, such as at least 95%, such as at least 96%, such as at least 97%, such as at least 98% or such as at least 99% identical to any one of SEQ ID NO:1-19.

In particular, said at least three native full length polypeptides from S. suis may be independently selected or selected from the group consisting of polypeptides having the amino acid sequence according to any one of SEQ ID NO:1-12 and 17-19 and polypeptides with at least 80% identity to any one of SEQ ID NO:1-12 and 17-19; such as the group consisting of polypeptides having an amino acid sequence according to any one of SEQ ID NO:1-12 and 17-19. In some embodiments, the polypeptides may comprise sequences which are at least 81%, such as at least 82% such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 93%, such as at least 95%, such as at least 96%, such as at least 97%, such as at least 98% or such as at least 99% identical to any one of SEQ ID NO:1-12 and 17-19.

In particular, said at least three native full length polypeptides from S. suis may be independently selected or selected from the group consisting of polypeptides having the amino acid sequence according to any one of SEQ ID NO:1-10 and 14-19 and polypeptides with at least 80% identity to any one of SEQ ID NO:1-10 and 14-19; such as the group consisting of polypeptides having an amino acid sequence according to any one of SEQ ID NO:1-10 and 14-19. In some embodiments, the polypeptides may comprise sequences which are at least 81%, such as at least 82% such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 93%, such as at least 95%, such as at least 96%, such as at least 97%, such as at least 98% or such as at least 99% identical to any one of SEQ ID NO:1-10 and 14-19.

In particular, said at least three native full length polypeptides from S. suis may be independently selected or selected from the group consisting of polypeptides having the amino acid sequence according to any one of SEQ ID NO:1-13 and 17-19 and polypeptides with at least 80% identity to any one of SEQ ID NO:1-13 and 17-19; such as the group consisting of polypeptides having an amino acid sequence according to any one of SEQ ID NO:1-13 and 17-19. In some embodiments, the polypeptides may comprise sequences which are at least 81%, such as at least 82% such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 93%, such as at least 95%, such as at least 96%, such as at least 97%, such as at least 98% or such as at least 99% identical to any one of SEQ ID NO:1-13 and 17-19.

It will be understood that one of the fusion polypeptides comprised in the immunogenic composition as disclosed herein may comprise of fragments of at least three zinc-binding proteins, fragments of at least three proteases, or fragments of at least three nucleotidases as well as fragments of polypeptides that exhibit at least 80% identity thereto. It will be appreciated that said one of the fusion polypeptides may be designated the first, second, third, fourth or fifth polypeptide as explained above. Thus, in one embodiment there is provided an immunogenic composition as disclosed herein, wherein said first fusion polypeptide comprises at least three fragments selected from fragments of the zinc-binding proteins having an amino acid sequence according to any one of SEQ ID NO:1-5 and polypeptides with at least 80% identity to any one of SEQ ID NO:1-5; from fragments of the proteases having an amino acid sequence according to any one of SEQ ID NO:6-10 and polypeptides with at least 80% identity to any one of SEQ ID NO:6-10; from fragments of the nucleotidases having an amino acid sequence according to any one of SEQ ID NO:11-13 and polypeptides with at least 80% identity to any one of SEQ ID NO:11-13; or from fragments of proteins comprising LPXTG-motif according to any one of SEQ ID NO: 17-19 and polypeptides with at least 80% identity to any one of SEQ ID NO:17-19. In one embodiment there is provided an immunogenic composition as disclosed herein, wherein said second fusion polypeptide comprises at least three fragments selected from fragments of the zinc-binding proteins having an amino acid sequence according to any one of SEQ ID NO:1-5 and polypeptides with at least 80% identity to any one of SEQ ID NO:1-5; from fragments of the proteases having an amino acid sequence according to any one of SEQ ID NO:6-10 and polypeptides with at least 80% identity to any one of SEQ ID NO:6-10; from fragments of the nucleotidases having an amino acid sequence according to any one of SEQ ID NO:11-13 and polypeptides with at least 80% identity to any one of SEQ ID NO:11-13 or from fragments of proteins comprising LPXTG-motif according to any one of SEQ ID NO: 17-19 and polypeptides with at least 80% identity to any one of SEQ ID NO:17-19.

In one embodiment there is provided an immunogenic composition as disclosed herein, wherein said third fusion polypeptide comprises at least three fragments selected from fragments of the zinc-binding proteins having an amino acid sequence according to any one of SEQ ID NO:1-5 and polypeptides with at least 80% identity to any one of SEQ ID NO:1-5; from fragments of the proteases having an amino acid sequence according to any one of SEQ ID NO:6-10 and polypeptides with at least 80% identity to any one of SEQ ID NO:6-10; from fragments of the nucleotidases having an amino acid sequence according to any one of SEQ ID NO:11-13 and polypeptides with at least 80% identity to any one of SEQ ID NO:11-13 or from fragments of proteins comprising LPXTG-motif according to any one of SEQ ID NO: 17-19 and polypeptides with at least 80% identity to any one of SEQ ID NO:17-19.

In one embodiment there is provided an immunogenic composition as disclosed herein, wherein said fourth fusion polypeptide comprises at least three fragments selected from fragments of the zinc-binding proteins having an amino acid sequence according to any one of SEQ ID NO:1-5 and polypeptides with at least 80% identity to any one of SEQ ID NO:1-5; from fragments of the proteases having an amino acid sequence according to any one of SEQ ID NO:6-10 and polypeptides with at least 80% identity to any one of SEQ ID NO:6-10; from fragments of the nucleotidases having an amino acid sequence according to any one of SEQ ID NO:11-13 and polypeptides with at least 80% identity to any one of SEQ ID NO:11-13; or from fragments of proteins comprising LPXTG-motif according to any one of SEQ ID NO:17-19 and polypeptides with at least 80% identity to any one of SEQ ID NO:17-19.

In one embodiment there is provided an immunogenic composition as disclosed herein, wherein said fifth fusion polypeptide comprises at least three fragments selected from fragments of the zinc-binding proteins having an amino acid sequence according to any one of SEQ ID NO:1-5 and polypeptides with at least 80% identity to any one of SEQ ID NO:1-5; from fragments of the proteases having an amino acid sequence according to any one of SEQ ID NO:6-10 and polypeptides with at least 80% identity to any one of SEQ ID NO:6-10; from fragments of the nucleotidases having an amino acid sequence according to any one of SEQ ID NO:11-13 and polypeptides with at least 80% identity to any one of SEQ ID NO:11-13 or from fragments of proteins comprising LPXTG-motif according to any one of SEQ ID NO:17-19 and polypeptides with at least 80% identity to any one of SEQ ID NO:17-19.

Also encompassed are polypeptides which exhibit at least 80% identity with said zinc-binding proteins, proteases, nucleotidases or proteins comprising an LPXTG motif. Hence, fragments may be derived from such polypeptides. In one embodiment, the zinc-binding proteins, proteases, nucleotidases and proteins comprising LPXTG-motif may comprise sequences which are at least 81%, such as at least 82% such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 93%, such as at least 95%, such as at least 96%, such as at least 97%, such as at least 98% or such as at least 99% identical to said zinc-binding proteins, proteases, nucleotidases and proteins comprising an LPXTG-motif, respectively. The skilled person will appreciate that said % identity feature may apply to any one of the first, second, third, fourth or fifth polypeptide discussed above. It will be appreciated that recitation of % identity levels above is equally relevant for any embodiment of this first aspect and second aspects, wherein an % identity feature is recited. For the sake of brevity it will not be repeated each time.

To illustrate by a non-limiting example, said immunogenic composition may comprise a first fusion polypeptide comprising fragments of SEQ ID NO:1, SEQ ID NO: 2 and SEQ ID NO:3; a second fusion polypeptide comprising fragments of SEQ ID NO: 6, SEQ ID NO:7, SEQ ID NO:8, SEQ ID NO:9 and SEQ ID NO:10; a third fusion polypeptide comprising fragments of SEQ ID NO:1, SEQ ID NO:2, SEQ ID NO:3, SEQ ID NO:4 and SEQ ID NO:5; a fourth fusion polypeptide comprising fragments of SEQ ID NO:11, SEQ ID NO:12 and SEQ ID NO:13; and a fifth fusion polypeptide comprising fragments of SEQ ID NO:17, SEQ ID NO:18 and SEQ ID NO:19. One or more of said first, second, third, fourth or fifth fusion polypeptide may comprise additional fragments from polypeptides, such as fragments from one or more of SEQ ID NO:14, 15 and 16. For example, said third fusion polypeptide may comprise fragments of one, two or three of SEQ ID NO: 14, 15 and 16. For example, said third fusion polypeptide may comprise fragments of SEQ ID NO:14 and 15; or fragments of SEQ ID NO:14 and 16; or fragments of SEQ ID NO:15 and 16. Thus, it will be appreciated that any one of said first, second, third, fourth and fifth polypeptides may comprise additional fragments from other virulence factors, for example fragments of any one of SEQ ID NO:14, SEQ ID NO:15 and SEQ ID NO:16 or polypeptides with at least 80% identity to any one of SEQ ID NO:14-16.

In one embodiment said first and second, and optional third, fourth and/or fifth, fusion polypeptides comprise different fragments. In one embodiment, said first and second, and optional third, fourth and/or fifth, fusion polypeptides comprise fragments from different native polypeptides of S. suis.

In one particular embodiment, said first fusion polypeptide comprises at least three fragments selected from fragments of the zinc-binding proteins having an amino acid sequence according to any one of SEQ ID NO:1-5; from fragments of the proteases having an amino acid sequence according to any one of SEQ ID NO:6-10; from fragments of the nucleotidases having an amino acid sequence according to any one of SEQ ID NO:11-13; and from proteins from S. suis which comprise an LPXTG-motif having an amino acid sequence according to any one of SEQ ID NO:17-19. In one particular embodiment, said second fusion polypeptide comprises at least three fragments selected from fragments of the zinc-binding proteins having an amino acid sequence according to any one of SEQ ID NO:1-5; from fragments of the proteases having an amino acid sequence according to any one of SEQ ID NO:6-10; from fragments of the nucleotidases having an amino acid sequence according to any one of SEQ ID NO:11-13; and from proteins from S. suis which comprise an LPXTG-motif having an amino acid sequence according to any one of SEQ ID NO:17-19. In one embodiment, said third fusion polypeptide comprises at least three fragments selected from fragments of the zinc-binding proteins having an amino acid sequence according to any one of SEQ ID NO:1-5; from fragments of the proteases having an amino acid sequence according to any one of SEQ ID NO:6-10; from fragments of the nucleotidases having an amino acid sequence according to any one of SEQ ID NO:11-13 and from proteins from S. suis which comprise an LPXTG-motif having an amino acid sequence according to any one of SEQ ID NO:17-19. In one embodiment, said fourth fusion polypeptide comprises at least three fragments selected from fragments of the zinc-binding proteins having an amino acid sequence according to any one of SEQ ID NO:1-5; from fragments of the proteases having an amino acid sequence according to any one of SEQ ID NO:6-10; from fragments of the nucleotidases having an amino acid sequence according to any one of SEQ ID NO:11-13; and from proteins from S. suis which comprise an LPXTG-motif having an amino acid sequence according to any one of SEQ ID NO:17-19. In one embodiment, said fifth fusion polypeptide comprises at least three fragments selected from fragments of the zinc-binding proteins having an amino acid sequence according to any one of SEQ ID NO:1-5; from fragments of the proteases having an amino acid sequence according to any one of SEQ ID NO:6-10; from fragments of the nucleotidases having an amino acid sequence according to any one of SEQ ID NO:11-13; and from proteins from S. suis which comprise an LPXTG-motif having an amino acid sequence according to any one of SEQ ID NO:17-19. In one embodiment, said first, second, third, fourth and fifth fusion polypeptide do not comprise the same fragments. In one embodiment, said first, second, and optional third, fourth and fifth, fusion polypeptides comprise at least three fragments selected from fragments of the zinc-binding proteins having an amino acid sequence according to any one of SEQ ID NO:1-5; or from fragments of the proteases having an amino acid sequence according to any one of SEQ ID NO:6-10; or from fragments of the nucleotidases having an amino acid sequence according to any one of SEQ ID NO:11-13; or from proteins which comprise an LPXTG-motif having an amino acid sequence according to any one of SEQ ID NO:17-19.

To express it in other words, the immunogenic composition disclosed herein may be such that each of said first, second, third, fourth and fifth fusion polypeptides comprises fragments independently selected or selected from fragments of the zinc-binding proteins having an amino acid sequence according to any one of SEQ ID NO:1-5 and polypeptides with at least 80% identity to any one of SEQ ID NO:1-5; or from fragments of the proteases having an amino acid sequence according to any one of SEQ ID NO:6-10 and polypeptides with at least 80% identity to any one of SEQ ID NO:6-10; or from fragments of the nucleotidases having an amino acid sequence according to any one of SEQ ID NO:11-13 and polypeptides with at least 80% identity to any one of SEQ ID NO:11-13; or from proteins which comprise an LPXTG-motif having an amino acid sequence according to any one of SEQ ID NO:17-19. In particular, said fragments may be independently selected or selected from fragments of the zinc-binding proteins having an amino acid sequence according to any one of SEQ ID NO:1-5; or from fragments of the proteases having an amino acid sequence according to any one of SEQ ID NO:6-10; or from fragments of the nucleotidases having an amino acid sequence according to any one of SEQ ID NO:11-13; or from proteins which comprise an LPXTG-motif having an amino acid sequence according to any one of SEQ ID NO:17-19. In one embodiment, said first, second, third, fourth and fifth fusion polypeptide do not comprise the same fragments.

As illustrated by the non-limiting example above, it is envisioned that the immunogenic composition as disclosed herein may comprise several fusion polypeptides which comprise fragments of the same native full length polypeptides from Streptococcus suis. For example, said fusion polypeptides may comprises different fragments from the same native full length polypeptides. Alternativelty said fusion polypeptides may comprise the same fragments in combination with fragments from different virulence factors. For example, the fusion polypeptide denoted 4Zn+3 (SEQ ID NO:44) comprises five fragments of zinc-binding polypeptides. In addition, said fusion polypeptide comprises three fragments of other virulence factors.

It is envisioned that in some embodiments the fusion polypeptide may comprise more than one fragment from the same native full length polypeptide, the fragments being the same or different. For example, two identical fragments may be present in the fusion polypeptide or two different fragments (non-overlapping or partially overlapping) may be present in the fusion polypeptide, which fragments are derived from the same native full length polypeptide. A non-limiting example of a fusion polypeptide comprising two fragments of the same native polypeptide is 5AsL (SEQ ID NO:45), which comprises two different fragments of the M2 protein (SEQ ID NO:9).

In one embodiment there is provided an immunogenic composition as disclosed herein, wherein at least one of said first, second, third, fourth and fifth fusion polypeptide comprises at least three or at least four or least five fragments from zinc-binding proteins from S. suis, such as at least three fragments from different zinc-binding proteins from S. suis. In one particular embodiment, said at least one of first, second, third, fourth and fifth fusion polypeptide comprises at least three or at least four or at least five fragments of the zinc-binding proteins having an amino acid sequence according to any one of SEQ ID NO:1-5 and polypeptides with at least 80% identity to any one of SEQ ID NO:1-5, such as at least three or at least four or at least five fragments of the zinc-binding proteins having an amino acid sequence according to any one of SEQ ID NO:1-5. In one embodiment, said at least one of first, second, third, fourth and fifth fusion polypeptide comprises at least three or at least four fragments of the zinc-binding proteins having an amino acid sequence according to any one of SEQ ID NO:1-4 and polypeptides with at least 80% identity to any one of SEQ ID NO:1-4, such as at least three fragments of the zinc-binding proteins having an amino acid sequence according to any one of SEQ ID NO:1-4. In one embodiment, said at least one of first, second, third, fourth and fifth fusion polypeptide comprises at least three fragments of the zinc-binding proteins having an amino acid sequence according to any one of SEQ ID NO:1-3 and polypeptides with at least 80% identity to any one of SEQ ID NO:1-3, such as at least three fragments of the zinc-binding proteins having an amino acid sequence according to any one of SEQ ID NO:1-3.

In one embodiment there is provided an immunogenic composition as disclosed herein, wherein at least one of said first, second, third, fourth and fifth fusion polypeptide comprises at least three fragments from proteases from S. suis, such as at least three fragments from different proteases from S. suis. In particular, said least one of said first, second, third, fourth and fifth fusion polypeptide may comprise at least three fragments of the proteases having an amino acid sequence according to any one of SEQ ID NO:6-10 and polypeptides with at least 80% identity to any one of said amino acid sequences, such as at least three fragments of the proteases having an amino acid sequence according to any one of SEQ ID NO:6-10. In one embodiment, said first, second, third, fourth and fifth fusion polypeptides may comprise at least four, such as at least five, such as at least six fragments of said proteases.

In one embodiment there is provided an immunogenic composition as disclosed herein, wherein at least one of said first, second, third, fourth and fifth fusion polypeptide comprises at least three fragments from nucleotidases proteins from S. suis, such as at least three fragments from different nucleotidases from S. suis. In particular, said at least one of said first, second, third, fourth and fifth fusion polypeptide may comprise at least three fragments of the nucleotidases having an amino acid sequence according to any one of SEQ ID NO:11-13 and polypeptides with at least 80% identity to any one of said amino acid sequences, such as at least three fragments of the nucleotidases having an amino acid sequence according to any one of SEQ ID NO:11-13.

In one embodiment there is provided an immunogenic composition as disclosed herein, wherein at least one of said first, second, third, fourth and fifth fusion polypeptide comprises at least three fragments from proteins comprising an LPXTG-motif from S. suis, such as at least three fragments from different proteins from proteins comprising an LPXTG-motif from S. suis. In particular, said at least one of said first, second, third, fourth and fifth fusion polypeptide may comprise at least three fragments from proteins comprising an LPXTG-motif from S. suis having an amino acid sequence according to any one of SEQ ID NO:17-19 and polypeptides with at least 80% identity to any one of said amino acid sequences, such as at least three fragments of the proteins comprising an LPXTG-motif from S. suis having an amino acid sequence according to any one of SEQ ID NO:17-19.

As explained above, it is envisioned that the fusion polypeptides comprised in the immunogenic composition as disclosed herein, may comprise further fragments of virulence factors, such as of any one of SEQ ID NO:1-19 or other suitable factors. In one particular embodiment, said at least one of said first, second, third, fourth and fifth fusion polypeptides further comprises at least one fragment, such as at least two fragments, such as at least three fragments, of at least one native full length polypeptide having an amino acid sequence according to any one of SEQ ID NO:1-19 or any amino acid sequences with at least 80% identity to any one of SEQ ID NO:1-19. In particular embodiments, said at least one of said first, second, third, fourth and fifth fusion polypeptides further comprises a fragment of at least one native full length polypeptide having an amino acid sequence according to any one of SEQ ID NO:14-16 or any amino acid sequences with at least 80% identity to any one of SEQ ID NO:14-16; such as a fragment of each of at least two native full length polypeptides having an amino acid sequence according to any one of SEQ ID NO:14-16 or any amino acid sequences with at least 80% identity to any one of SEQ ID NO:14-16; such as a fragment of each of all three native full length polypeptides having an amino acid sequence according to any one of SEQ ID NO:14-16 or any amino acid sequences with at least 80% identity to any one of SEQ ID NO:14-16.

In one embodiment, said at least one of said first, second, third, fourth and fifth fusion polypeptides further comprises a fragment of at least one native full length polypeptide having an amino acid sequence according to any one of SEQ ID NO:14-16, such as a fragment of each of at least two native full length polypeptides having an amino acid sequence according to any one of SEQ ID NO:14-16; such as a fragment of each of all three native full length polypeptides having an amino acid sequence according to any one of SEQ ID NO:14-16. For example, said fusion polypeptide may further comprise fragments of SEQ ID NO:14 and 15; or fragments of SEQ ID NO:14 and 16; or fragments of SEQ ID NO:15 and 16.

In some embodiment, there is provided an immunogenic composition as disclosed herein, wherein said at least one of said first, second, third, fourth and fifth fusion polypeptides comprises at least two fragments from the same native full length polypeptide from S. suis and wherein the fragments are different fragments from the same native full length polypeptide from S. suis. One example of such a fusion polypeptide is SEQ ID NO:45, which comprises two different fragments of SEQ ID NO:9, corresponding to the full length M2 protein. Thus, in one embodiment, said native full length polypeptide from S. suis is a protease from S. suis or a zinc-binding protein of S. suis. Said fragments may be the same fragment occurring more than one time in the fusion polypeptide or fragments which exhibit at last 90%, such as at least 95%, such as at least 97% or higher identity to each other. Alternatively, said fragments may be different fragments of the same native polypeptides, such as fragments which exhibit less than 10% identity to each other.

In one particular embodiment, said native full length polypeptide is SEQ ID NO:9 or any amino acid sequence having at least 80% identity to SEQ ID NO:9, such as wherein said native full length polypeptide is SEQ ID NO:9.

In one embodiment of the immunogenic composition provided herein, said at least two of said first, second, third, fourth and fifth fusion polypeptides comprise at least one fragment each from the same native full length polypeptide from S. suis, such as from the same two native full length polypeptides from S. suis, such as from the same three native full length polypeptides from S. suis; and wherein the fragments are different fragments from the same native full length polypeptide from S. suis. For example, the fusion polypeptides according to SEQ ID NO:43 and SEQ ID NO:44, both comprise fragments of the native full length polypeptides according to SEQ ID NO:1, SEQ ID NO:2 and SEQ ID NO:3. As explained above, SEQ ID NO:1, 2 and 3 are zinc binding proteins. Thus, in one embodiment, said native full length polypeptide from S. suis is a zinc-binding protein from S. suis. In one embodiment, said native full length polypeptides from S. suis are zinc-binding proteins from S. suis.

It will be appreciated that the immunogenic composition as disclosed herein may comprise more than two fusion polypeptides. Without being bound by theory, it may be advantageous that several fusion polypeptides are present in the composition as the total number of different antigenic and/or immunogenic fragments included in the composition is increased. Whereas it may be possible to include more fragments into fewer fusion polypeptides, it may be preferable to maintain a balance between the number of fusion polypeptides and their length. Very long polypeptides may for example be harder to produce, while on the other hand a composition comprising many small fragments may also be difficult to produce.

Thus, in one embodiment, there is provided an immunogenic composition as disclosed herein, which composition comprises three, four or five fusion polypeptides. Said composition may comprise a first fusion polypeptide, a second fusion polypeptide and a third fusion polypeptide as defined herein; or a first fusion polypeptide, a second fusion polypeptide and a fourth fusion polypeptide as defined herein; or a first fusion polypeptide, a second fusion polypeptide, a third fusion polypeptide and a fourth fusion polypeptide as defined herein. Said composition may comprise a first, second, third and fourth fusion polypeptide; or a first, second, third and fifth fusion polypeptide; or a first, second, fourth and fifth fusion polypeptide; or a first, third, fourth and fifth fusion polypeptide; or a second, third, fourth and fifth fusion polypeptide. The composition may comprise all five of said first, second, third, fourth and fifth fusion polypeptides.

As explained above, the designations first, second, third, fourth and fifth in relation to the fusion polypeptides is merely for distinguishing said fusion polypeptides from each other and is not limiting in terms of their presents in the composition or order of addition to the composition.

In one embodiment, said immunogenic composition comprises fusion polypeptides which are less than 1000 aa long, such as less than 800 aa long, such as less than 600 aa long. In particular, at least to two or at least three fusion polypeptides may be less than 800 aa long, such as less than 600 aa long.

In one embodiment of the immunogenic composition provided herein, said fragments of said zinc-binding polypeptides are selected from the group consisting of fragments of amino acid sequence SEQ ID NO:1, SEQ ID NO:2, SEQ ID NO:3, SEQ ID NO:4, SEQ ID NO:5 and any amino acid sequences having at least 80% identity to said fragments, such as the group consisting of fragments of amino acid sequence SEQ ID NO:1, SEQ ID NO:2, SEQ ID NO:3, SEQ ID NO:4 and any amino acid sequences having at least 80% identity to said fragments, such as the group consisting of fragments of amino acid sequence SEQ ID NO:1, SEQ ID NO:2, SEQ ID NO:3 and any amino acid sequences having at least 80% identity to said fragments.

In particular, said fragments of said zinc-binding protein may be selected from the group consisting of polypeptide fragments comprising or consisting of an amino acid sequence selected from SEQ ID NO:20-27 and any amino acid sequences with at least 80% identity to any one of SEQ ID NO:20-27, such as the group consisting of polypeptide fragments comprising or consisting of an amino acid sequence selected from SEQ ID NO:20-27. In particular, said fragments of said zinc-binding protein may be selected from the group consisting of polypeptide fragments comprising or consisting of the amino acid sequences SEQ ID NO:20, 21, 22, 23, 24, 25, 26 and 27 and any amino acid sequences with at least 80% identity to any one of SEQ ID NO:20, 21, 22, 23, 24, 25, 26 and 27, such as the group consisting of polypeptide fragments comprising or consisting of an amino acid sequences SEQ ID NO:20, 21, 22, 23, 24, 25, 26 and 27.

In one embodiment, wherein said fragments of said proteases are selected from the group consisting of fragments of amino acid sequence SEQ ID NO:6, SEQ ID NO:7, SEQ ID NO:8, SEQ ID NO:9, SEQ ID NO:10 and any amino acid sequences having at least 80% identity to said fragments. In particular, said fragments of said proteases may be selected from the group consisting of polypeptide fragments comprising or consisting of an amino acid sequence selected from SEQ ID NO:28-33 and any amino acid sequences with at least 80% identity to any one of SEQ ID NO:28-33, such as the group consisting of polypeptide fragments comprising or consisting of an amino acid sequence selected from SEQ ID NO:28-33.

In particular, said fragments of said proteases may be selected from the group consisting of polypeptide fragments comprising or consisting the amino acid sequences SEQ ID NO:28, 29, 30, 31, 32 and 33 and any amino acid sequences with at least 80% identity to any one of SEQ ID NO: 28, 29, 30, 31, 32 and 33, such as the group consisting of polypeptide fragments comprising or consisting of an amino acid sequences SEQ ID NO: 28, 29, 30, 31, 32 and 33.

In one embodiment, said fragments of said nucleotidases are selected from the group consisting of fragments of amino acid sequence SEQ ID NO:11, SEQ ID NO:12, SEQ ID NO:13 and any amino acid sequences having at least 80% identity to said fragments. In particular, said fragments of said nucleotidases may be selected from the group consisting of polypeptide fragments comprising or consisting of an amino acid sequence selected from SEQ ID NO:34-36 and any amino acid sequences with at least 80% identity to any one of SEQ ID NO:34-36, such as the group consisting of polypeptide fragments comprising or consisting of an amino acid sequence selected from SEQ ID NO:34-36. In particular, said fragments of said nucleotidases may be selected from the group consisting of polypeptide fragments comprising or consisting the amino acid sequences SEQ ID NO:34, 35 and 36 and any amino acid sequences with at least 80% identity to any one of SEQ ID NO: 34, 35 and 36, such as the group consisting of polypeptide fragments comprising or consisting of an amino acid sequences SEQ ID NO:34, 35 and 36.

In one embodiment, said fragments of said proteins comprising an LPXTG-motif are selected from the group consisting of fragments comprising or consisting of amino acid sequence SEQ ID NO:17, SEQ ID NO:18, SEQ ID NO:19 and any amino acid sequences having at least 80% identity to said fragments.

In particular, said fragments of said proteins comprising an LPXTG-motif may be selected from the group consisting of polypeptide fragments comprising or consisting of an amino acid sequence selected from SEQ ID NO:40-42 and any amino acid sequences with at least 80% identity to any one of SEQ ID NO:40-42, such as the group consisting of polypeptide fragments comprising or consisting of an amino acid sequence selected from SEQ ID NO:40-42.

In particular, said fragments of said proteins comprising an LPXTG-motif may be selected from the group consisting of polypeptide fragments comprising or consisting the amino acid sequences SEQ ID NO:40, 41 and 42 and any amino acid sequences with at least 80% identity to any one of SEQ ID NO: 40, 41 and 42, such as the group consisting of polypeptide fragments comprising or consisting of an amino acid sequences SEQ ID NO: 40, 41 and 42.

In one embodiment, said fragments are selected from the group consisting of fragments of amino acid sequence SEQ ID NO:14, SEQ ID NO:15, SEQ ID NO:16 and any amino acid sequences having at least 80% identity to said fragments. In particular, said fragments may be selected from the group consisting of SEQ ID NO:37-39 and any amino acid sequences with at least 80% identity to any one of SEQ ID NO:37-39, such as the group consisting of polypeptide fragments comprising an amino acid sequence selected from SEQ ID NO:37-39. In particular, said fragments may be selected from the group consisting of polypeptide fragments comprising or consisting the amino acid sequences SEQ ID NO:37, 38 and 39 and any amino acid sequences with at least 80% identity to any one of SEQ ID NO: 37, 38 and 39, such as the group consisting of polypeptide fragments comprising or consisting of an amino acid sequences SEQ ID NO: 37, 38 and 39.

In one embodiment there is provided an immunogenic composition as disclosed herein, wherein one of said first, second, third, fourth and fifth fusion polypeptides comprises at least one, such as at least two, such as at least three, of SEQ ID NO:20, 21 and 22 and any amino acid sequence with at least 80% identity to any one of SEQ ID NO: 20, 21 and 22. In one embodiment, one of said first, second, third, fourth or fifth fusion polypeptides comprises SEQ ID NO:20 and 22, SEQ ID NO:20 and 21; or SEQ ID NO:21 and 22. In one embodiment, one of said first, second, third, fourth or fifth fusion polypeptides comprises SEQ ID NO:20, 21 and 22.

In one embodiment there is provided an immunogenic composition as disclosed herein, wherein one of said first, second, third, fourth or fifth fusion polypeptides comprises or consists of SEQ ID NO:43 or an amino acid sequence with at least 80% identity to SEQ ID NO:43.

In one embodiment, there is provided an immunogenic composition wherein one of said first, second, third, fourth and fifth fusion polypeptides comprises or consists of SEQ ID NO:43 wherein independently of each other the amino acid residue in position 303 may be absent or present or not G and the amino acid residue in position 304 may be absent or present or not S. In one particular embodiment, said fusion polypeptide further comprises one or several additional N-terminal and/or C-terminal amino acids, such as an N-terminal M or MT or MTG or MTGS (SEQ ID NO:103), and optionally comprises a C-terminal His-tag, such as a HHHHHH. For example, said one of said first, second, third, fourth or fifth fusion polypeptides may comprise said SEQ ID NO:43 wherein independently of each other the amino acid residue in position 303 may be absent or present or not G and the amino acid residue in position 304 may be absent or present or not S and said fusion polypeptide may comprise the additional N-terminal and/or C-terminal amino acids. In other words, said one of said first, second, third, fourth or fifth fusion polypeptides comprising SEQ ID NO:20, 21 and 22 may have the amino acid sequence as shown in SEQ ID NO:106, wherein the above mentioned positions are indicated with X, wherein said X represents any amino acid residue or the absence of the amino acid residue in said positions. In one particular embodiment, said one of said first, second, third, fourth or fifth fusion polypeptides comprises or consists of SEQ ID NO:106, or an amino acid sequence with at least 80% identity to SEQ ID NO:106, In one particular embodiment, said one of said first, second, third, fourth or fifth fusion polypeptides comprises or consists of SEQ ID NO:49 or an amino acid sequence with at least 80% identity to SEQ ID NO:49. In one embodiment, one of said first, second, third, fourth or fifth fusion polypeptides comprises or consists of SEQ ID NO:49 wherein independently of each other the amino acid residue in position 304 may be absent or present or not G and the amino acid residue in position 305 may be absent or present or not S. In other words, said one of said first, second, third, fourth or fifth fusion polypeptides comprising SEQ ID NO:20, 21 and 22 may have the amino acid sequence as shown in SEQ ID NO:109, wherein the above mentioned positions are indicated with X, wherein said X represents any amino acid residue or the absence of the amino acid residue in said positions. In one particular embodiment, said one of said first, second, third, fourth or fifth fusion polypeptides comprises or consists of SEQ ID NO:109, or an amino acid sequence with at least 80% identity to SEQ ID NO:109.

In one embodiment there is provided an immunogenic composition as disclosed herein, wherein one of said first, second, third, fourth and fifth fusion polypeptides comprises at least one, such as at least two, such as at least three, such as at least four, such as at least five, such as at least six, such as at least seven, such as all, of SEQ ID NO:23-27 and SEQ ID NO:37-39 and any amino acid sequence with at least 80% identity to any one of SEQ ID NO:23-27 and SEQ ID NO:37-39. In one embodiment, of said first, second, third, fourth or fifth fusion polypeptides comprises SEQ ID NO:23-27 and SEQ ID NO:37-39. In one particular embodiment, one of said first, second, third, fourth or fifth fusion polypeptides comprises or consists of SEQ ID NO:44 or an amino acid sequence with at least 80% identity to SEQ ID NO:44. In one particular embodiment, said SEQ ID NO:44 further comprises one or several additional N-terminal and/or C-terminal amino acids. In one embodiment, said SEQ ID NO:44 further comprises an N-terminal M, such as N-terminal amino acid residues MT or MTG or MTGS. In one particular embodiment, said one of said first, second, third, fourth or fifth fusion polypeptides comprises or consists of SEQ ID NO:50 or an amino acid sequence with at least 80% identity to SEQ ID NO:50. In other words, said one of said first, second, third, fourth or fifth fusion polypeptides comprising SEQ ID NO:23-27 and SEQ ID NO:37-39 may have the amino acid sequence as shown in SEQ ID NO:110, wherein the above mentioned positions are indicated with X, wherein said X represents any amino acid residue or the absence of the amino acid residue. In one particular embodiment, said one of said first, second, third, fourth or fifth fusion polypeptides comprises or consists of SEQ ID NO:110, or an amino acid sequence with at least 80% identity to SEQ ID NO:110.

In one embodiment there is provided an immunogenic composition as disclosed herein, wherein one of said first, second, third, fourth or fifth fusion polypeptides comprises at least one, such as at least two, such as at least three, such as at least four, such as at least five, such as at least six of SEQ ID NO:28-33 or amino acid sequences with at least 80% identity to any one of SEQ ID NO:28-33. In one embodiment, one of said first, second, third, fourth or fifth fusion polypeptides comprises SEQ ID NO:28-33. In one particular embodiment, said one of said first, second, third, fourth or fifth fusion polypeptides comprises or consists of SEQ ID NO:45 or an amino acid sequence with at least 80% identity to SEQ ID NO:45. In one particular embodiment, said fusion polypeptide further comprises one or several additional N-terminal and/or C-terminal amino acids. In one embodiment, said fusion polypeptide further comprises an N-terminal M, such as N-terminal amino acid residues MT, MTG or MTGS. In one particular embodiment, said one of said first, second, third, fourth or fifth fusion polypeptides comprises or consists of SEQ ID NO:51 or an amino acid sequence with at least 80% identity to SEQ ID NO:51. In other words, said one of said first, second, third, fourth or fifth fusion polypeptides comprising SEQ ID NO: 28-33 may have the amino acid sequence as shown in SEQ ID NO:111, wherein the above mentioned positions are indicated with X, wherein said X represents any amino acid residue or the absence of the amino acid residue in said positions. In one particular embodiment, said one of said first, second, third, fourth or fifth fusion polypeptides comprises or consists of SEQ ID NO:111, or an amino acid sequence with at least 80% identity to SEQ ID NO:111.

In one embodiment there is provided an immunogenic composition as disclosed herein, wherein one of said first, second, third, fourth or fifth fusion polypeptides comprises at least one, such as at least two, such as at least three of SEQ ID NO:34-36 or amino acid sequences with at least 80% identity to any one of SEQ ID NO:34-36. In one embodiment, one of said first, second, third, fourth or fifth fusion polypeptides comprises SEQ ID NO:30-32. In one particular embodiment, said one of said first, second, third, fourth or fifth fusion polypeptides comprises or consists of SEQ ID NO:46 or an amino acid sequence with at least 80% identity to SEQ ID NO:46; or one of said first, second, third, fourth or fifth fusion polypeptides comprises or consists of SEQ ID NO:47 or an amino acid sequence with at least 80% identity to SEQ ID NO:47. In one embodiment there is provided an immunogenic composition, wherein one of said first, second, third, fourth and fifth fusion polypeptides comprises or consists of SEQ ID NO:46 or 47 wherein independently of each other the amino acid residue in position 317 may be absent or present or not E; the amino acid residue in position 318 may be absent or present or not F; the amino acid residue in position 608 may be absent or present or not G; the amino acid residue in position 609 may be absent or present or not T; the amino acid residue in position 944 may be absent or present or not L; and the amino acid residue in position 945 may be absent or present or not E. For clarity, the above mentioned positions 317, 318, 608, 609 refer to both SEQ ID NO:46 and 47, while positions 944 and 945 refer to SEQ ID NO:47. In one particular embodiment, said fusion polypeptide further comprises one or several additional N-terminal and/or C-terminal amino acids. In other words, said one of said first, second, third, fourth or fifth fusion polypeptides comprising SEQ ID NO: 34-36 may have the amino acid sequence as shown in SEQ ID NO:107 or 108, wherein the above mentioned positions are indicated with X, wherein said X represents any amino acid residue or the absence of the amino acid residue in said positions. In one embodiment, said fusion polypeptide further comprises an N-terminal M, such as N-terminal amino acid residues MT or MTG or MTGS. In one embodiment, SEQ ID NO:46 further comprises a C-terminal SL2-tag (SEQ ID NO:80).

In one particular embodiment, said one of said first, second, third, fourth or fifth fusion polypeptides comprises or consists of SEQ ID NO:52 or 53 or an amino acid sequence with at least 80% identity to SEQ ID NO:52 or 53. In one embodiment, one of said first, second, third, fourth or fifth fusion polypeptides comprises or consists of SEQ ID NO:52 or 53 wherein independently of each other the amino acid residue in position 321 may be absent or present or not E; the amino acid residue in position 322 may be absent or present or not F; the amino acid residue in position 612 may be absent or present or not G; the amino acid residue in position 613 may be absent or present or not T; the amino acid residue in position 948 may be absent or present or not L; and the amino acid residue in position 949 may be absent or present or not E. For clarity, the above mentioned positions 321, 322, 612, 613 refer to both SEQ ID NO:52 and 53, while positions 948 and 949 refer to SEQ ID NO:47. In other words, said one of said first, second, third, fourth or fifth fusion polypeptides comprising SEQ ID NO: 34-36 may have the amino acid sequence as shown in SEQ ID NO:112 or 113, wherein the above mentioned positions are indicated with X, wherein said X represents any amino acid residue or the absence of the amino acid residue in said positions. In one particular embodiment, said one of said first, second, third, fourth or fifth fusion polypeptides comprises or consists of SEQ ID NO: 112 or 113, or an amino acid sequence with at least 80% identity to SEQ ID NO:112 or 113.

In one embodiment there is provided an immunogenic composition as disclosed herein, wherein one of said first, second, third, fourth or fifth fusion polypeptides comprises at least one, such as at least two, such as at least three of SEQ ID NO:40-42 or amino acid sequences with at least 80% identity to any one of SEQ ID NO:40-42. In one embodiment, one of said first, second, third, fourth or fifth fusion polypeptides comprises SEQ ID NO:40-42. In one particular embodiment, said one of said first, second, third, fourth or fifth fusion polypeptides comprises or consists of SEQ ID NO:48 or an amino acid sequence with at least 80% identity to SEQ ID NO:48. In one particular embodiment, said fusion polypeptide comprising SEQ ID NO:48 further comprises one or several additional N-terminal and/or C-terminal amino acids. In one embodiment, said fusion polypeptide comprising SEQ ID NO:48 further comprises an N-terminal M, such as N-terminal amino acid residues MT or MTG or MTGS, or comprises N-terminal GPLGS. The N-terminal amino acid residues may be G, GP, GLP or GLPG. In one particular embodiment, said one of said first, second, third, fourth or fifth fusion polypeptides comprises SEQ ID NO:54 or 55 or an amino acid sequence with at least 80% identity to SEQ ID NO:54 or 55. In one particular embodiment, said one of said first, second, third, fourth or fifth fusion polypeptides comprises or consists of SEQ ID NO:114 or 115, or an amino acid sequence with at least 80% identity to SEQ ID NO:114 or 115. Herein, positions are indicated with X represents any amino acid residue or the absence of the amino acid residue in said positions.

In particular embodiments, said at least two, at least three, four or all five of said first, second, third, fourth or fifth fusion polypeptides are selected from the group consisting of:
fusion polypeptides comprising SEQ ID NO:20, 21 and 22 or amino acid sequences with at least 80% identity to any one of SEQ ID NO:20, 21 and 22;
fusion polypeptides comprising SEQ ID NO:23-27 and SEQ ID NO:37-39 or amino acid sequences with at least 80% identity to any one of SEQ ID NO:23-27 and SEQ ID NO:37-39;
fusion polypeptides comprising SEQ ID NO:28-33 or amino acid sequences with at least 80% identity to any one of SEQ ID NO:28-33;
fusion polypeptides comprising SEQ ID NO:34-36 or amino acid sequences with at least 80% identity to any one of SEQ ID NO:34-36; and
fusion polypeptides comprising SEQ ID NO:40-42 or amino acid sequences with at least 80% identity to any one of SEQ ID NO:40-42.

In one embodiment, said at least two, at least three, four or all five of said first, second, third, fourth and fifth fusion polypeptides are selected from the group consisting of:
fusion polypeptides comprising SEQ ID NO:20-22;
fusion polypeptides comprising SEQ ID NO:23-27 and SEQ ID NO:37-39;
fusion polypeptides comprising SEQ ID NO:28-33;
fusion polypeptides comprising SEQ ID NO:34-36; and
fusion polypeptides comprising SEQ ID NO:40-42.

In one embodiment of the immunogenic composition as disclosed herein, the composition comprises two fusion polypeptides. Thus the composition may comprise: a fusion polypeptide comprising SEQ ID NO:20-22 and a fusion polypeptide comprising SEQ ID NO:23-27 and 37-39; or a fusion polypeptide comprising SEQ ID NO:20-22 and a fusion polypeptide comprising SEQ ID NO:28-33; or a fusion polypeptide comprising SEQ ID NO:20-22 and a fusion polypeptide comprising SEQ ID NO:34-36; or a fusion polypeptide comprising SEQ ID NO:20-22 and a fusion polypeptide comprising SEQ ID NO:40-42; or a fusion polypeptide comprising SEQ ID NO:23-27 and 37-39 and a fusion polypeptide comprising SEQ ID NO:28-33; or a fusion polypeptide comprising SEQ ID NO:23-27 and 37-39 and a fusion polypeptide comprising SEQ ID NO:34-36; or a fusion polypeptide comprising SEQ ID NO:23-27 and 37-39 and a fusion polypeptide comprising SEQ ID NO:40-42; or a fusion polypeptide comprising SEQ ID NO:28-33 and a fusion polypeptide comprising SEQ ID NO:34-36; or a fusion polypeptide comprising SEQ ID NO:28-33 and a fusion polypeptide comprising SEQ ID NO:40-42; or a fusion polypeptide comprising SEQ ID NO:34-36 and a fusion polypeptide comprising SEQ ID NO:40-42.

In one embodiment of the immunogenic composition as disclosed herein, the composition comprises three fusion polypeptides. Thus the composition may comprise: a fusion polypeptide comprising SEQ ID NO:20-22, a fusion polypeptide comprising SEQ ID NO:23-27 and 37-39 and a fusion polypeptide comprising SEQ ID NO:28-33; or a fusion polypeptide comprising SEQ ID NO:20-22, a fusion polypeptide comprising SEQ ID NO:23-27 and 37-39 and a fusion polypeptide comprising SEQ ID NO:34-36; or a fusion polypeptide comprising SEQ ID NO:20-22, a fusion polypeptide comprising SEQ ID NO:23-27 and 37-39 and a fusion polypeptide comprising SEQ ID NO:40-42; or a fusion polypeptide comprising SEQ ID NO:20-22, a fusion polypeptide comprising SEQ ID NO:28-33 and a fusion polypeptide comprising SEQ ID NO:34-36; or a fusion polypeptide comprising SEQ ID NO:20-22, a fusion polypeptide comprising SEQ ID NO:28-33 and a fusion polypeptide comprising SEQ ID NO:40-42; or a fusion polypeptide comprising SEQ ID NO:20-22 and a fusion polypeptide comprising SEQ ID NO:34-36 and a fusion polypeptide comprising SEQ ID NO:40-42; or a fusion polypeptide comprising SEQ ID NO:23-27 and 37-39, a fusion polypeptide comprising SEQ ID NO:28-33 and a fusion polypeptide comprising SEQ ID NO:34-36; or a fusion polypeptide comprising SEQ ID NO:23-27 and 37-39, a fusion polypeptide comprising SEQ ID NO:28-33 and a fusion polypeptide comprising SEQ ID NO:40-42; or a fusion polypeptide comprising SEQ ID NO:23-27 and 37-39, a fusion polypeptide comprising SEQ ID NO:34-36 and a fusion polypeptide comprising SEQ ID NO:40-42, or a fusion polypeptide comprising SEQ ID NO:28-33, a fusion polypeptide comprising SEQ ID NO:34-36 and a fusion polypeptide comprising SEQ ID NO:40-42.

In one embodiment of the immunogenic composition as disclosed herein, the composition comprises four fusion polypeptides. Thus the composition may comprise: a fusion polypeptide comprising SEQ ID NO:20-22, a fusion polypeptide comprising SEQ ID NO:23-27 and 37-39, a fusion polypeptide comprising SEQ ID NO:28-33 and a fusion polypeptide comprising SEQ ID NO:34-36; or fusion polypeptide comprising SEQ ID NO:20-22, a fusion polypeptide comprising SEQ ID NO:23-27 and 37-39, a fusion polypeptide comprising SEQ ID NO:28-33 and a fusion polypeptide comprising SEQ ID NO:40-42; or a fusion polypeptide comprising SEQ ID NO:20-22, a fusion polypeptide comprising SEQ ID NO:23-27 and 37-39, a fusion polypeptide comprising SEQ ID NO:34-36 and a fusion polypeptide comprising SEQ ID NO:40-42; or a fusion polypeptide comprising SEQ ID NO:20-22, a fusion polypeptide comprising SEQ ID NO:28-33, a fusion polypeptide comprising SEQ ID NO:34-36 and a fusion polypeptide comprising SEQ ID NO:40-42; or a fusion polypeptide comprising SEQ ID NO:23-27 and 37-39, a fusion polypeptide comprising SEQ ID NO:28-33, a fusion polypeptide comprising SEQ ID NO:34-36 and a fusion polypeptide comprising SEQ ID NO:40-42. In one embodiment, of the immunogenic composition as disclosed herein, the composition comprises five fusion polypeptides. Thus the composition may comprise: a fusion polypeptide comprising SEQ ID NO:20-22, a fusion polypeptide comprising SEQ ID NO:23-27 and 37-39, a fusion polypeptide comprising SEQ ID NO:28-33, a fusion polypeptide comprising SEQ ID NO:34-36 and a fusion polypeptide comprising SEQ ID NO:40-42.

The skilled person will appreciate the % identity feature applies to any of said fusion polypeptides as explained above, but is not repeated here merely for the sake of brevity. Thus, encompassed are also immunogenic compositions comprising fusion polypeptides comprising fragments which exhibit at least 80% identity to the recited fragments, such as for example immunogenic composition comprising a fusion polypeptide comprising SEQ ID NO:28-33 or amino acid sequences with at least 80% identity to any one of SEQ ID NO:28-33, a fusion polypeptide comprising SEQ ID NO:34-36 or amino acid sequences with at least 80% identity to any one of SEQ ID NO:34-36, and a fusion polypeptide comprising SEQ ID NO:40-42 or amino acid sequences with at least 80% identity to any one of SEQ ID NO:40-42.

In one embodiment, there is provided an immunogenic composition as disclosed herein, wherein at least two, at least three or all four of said first, second, third, fourth or fifth fusion polypeptides are selected from the group consisting of:
fusion polypeptides comprising SEQ ID NO:43 or an amino acid sequence with at least 80% identity to SEQ ID NO:43;
fusion polypeptides comprising SEQ ID NO:44 or an amino acid sequence with at least 80% identity to SEQ ID NO:44;
fusion polypeptides comprising SEQ ID NO:45 or an amino acid sequence with at least 80% identity to SEQ ID NO:45;
fusion polypeptides comprising SEQ ID NO:46 or an amino acid sequence with at least 80% identity to SEQ ID NO:46, and
fusion polypeptides comprising SEQ ID NO:48 or an amino acid sequence with at least 80% identity to SEQ ID NO:48;
or from the group consisting of:
fusion polypeptides comprising SEQ ID NO:43 or an amino acid sequence with at least 80% identity to SEQ ID NO:43;
fusion polypeptides comprising SEQ ID NO:44 or an amino acid sequence with at least 80% identity to SEQ ID NO:44;
fusion polypeptides comprising SEQ ID NO:45 or an amino acid sequence with at least 80% identity to SEQ ID NO:45; and
fusion polypeptides comprising SEQ ID NO:47 or an amino acid sequence with at least 80% identity to SEQ ID NO:47 and
fusion polypeptides comprising SEQ ID NO:48 or an amino acid sequence with at least 80% identity to SEQ ID NO:48.

In one embodiment, there is provided an immunogenic composition as disclosed herein, wherein at least two, at least three or all four of said first, second, third, fourth or fifth fusion polypeptides are selected from the group consisting of:
fusion polypeptides comprising SEQ ID NO:106 or an amino acid sequence with at least 80% identity to SEQ ID NO:106;
fusion polypeptides comprising SEQ ID NO:44 or an amino acid sequence with at least 80% identity to SEQ ID NO:44;
fusion polypeptides comprising SEQ ID NO:45 or an amino acid sequence with at least 80% identity to SEQ ID NO:45;
fusion polypeptides comprising SEQ ID NO:107 or an amino acid sequence with at least 80% identity to SEQ ID NO:107, and
fusion polypeptides comprising SEQ ID NO:48 or an amino acid sequence with at least 80% identity to SEQ ID NO:48;
or from the group consisting of:
fusion polypeptides comprising SEQ ID NO:106 or an amino acid sequence with at least 80% identity to SEQ ID NO:106;
fusion polypeptides comprising SEQ ID NO:44 or an amino acid sequence with at least 80% identity to SEQ ID NO:44;
fusion polypeptides comprising SEQ ID NO:45 or an amino acid sequence with at least 80% identity to SEQ ID NO:45; and
fusion polypeptides comprising SEQ ID NO:108 or an amino acid sequence with at least 80% identity to SEQ ID NO:108 and
fusion polypeptides comprising SEQ ID NO:48 or an amino acid sequence with at least 80% identity to SEQ ID NO:48.

In one embodiment, there is provided an immunogenic composition as disclosed herein, wherein at least two, at least three, at least four or all five of said first, second, third, fourth or fifth fusion polypeptides are selected from the above mentioned group.

In one embodiment, said immunogenic composition comprises a fusion polypeptide comprising SEQ ID NO:43 or an amino acid sequence with at least 80% identity to SEQ ID NO:43; a fusion polypeptide comprising SEQ ID NO:44 or an amino acid sequence with at least 80% identity to SEQ ID NO:44; a fusion polypeptide comprising SEQ ID NO:45 or an amino acid sequence with at least 80% identity to SEQ ID NO:45; a fusion polypeptide comprising SEQ ID NO:47 or an amino acid sequence with at least 80% identity to SEQ ID NO:47 and fusion polypeptides comprising SEQ ID NO:48 or an amino acid sequence with at least 80% identity to SEQ ID NO:48. In one embodiment, said immunogenic composition comprises a fusion polypeptide comprising SEQ ID NO:43 or an amino acid sequence with at least 80% identity to SEQ ID NO:43; a fusion polypeptide comprising SEQ ID NO:44 or an amino acid sequence with at least 80% identity to SEQ ID NO:44; a fusion polypeptide comprising SEQ ID NO:45 or an amino acid sequence with at least 80% identity to SEQ ID NO:45; a fusion polypeptide comprising SEQ ID NO:46 or an amino acid sequence with at least 80% identity to SEQ ID NO:46 and fusion polypeptides comprising SEQ ID NO:48 or an amino acid sequence with at least 80% identity to SEQ ID NO:48. In one embodiment, said immunogenic composition comprises a fusion polypeptide comprising SEQ ID NO:43 or an amino acid sequence with at least 80% identity to SEQ ID NO:43; a fusion polypeptide comprising SEQ ID NO:44 or an amino acid sequence with at least 80% identity to SEQ ID NO:44; a fusion polypeptide comprising SEQ ID NO:45 or an amino acid sequence with at least 80% identity to SEQ ID NO:45 and a fusion polypeptide comprising SEQ ID NO:46 or an amino acid sequence with at least 80% identity to SEQ ID NO:46. In one embodiment, said immunogenic composition comprises a fusion polypeptide comprising SEQ ID NO:43 or an amino acid sequence with at least 80% identity to SEQ ID NO:43; a fusion polypeptide comprising SEQ ID NO:44 or an amino acid sequence with at least 80% identity to SEQ ID NO:44; a fusion polypeptide comprising SEQ ID NO:45 or an amino acid sequence with at least 80% identity to SEQ ID NO:45 and a fusion polypeptide comprising SEQ ID NO:47 or an amino acid sequence with at least 80% identity to SEQ ID NO:47.

In particular embodiments, said at least two, at least three, four or all five of said first, second, third, fourth or fifth fusion polypeptides may be selected from the group consisting of:
fusion polypeptides comprising SEQ ID NO:43; fusion polypeptides comprising SEQ ID NO:44; fusion polypeptides comprising SEQ ID NO:45; fusion polypeptides comprising SEQ ID NO:46; and fusion polypeptides comprising SEQ ID NO:48
or from the group consisting of: fusion polypeptides comprising SEQ ID NO:43; fusion polypeptides comprising SEQ ID NO:44; fusion polypeptides comprising SEQ ID NO:45; fusion polypeptides comprising SEQ ID NO:47; and fusion polypeptides comprising SEQ ID NO:48. In one particular embodiment, said immunogenic composition comprises a fusion polypeptide comprising SEQ ID NO:43; a fusion polypeptide comprising SEQ ID NO:44; a fusion polypeptide comprising SEQ ID NO:45; a fusion polypeptide comprising SEQ ID NO:47; and a fusion polypeptide comprising SEQ ID NO:48. In one particular embodiment, said immunogenic composition comprises a fusion polypeptide comprising SEQ ID NO:43; a fusion polypeptide comprising SEQ ID NO:44; a fusion polypeptide comprising SEQ ID NO:45; a fusion polypeptide comprising SEQ ID NO:46; and a fusion polypeptide comprising SEQ ID NO:48. In one particular embodiment, said immunogenic composition comprises a fusion polypeptide comprising SEQ ID NO:43; a fusion polypeptide comprising SEQ ID NO:44; a fusion polypeptide comprising SEQ ID NO:45 and a fusion polypeptide comprising SEQ ID NO:47. In one particular embodiment, said immunogenic composition comprises a fusion polypeptide comprising SEQ ID NO:43; a fusion polypeptide comprising SEQ ID NO:44; a fusion polypeptide comprising SEQ ID NO:45 and a fusion polypeptide comprising SEQ ID NO:46.

It will be appreciated that said SEQ ID NO:43 may equally well be a SEQ ID NO:43 wherein independently of each other the amino acid residue in position 303 may be absent or present or not G and the amino acid residue in position 304 may be absent or present or not S and said SEQ ID NO:43 may comprise the additional N-terminal and/or C-terminal amino acids.

Thus, the skilled person appreciates that the amino acid sequences of SEQ ID NO:43, wherein amino acid residue may be present of absent of different in positions 303 and 304 as indicated above may be replaced by SEQ ID NO: 106 in the herein listed embodiments, wherein the above mentioned positions are indicated with X, wherein said X represents any amino acid residue or the absence of the amino acid residue in said positions.

It will be appreciated that said SEQ ID NO:46 and/or 47 may equally well be a SEQ ID NO:46 and/or 47 wherein independently of each other the amino acid residue in position 317 may be absent or present or not E; the amino acid residue in position 318 may be absent or present or not F; the amino acid residue in position 608 may be absent or present or not G; the amino acid residue in position 609 may be absent or present or not T; the amino acid residue in position 944 may be absent or present or not L; and the amino acid residue in position 945 may be absent or present or not E. Thus, the skilled person appreciates that the amino acid sequences of SEQ ID NO:46, wherein amino acid residue may be present of absent or different in positions 317, 318, 608, and 609 as indicated above may be replaced by SEQ ID NO:107, in the herein listed embodiments, wherein said X represents any amino acid residue or the absence of the amino acid residue in said positions. Thus, the skilled person appreciates that the amino acid sequences of SEQ ID NO:47, wherein amino acid residue may be present of absent or different in positions 317, 318, 608, 609, 944 and 945 as indicated above may be replaced by SEQ ID NO: 108.

In one embodiment, said fusion polypeptide comprising SEQ ID NO:48 is SEQ ID NO:54 or SEQ ID NO:55. In one embodiment, said fusion polypeptide comprising SEQ ID NO:48 is SEQ ID NO:114 or SEQ ID NO:115 In one embodiment of the immunogenic composition as disclosed herein, the composition comprises two fusion polypeptides. Thus the composition may comprise: SEQ ID NO:43, wherein independently of each other the amino acid residue in position 303 may be absent or present or not G and the amino acid residue in position 304 may be absent or present or not S, and SEQ ID NO:44;
or SEQ ID NO:43, wherein independently of each other the amino acid residue in position 303 may be absent or present or not G and the amino acid residue in position 304 may be absent or present or not S, and SEQ ID NO:45;
or SEQ ID NO:43, wherein independently of each other the amino acid residue in position 303 may be absent or present or not G and the amino acid residue in position 304 may be absent or present or not S, and SEQ ID NO:46, wherein independently of each other the amino acid residue in position 317 may be absent or present or not E, the amino acid residue in position 318 may be absent or present or not F, the amino acid residue in position 608 may be absent or not G and the amino acid residue in position 609 may be absent or present or not T;
or SEQ ID NO:43, wherein independently of each other the amino acid residue in position 303 may be absent or present or not G and the amino acid residue in position 304 may be absent or present or not S, and SEQ ID NO:48;
or SEQ ID NO:44 and SEQ ID NO:45;
or SEQ ID NO:44 and SEQ ID NO:46, wherein independently of each other the amino acid residue in position 317 may be absent or present or not E, the amino acid residue in position 318 may be absent or present or not F, the amino acid residue in position 608 may be absent or not G and the amino acid residue in position 609 may be absent or present or not T;
or SEQ ID NO:44 and SEQ ID NO:48;
or SEQ ID NO:45 and SEQ ID NO:46, wherein independently of each other the amino acid residue in position 317 may be absent or present or not E, the amino acid residue in position 318 may be absent or present or not F, the amino acid residue in position 608 may be absent or not G and the amino acid residue in position 609 may be absent or present or not T;
or SEQ ID NO:45 and SEQ ID NO:48;
or SEQ ID NO:46, wherein independently of each other the amino acid residue in position 317 may be absent or present or not E, the amino acid residue in position 318 may be absent or present or not F, the amino acid residue in position 608 may be absent or not G and the amino acid residue in position 609 may be absent or present or not T, and SEQ ID NO:48.

In one embodiment of the immunogenic composition as disclosed herein, the composition comprises three fusion polypeptides. Thus the composition may comprise: SEQ ID NO:43, wherein independently of each other the amino acid residue in position 303 may be absent or present or not G and the amino acid residue in position 304 may be absent or present or not S, and SEQ ID NO:44 and SEQ ID NO:45;
or SEQ ID NO:43, wherein independently of each other the amino acid residue in position 303 may be absent or present or not G and the amino acid residue in position 304 may be absent or present or not S, and SEQ ID NO:44 and SEQ ID NO:46, wherein independently of each other the amino acid residue in position 317 may be absent or present or not E, the amino acid residue in position 318 may be absent or present or not F, the amino acid residue in position 608 may be absent or not G and the amino acid residue in position 609 may be absent or present or not T;
or SEQ ID NO:43, wherein independently of each other the amino acid residue in position 303 may be absent or present or not G and the amino acid residue in position 304 may be absent or present or not S, and SEQ ID NO:44 and SEQ ID NO:48;
or SEQ ID NO:43, wherein independently of each other the amino acid residue in position 303 may be absent or present or not G and the amino acid residue in position 304 may be absent or present or not S, and SEQ ID NO:45 and SEQ ID NO:46, wherein independently of each other the amino acid residue in position 317 may be absent or present or not E, the amino acid residue in position 318 may be absent or present or not F, the amino acid residue in position 608 may be absent or not G and the amino acid residue in position 609 may be absent or present or not T;
or SEQ ID NO:43, wherein independently of each other the amino acid residue in position 303 may be absent or present or not G and the amino acid residue in position 304 may be absent or present or not S, and SEQ ID NO:45 and SEQ ID NO:48;
or SEQ ID NO:43, wherein independently of each other the amino acid residue in position 303 may be absent or present or not G and the amino acid residue in position 304 may be absent or present or not S, and SEQ ID NO:46, wherein independently of each other the amino acid residue in position 317 may be absent or present or not E, the amino acid residue in position 318 may be absent or present or not F, the amino acid residue in position 608 may be absent or not G and the amino acid residue in position 609 may be absent or present or not T, and SEQ ID NO:48;
or SEQ ID NO:44 and SEQ ID NO:45 and SEQ ID NO:46, wherein independently of each other the amino acid residue in position 317 may be absent or present or not E, the amino acid residue in position 318 may be absent or present or not F, the amino acid residue in position 608 may be absent or not G and the amino acid residue in position 609 may be absent or present or not T;
or SEQ ID NO:44 and SEQ ID NO:45 and SEQ ID NO:48;
or SEQ ID NO:44 and SEQ ID NO:46, wherein independently of each other the amino acid residue in position 317 may be absent or present or not E; the amino acid residue in position 318 may be absent or present or not F; the amino acid residue in position 608 may be absent or not G and the amino acid residue in position 609 may be absent or present or not T, and SEQ ID NO:48;
or SEQ ID NO:45 and SEQ ID NO:46, wherein independently of each other the amino acid residue in position 317 may be absent or present or not E, the amino acid residue in position 318 may be absent or present or not F, the amino acid residue in position 608 may be absent or not G and the amino acid residue in position 609 may be absent or present or not T, and SEQ ID NO:48.

In one embodiment of the immunogenic composition as disclosed herein, the composition comprises four fusion polypeptides. Thus the composition may comprise: SEQ ID NO:43, wherein independently of each other the amino acid residue in position 303 may be absent or present or not G and the amino acid residue in position 304 may be absent or present or not S, and SEQ ID NO:44 and SEQ ID NO:45 and SEQ ID NO:46, wherein independently of each other the amino acid residue in position 317 may be absent or present or not E, the amino acid residue in position 318 may be absent or present or not F, the amino acid residue in position 608 may be absent or not G and the amino acid residue in position 609 may be absent or present or not T; or
SEQ ID NO:43, wherein independently of each other the amino acid residue in position 303 may be absent or present or not G and the amino acid residue in position 304 may be absent or present or not S, and SEQ ID NO:44 and SEQ ID NO:45 and SEQ ID NO:48;
or SEQ ID NO:43, wherein independently of each other the amino acid residue in position 303 may be absent or present or not G and the amino acid residue in position 304 may be absent or present or not S, and SEQ ID NO:44 and SEQ ID NO:46, wherein independently of each other the amino acid residue in position 317 may be absent or present or not E, the amino acid residue in position 318 may be absent or present or not F, the amino acid residue in position 608 may be absent or not G and the amino acid residue in position 609 may be absent or present or not T and SEQ ID NO:48;
or SEQ ID NO:43, wherein independently of each other the amino acid residue in position 303 may be absent or present or not G and the amino acid residue in position 304 may be absent or present or not S, and SEQ ID NO:45 and SEQ ID NO:46, wherein independently of each other the amino acid residue in position 317 may be absent or present or not E, the amino acid residue in position 318 may be absent or present or not F, the amino acid residue in position 608 may be absent or not G and the amino acid residue in position 609 may be absent or present or not T and SEQ ID NO:48;
or SEQ ID NO:44 and SEQ ID NO:45 and SEQ ID NO:46, wherein independently of each other the amino acid residue in position 317 may be absent or present or not E, the amino acid residue in position 318 may be absent or present or not F, the amino acid residue in position 608 may be absent or not G and the amino acid residue in position 609 may be absent or present or not T, and SEQ ID NO:48.

In one embodiment said composition comprises SEQ ID NO:43, wherein independently of each other the amino acid residue in position 303 may be absent or present or not G and the amino acid residue in position 304 may be absent or present or not S, SEQ ID NO:44 and SEQ ID NO:45 and SEQ ID NO:46, wherein independently of each other the amino acid residue in position 317 may be absent or present or not E, the amino acid residue in position 318 may be absent or present or not F, the amino acid residue in position 608 may be absent or not G and the amino acid residue in position 609 may be absent or present or not T, and SEQ ID NO:48.

The skilled person will appreciate the % identity feature applies to any of said fusion polypeptides as explained above, but is not repeated here merely for the sake of brevity.

It will also be appreciated that said % identity feature may apply to any one of the first, second, third, fourth or fifth polypeptide discussed above. In particular, encompassed are also amino acid sequences which are at least 81%, such as at least 82% such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 93%, such as at least 95%, such as at least 96%, such as at least 97%, such as at least 98% or such as at least 99% identical to the recited fragments of native proteins of S. suis or fusion polypeptides referred to in the embodiments above. The skilled person will appreciate that recitation of % identity levels above is equally relevant for any embodiment of this first aspect and second aspects, wherein an % identity feature is recited. For the sake of brevity it will not be repeated each time.

The fusion polypeptides of the present disclosure may be unstructured comprising partially or completely unstructured regions of native polypeptides, in other word regions which lack a well-defined tertiary structure.

As used herein, the term “unstructured” or “disordered” in relation to a polypeptide, protein or fragment thereof refers to polypeptide, protein or fragment thereof which do not have single well-defined tertiary structure under native conditions. Several different computer programs may be used for prediction of unstructured features of polypeptide, protein or fragment thereof. Examples of such programs are IUPred, IUPred2 and IUPred2A. The skilled person is familiar with said programs and how to use them to predict structural features of polypeptide, protein or fragment thereof. Generally speaking, unstructured regions are characterized by a compositional bias in their amino-acid sequence, in that they contain a significantly larger proportion of small and hydrophilic amino acids and proline residues than structured regions.

In one embodiment of the immunogenic composition as disclosed herein, at least one, such as at least two or at least three, of said fusion polypeptides is/are not denatured and/or not precipitated by heating to approximately 100° C. In the context of the present disclosure, the lack of effect of heating may be related to the tertiary structure of the fusion polypeptides. Thus heating to approximately 100° C. does not lead to a denaturation effect on the structural properties, in particular tertiary structure properties, of the fusion polypeptides and/or does not lead to precipitation of the fusion polypeptides. In particular, the fusion polypeptides may beneficially comprise unstructured (also known as disordered) fragments of native polypeptides and therefore also the fusion polypeptides are unstructured to a large degree or completely unstructured.

Without being bound by theory unstructured or disordered proteins have been described as important antigens in a range of infectious diseases and disordered epitopes have been reported to as likely to be recognized by antibodies as ordered epitopes (MacRaild C et al., Structure 2016, January 5; 24(1):148-157). Furthermore, studies have shown that disordered epitopes are smaller than their ordered counterparts, but are more efficient in their interactions with antibody and that recognition of disordered epitopes by antibodies is particularly sensitive to epitope variation. Without being bound by theory, unstructured protein parts may be combined as fusion proteins in all possible ways. That is, since neither the fusion protein that is to be used as an antigen or the native protein on the bacterial surface have a defined structure, the present inventors consider that the antibodies obtained will most likely recognize the native protein. Thus, it may be advantageous to use unstructured proteins or fragments as antigens in immunogenic compositions or in vaccines compared to structured proteins. Structured proteins must contain a full domain to be correctly folded or the obtained antibodies will not recognize the native target. Additionally, it may be advantageous to use unstructured proteins or unstructured fragments of proteins to make a fusion protein as shorter fragments about 20-30 amino acids can be used and thus many more different protein parts can be included in the fusion polypeptide compared to if structured proteins or structured fragments are used. Higher numbers of bacterial proteins included in immunogenic compositions are envisioned to be capable of generating broader immune responses that can interfere with the function of multiple proteins that are important to the disease process, provide enhanced targeting of the pathogen for killing by the immune response, account for the complexity of bacterial pathogens and the multiple pathways they employ to cause disease and/or safeguard against the effects of strain variation, which is a particular problem for S. suis. It is considered that the combination of unstructured protein parts in fusion polypeptides makes it possible to obtain antibodies directed against a high number of bacterial proteins.

As explained above in the context of the first aspect relating to the immunogenic composition, fragments of amongst others zinc binding proteins, proteases, proteins comprising an LPXTG-motif and other polypeptides from S. suis may be provided in the form of fusion polypeptides. Said fusion polypeptides may be useful in immunogenic and/or vaccine compositions for prevention of infection by S. suis. Thus, in a second aspect of the present disclosure, there is provided a fusion polypeptide comprising fragments from at least 3, such as at least 4, such as at least 5, such as at least 6, such as at least 7, native full length polypeptides from Streptococcus suis, wherein said native full length polypeptides are independently selected or selected from the group consisting of amino acid sequences according to SEQ ID NO:1-10 and SEQ ID NO:14-19 and amino acid sequences having at least 80% identity to any one of SEQ ID NO:1-10 and SEQ ID NO:14-19, such as such as the group consisting of amino acid sequences according to SEQ ID NO:1-10 and SEQ ID NO:14-19.

In one embodiment, said native full length polypeptides are independently selected or selected from the group consisting of amino acid sequences according to SEQ ID NO:1-3 and amino acid sequences having at least 80% identity to any one of SEQ ID NO:1-3, such as the group consisting of amino acid sequences according to SEQ ID NO:1-3. In one embodiment, said native full length polypeptides are independently selected or selected from the group consisting of amino acid sequences according to SEQ ID NO:1-5 and 14-16 and amino acid sequences having at least 80% identity to any one of SEQ ID NO:1-5 and 14-16, such as the group consisting of amino acid sequences according to SEQ ID NO:1-5 and 14-16. In one embodiment, said native full length polypeptides are independently selected or selected from the group consisting of amino acid sequences according to SEQ ID NO:6-10 and amino acid sequences having at least 80% identity to any one of SEQ ID NO:6-10, such as the group consisting of amino acid sequences according to SEQ ID NO:6-10. In one embodiment, said native full length polypeptides are independently selected or selected from the group consisting of amino acid sequences according to SEQ ID NO:17-19 and amino acid sequences having at least 80% identity to any one of SEQ ID NO:17-19, such as the group consisting of amino acid sequences according to SEQ ID NO:17-19, such as wherein said native full length polypeptides are SEQ ID NO:17-19.

In particular, in one embodiment, said fragments are independently selected or selected from the group consisting of fragments having amino acid sequences according to SEQ ID NO: 20-33 and 37-42 and amino acid sequences having at least 80% identity to any one of SEQ ID NO: 20-33 and 37-42, such as the group consisting of amino acid sequences according to SEQ ID NO: 20-33 and 37-42.

In one embodiment, said fragments are selected from a group consisting of SEQ ID NO:20-22 and amino acid sequences having at least 80% identity to any one of SEQ ID NO:20-22. In one particular embodiment, said fragments have amino acid sequences according to SEQ ID NO:20-22.

In one embodiment, there is provided a fusion polypeptide comprising or consisting of the amino acid sequence SEQ ID NO:43 or an amino acid sequence having 80% identity to SEQ ID NO:43. In one embodiment, said fusion polypeptide comprises or consists of the amino acid sequence SEQ ID NO:43.

In one embodiment, said fusion polypeptide comprises SEQ ID NO:43 wherein independently of each other the amino acid residue in position 303 may be absent or present or not G and the amino acid residue in position 304 may be absent or present or not S. In other words, said fusion polypeptide comprises or consists of SEQ ID NO:106.

In one particular embodiment, said fusion polypeptide further comprises one or several additional N-terminal and/or C-terminal amino acids, such as an N-terminal M or MT, such as M or MT or MTG or MTGS, and optionally comprises a C-terminal His-tag, such as a HHHHHH. For example, said fusion polypeptide may comprise said SEQ ID NO:43 wherein independently of each other the amino acid residue in position 303 may be absent or present or not G and the amino acid residue in position 304 may be absent or present or not S and said fusion polypeptide may comprise the additional N-terminal and/or C-terminal amino acids. In one particular embodiment, said fusion polypeptide comprises or consists of SEQ ID NO:49 or an amino acid sequence with at least 80% identity to SEQ ID NO:49. In one embodiment, said fusion polypeptide comprises or consists of SEQ ID NO:49 wherein independently of each other the amino acid residue in position 304 may be absent or present or not G and the amino acid residue in position 305 may be absent or present or not S. In other words, said fusion polypeptide comprises or consists of SEQ ID NO:109 or an amino acid sequence with at least 80% identity thereto.

In one embodiment there is provided a fusion polypeptide, wherein said fragments are selected from a group of fragments having amino acid sequences according to SEQ ID NO: 23-27 and 37-39 and amino acid sequences having at least 80% identity to any one of SEQ ID NO: 23-27 and 37-39. In one embodiment, said fragments are selected from a group of fragments having amino acid sequences according to SEQ ID NO: 23-27 and 37-39. In one particular embodiment, said fusion polypeptide comprises or consists of the amino acid sequence SEQ ID NO:44 or an amino acid sequence having 80% identity to SEQ ID NO:44. In one embodiment, said fusion polypeptide comprises or consists of the amino acid sequence SEQ ID NO:44. In one particular embodiment, said fusion polypeptide further comprises one or several additional N-terminal and/or C-terminal amino acids.

In one embodiment, said fusion polypeptide further comprises an N-terminal M, such as N-terminal amino acid residues MT or MTG or MTGS. In one particular embodiment, said fusion polypeptide comprises or consists of SEQ ID NO:50 or an amino acid sequence with at least 80% identity to SEQ ID NO:50. In other words, said fusion polypeptide comprises or consists of SEQ ID NO:110 or an amino acid sequence with at least 80% identity thereto.

In one embodiment there is provided a fusion polypeptide as disclose herein, wherein said fragments are selected from a group of fragments having amino acid sequences according to SEQ ID NO: 28-33 and amino acid sequences having at least 80% identity to any one of SEQ ID NO: 28-33. In one embodiment, said fragments are selected from a group of fragments having amino acid sequences according to SEQ ID NO: 28-33. In one particular embodiment said fusion polypeptides comprises or consists of the amino acid sequence SEQ ID NO:45 or an amino acid sequence having 80% identity to SEQ ID NO:45. In one embodiment, said fusion polypeptide comprises or consists of the amino acid sequence SEQ ID NO:45. In one particular embodiment, said fusion polypeptide further comprises one or several additional N-terminal and/or C-terminal amino acids. In one embodiment, said fusion polypeptide further comprises an N-terminal M, such as N-terminal amino acid residues MT or MTG or MTGS. In one particular embodiment, said fusion polypeptide comprises or consists of SEQ ID NO:51 or an amino acid sequence with at least 80% identity to SEQ ID NO:51. In other words, said fusion polypeptide comprises or consists of SEQ ID NO:111 or an amino acid sequence with at least 80% identity thereto.

In one embodiment there is provided a fusion polypeptide as disclose herein, wherein said fragments are selected from a group of fragments having amino acid sequences according to SEQ ID NO:17-19 and amino acid sequences having at least 80% identity to any one of SEQ ID NO:17-19, such as the group consisting of amino acid sequences according to SEQ ID NO:17-19, such as wherein said native full length polypeptides are SEQ ID NO:17-19.

In one particular embodiment said fusion polypeptides comprises or consists of the amino acid sequence. In one particular embodiment, said fusion polypeptide comprises or consists of the amino acid sequence SEQ ID NO:48 or an amino acid sequence having 80% identity to SEQ ID NO:48. In one particular embodiment, said fusion polypeptide further comprises one or several additional N-terminal and/or C-terminal amino acids. In one embodiment, said fusion polypeptide further comprises an N-terminal M, such as N-terminal amino acid residues MT or MTG or MTGS. In one embodiment, said fusion polypeptide further comprises an N-terminal G, such as N-terminal amino acid residues GPLGS. Said N-terminal amino acid residues may be G, GP, GPL or GPLG. In one embodiment, said fusion polypeptide comprises or consists of SEQ ID NO:54 or SEQ ID NO:55 or amino acid sequences having at least 80% identity to SEQ ID NO:54 or SEQ ID NO:55. In other words, said fusion polypeptide comprises or consists of SEQ ID NO:114 or 115 or an amino acid sequence with at least 80% identity thereto.

Again, it will be appreciated that said % identity feature may apply to any one of the fusion polypeptides disclosed above. In particular, encompassed by the present disclosure are fusion polypeptides which are at least 81%, such as at least 82% such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 93%, such as at least 95%, such as at least 96%, such as at least 97%, such as at least 98% or such as at least 99% identical fusion polypeptides referred to in the embodiments above. It will be appreciated that recitation of % identity levels above is equally relevant for any embodiment, wherein an % identity feature is recited. For the sake of brevity it will not be repeated each time.

As discussed in the context of the first aspect of the present disclosure, the fusion polypeptides of the present disclosure may be unstructured comprising partially or completely unstructured regions of native polypeptides, in other word regions which lack tertiary structure. Generally speaking, unstructured regions are characterized by a compositional bias in their amino-acid sequence, in that they contain a significantly larger proportion of small and hydrophilic amino acids and proline residues than structured regions. In this respect, the particular fragments according to any one of SEQ ID NO:20-29 and SEQ ID NO:33-35 are unstructured regions of their respective native polypeptides. Such unstructured regions are not affected by heating to high temperatures which generally denatures structured polypeptides and structured fragments thereof. Even boiling at 100° C. does not affect the structure. Thus, in one embodiment the fusion polypeptide as disclosed herein, there is provided a fusion polypeptide as defined herein, wherein the tertiary structure of said fusion polypeptide is not denatured and/or said fusion polypeptide is not precipitated by heating to approximately 100° C.

In the context of the present disclosure, the lack of affect may be related to the tertiary structure of the fusion polypeptides. Thus heating to approximately 100° C. does not have any effect on the structural properties of the fusion polypeptides. In particular, the fusion polypeptides may beneficially comprise unstructured fragments of native polypeptides and therefore also the fusion polypeptides are unstructured to a large degree or completely unstructured. Advantages of unstructured proteins or fragments are discussed in the context of the first aspect as disclosed herein and is not repeated there merely for the sake of brevity. The skilled person appreciates that said advantages are equally relevant for this second aspect.

In one embodiment, the fusion polypeptides comprising SEQ ID NO:20-22, the fusion polypeptides comprising SEQ ID NO: 23-27 and 37-39; and/or the fusion polypeptides comprising SEQ ID NO:28-33 as disclosed herein comprise or consist of unstructured fragments.

It will be appreciated that the fusion polypeptides comprised in the immunogenic compositions according to the first aspect as described herein and the fusion polypeptides as such according to the second aspect as described herein, may further comprise additional amino acid residues at the N- and/or C-terminus. For the sake of brevity these will not be repeated here or will only be briefly mentioned. For the sake of clarity, the presence of additional amino acid residues at the N- and/or C-terminus of fusion polypeptide does not preclude the presence of additional N- or C-terminal extensions, for example in the form a “tag” for purification as described below. The skilled person will understand that various modifications and/or additions can be made to a fusion polypeptide as defined herein, in order to tailor the polypeptide to a specific application without departing from the scope of the present disclosure. For example, the fusion polypeptide as defined herein may comprise additional amino acid residues for the purpose of improving production, purification and/or stabilization in vivo or in vitro of the fusion polypeptide. Thus, a fusion polypeptide may comprise any suitable number of additional amino acid residues, for example at least one additional amino acid residue. Each additional amino acid residue may individually or collectively be added in order to, for example, improve production, purification, solubility and/or stabilization in vivo or in vitro. Such additional amino acid residues may also provide a “tag” for purification, such as a His-tag (such as for example a His6-tag or a His7-tag) or a “myc” (c-myc) tag or a “FLAG” tag for interaction with antibodies specific to the tag or immobilized metal affinity chromatography (IMAC) in the case of the His-tag. The additional amino acid residues may also comprise a SL2-tag or LSL-tag as described herein.

Thus, in one embodiment there is provided a fusion polypeptide as described herein, wherein said fusion polypeptide further comprises additional amino acid residues at the N- and/or C-terminus. Such a fusion polypeptide should be understood as a polypeptide having one or more additional amino acid residues at the very first and/or the very last position in the polypeptide chain, i.e. at the N- and/or C-terminus. In one embodiment, said fusion polypeptide, comprises a methionine M residue at the N-terminus. In one embodiment, said fusion polypeptide, comprises the amino acids residues MT, MTG or MTGS at the N-terminus. In one embodiment said fusion polypeptide of comprises the amino acid residues LE at the C-terminus.

In one embodiment, there is provided a fusion polypeptide as described herein, wherein said additional amino acid residue(s) at the N-terminus and/or C-terminus or at the N-terminal and/or C-terminal improve production, purification and/or stabilization in vivo or in vitro of said immunogenic polypeptide. In one embodiment, said additional amino acid residue(s) comprise a tag or several tags, such as a tag selected from the group consisting of a His-tag (SEQ ID NO:81), a “myc” (c-myc) tag (SEQ ID NO:84), a SL2-tag (SEQ ID NO:80), a LSL-tag (SEQ ID NO:82) and a “FLAG” tag (SEQ ID NO:83). In one embodiment, said fusion polypeptide, comprises an amino acid sequence capable of binding to silica. In one embodiment, said fusion polypeptide, comprises an amino acid sequence selected from SEQ ID NO:80, SEQ ID NO:82 and amino acids sequences with at least 80%-identity to SEQ ID NO:80 or 82, such as wherein said amino acid sequence is SEQ ID NO:80 or 82. In one embodiment, said amino acid sequence capable of binding to silica SEQ ID NO:80. In another embodiment, said amino acid sequence capable of binding to silica SEQ ID NO:82.

As the skilled person understands, the construction of a fusion polypeptide often, but not always, involves the use of linkers between functional moieties to be fused. The skilled person is aware of different kinds of linkers with different properties, such as flexible amino acid linkers, rigid amino acid linkers and cleavable amino acid linkers. Linkers have been used to for example increase stability or improve folding of fusion polypeptides, to increase expression, improve biological activity, enable targeting and alter pharmacokinetics of fusion polypeptides.

Thus, in one embodiment of the first aspect there is provided an immunogenic composition comprising fusion polypeptides as disclosed herein or in one embodiment of the second aspect there is provided a fusion polypeptide, the fusion polypeptide comprises at least one linker selected from the group consisting of flexible amino acid linkers, rigid amino acid linkers and cleavable amino acid linkers. It will be appreciated that in the immunogenic composition any of the fusion polypeptide(s) present may comprise at least one linker. The skilled person will appreciate that the presence of a linker arranged between the two polypeptide fragments does not exclude the presence of additional linkers between other polypeptide fragments.

Flexible linkers are often used in the art when the joined domains require a certain degree of movement or interaction, and may be useful in some embodiments of the fusion polypeptide as described herein. Such linkers are generally composed of small, non-polar (for example G) or polar (for example S or T) amino acids. Some flexible linkers primarily consist of stretches of G and S residues, for example (GGGGS)ₚ and (SSSSG)ₚ (wherein GGGGS corresponds to SEQ ID NO:104 and SSSSG corresponds to SEQ ID NO:105). Adjusting the copy number “p” allows for optimization of the linker in order to achieve appropriate separation between the functional moieties or to maintain necessary inter-moiety interaction. Apart from G and S linkers, other flexible linkers are known in the art, such as G and S linkers containing additional amino acid residues, such as T, A, K and E, to maintain flexibility, as well as polar amino acid residues to improve solubility.

Additional non-limiting examples of linkers include GGGGSLVPRGSGGGGS (SEQ ID NO:85), (GS)₃ (SEQ ID NO:86), (GS)₄ (SEQ ID NO:87), (GS)₈ (SEQ ID NO:88), GGSGGHMGSGG (SEQ ID NO:89), GGSGGSGGSGG (SEQ ID NO:90), GGSGG (SEQ ID NO:91), GGSGGGGG (SEQ ID NO:92), GGGSEGGGSEGGGSEGGG (SEQ ID NO:93), AAGAATAA (SEQ ID NO:94), GGGGG (SEQ ID NO:95), GGSSG (SEQ ID NO:96), GSGGGTGGGSG (SEQ ID NO:97), GSGGGTGGGSG (SEQ ID NO:98), GT, GSGSGSGSGGSG (SEQ ID NO:99), GSGGSGGSGGSGGS (SEQ ID NO:100) and GSGGSGSGGSGGSG (SEQ ID NO:101). The skilled person is aware of other suitable linkers.

In one embodiment, said linker is a flexible linker comprising glycine (G), serine (S) and/or threonine (T) residues. In one embodiment, said linker has a general formula selected from (GₙSₘ)ₚ and (SₘGₙ)ₚ, wherein, independently, n=1-7, m=0-7, n+m≤8 and p=1-7. In one embodiment, n=1-5. In one embodiment, m=0-5. In one embodiment, p=1-5. In a more specific embodiment, n=4, m=1 and p=1-4. In one embodiment, said linker is selected from the group consisting of S₄G, (S₄G)₃ and (S₄G)₄. In one embodiment, said linker is selected from the group consisting of G₄S and (G₄S)₃. In one particular embodiment, said linker is G₄S and in another embodiment said linker is (G₄S)₃. In one embodiment, said linker has the general formula (GₑT_{f})_{q}, wherein, independently, e=1-3, f=1-3, and q=1-4. In one embodiment, q=1-3. In a more specific embodiment, e=1, f=1 and q=1-3. In one embodiment, said linker is selected from the group consisting of GT, (GT)₂ and (GT)₃. In one embodiment, said linker is GT. In one embodiment, the linker is EF.

In a third aspect of the present disclosure, there is provided a polynucleotide encoding an immunogenic polypeptide for use as defined herein, an immunogenic polypeptide fragment as described herein or a fusion polypeptide as described herein. Also encompassed by this disclosure is an expression vector comprising the polynucleotide and a host cell comprising the expression vector. Encompassed is also a method of producing an immunogenic polypeptide, an immunogenic polypeptide fragment or a fusion polypeptide, comprising culturing said host cell under conditions permissive of expression of said polypeptide from its expression vector, and isolating the polypeptide. Alternatively, the immunogenic polypeptide, immunogenic polypeptide fragment or fusion polypeptide of the present disclosure may alternatively be produced by non-biological peptide synthesis using amino acids and/or amino acid derivatives having protected reactive side-chains, the non-biological peptide synthesis comprising
step-wise coupling of the amino acids and/or the amino acid derivatives to form a polypeptide according to the first, second or third aspect having protected reactive side-chains,
removal of the protecting groups from the reactive side-chains of the polypeptide, and thereby
obtaining of the polypeptide in aqueous solution.

It will be appreciated that due to the non-structured nature of the fusion polypeptides according to aspect two, a method of production of said polypeptides may comprise for example contain a step of heating the polypeptides, such as a step of isolating or purifying the fusion polypeptides. Thus, in one embodiment, the method of producing a fusion polypeptide as defined herein is provided, wherein said method comprises heating the fusion polypeptide to approximately 100° C.

It will be appreciated that the polypeptide components of the immunogenic composition as described herein may be isolated or purified from S. suis. Alternatively, they may be produced according to recombinant techniques. Thus, in one embodiment, there is provided an immunogenic composition, comprising one or several fusion polypeptides which are recombinantly produced. The skilled person will appreciate that for the purpose of recombinant production, it may be beneficial in terms of cost to produce shorter polypeptides, such as fragments of full length proteins or fragments derived from full length proteins, as compared to polypeptides which correspond to said full length proteins. Additionally, it will be appreciated that it may also be beneficial to produce one polypeptide chain instead of two or more polypeptide chains, in order to reduce production costs. For example, it may be beneficial to produce one polypeptide chain comprising a fusion of several immunogenic polypeptide fragments instead of producing said several immunogenic polypeptide fragments separately.

The skilled person will appreciate that the fusion polypeptide(s) described herein according to the second aspect may be useful in immunogenic compositions. Such compositions may comprise one, two, three of all four of said fusion polypeptides according to the second aspect. Additionally, said immunogenic compositions may further comprise an additional fusion polypeptide comprising at least one, such as at least two, such as at least three of SEQ ID NO:34-36 or amino acid sequences with at least 80% identity to any one of SEQ ID NO:34-36. For example, said additional fusion polypeptide may comprises SEQ ID NO:46 or an amino acid sequence with at least 80% identity to SEQ ID NO:46; or may comprise SEQ ID NO:47 or an amino acid sequence with at least 80% identity to SEQ ID NO:47. said additional fusion polypeptide may comprises SEQ ID NO:107 or an amino acid sequence with at least 80% identity to SEQ ID NO:107; or may comprise SEQ ID NO:108 or an amino acid sequence with at least 80% identity to SEQ ID NO:108. Said additional fusion polypeptide may be as defined in as disclosed in context of the first aspect, the details thereof are not repeated here for the sake of brevity but are understood to be relevant here.

The skilled person will appreciate that the fusion polypeptide described herein or the immunogenic composition as disclosed herein may be useful as a medicament, for example as a medicament for use in prophylactic treatment of a S. suis infection, such as in vaccination. Thus in a fourth aspect of the present disclosure, there is provided a fusion polypeptide as defined herein, for use as a medicament. In another embodiment, said fusion polypeptide may be used in the prophylactic treatment of a S. suis infection. In one embodiment, said prophylactic treatment is of a S. suis infection selected from the group of infections caused by any one of the 35 identified serotypes of S. suis. In another embodiment, said infection is selected from the group of infections caused by any one of serotypes 1, 2, 7, 9 and ½, such as the group of infections caused by any one of serotypes 1, 2 and 7, such as the group of infections caused by serotype 2 and 7. In one embodiment, said infection is caused by S. suis serotype 2 and in another embodiment said infection is caused by S. suis serotype 7. In one embodiment, there is provided a fusion polypeptide as described herein, for use in the prophylactic treatment of a S. suis infection, which fusion polypeptide is effective in the prophylactic treatment of infection caused by any one of at least 2, such as at least 3, such as at least 5, such as at least 7, such as at least 10 of the 35 identified serotypes.

The skilled person will appreciate that any one of the above mentioned fusion polypeptides may be useful in an immunogenic composition, such as an immunogenic composition according to the first aspect as disclosed herein. In particular, the immunogenic composition may comprise at least one, such as two or three, fusion polypeptide(s) as disclosed in the second aspect of the present disclosure. In one embodiment, there is provided an immunogenic composition comprising at least one fusion polypeptide according to the second aspect disclosed herein. As explained in more detail above, said fusion polypeptide(s) is/are isolated or purified. Said fusion polypeptide(s) may be recombinantly produced.

It will be appreciated that the choice of suitable immunogenic polypeptides and fragments thereof is far from trivial and additionally the selection combinations thereof in fusion polypeptides adds an additional level of complexity. Immunogenic polypeptides and immunogenic polypeptide fragments, have to be selected taking into account the identity of the polypeptides, identity of the fragments thereof, the individual functionality of said polypeptides and fragments thereof (for example protease activity), their potential interaction (for example in terms of degradation), their stability in vivo and in vitro in the form of a fusion polypeptide, their solubility in vivo and in vitro in the form of a fusion polypeptide, their immunogenic properties, their ability to elicit an antibody response of the desired kind and their protective properties, just to mention a few.

The skilled person is aware of the fact that in order to elicit an immune response in a subject, an agent with adjuvant properties may be provided to said subject together with one or more immunogenic polypeptides, fragments or fusion polypeptides. Thus, in one embodiment, said immunogenic composition as described herein further comprises an agent with adjuvant effect. In particular, the agent with an adjuvant effect may be present in an immune-effective amount.

It will be appreciated that the immunogenic compositions as disclosed herein, may be useful as a medicament. Thus a related aspect of the present disclosure, there is provided the immunogenic composition for use as a medicament. In one embodiment, said immunogenic composition may be used in the prophylactic treatment of a S. suis infection. In one embodiment, said prophylactic treatment is of an S. suis infection selected from the group of infections caused by any one of the 35 identified serotypes of S. suis. In another embodiment, said infection is selected from the group of infections caused by any one of serotypes 1, 2, 7, 9 and ½, such as the group of infections caused by any one of serotypes 1, 2 and 7, such as the group of infections caused by serotype 2 and 7. In one embodiment, said infection is cause by S. suis serotype 2 and in another embodiment said infection is cause by S. suis serotype 7. In one embodiment, there is provided a fusion polypeptide as described herein, for use in the prophylactic treatment of a S. suis infection, which fusion polypeptide is effective in the prophylactic treatment of infection caused by any one of at least 2, such as at least 3, such as at least 5, such as at least 7, such as at least 10 of the 35 identified serotypes.

Thus, in a fifth aspect of the present disclosure, there is provided a vaccine composition comprising an immunogenic composition as defined herein and a pharmaceutically acceptable carrier or excipient. Said vaccine may be used in the prophylactic treatment of a S. suis infection as described above for the immunogenic composition. In one embodiment, said vaccine composition further comprises an agent with adjuvant effect. In one embodiment, said vaccine composition further comprises an immune-effective amount of an agent with adjuvant effect.

As used herein, the term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, sweetening agents, flavoring agents, dyes, and the like and combinations thereof, as would be known to those skilled in the art (see, for example, in Alfonso R Gennaro, Remington: The Science and Practice of Pharmacy. 20th edition, ISBN: 0683306472). Examples of pharmaceutically acceptable carriers include, but are not limited to sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; tale; excipients such as cocoa butter and suppository waxes; oils such as peanut oil, cottonseed oil, safflower oil, sesame oil, olive oil, corn oil and soybean oil; glycols such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate.

As used herein, the term “immune-effective” refers a sufficient amount of an adjuvant to increase the vaccine's immunogenicity to a level high enough to effectively vaccinate a typical patient.

As discussed above, an immunogenic composition as disclosed herein and/or a vaccine composition as disclosed herein may comprise an agent with adjuvant effect in an amount that is immuno-effective. Suitably, said adjuvant stimulates systemic or mucosal immunity. The skilled person is aware of suitable adjuvants. Non-limiting examples of suitable adjuvant in the context of the present disclosure include polymers of acrylic or methacrylic acid, maleic anhydride and alkenyl derivative polymers, immunostimulating sequences (ISS), an oil in water emulsion, cation lipids containing a quaternary ammonium salt, cytokines, aluminum hydroxide or aluminum phosphate, saponin or nanoparticles or any combinations or mixtures thereof. Further examples of suitable adjuvants may also be found in literature cited in WO 2007/115059.

A suitable adjuvant for use according to the present invention is the adjuvant Matrix V, Abisco/Matrix M, Matrix C or Matrix Q from Novavax, Sweden. Another suitable adjuvant is Ginseng. Ginseng is a dry extract prepared from the root of the plant Panax ginseng, C. A. Meyer. Ginseng contains a number of active substances named ginsenosides that are a kind of saponins, chemically tri-terpenoid glycosides of the dammaran series. The ginsenosides have adjuvant properties and one of the most active adjuvants is the fraction named Rb1. It has been proved that the fraction Rb1 elicits a balanced Th1 and Th2 immune response as determined by measuring the levels of the cytokines IFN-γ, IL-2, IL-4, IL-10 secreted post vaccination with a Rb1 adjuvanted vaccine. In addition ginseng and the fraction Rb1 stimulate a strong antigen specific antibody response.

In one embodiment, said agent with adjuvant effect is selected from the group consisting of polymers of acrylic or methacrylic acid, maleic anhydride and alkenyl derivative polymers, immunostimulating sequences (ISS), an oil in water emulsion, cation lipids containing a quaternary ammonium salt, cytokines, aluminum hydroxide, aluminum phosphate, saponin, nanoparticles, silica, Matrix V, Abisco/Matrix M, Matrix C, Matrix Q and silica. In one particular embodiment, said agent with adjuvant effect is Matrix V.

In one particular embodiment, said agent with adjuvant effect is selected from the group consisting of polymers of acrylic or methacrylic acid, maleic anhydride and alkenyl derivative polymers, immunostimulating sequences (ISS), an oil in water emulsion, cation lipids containing a quaternary ammonium salt, cytokines, aluminum hydroxide, aluminum phosphate, saponin, nanoparticles and silica. In one embodiment, said agent in selected from the group consisting of Matrix V, Abisco/Matrix M, Matrix C and Matrix Q. In one particular embodiment, said agent with adjuvant effect is Matrix V.

The vaccine composition of the present disclosure is formulated in a form suitable for physiological administration. Thus in one embodiment, there is provided a vaccine composition as described herein formulated for intramuscular, subcutaneous, intradermal or intranasal administration, such as for intramuscular administration.

In one embodiment, the vaccine composition as described herein, is, upon administration, capable of eliciting serum and/or mucosal antibody responses in a mammalian subject, such as a porcine or human subject, such as a porcine subject. In one embodiment, said antibody response is in the form of IgG, IgA and/or IgM antibodies in the serum and/or mucosa and/or colostrum. In one embodiment of the present aspect, there is provided a vaccine composition as described above, for use in the prophylactic treatment of a mammalian subject susceptible to S. suis infection, such as a human subject or porcine subject, such as a porcine subject. According to one embodiment, the vaccine composition is a vaccine that protects susceptible mammalian subjects, such as human or porcine subjects, against an infection caused by S. suis.

Suitably, the vaccine composition of the present invention stimulates serum, mucosal and/or bronchial antibody responses directed to S. suis antigens in mammalian subjects susceptible to these bacteria, such as in human or porcine subjects, such as porcine subjects.

As mentioned above, the present disclosure provides a vaccine composition comprising fusion polypeptides, which have been prepared according to the present method using E. coli as host cells, however other host cells may be used. The source of said fusion polypeptides might also be the native bacteria, if methods are developed for expression and purification thereof. Alternatively, the fusion strategies where various parts of the respective antigen are recombined may be employed resulting in a fusion polypeptide comprised of parts from different immunogenic polypeptides as described herein. This fusion strategy may also be suitable for introducing an immune reactive part(s), e.g. T-cell epitopes or attenuated toxins (or parts thereof), thereby introducing other features suitable for optimizing the antigen presentation or localization.

In a sixth aspect, the present disclosure also relates to a method for the production of an antiserum or colostrum, said method comprising administering an immunogenic composition as described herein to a mammalian host to produce antibodies in said host and recovering antiserum or colostrum containing the antibodies produced in said animal host. Within the scope of the present disclosure is also an antiserum or colostrum obtainable by said method. It is envisioned that the antiserum or colostrum may be administered to subject in need thereof, such as a mammalian subject susceptible to S. suis infection. For example said colostrum may be administered by feeding to piglets from unvaccinated sows, where piglets do not receive antibodies protective against S. suis infection by suckling the sow that gave birth to them.

Furthermore, antibodies may be recovered from said antiserum or colostrum as is appreciated by the person skilled in the art. Thus, also encompassed by the present disclosure is an antibody or fragment thereof, which is capable of binding to a fusion polypeptide as defined in herein, which antibody or fragment thereof is polyclonal or monoclonal. Also encompassed are antibody preparations comprising one antibody or several antibodies or fragments thereof. As well as said antibody preparation for use as a medicament, such as for use in the prophylactic treatment of a mammalian subject susceptible to Streptococcus suis infection.

Encompassed by the scope of the present disclosure are also antibody preparations comprising at least one, and suitably at least two, antibodies specific for a component of the immunogenic composition as described herein, which antibody/antibodies is/are polyclonal or monoclonal; or which preparation comprises a fragment of said antibodies. It is contemplated that said preparation could be used prophylactically against S. suis and provide passive immunization when administered to a mammalian subject susceptible to S. suis infection.

Thus, in one embodiment of seventh aspect of the present disclosure, there is provided an antibody or fragment thereof, which is specific for a fusion polypeptide as described herein, which antibody or fragment thereof is polyclonal or monoclonal. In another embodiment, there is provided an antibody preparation comprising one of several of said antibody/antibodies. Related hereto is a method for prophylactic treatment of a S. suis infection in a mammalian subject, comprising passive immunization by administering to said mammalian subject in need thereof said antibody preparation.

Thus, in one aspect there is provided an immunologically effective amount of an immunogenic composition as described above, a fusion polypeptide as described above, vaccine composition as described above, an antiserum or colostrum as obtainable by the method described above or an antibody preparation as described above, for use as medicament. In particular, there is provided an immunologically effective amount of an immunogenic composition as described above, a fusion polypeptide as described above, vaccine composition as described above, an antiserum or colostrum as obtainable by the method described above or an antibody preparation as described above, for use in the prophylactic treatment of a mammalian subject susceptible to S. suis infection, such as a human subject or porcine subject, such as a porcine subject.

In one embodiment, said use comprises administering on one single occasion or on multiple separate occasions. In particular, said use may comprise administration to piglets, gilts or sows. In one embodiment, said administration is to gilts or sows. In one embodiment, said administration is to piglets.

Said administration may be intramuscular, intradermal, subcutaneous or intranasal administration. Thus, in one embodiment said administration is selected from the group consisting of intramuscular, intradermal, subcutaneous or intranasal administration. In one embodiment, said administration is intramuscular administration.

In some embodiments, upon said administration of an immunogenic composition as described above, a fusion polypeptide as described above or vaccine composition as described above, a serum and/or mucosal antibody response is elicited in said mammalian subject. In particular said antibody response may be in the form of IgG, IgA and/or IgM antibodies in the serum and/or mucosa. Thus, in some embodiments of said use, the use comprises administering of an immunologically effective amount of said immunogenic composition, said fusion polypeptide, said vaccine composition, said antiserum, said colostrum or said antibody preparation at one single occasion.

In other embodiments, said use comprises administering of an immunologically effective amount of said immunogenic composition, said fusion polypeptide, said vaccine composition, said antiserum, said colostrum or said antibody preparation on multiple separate occasions, such as two, three, four or more separate occasions. In one embodiment the uses comprises administering on two separate occasions. In one embodiment, said administration is of said immunogenic composition or of said vaccine composition, such as of said vaccine composition. In one embodiment said multiple separate occasions are at least 2 weeks apart, such as 3-6 weeks apart, such as 3-5 weeks apart, such as 3-4 weeks apart, such as 3 or 4 weeks apart. In one embodiment, said administration is at two or three separate occasions. Said occasions may be for example at least approximately one week apart, such as at least approximately two weeks apart, such as at least approximately three weeks apart, such as at least approximately four weeks apart. In one particular embodiment said administration is approximately three weeks apart.

In one embodiment, said administration is in an effective amount of said immunogenic composition, said fusion polypeptide or said vaccine composition, which elicits an immune response resulting in protection. In one embodiment, said administration is in an amount of said antiserum, said colostrum or said antibody preparation, which results in protection. The immunogenic composition or vaccine compositions may be administered in a dose such that the amount of each fusion polypeptides is immunologically effective. In a particular embodiment, said administration is at a dose in the range of approximately 4-300 μg per fusion polypeptide. For example, said administration may be in the range of approximately 10-300 μg, such as in the range of approximately 10-250 μg, such as in the range of approximately 10-200 μg, such as in the range of approximately 50-150 μg, such as in the range of approximately 60-140 μg, such as in the range of approximately 70-130 μg, such as in the range of approximately 80-120 μg per fusion polypeptide. In one embodiment, said dose is in the range of approximately 90-110 μg, such as approximately 100 μg, per fusion polypeptide.

In one embodiment, said administration is to piglets, gilts or sows, such as to pregnant gilts or sows. In one embodiment, at least one administration is to a non-pregnant gilt or sow. In one embodiment, at least one administration is to a pregnant gilt or sow.

In the case wherein the priming dose alone elicits sufficient protective levels of immunity, the administration to piglets may be between postnatal day 1-28. For example said administration may be during the first postnatal week, for example at postnatal day 4. Alternatively, said administration may also be during postnatal week 2, 3 or 4. Thus, in one embodiment, said administration to piglets at one single occasion is during any one of postnatal weeks 1-4, such as any one of postnatal weeks 2-4, such as any one of postnatal weeks 3-4, such as during postnatal week 3 or 4. In one embodiment, said administration is during postnatal week 1.

In embodiments wherein a priming dose and a boosting dose are administered, the first administration to piglets may be during postnatal week 2-4, such as during postnatal week 3-4. In one embodiment, said administration is during postnatal week 1. In such embodiments, the second administration to piglets is at least 2 weeks, for example 3 or 4 or 5 weeks, after the first administration as described above. For example, the second administration to piglets may be during postnatal week 6-8.

It may be beneficial to make use of the immunogenic composition, fusion polypeptide or vaccine composition as described herein, wherein said immunogenic composition, fusion polypeptide or vaccine composition is administrated to a sow or gilt, to protect a piglet through the intake of colostrum from the said sow or gilt, against an infection caused by S. suis. Thus in another embodiment of the use disclosed herein, said administration is to pregnant or non-pregnant sows or gilts at one or two or three occasions at a time such as to obtain an immune response in colostrum at time of partum. For example such administration may be on two or three occasions. In one embodiment, the first administration is to pregnant sows or gilts approximately 6-8 weeks prior to partum and the second administration is approximately 2-4 weeks prior to partum. In one embodiment, the first administration is to pregnant sows or gilts approximately 9-10 weeks prior to partum and the second administration is approximately 7-6 weeks prior to partum and the third administration is approximately 4-3 weeks prior to partum. In one embodiment, the first administration is to pregnant sows or gilts approximately 9 weeks prior to partum and the second administration is approximately 6 weeks prior to partum and the third administration is approximately 3 weeks prior to partum. Approximately, in reference to the time point of administration discussed here, is to be interpreted as meaning within less than 1 week, such as less than 4 days, of said number of weeks prior to partum.

In one embodiment, said use comprises administration of an immunologically effective amount of said immunogenic composition, said fusion polypeptide or said vaccine composition to sows or gilts, such as pregnant sows or gilts, for the prophylactic treatment of piglets susceptible to S. suis infection. In one embodiment, said administration is at three separate occasions. In this way an immune response in colostrum at the time of partum is obtained. In one embodiment, said piglets obtain protective antibodies via the colostrum after birth. In one embodiment, said antibodies are protective antibodies which confer protection against S. suis infection, such as antibodies directed to one or more of the antigens present in the immunogenic composition, fusion polypeptide or said vaccine composition as defined herein.

In one embodiment, said piglets are protected at least at the age of 3 weeks, such as at the age of 4 weeks, such as at the age of 6 weeks, such as at the age of 7 weeks, such as at the age of 8 weeks, such at the age of 9 weeks, such as at the age of 10 weeks, or more. Piglets are commonly infected with S. suis from birth, but become most susceptible to disease after weaning at 3 to 4 weeks of age and at the age of 10-12 weeks the animals generally suffer from far fewer problems associated with S. suis infection. Thus, at the age of approximately 4-12, or approximately 4-10 weeks, piglets are particularly susceptible to infection. As shown in the appended Examples, piglets subjected to challenge with S. suis at the age of 4 or 7 weeks exhibited a significant level of protection and improved clinical scores compared to the control animals.

Non-pregnant gilts or sows may be vaccinated with at least one dose, such as two doses, and then a booster dose may be provided to pregnant gilt or sow 2 weeks before farrowing. Thus, the vaccination (prime and potential first booster dose) is administered to a non-pregnant animal and once pregnant the same animal received a booster (being a first or second booster dose) prior to farrowing. This is envisioned to maximize the immune responses provided to piglets in colostrum and reduce shedding of S. suis from sows. For example, gilts may be vaccinated with a priming dose at approximately 6 months of age and then vaccinated with a boosting dose 3 to 6 weeks later. Gilts or sows may then be then inseminated and would receive a third vaccination (second boosting dose) approximately 3 months and 1 week later, which corresponds to approximately 2 weeks prior to farrowing. Thus, in one embodiment, there is provided said immunogenic composition, said fusion polypeptide or said vaccine composition for use as described herein, wherein said use comprises at least one administration of an immunologically effective dose of said immunogenic composition, said fusion polypeptide or said vaccine composition to gilt or sow in a non-pregnant state and at least one administration of an additional immunologically effective dose said gilt or sow in a pregnant state. In one embodiment, said use comprises two administrations to the gilt or sow in the non-pregnant state. In one embodiment, said non-pregnant gilt is approximately 4-8, such as 5-7, such as approximately 6 months when the first effective dose is administered. In one embodiment, the second dose is administered approximately after approximately 3-7 weeks, such as after approximately 3, 4, 5, 6 or 7 weeks. In one embodiment, said use comprises administration of an additional effective dose after the insemination of the gilt or sow, thus to the gilt or sow in a pregnant state. Said administration of an additional effective dose could be approximately 2 weeks prior to farrowing. In this way an immune response in colostrum at the time of partum is obtained. In one embodiment, said piglets obtain protective antibodies via the colostrum after birth.

As explained above, said administration is at a dose in the range of approximately 4-300 μg per fusion polypeptide. For example, said administration may be in the range of approximately 10-300 μg, such as in the range of approximately 10-250 μg, such as in the range of approximately 10-200 μg, such as in the range of approximately 50-150 μg, such as in the range of approximately 60-140 μg, such as in the range of approximately 70-130 μg, such as in the range of approximately 80-120 μg per fusion polypeptide. In one embodiment, said dose is in the range of approximately 90-110 μg, such as approximately 100 μg, per fusion polypeptide.

In an eighth aspect of the present disclosure, there is provided a method for prophylactic treatment of a S. suis infection in a mammalian subject, comprising administering to said mammalian subject in need thereof an immunologically effective amount of an immunogenic composition as described above, a fusion polypeptide as described above, a vaccine composition as described above, an antiserum or colostrum as obtainable by the method described above or an antibody preparation as described above. In one embodiment, said mammalian subject is a porcine or human subject, such as a porcine subject.

The skilled person will appreciate that many vaccines require administration of more than one dose in order to elicit a protective immune response. The first dose administered to a naïve subject, often referred to as a priming dose, directs the immune system to recognize the foreign antigen. In some cases, the priming dose alone may elicit sufficient protective levels of immunity. However, in other cases the priming dose may not elicit protective levels of immunity. Therefore, priming doses may be followed by one or several subsequently administrations of the identical vaccine in order to increase the magnitude of the antigen specific immune responses. These subsequently administered doses are referred to as boosting doses. Suitably, the elicited immune response is in the form of IgG, IgA and/or IgM antibodies in the mucus and/or serum of piglets or sows/gilts, and/or colostrum of sows.

In a related aspect as disclosed herein, there is provided a method for prophylactic treatment of a S. suis infection in a mammalian subject, comprising administering to said mammalian subject in need thereof an immunologically effective amount of an immunogenic composition, a fusion polypeptide, a vaccine composition, an antiserum or colostrum or an antibody preparation as disclosed herein. It will be appreciated that the embodiments disclosed in the context of aspects relating to the first or second medical use according to the present disclosure are equally relevant for the method for prophylactic treatment of a S. suis infection and will not be repeated for the sake of brevity, but only mentioned briefly. In one embodiment, there is provided a method for prophylactic treatment, wherein said mammalian subject is a porcine or human subject, such as a porcine subject.

In one embodiment, said method comprises administering on one single occasion or on multiple separate occasions. In one embodiment, said administration is to piglets, gilts or sows, such as pregnant gilts or sows or non-pregnant gilts or sows. Said administration may be intramuscular, intradermal, subcutaneous or intranasal administration, such as intramuscular administration. In one embodiment, wherein said administration is of an immunogenic composition, a fusion polypeptide or a vaccine composition, upon administration, serum and/or mucosal antibody response is elicited in said mammalian subject. In particular, said antibody response is in the form of IgG, IgA and/or IgM antibodies in the serum and/or mucosa and/or colostrum. In one embodiment, said method comprises administration of an effective amount of said immunogenic composition, said fusion polypeptide or said vaccine composition to sows or gilts, such as pregnant sows or gilts, for the prophylactic treatment of piglets susceptible to S. suis infection. In one embodiment, said administration is at three separate occasions.

In a particular embodiment, there is provided a method of prophylactic treatment of piglets susceptible to S. suis infection, comprising the steps of administering at least two, such as at least three, separate occasions an immunologically effective amount of an immunogenic composition as described above, a fusion polypeptide as described above, vaccine composition as described above to a pregnant gilt or sow. In this way an immune response in colostrum at time of partum is obtained. In one embodiment, said piglets obtain protective antibodies via the colostrum after birth. Additionally, there is provided a method of prophylactic treatment of piglets susceptible to S. suis infection, comprising the steps of administering at least one or at least two separate occasions an immunologically effective amount of an immunogenic composition as described above, a fusion polypeptide as described above, vaccine composition as described above to a gilt or sow in a non-pregnant state and at least one administration to an immunologically effective amount of an immunogenic composition as described above, a fusion polypeptide as described above, vaccine composition as described above to said gilt or sow in a pregnant state prior to farrowing. For example, such as approximately 4-1 week, such as approximately 3-2 weeks prior to farrowing. The immunogenic composition or vaccine compositions may be administered in a dose such that the amount of each fusion polypeptide is immunologically effective. Thus, in one embodiment, said administration is at a dose in the range of approximately 4-300 μg per fusion polypeptide. For example, said administration may be in the range of approximately 10-300 μg, such as in the range of approximately 10-250 μg, such as in the range of approximately 10-200 μg, such as in the range of approximately 50-150 μg, such as in the range of approximately 60-140 μg, such as in the range of approximately 70-130 μg, such as in the range of approximately 80-120 μg per fusion polypeptide. In one embodiment, said dose is in the range of approximately 90-110 μg, such as approximately 100 μg, per fusion polypeptide.

Also, there is provided a method for prophylactic treatment of a S. suis infection in a mammalian subject, comprising passive immunization by administering to said mammalian subject in need thereof an antibody preparation as disclosed herein or antiserum or colostrum comprising said antibodies as described herein.

In a related aspect there is provided a method for immunizing a mammalian subject against a Streptococcus suis infection, comprising administering to said mammalian subject in need thereof an immunologically effective amount of an immunogenic composition as defined herein, a fusion polypeptide as defined herein, a vaccine composition as defined herein, an antiserum or colostrum as defined herein or an antibody preparation as defined herein. In one embodiment, said method comprising administering to said mammalian subject in need thereof an immunologically effective amount of an immunogenic composition as defined herein, a fusion polypeptide as defined herein or a vaccine composition as defined herein. It will be appreciated that all embodiments defined in relation to the aspect of a method for prophylactic treatment of a S. suis infection in a mammalian subject above, are equally relevant for the method for immunizing according to this aspect.

In yet another aspect of the present disclosure, there is provided the use of an immunogenic composition, fusion polypeptide, vaccine composition, antiserum, colostrum or antibody preparation as described herein, for the manufacture of a medicament for use in the prophylactic treatment of a mammalian subject susceptible to S. suis infection. The skilled person will appreciate that embodiment discussed in relation to aspects four to eight as disclosed herein are equally relevant for this aspect.

While the invention has been described with reference to various exemplary aspects and embodiments, it will be understood by those skilled in the art that various changes may be made and equivalents may be substituted for elements thereof without departing from the scope of the invention. In addition, many modifications may be made to adapt a particular situation or molecule to the teachings of the invention without departing from the essential scope thereof. Therefore, it is intended that the invention not be limited to any particular embodiment contemplated, but that the invention will include all embodiments falling within the scope of the appended claims.

### LIST OF REFERENCES

Mészáros et al. (2018) Nucleic Acids Research; 46(W1):W329-W337.
Erdös and Dosztányi (2020) Current Protocols in Bioinformatics; 70(1):e99.
Fittipaldi et al. (2012), Future Microbiol. 7 (2); 259-279.
Rasmussen and Andresen (1998), Int. J. Syst. Bacteriol. 48, 1063-1065,
Berthelot-Herault et al. (2005) Can J Vet Res. July; 69(3):236-40.
Quessy et al. (1995) Infect Immun. May; 63(5):1975-9.
Vecht et al. (1992) Infect Immun. February; 60(2):550-6.
Li et al. (2011) FEMS Micro Letters 316: 115-122.
Gómez-Gascóna et al. (2012), Journal of Proteomics October 22; 75(18):5654-66.
Seele et al. (2015) Vaccine, 33, 2207-2212.
MacRaild C et al. (2016) Structure, January 5; 24(1):148-157.
Kebede et al. (1990), Vet. Microbiol. 22: 249-257
Liu et al. (2014) The Journal of Infectious Diseases, Vol 210, Issue 1, p. 35-45

### BRIEF DESCRIPTION OF FIGURES

FIG. 1 is a schematic drawing of the five fusion polypeptides chosen as vaccine candidates.

FIG. 2A-E shows the results from a stability study of fusion polypeptides SP274C-S, BCA, 4Zn+3, 5AsL and 3PCS, corresponding to SEQ ID NO:52, 49, 50, 51 and 54, respectively.

FIG. 3 show the results from immunizations of rabbits with SP274C-S, BCA, 4Zn+3 and 5AsL, corresponding to SEQ ID NO:52, 49, 50, and 51.

FIG. 3A shows the SDS PAGE gel of the fusion polypeptides and single proteins; FIGS. 3B and 3C shows the results from Western-blots of pre-immunization and post-immunization sera.

FIG. 4. Shows the results from immunization of sows, transfer of immunoglobulin to piglets and immunization of piglets by measuring the amount of antibody directed to the components of Piggivac4.

FIG. 5 is a Kaplan-Meier survival curve for piglets in groups 3 and 4. The data shows that more piglets from the Piggivac4-vaccinated sow reached the end of the study at 7 days post-challenge.

FIG. 6 is a graph of the mean rectal temperatures of piglets in groups 3 and 4. The arrow indicates time of challenge with significant differences highlighted. The data shows that piglets from the Piggivac4-vaccinated sow had significantly lower rectal temperatures on day 1 and 2 post-challenge.

FIG. 7 is a Kaplan-Meier survival curve of piglets without pyrexia (defined as a rectal temperature of >40° C. for two out of three consecutive observations) in groups 3 and 4. The data shows that the onset of rises in rectal temperature was also significantly delayed in piglets from the Piggivac4-vaccinated sow compared to the control.

FIG. 8 is graph showing the comparison of demeanour scores in the piglets in groups 3 and 4 post-challenge. The arrow indicates the time of challenge. The data shows that piglets from the Piggivac4-vaccinated sow had lower demeanour scores post-challenge.

FIG. 9 is a Kaplan-Meier survival curve of piglets with demeanour scores below 2 (for two out of three consecutive observations) in groups 3 and 4.

FIG. 10 is a graph showing the comparison of the CNS scores in the piglets in groups 3 and 4 post-challenge. The arrow indicates the time of challenge. The data shows that piglets from the Piggivac4-vaccinated sow had lower CNS scores post-challenge.

FIG. 11 is a Kaplan-Meier survival curve of piglets with CNS scores below 1 (for two out of three consecutive observations) in groups 3 and 4.

FIG. 12 is a graph showing a comparison of the mobility scores in the piglets in groups 3 and 4 post-challenge. The arrow indicates the time of challenge. The data shows that piglets from the Piggivac4-vaccinated sow had lower mobility scores post-challenge.

FIG. 13 is a Kaplan-Meier survival curve of piglets with mobility scores below 1 (for two out of three consecutive observations) in groups 3 and 4.

FIG. 14 is a graph showing the comparison of the total clinical scores in the piglets in groups 3 and 4 post-challenge. The arrow indicates the time of challenge. The data shows that piglets from the Piggivac4-vaccinated sow had lower total clinical scores post-challenge.

FIG. 15 is a Kaplan-Meier survival curve of piglets with total scores below 5 (for two out of three consecutive observations) in groups 3 and 4.

FIG. 16 shows a Kaplan-Meier survival curve for piglets in Groups 3 and 4. Piglets were euthanized on reaching the humane endpoint. The data shows that piglets from the Piggivac5-vaccinated sows tended to take longer to reach the humane endpoint.

FIG. 17 shows mean rectal temperatures of piglets in Groups 3 and 4. Significant differences are highlighted. The data shows that piglets from the Piggivac5-vaccinated sows had significantly lower body temperatures for up to 6 days post-challenge.

FIG. 18 is a Kaplan-Meier survival curve showing the fraction of piglets without pyrexia (defined as a rectal temperature of >40.5° C.) in Groups 3 and 4 over time. The data shows that piglets from the Piggivac5-vaccinated sows had a highly significant delay in the onset of fever (P=0.00003).

FIG. 19 shows the comparison of demeanour scores in the piglets in Groups 3 and 4 post-challenge. Significant differences are highlighted. The data shows that piglets from the Piggivac5-vaccinated sows had significantly lower demeanour scores for up to 12 days post-challenge.

FIG. 20 is a Kaplan-Meier survival curve showing the fraction of piglets with demeanour scores below 1 in Groups 3 and 4 over time. The data shows that piglets from the Piggivac5-vaccinated sows had a highly significant delay in the onset of changes in demeanour (P=0.000004).

FIG. 21 shows a comparison of the CNS scores in the piglets in Groups 3 and 4 post-challenge. Significant differences are highlighted. The data shows that piglets from the Piggivac5-vaccinated sows had significantly lower CNS scores for up to 10 days post-challenge.

FIG. 22 is a Kaplan-Meier survival curve showing the fraction of piglets with CNS scores below 1 in Groups 3 and 4. The data shows that piglets from the Piggivac5-vaccinated sows had a highly significant delay in the onset of changes in CNS signs (P=0.0005).

FIG. 23 shows a comparison of the mobility scores in the piglets in Groups 3 and 4 post-challenge. Significant differences are highlighted. The data shows that piglets from the Piggivac5-vaccinated sows had significantly lower mobility scores post-challenge.

FIG. 24 is a Kaplan-Meier survival curve of the fraction of piglets with mobility scores <2 in Groups 3 and 4. The data shows that piglets from the Piggivac5-vaccinated sows had a highly significant delay in the onset of changes in mobility signs (P=0.003).

FIG. 25 shows a comparison of the total clinical scores in the piglets in Groups 3 and 4 post-challenge. Significant differences are highlighted. The data shows that piglets from the Piggivac5-vaccinated sows had significantly lower total clinical scores for up to 10 days post-challenge.

FIG. 26 is a Kaplan-Meier survival curve of the fraction of piglets with total scores below 5 in Groups 3 and 4. The data shows that piglets from the Piggivac5-vaccinated sows had a highly significant delay in the onset of changes in total clinical signs (P=0.00006).

FIG. 27 shows a comparison of the weights of piglets in Groups 3 and 4 post-challenge. Significant differences are highlighted. The data shows that piglets from the Piggivac5-vaccinated sows gained significantly more weight over the 14 days post-challenge (P=0.03).

FIG. 28 shows a Kaplan-Meier survival curve for piglets in Groups 5 and 6. Piglets were euthanized on reaching the humane endpoint. The data shows that piglets from the Piggivac5-vaccinated sows tended to take longer to reach the humane endpoint.

FIG. 29 shows mean rectal temperatures of piglets in Groups 5 and 6. The data shows that piglets from the Piggivac5-vaccinated sows tended to have lower body temperatures post-challenge.

FIG. 30 is a Kaplan-Meier survival curve showing the fraction of piglets without pyrexia (defined as a rectal temperature of 41° C.) in Groups 5 and 6 over time. The data shows that piglets from the Piggivac5-vaccinated sows tended to have a delay in the onset of fever.

FIG. 31 shows the comparison of demeanour scores in the piglets in Groups 5 and 6 after challenge. Significant differences are highlighted. The data shows that piglets from the Piggivac5-vaccinated sows had significantly lower demeanour scores on 10 days post-challenge.

FIG. 32 is a Kaplan-Meier survival curve showing the fraction of piglets with demeanour scores below 3 in Groups 5 and 6 over time. The data shows that piglets from the Piggivac5-vaccinated sows had a significant delay in the onset of changes in demeanour (P=0.01).

FIG. 33 shows a comparison of the CNS scores in the piglets in Groups 5 and 6 after challenge. Significant differences are highlighted. The data shows that piglets from the Piggivac5-vaccinated sows had significantly lower CNS scores on day 2 post-challenge.

FIG. 34 is a Kaplan-Meier survival curve showing the fraction of piglets with CNS scores below 1 in Groups 5 and 6. The data shows that piglets from the Piggivac5-vaccinated sows had a significant delay in the onset of changes in CNS signs (P=0.005).

FIG. 35 shows a comparison of the mobility scores in the piglets in Groups 5 and 6 after challenge. Significant differences are highlighted. The data shows that piglets from the Piggivac5-vaccinated sows had significantly lower mobility scores on 11 days post-challenge.

FIG. 36 is a Kaplan-Meier survival curve of the fraction of piglets with mobility scores <3 in Groups 5 and 6. The data shows that piglets from the Piggivac5-vaccinated sows had a significant delay in the onset of changes in mobility signs (P=0.02).

FIG. 37 shows a comparison of the total clinical scores in the piglets in Groups 5 and 6 after challenge. Significant differences are highlighted. The data shows that piglets from the Piggivac5-vaccinated sows had significantly lower total clinical scores on 10 days post-challenge.

FIG. 38 is a Kaplan-Meier survival curve of the fraction of piglets with total scores below 7 in Groups 5 and 6. The data shows that piglets from the Piggivac5-vaccinated sows had a significant delay in the onset of changes in total clinical signs (P=0.03).

FIG. 39 shows a comparison of the weights of piglets in Groups 5 and 6 after challenge on Day 23. The data shows that piglets from the Piggivac5-vaccinated sows had gained more weight over the 14 days post-challenge, but that this did not achieve statistical significance (P=0.19).

FIG. 40 shows the amino acid sequences of the polypeptides disclosed herein. In particular, SEQ ID NO 1-19 shows full length polypeptides, SEQ ID NO: 20-42 shows fragments thereof and SEQ ID NO: 43-55 shows fusion polypeptides as disclosed herein. In particular in SEQ ID NO:49-53 and 55 show the fusion polypeptides translated from the first methionine to the stop codon. Note that amino acid residues in bold originate from vector and/or cloning site and amino acid residues in italics indicate either a tag, such as a His-tag or SL2-tag. Importantly, note that the amino acids in bold originate from the construction work of the fusion polypeptide and/or from the expression vector utilized and that these amino acids could be changed or even absent if another fusion strategy is used.

### EXAMPLES

In the following examples the identification, cloning and purifications of antigen fragments as well as construction of fusion polypeptides comprising said fragments for use in a vaccine against S. suis infection is disclosed. Also disclosed is data from a vaccine study in porcine subjects using said fusion polypeptides.

### General Materials and Methods

The molecular work has been done according to the information in “Molecular Cloning: A Laboratory Manual” by J. Sambrook, E. F. Fritsch, T. Maniatis. The different methods used are described in short below.

Electroporation of E. coli: In all instances the strain E. coli BL21 have been used as recipient of plasmids. 1 μl of plasmid was added to 50 μl of electrocompetent cells (on ice) and the mixture was transferred to cold cuvettes (1 mm gap). The conditions for the electroporation were; 200-400 ohm, 25 microfarad and 2.5 kV. After the electroporation the cells were resuspended in 1 ml LB-medium and incubated for one hour at 37° C. (phenotypic expression) after which the cells are spread on LB-plates with the appropriated antibiotics.

Liqations: The ligations were performed using T4 DNA ligase (New England Biolabs) in a DNA concentration of approximately 20-50 μg/ml for at least one hour. Thereafter the DNA was EtOH precipitated, washed with 70% EtOH and dried. The pellet was dissolved in H₂O and used for electroporation.

Purification of DNA: For plasmid purification, an overnight growth culture of E. coli BL-21 was harvested of which 2-4 ml was used for plasmid preparation. The plasmid was purified using QiAprep Spin Miniprep kit (Qiagen) according to the supplier's instructions. Purification of DNA after CIP treatment of PCR products was done by using QIAquick PCR purification kit (QIAGEN).

Cleavage with restriction enzymes: The appropriated buffer was added to the DNA and DTT (Dithiothreitol; Amersham Biosciences) was added to a final concentration of 1 mM. Thereafter the restriction enzyme(s) (New England Biolabs) was added to a concentration of 2-10 units/μg and the mixture was incubated for 2-3 hours.

CIP treatment of vectors: All vectors used for cloning were treated with Calf Intestinal Alkaline Phosphatase (CIP) (New England Biolabs) for one hour in the same buffer used for restriction enzyme cleavage. Thereafter the vector was purified using QIAquick PCR purification kit (QIAGEN).

PCR conditions: Fidelity Taq PCR Master Mix (USB, Affymetrix) was been used according to the instructions. If not stated otherwise, the annealing temperature has been 5-10° C. under the melting point for the primers, the number of cycles has been 30 and the extension time has been approximately 1 minute per 1 kbp of DNA.

DNA sequencing: All DNA sequencing was performed by the Uppsala Genome Center Sequencing Service (Uppsala, Sweden).

SDS-PAGE analysis: SDS-PAGE analyses were performed using the PhastSystem (Cytiva). Samples were analyzed under reducing conditions using precasted 8-25% gradient gels.

Expression of recombinant proteins: In general, similar conditions have been used for all proteins expressed in this application. A single colony was inoculated into 20-30 ml LB medium supplemented with kanamycin (25-50 μg/ml) and the culture was incubated while shaking over night at 25° C. From the overnight culture 10-20 ml was inoculated into 1|LB supplemented with kanamycin (25-50 μg/ml) and the culture was incubated with shaking at 25° C. for a few hours (5-7 hours). The expression of recombinant protein was induced by addition of 1|LB supplemented with kanamycin (25-50 μg/ml) and 0.2 mM isopropyl β-D-1-thiogalactopyranoside (IPTG) and the culture was incubated while shaking over night at 25° C.

Protein lysate: After the induction phase, the culture was harvested by centrifugation and the cells are resuspended in lysis buffer (100 mM NaCl, 20 mM Tris (pH 7-7.5) and 0.05% Tween 20) and 10-100 μg/ml lysozyme was added after which the cells were lysed using freeze/thaw cycles. The lysate was then sonicated (40-60% amplitude) on ice-water for 2×10 minutes with 1 second pulses. The lysate was centrifuged for 20-30 minutes at 10K RPM. The supernatant was collected and sterile filtered (using a 0.45 μm filter) after which the lysate was frozen in aliquots.

Plasmid vectors: The plasmid vectors used in the present Examples are the following:
pBmKny, BmKSL2,pUC57-mini and PGex6P-1.

The construction and details on pBmKny and BmKSL2 have been previously described in WO 2017/005913.

The pUC57-mini vector and the PGex6P-1 (both obtained from GenScript) were also used. Details on said vectors can be found at GenScript (http://genscript.com/)

Primers: Primers used in the experiments described in the following examples are shown in Table 1 below.

TABLE 1
List of primers/oligonucleotides used herein.
SEQ ID NO:

| | | |
|---|---|---|
| 68 | Tac5 | GCCGACATCATAACGGTTCTGG |
| 69 | NP7Kpn | TATAGGTACCGGTTTGGTTGCCACTTGC |
| 70 | SP4CKpnI | ATATGGTACCGGCGAAACGACGGC |
| 71 | BmR2 | GGTGGTCCCACCTGACGTC |
| 72 | SP2Cr | TATAGAATTCACCTGCTTTGGTCGTTTTTGC |
| 73 | SP74Cs | ATATGAATTCGAAATTCAAGCCATTGTGGACG |

### Example 1

### General Description of Antigens Chosen

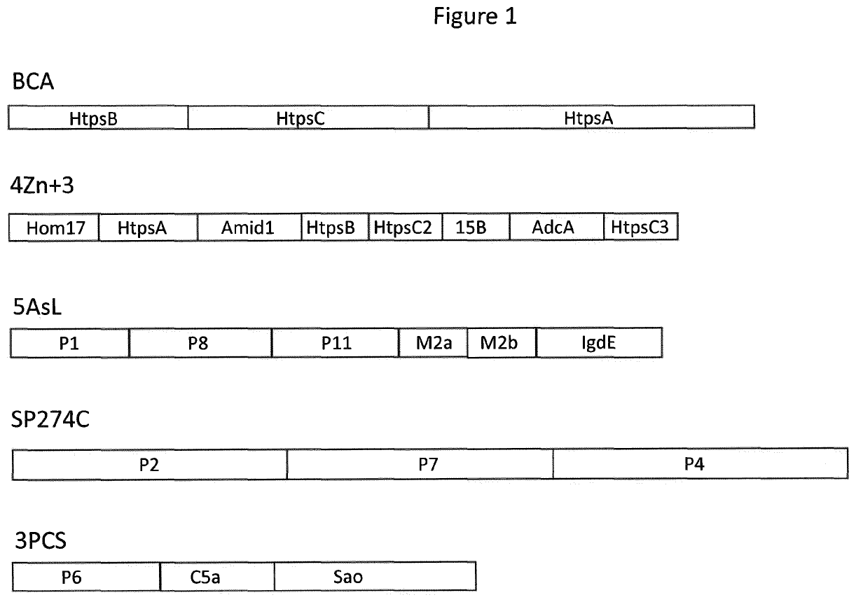

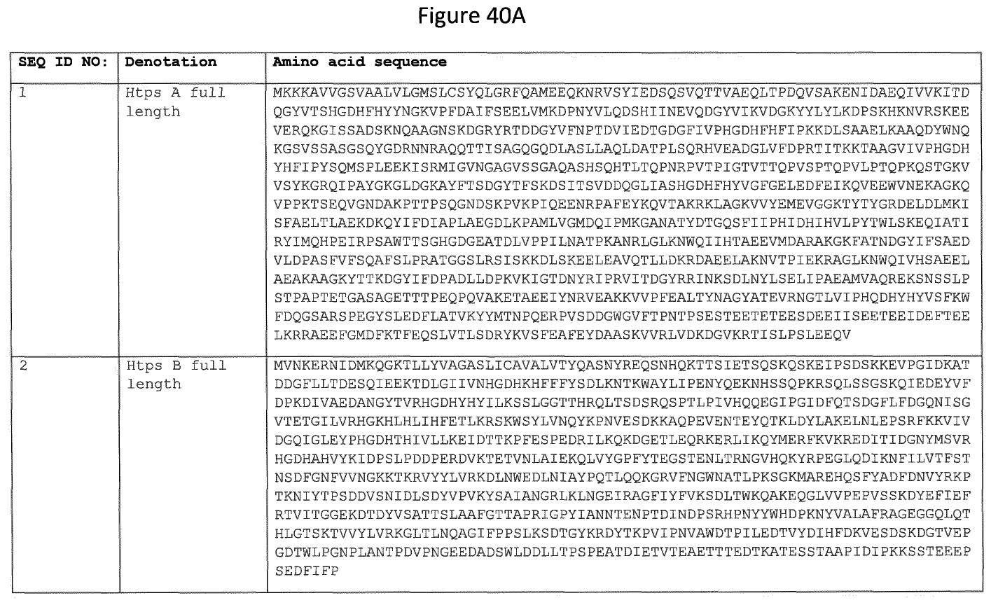

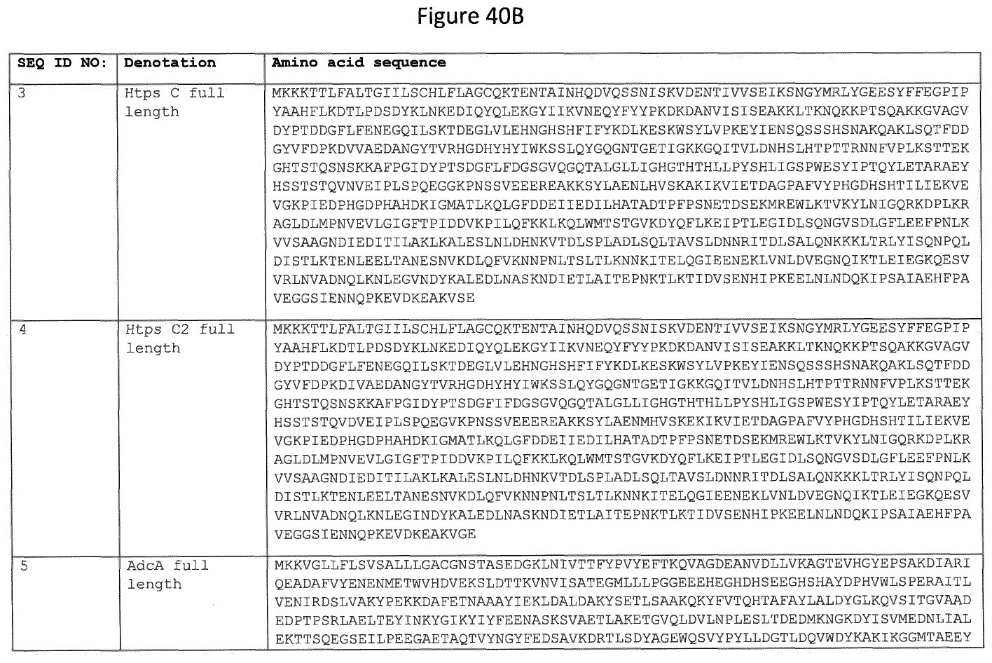

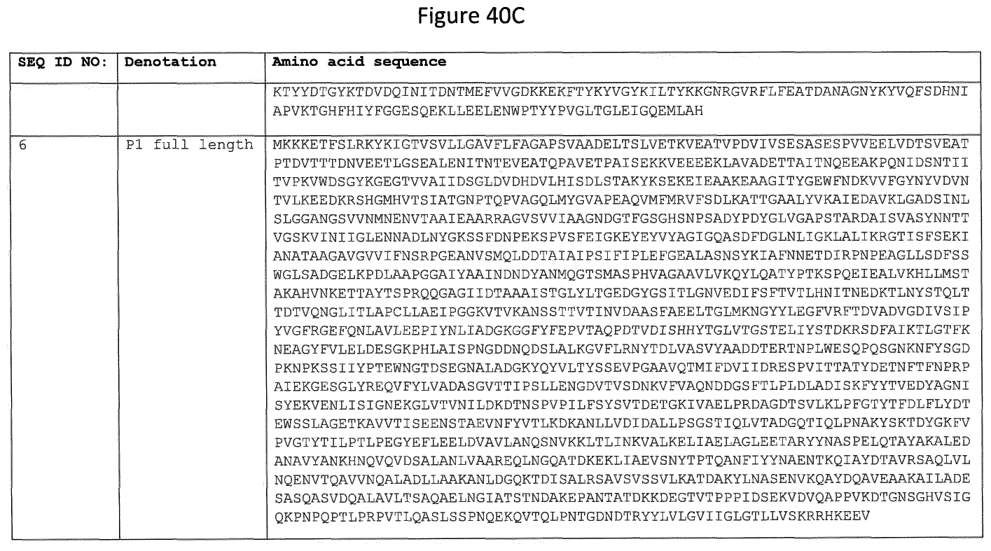

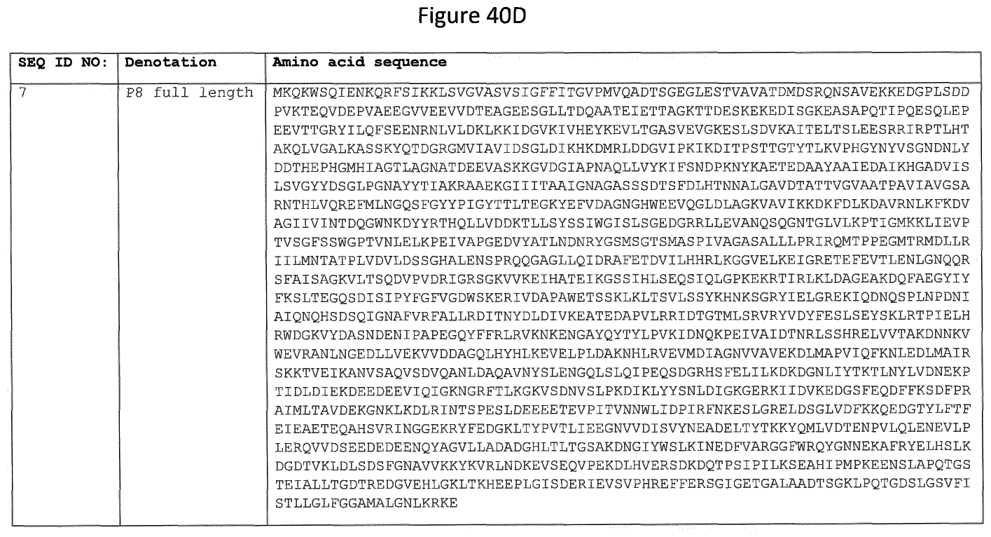

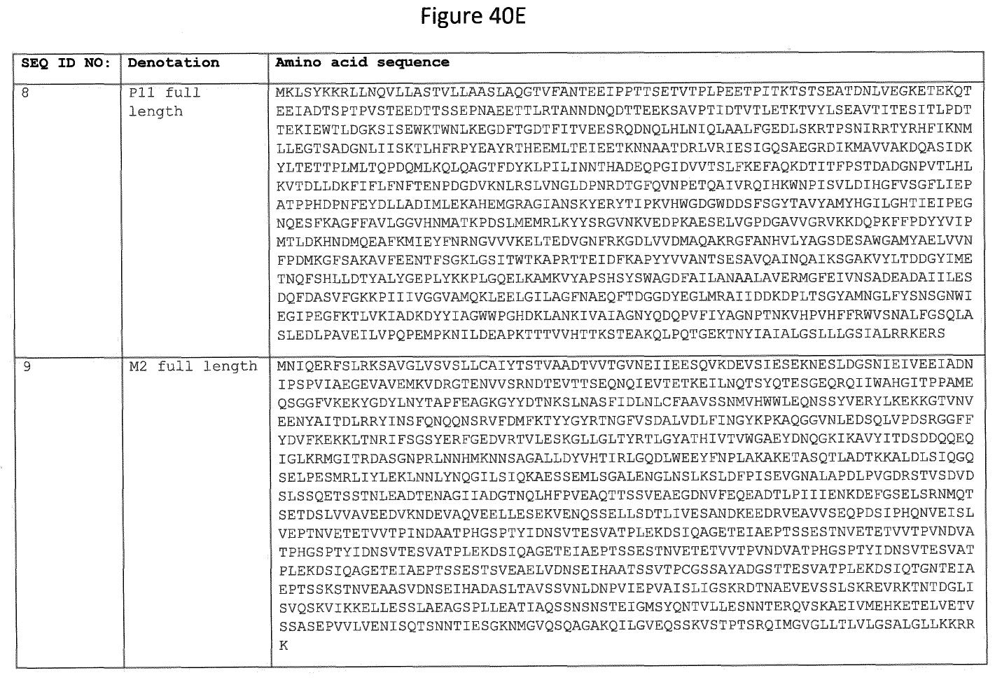

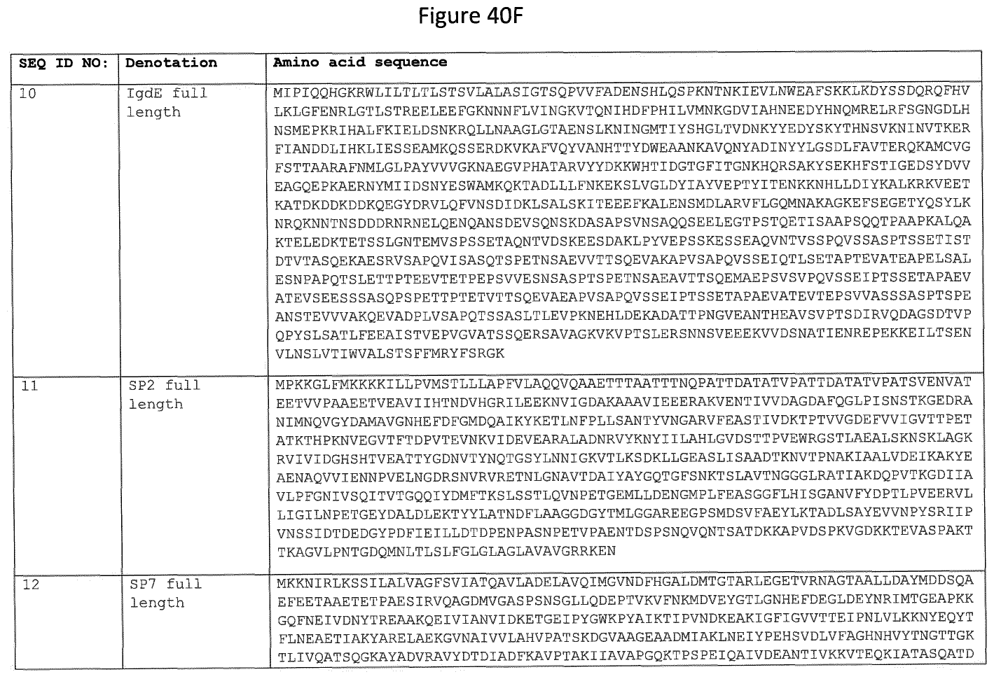

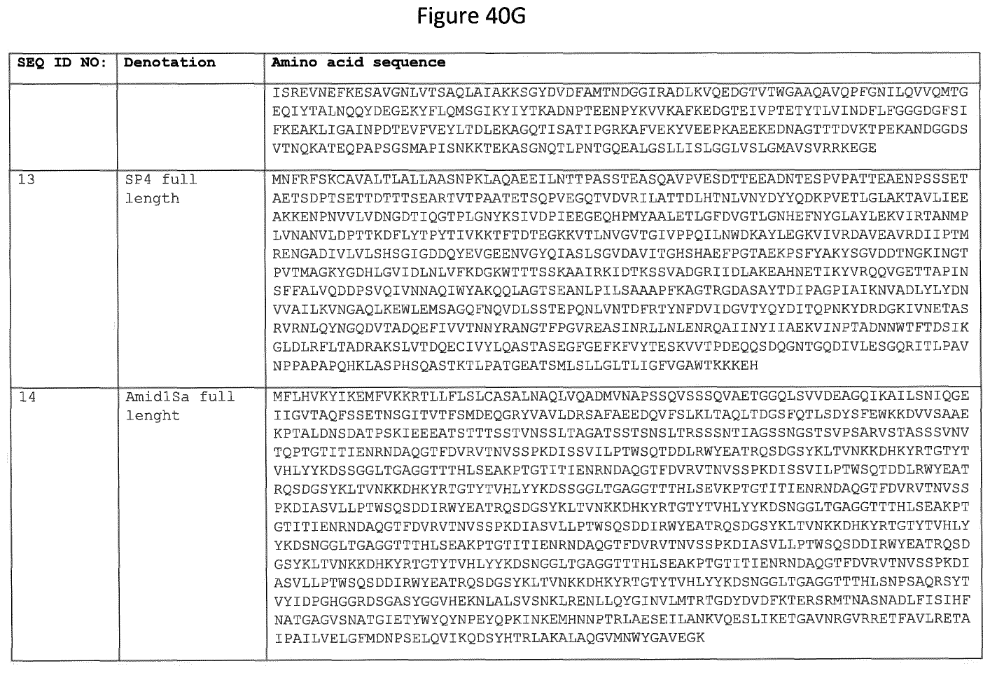

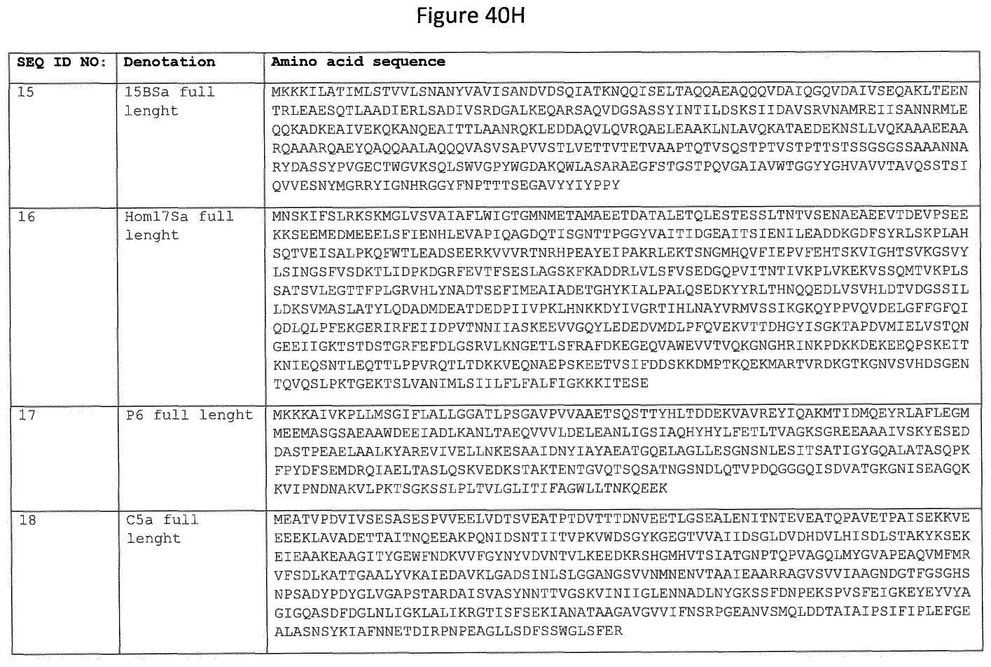

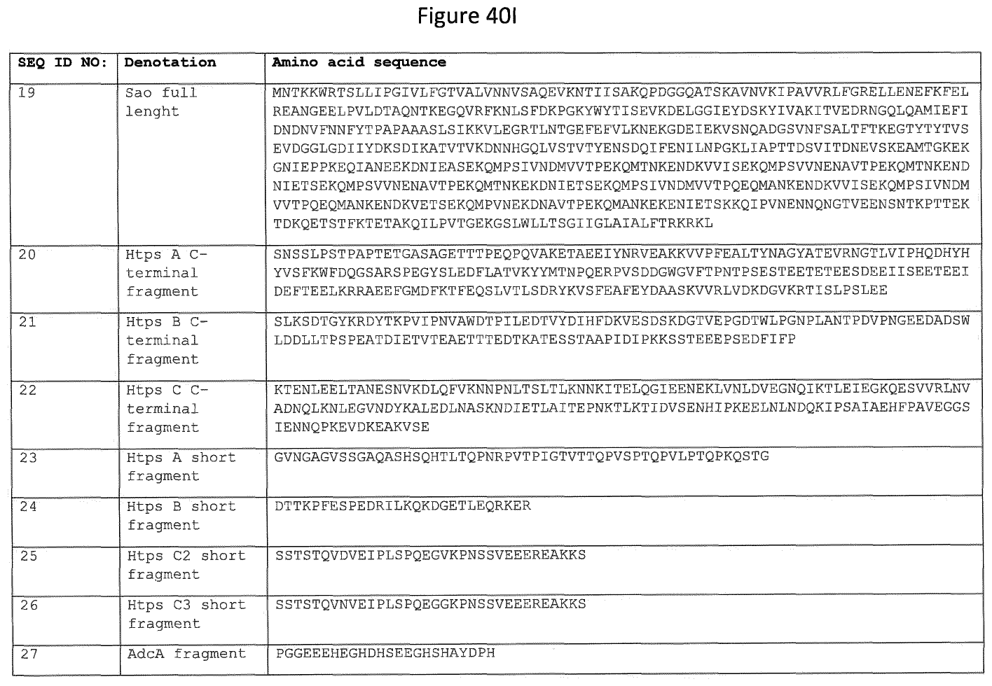

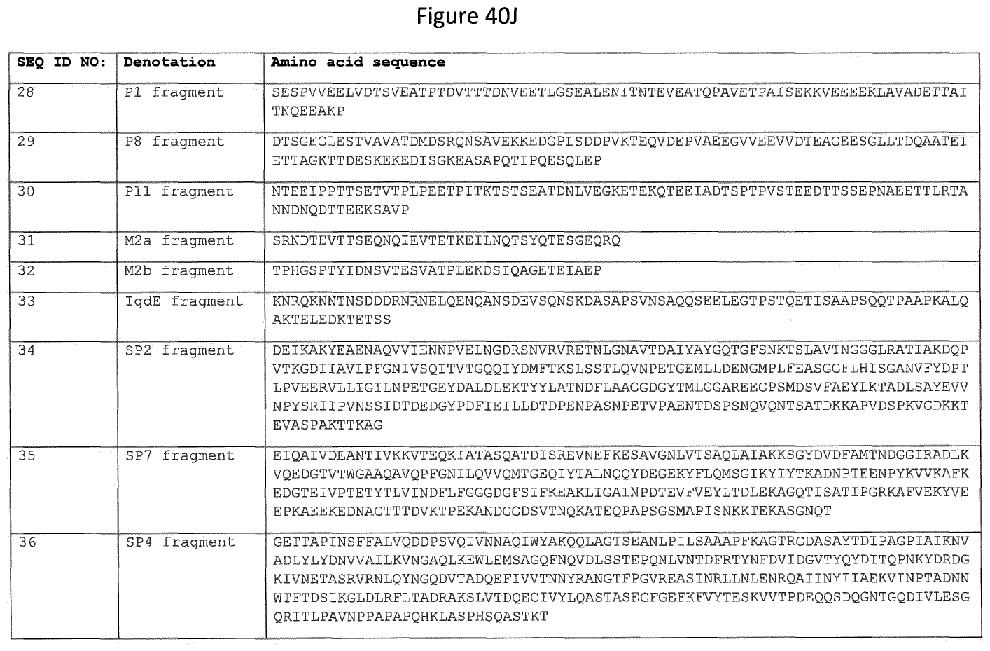

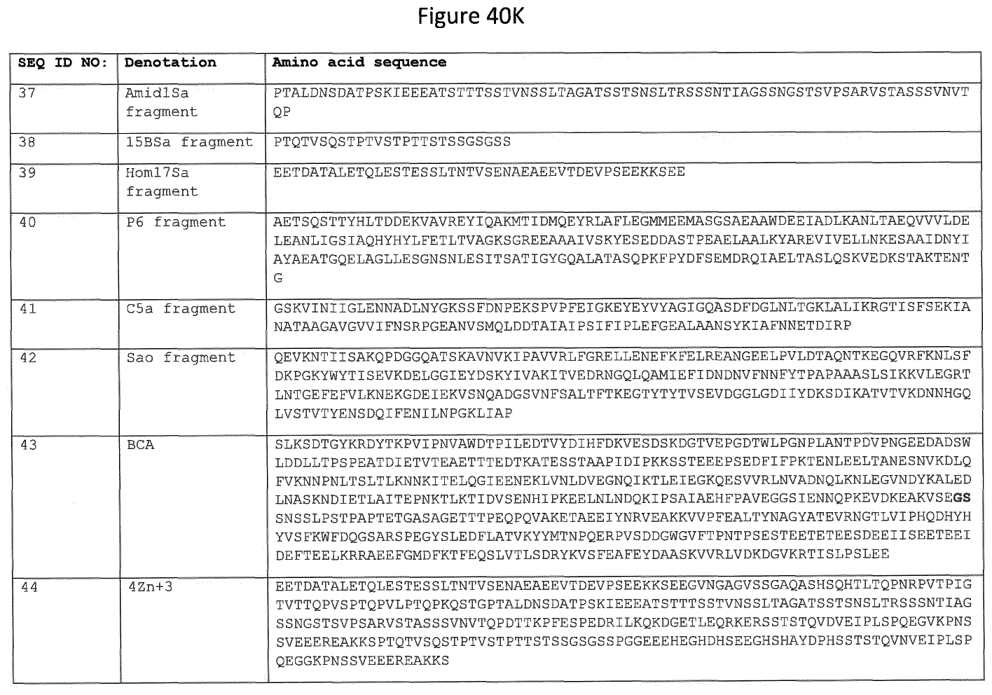

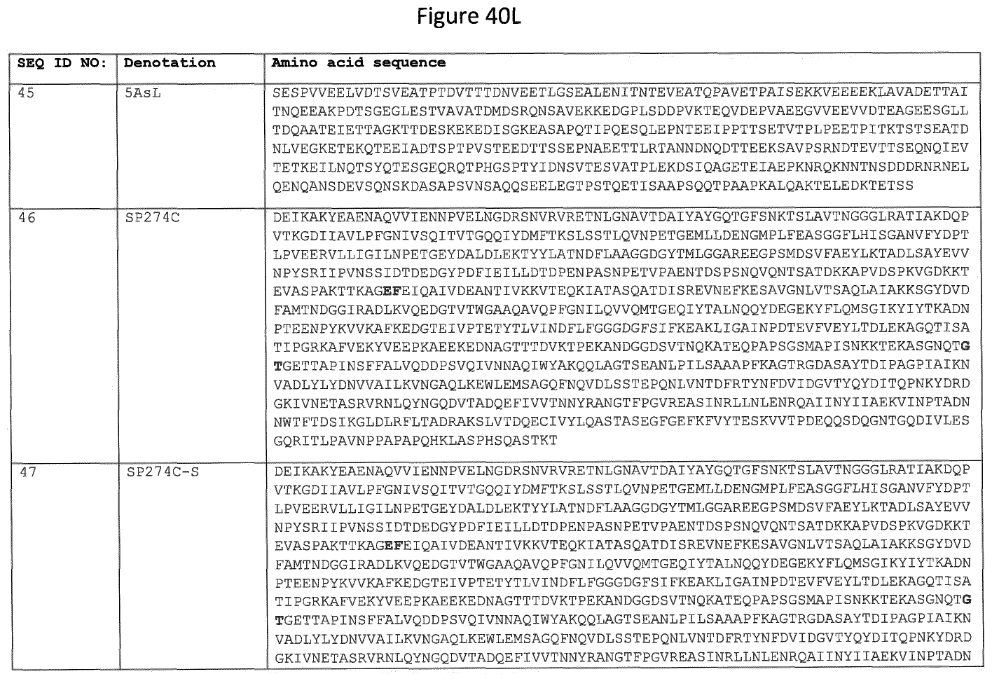

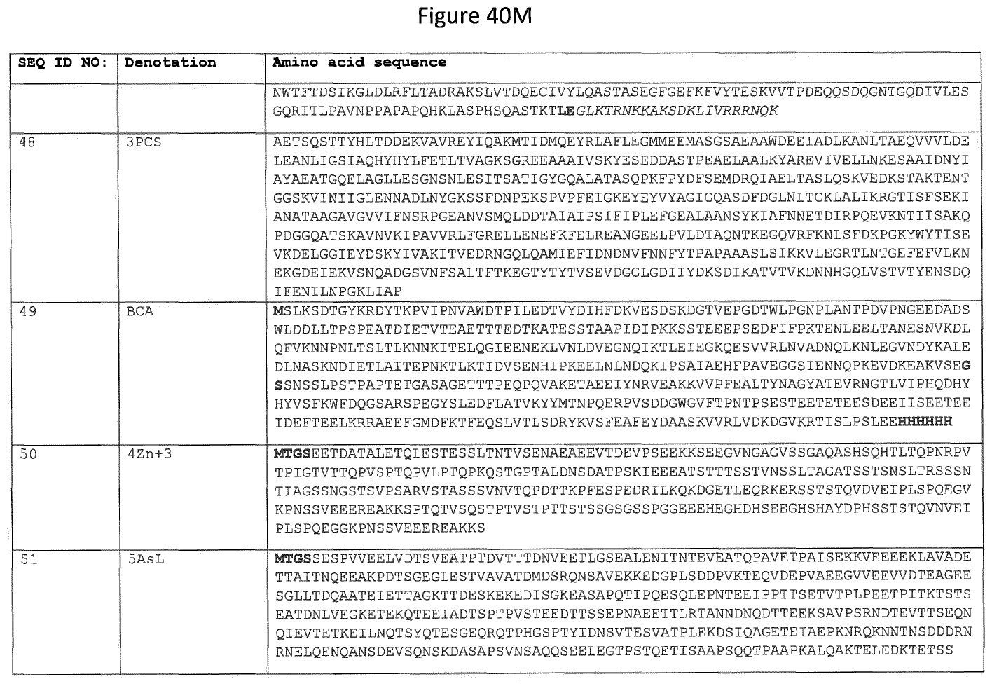

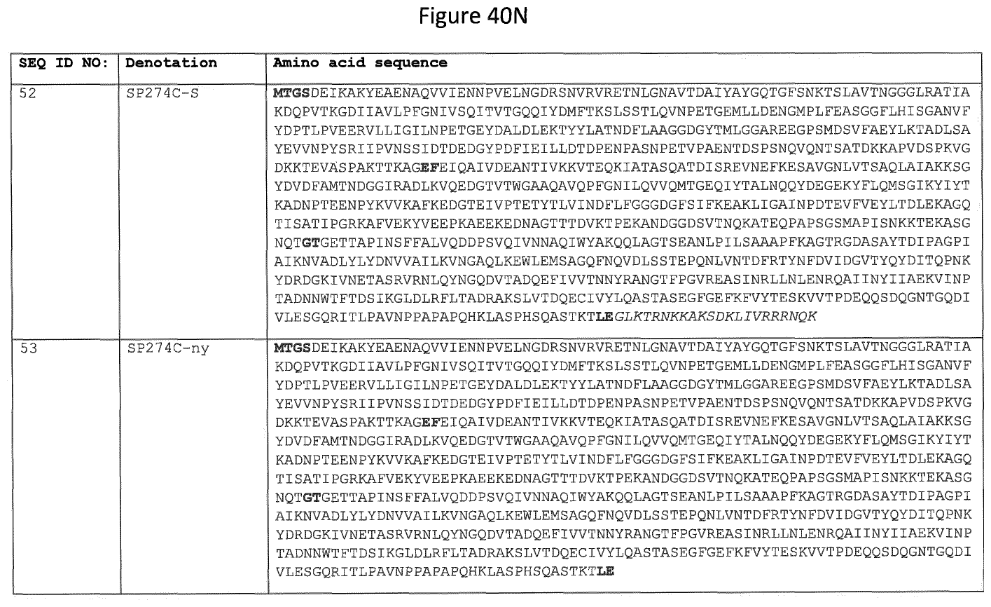

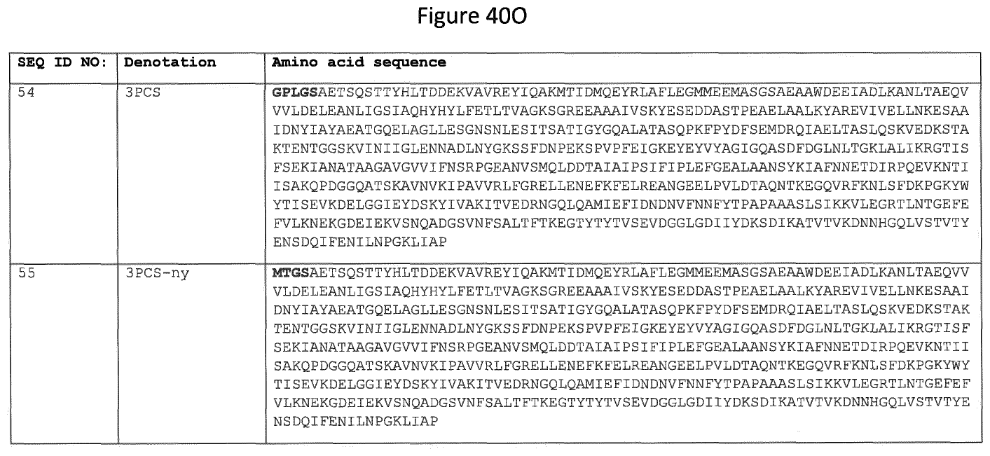

A schematically drawing of the fusion polypeptides BCA (SEQ ID NO:49), 4Zn+3 (SEQ ID NO:50), 5AsL (SEQ ID NO:51) and SP274C (SP274C-ny (SEQ ID NO:53) and SP724C-S (SEQ ID NO:52) and 3PCS (3PCS (SEQ ID NO:54) and 3PCS-ny (SEQ ID NO:55)), comprising to SEQ ID NO: 43, 44, 45, 46, 47, 48 and 48, respectively, is shown in FIG. 1. The border for each fragment of the fusion polypeptides is indicated in the figures by bars. Sequences of said fusion polypeptides are shown in FIG. 40. In FIG. 40 amino acids indicated in bold originate from the construction work of the fusion polypeptide and it should be noted that these amino acids could be changed or even absent if another fusion strategy is used. Such amino acid residues are indicated by X in SEQ ID NO:106-115. In particular, said amino acids in bold indicate amino acid residues which originate from vector and cloning site and amino acids in italics indicate either a His-tag, SL2-tag or LSL-tag sequence, where relevant.

BCA, 4Zn+3 and 5AsL comprise unstructured domains, while SP274C-S and 3PCS comprise structured domains. In particular, 4Zn+3 and 5AsL comprise unstructured domains only.

SP274C-S (SEQ ID NO:47) and SP274C-ny (SEQ ID NO:46) comprise antigens which are fragments of three nucleotidases SP2 (SEQ ID NO: 11), SP4 (SEQ ID NO:13) and SP7 (SEQ ID NO:12). SP274C-S comprises SEQ ID NO:34, 35 and 36. The sequence of SP274C-S and SP274C-ny including amino acid residues derived from vector, cloning site and/or SL2-tag is shown in SEQ ID NO:52 and 53 in bold and/or italics.

5AsL (SEQ ID NO:45) comprises six fragments from five different putative proteases P1, P8, P11, M2 and IgdE (SEQ ID NO:6-10). The six fragments comprise the amino acid sequences as defined in SEQ ID NO: 28-33. The sequence of 5AsL including amino acid residues derived from vector, cloning site and/or any tag is shown in SEQ ID NO:51 in bold and/or italics.

BCA (SEQ ID NO:43) comprises fragments of the three proteins involved in Zinc uptake; HtpsA (SEQ ID NO: 1), HtpsB (SEQ ID NO:2) and HtpsC (SEQ ID NO:3). The three fragments comprise the amino acid sequences as defined in SEQ ID NO:20, 21 and 22. The sequence of BCA including amino acid residues derived from vector, cloning site and/or any tag is shown in SEQ ID NO:49 in bold and/or italics.

4Zn+3 (SEQ ID NO:44) comprises part of eight different proteins; four proteins involved in zinc uptake, HtpsA (SEQ ID NO:1), HtpsB (SEQ ID NO:2), HtpsC (SEQ ID NO:3), HtpsC2 (SEQ ID NO:4) and AdcA (SEQ ID NO:5), and three unrelated proteins Amid1 Sa (SEQ ID NO:14), 15BSa (SEQ ID NO:15) and Hom17Sa (SEQ ID NO:16). 4Zn+3 comprises fragments having amino acid sequences as defined in SEQ ID NO: 23-26 and 37-39. The sequence of 4Zn+3 including amino acid residues derived from vector, cloning site and/or any tag is shown in SEQ ID NO:50 in bold and/or italics. 3PCS (SEQ ID NO:48) comprises fragments of the three proteins, which comprise LPXTG-motifs; P6 (SEQ ID NO:17), C5a (SEQ ID NO:18) and Sao (SEQ ID NO:19). 3PCS comprises fragments having amino acid sequences as defined in SEQ ID NO:40-42. The sequence of 3PCS including amino acid residues derived from vector, cloning site and/or any tag is shown in SEQ ID NO:54 (3PCS) and SEQ ID NO:55 (3PCS-ny) in bold and/or italics.

Determination of if the proteins are structured or unstructured were predicted using IUPred2 (Mészáros et al., 2018 and Erdös et al., 2020).

### Example 2

### Cloning, Expression and Purification of Fragments SP2, SP4, SP7

This Example describes the cloning, expression and purification of fragments of the antigens SP2, SP4, SP7 in SP274C-S and SP274C-ny.

Materials and methods: The C-terminal fragment of SP2 (SEQ ID NO:11), SP4 (SEQ ID NO:13), and SP7 (SEQ ID NO:12) and denoted SP2 fragment, SP4 fragment, SP7 fragment, corresponding to SEQ ID NO:34, 36 and 35, respectively were combined into fusion polypeptide SP274C-S (SEQ ID NO:52) with an SL2-tag as described herein and into fusion polypeptide SP274-ny (SEQ ID NO:53). For clarity, SEQ ID NO:52 and 53 comprises SEQ ID NO:46 which lacks the SL2-tag (SEQ ID NO:80).

Firstly, the gene fragments were obtained from external providers as indicated below and cloned into the BmKny vector in fusion with 6 histidines (indicated herein by the letter H (SEQ ID NO:81)) according to the following essentially as described in WO 2017/005913. Briefly:

The gene encoding the SP2 fragment, encoding the amino acid sequence according to SEQ ID NO:34, was ordered codon optimized from GenScript and was flanked by the restriction enzymes sites BamHI and XhoI. In addition, the fragment encodes six histidines after the BamHI site. The fragment was cloned into the vector BmKny using the sites BamHI and XhoI. The obtained SP2 fragment was His-tagged.

The gene encoding the SP4 fragment, encoding the amino acid sequence according to SEQ ID NO:36, was ordered codon optimized from GenScript and was flanked in the N-terminal by the restriction enzymes site NdeI followed by a DNA sequence encoding six histidines after which a BamHI site follows and the C-terminal ends with the restriction site XhoI. The fragment was cleaved with NdeI and XhoI and cloned into the vector BmKny in the corresponding sites. The obtained SP4 fragment was His-tagged.

The gene encoding the SP7 fragment, encoding the amino acid sequence according to SEQ ID NO:35, was ordered codon optimized from GenScript and was flanked N-terminally by the restriction enzymes site NdeI followed by a DNA sequence encoding six histidines after which a BamHI site follows and the C-terminal ends with the restriction site XhoI. The fragment was cleaved with NdeI and XhoI and cloned into the vector BmKny in the corresponding sites. The obtained SP7 fragment was His-tagged.

The E. coli clones were grown and the protein expression induced after which protein lysates was made and purified on Talon columns (as described in Example 4) and analyzed by SDS-PAGE as described above.

Results: In conclusion, the present inventors were able to successfully express and purify SP2 fragment, SP4 fragment, SP7 fragment, corresponding to SEQ ID NO:34, 36 and 35, respectively, in fusion with the His-tag.

### Example 3

### Cloning of Fusion Polypeptides

In this Example, the cloning of fusion polypeptides BCA (SEQ ID NO:49), 4Zn+3 (SEQ ID NO:50), 5AsL (SEQ ID NO:51), SP274C-S (SEQ ID NO:52) SP274C-ny (SEQ ID NO:53), 3PCS (SEQ ID NO:54) and 3PCS-ny (SEQ ID NO:55), comprising SEQ ID NO:43, 44, 45, 47, 46, 48 and 48, respectively, is described. Additionally, the fusion of polypeptide SP274C (SEQ ID NO:46) with SL2-tag (SEQ ID NO:80) resulting in SP274C-S (SEQ ID NO:47) is disclosed herein.

### Cloning

Materials and methods: The gene fragments encoding the protein fragments included in BCA, 4Zn+3, 5AsL,SP274C and 3PSC were cloned into expression vectors as described below. SP274 was cloned into the BmKny vector in fusion with the SL2-tag (GLKTRNKKAKSDKLIVRRRNQK (SEQ ID NO:80)), indicated herein by the letter S and located in the C-terminus according to the following:

SP274C-S: The nucleotide sequence of SP274C-S (SEQ ID NO:60) and SP274C-ny (SEQ ID NO:61) is a fusion of the gene fragments SP2C and SP74C and contain parts of the three genes SP2, SP4 and SP7 as described in WO 2017/005913. Those genes were synthesized and codon optimized by GenScript as described in Example 2. SP274C-S was constructed from SP74C and SP2C.

SP74C: The nucleotide sequence of SP74C-S (SEQ ID NO:59) is a fusion of the gene fragments SP7C (SEQ ID NO:76 and SP4C (SEQ ID NO:75) (described above). The SP7C fragment was PCR amplified using the clone SP7C-S (SEQ ID NO:78) as a template and the primers used were Tac5 (SEQ ID NO:68) and NP7Kpn (SEQ ID NO:69). The SP4C fragment was PCR amplified using clone SP4C-S (SEQ ID NO:77) as template and the primers were SP4CKpnl (SEQ ID NO:70) and BmR2 (SEQ ID NO:71). The PCR condition for the two reactions were 50° C. annealing temperature, 2 minutes extension and 30 cycles.

Primers for SP7C
Tac5:
(SEQ ID NO: 68)
GCCGACATCATAACGGTTCTGG, vector primer
NP7Kpn:
(SEQ ID NO: 69)
TATAGGTACCGGTTTGGTTGCCACTTGC,
Template: SP7C-S
(SEQ ID NO: 78)
Primers for SP4C
SP4CKpnI:
(SEQ ID NO: 70)
ATATGGTACCGGCGAAACGACGGC,
BmR2:
(SEQ ID NO: 71)
GGTGGTCCCACCTGACGTC, vector primer
Template: SP4C-S
(SEQ ID NO: 102)

The SP7C PCR product was cleaved with BamHI and KpnI and the SP4C PCR product was cleaved with KpnI and XhoI. The cleaved fragments were ligated into the vector BmKSL2. Before ligation, the vector was prepared by cleavage of BamHI and XhoI and treatment of calf Intestinal alkaline phosphatase (CIP). The fragment obtained was SP74C-S (SEQ ID NO:59) and its sequence was verified and encoded the amino acid sequence according to SEQ ID NO:79.

SP274C: The gene fragment SP2C (SEQ ID NO:74) (described above) was PCR amplified using SP2C-S (SEQ ID NO:58) as a template and the primers were Tac5 (SEQ ID NO:68) and SP2Cr (SEQ ID NO:72). The SP74C fragment was PCR amplified using SP74C-S (SEQ ID NO:59) as a template and the primers were SP74Cs (SEQ ID NO:73) and BmR2 (SEQ ID NO:71). The PCR condition for the two reactions were 50° C. annealing temperature, 2 minutes extension and 30 cycles. The SP2C fragment was cleaved with BamHI and EcoRI and the SP74C fragment was cleaved with EcoRI and XhoI. Both fragments were then ligated into the vector BmKSL2. Before ligation, the vector was prepared by cleavage of BamHI and XhoI and treatment of calf Intestinal alkaline phosphatase (CIP). The construct obtained was SP274C-S (SEQ ID NO:60) and its sequence was verified. The construct encoded the fusion polypeptide SEQ ID NO:47, which comprises SEQ ID NO:46. The amino acid sequence translated from the first Met to the stop codon of said construct obtained corresponds SEQ ID NO:52, wherein amino acid residues originating from the vector used, cloning site used as well as SL2-tag are indicated.

Primers for SP2C
Tac5:
(SEQ ID NO: 68)
GCCGACATCATAACGGTTCTGG, vector primer
SP2Cr:
(SEQ ID NO: 72)
TATAGAATTCACCTGCTTTGGTCGTTTTTGC
Template: SP2C-S
(SEQ ID NO: 58)
Primers for SP74C PCR
SP74Cs:
(SEQ ID NO: 73)
ATATGAATTCGAAATTCAAGCCATTGTGGACG
BmR2:
(SEQ ID NO: 71)
GGTGGTCCCACCTGACGTC, vector primer
Template:SP74C-S
(SEQ ID NO: 59)

The SP274C-ny construct was done using the same cleaved PCR products, as used for doing SP274C-S, but the PCR products was ligated into the vector BmKny cleaved with BamHI and XhoI. Before ligation, the vector was prepared by cleavage of BamHI and XhoI and treatment of calf Intestinal alkaline phosphatase (CIP). Kanamycin resistant colonies were isolated. The obtained clone is called SP274C-ny (SEQ ID NO:61) was sequence verified and encoded the fusion polypeptide SP274C-ny (SEQ ID NO:53). The amino acid sequence translated from the first Met to the stop codon of said construct obtained corresponds SEQ ID NO:53, wherein amino acid residues originating from the vector and cloning site used are indicated. SEQ ID NO:53 comprises SEQ ID NO:46.

BCA: BCA comprises fragments of genes encoding HtpsA (SEQ ID NO:1), HtpsB (SEQ ID NO:2), and HtpsC (SEQ ID NO:3), and was made from gene fragments Su219 (SEQ ID NO:62) and Su302 (SEQ ID NO:63) and was prepared as follows:

Su219: Codon optimized DNA sequence comprises part of the HtpsA gene, encoding fragment according to SEQ ID NO:20, cloned into the vector pUC57-mini (GenScript). The DNA contains a BamHI-site in the 5′-end and a stop codon and an XhoI-site at the 3′-end. DNA was cleaved with BamHI and XhoI, ligated into the vector BmKny. Before ligation, the vector was prepared by clevage of BamHI and XhoI and treatment of calf Intestinal alkaline phosphatase (CIP). Kanamycin resistant colonies were isolated and the protein expression after IPTG induction was investigated. One clone with high protein expression was chosen, the DNA sequence was determined, showing the expected sequence. The new clone in vector pBmKny was named pBm219.

Su302: The codon optimized DNA sequence (GenScript) contains parts of the HtpsB and HtpsC genes, encoding fragments according to SEQ ID NO:21 and 22, respectively. It was cloned into the vector pUC57mini. The DNA clone contains an NdeI site in 5′-end and a BamHI and XhoI site in the 3′-end.

BCA: The clone pBm219 was cleaved with NdeI and BamHI and thereafter treated with CIP. The clone Su302 was cleaved with NdeI and BamHI. After purification, the two cleaved plasmids were mixed and ligated together.

Kanamycin resistant colonies were isolated and the protein expression after IPTG induction was investigated.

One clone with high protein expression was chosen, the DNA sequence was determined, showing the expected sequence. Thus, the polynucleotide SEQ ID NO:64 encoding a fusion polypeptide comprising SEQ ID NO:43 was obtained.

The amino acid sequence translated from the first Met to the stop codon of said construct obtained corresponds SEQ ID NO:49, wherein amino acid residues originating from the vector used, cloning site used as well as his-tag are indicated.

5AsL: The ordered codon optimized DNA sequence of 5AsL (SEQ ID NO:65) (ordered from GenScript) contains six fragments from five different putative proteases P1 (SEQ ID NO:6), P8 (SEQ ID NO:7), P11 (SEQ ID NO:8) M2 (SEQ ID NO:9) and IgdE (SEQ ID NO:10). The six fragments have amino acid sequence as shown in SEQ ID NO:28-33. The ordered DNA, which contained a BamHI-site in the 5′-end and a stop codon and an XhoI-site in the 3′-end, was cloned into pGex6P-1. The ordered DNA was cleaved with BamHI and XhoI and ligated into the vector BmKny. Kanamycin resistant colonies were isolated and the protein expression after IPTG induction was investigated. One clone with high protein expression was chosen and its sequence was verified. Thus, the fusion polypeptide comprising SEQ ID NO:45 was obtained.

The amino acid sequence translated from the first Met to the stop codon of said construct obtained corresponds SEQ ID NO:51, wherein amino acid residues originating from the vector used, cloning site used as well as any tag are indicated.

4Zn+3: The codon optimized DNA sequence of 4Zn+3 (SEQ ID NO:66) (ordered from GenScript) contains nucleotide sequences encoding fragments the following eight different proteins; HtpsA (SEQ ID NO:1), HtpsB (SEQ ID NO:2), HtpsC (SEQ ID NO:3), HtpsC2 (SEQ ID NO:4), AdcA (SEQ ID NO:5), Amid1Sa (SEQ ID NO:14), 15BSa (SEQ ID NO:15) and Hom17Sa (SEQ ID NO:16). The eight fragments have amino acid sequence as shown in SEQ ID NO:23-27 and 37-39.

The ordered 4Zn+3 DNA, which contains a BamHI-site in the 5′-end and a stop codon and an XhoI-site in the 3′-end, was cloned into pGex6P-1. The DNA was cleaved with BamHI and XhoI and ligated into the vector BmKny. Before ligation, the vector was prepared by cleavage of BamHI and XhoI and treatment of CIP. Kanamycin resistant colonies were isolated and the protein expression after IPTG induction was investigated. One clone with high protein expression was chosen and its sequence was verified. Thus, a clone encoding fusion polypeptide SEQ ID NO:44 was obtained.

The amino acid sequence translated from the first Met to the stop codon of said construct obtained corresponds SEQ ID NO:50, wherein amino acid residues originating from the vector used, cloning site used as well as any tag are indicated.

3PCS: The ordered codon optimized DNA sequence (GenScript) contains part of three different LPXTG proteins P6 (SEQ ID NO:17), C5a (SEQ ID NO:18) and Sao (SEQ ID NO:19). The three fragments have amino acid sequences as defined in SEQ ID NO: 40-42. The ordered DNA contains a BamHI-site in the 5′-end and a stop codon and an XhoI-site in the 3′-end and it was cloned into pGex6P-1. Ampicillin resistant colonies were isolated and one clone with high protein expression containing 3PCS was chosen. The clone corresponded to SEQ ID NO:67 and its sequence was verified. Thus, a clone encoding fusion polypeptide comprising SEQ ID NO:48 was obtained.

The amino acid sequence of the construct obtained corresponded to SEQ ID NO:54, wherein amino acid residues originating from the vector used, cloning site used as well as any tag are indicated.

3PCS-ny: The 3PCS is re-cloned into the vector BmKny. The ordered DNA was cleaved with BamHI and XhoI and ligated into the vector BmKny (BamHI/XhoI and CIP). Kanamycin resistant colonies was isolated and the protein expression after IPTG induction was investigated.

One clone corresponding to SEQ ID NO:67 with high protein expression was chosen and its sequence was verified. Thus, a clone encoding fusion polypeptide comprising SEQ ID NO:48 was obtained. The amino acid sequence translated from the first Met to the stop codon of said construct obtained corresponded to SEQ ID NO:55, wherein amino acid residues originating from the vector used, cloning site used as well as any tag are indicated.

General Condition for Growing E. coli and Induction of Protein Expression

E. coli BL21 cells containing the different constructions was grown at 37° C. to OD 0.5-1 after which IPTG was added to 50 μg/ml and the induction phase was carried out overnight shaking at 25° C. Electroporation of E. coli was performed as described in Example 1.

### Example 4

### Purification of Fusion Polypeptides

In this Example, the purification of polypeptides BCA (SEQ ID NO:49 comprising SEQ ID NO:43), 4Zn+3 (SEQ ID NO:50 comprising SEQ ID NO:44), 5AsL (SEQ ID NO:51 comprising SEQ ID NO:45), 3PCS (SEQ ID NO:54 comprising SEQ ID NO:48), 3PCS-ny (SEQ ID NO:55 comprising SEQ ID NO:48), SP274C-S (SEQ ID NO:52 comprising SEQ ID NO:47, which in turn comprises SEQ ID NO:46) and SP274C-ny (SEQ ID NO:53 comprising SEQ ID NO:46) as described in Example 3 is disclosed.

### SP274C-S (SEQ ID NO:52)

The induced E. coli cells were centrifuged and resuspended in a solution containing 100 mM NaCl, 20 mM Tris pH 7.4 and 0.05% Tween 20. Thereafter lysozyme was added to a final concentration of 20 μg/ml and the cells were repeatedly freeze/thawed until lysis occurred. The lysate was sonicated, and the debris was removed by centrifugation. The clarified lysate was sterile filtered (0.45 μm filter) and frozen in aliquots until purification of the protein was performed. Purification was performed as described below.

### SP274C-ny (SEQ ID NO:53)

The induced E. coli cells were centrifuged and resuspended in a solution containing 100 mM NaCl, 20 mM Tris pH 7.4 and 0.05% Tween 20. Thereafter lysozyme was added to 20 μg/ml and the cells were repeatedly frozen/thawed until lysis. The lysate was sonicated and the debris was removed by centrifugation. The clarified lysate was sterile filtered (0.45 μm filter) and frozen in aliquots until the purification of the protein.

Eighty ml clarified lysate was shaken with 2.4 g Allantoin for binding of endotoxins and Allotoin crystals with bound endotoxins were removed by centrifugation. Purification was performed as described below.

### BCA, 5AsL, 4Zn+3 and 3PCS (SEQ ID NO:49, 51, 50 and 54)

The induced E. coli cells were centrifuged and resuspended in a solution containing 100 mM NaCl, 20 mM Tris pH 7.4 and 0.05% Tween 20. Thereafter lysozyme was added to a final concentration of 100 μg/ml (BCA and 3PCS) or 20 μg/ml (5AsL and 4Zn+3) and the cells were repeatedly freeze/thawed until lysis occurred. The solution comprising lysed cells containing proteins BCA, 5AsL and 4Zn+3 was boiled for 15 min. The solution was thereafter cooled to room temperature and the cell debris including denatured proteins was removed by centrifugation. The lysed cells containing 3PCS was just centrifuged after lysis had occurred and the supernatant collected. The clarified lysate was sterile filtered (0.45 μm filter) and frozen in aliquots until purification of the protein was performed. Purification was performed as described below.

### 3PCS-ny (SEQ ID NO:55)

The lysate is E. coli cell lysate is prepared essentially as described for 3PCS.

### Purification of Fusion Polypeptides on Columns.

Materials and methods: In order to obtain soluble proteins that can be used with adjuvants, the fusion polypeptides were purified.

Protein purification on Talon: For the protein SP2, SP4 and SP7 Talon was used. Premade columns with 1 ml Talon (Cytiva) were used. All proteins were purified according to the description below (step 1-11). The columns were reused but only the same protein was purified on each column.
1. The column was washed with 10 ml TN0.4 buffer (10 mM Tris pH 7.1, 0.4 M NaCl).
2. 10-15 ml lysate was applied two times on the column.
3. The column was washed with 2×5-10 ml TN0.4 buffer.
4. Sometimes an extra washing step with 5 ml 5 mM Imidazole in TN 0.4 buffer was applied.
5. The recombinant protein was eluted with 0.1 M Imidazole in TN 0.4 buffer.
6. The column was wash with 5 ml 8 M UREA.
7. The column was regenerated with 10 ml MES (20 mM)+NaCl (0.4 M).
8. The column was washed with 20 ml H₂O and saved for the next purification of the same protein.
9. The eluted protein samples were dialyzed against PBS (2×5 l).
10. The samples were sterile filtered (0.2 μm).
11. The absorbance was measured and the samples were frozen.

Protein purification by ion exchange chromatography: For the purification of SP274C-S, BCA, 5AsL and 4Zn+3, ion exchange chromatography (Q Sepharose, Cytiva) was used.

### Protein Purification of SP274C-S

Binding was performed in 150 nm NaCl, 20 mM Tris pH 7.4 and 0.05% Tween 20.
Washing was done with 0.25 mM NaCl and 20 mM Tris pH 7.4.
Elution was done in 0.4 mM NaCl and 20 mM Tris pH 7.4.
The eluted protein was diluted with H₂O to a NaCl concentration 150 mM and repurified on Q Sepharose with the same conditions as above.
The eluted protein was dialyzed against 20 mM Tris-HCl pH 7.5 and 100 mM NaCl.
Finally, the protein was sterile filtered using a 0.20 μm filter and frozen until use.

### Protein Purification of SP274C-Ny

Binding was performed in 100 nm NaCl, 20 mM Tris pH 7.4 and 0.05% Tween 20.
Washing was done with 150 mM NaCl and 20 mM Tris pH 7.4.
Elution was done in in two steps, first in 0.25 mM NaCl and 20 mM Tris pH 7.4 and then in 0.3 mM NaCl and 20 mM Tris pH 7.4
The eluted protein was dialysed against 20 mM Tris-HCl pH 7.5 and 100 mM NaCl.
Finally, the protein was sterile filtered using a 0.20 μm filter and frozen until use.

### Protein Purification of BCA, 5AsL and 4Zn+3

Binding was performed in 50 mM NaCl and 20 mM Tris pH 7.4.
Elution was done in 300 mM NaCl (for BCA), 250 mM NaCl (for 5AsL) or 200 mM NaCl (for 4Zn+3) and 20 mM Tris pH 7.4.
The eluted protein was dialyzed against 20 mM Tris-HCl pH 7.5 and 100 mM NaCl (for BCA) or against 10 mM Tris-HCl pH 7.4 (for 5AsL, 4Zn+3).
Finally, the protein was sterile filtered using a 0.20 μm filter and frozen until use.

### Protein Purification of 3PCS

Affinity chromatography (Glutathione Sepharose 4B) was used for 3PCS purification. The 3PCS was purified in batch and after careful washing;
Precision protease was used to cleave of 3PCS from the GST-tag.
Dialysis was performed in 20 mM Tris-HCl pH 7.4 and 100 mM NaCl.
Finally the protein was sterile filtered using a 0.20 μm filter and frozen until use.

### Protein Purification of 3PCS-ny

The 3PCS-ny is purified and the protein is sterile filtered using a 0.20 μm filter and frozen until use.

Results: Thus, the fusion polypeptides BCA, 4Zn+3, 5AsL, SP274C-S, SP274C-ny and 3PCS, corresponding to SEQ ID NO: 49, 50, 51, 52, 53 and 54, as disclosed herein were successfully cloned, expressed and purified. It is expected that and 3PCS-ny, corresponding to SEQ ID NO:55 is also successfully cloned, expressed and purified.

### Example 5

### Stability of Fusion Polypeptides

The stability of the purified fusion polypeptides obtained in Example 4 was tested. The fusion polypeptides were aliquoted and incubated as specified below. Analysis was performed by SDS-PAGE.

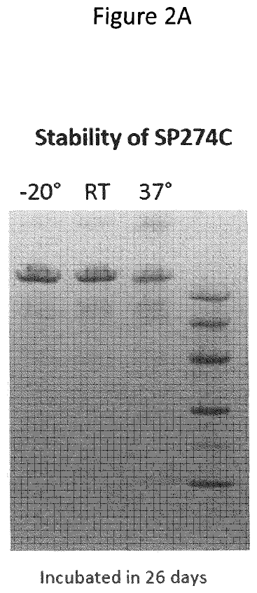

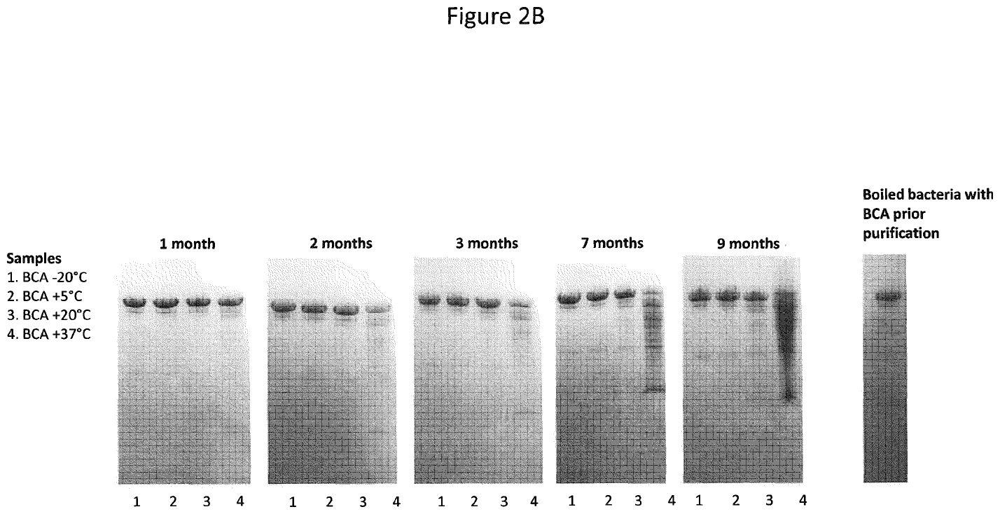

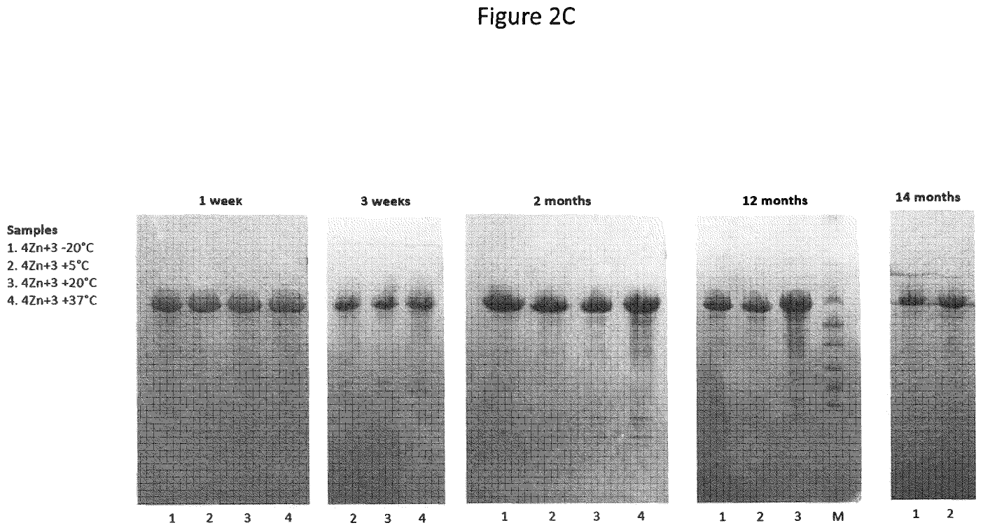

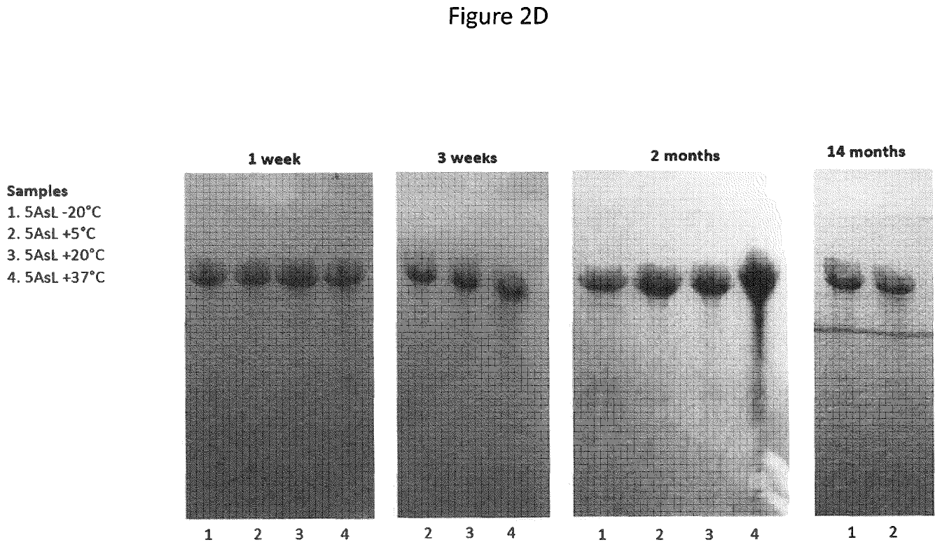

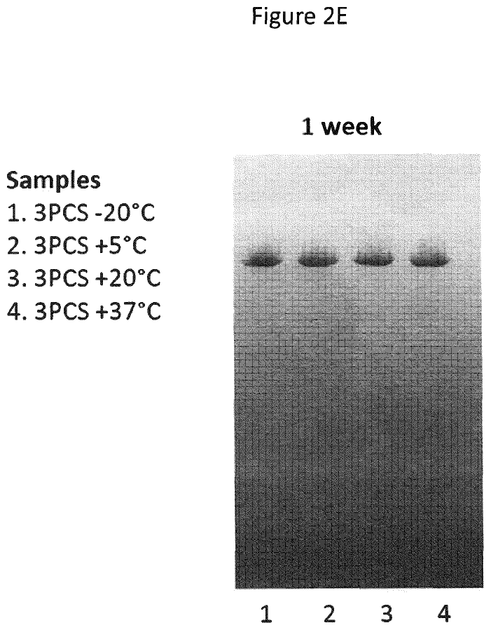

Aliquots of the fusion polypeptide SP274C-S (SEQ ID NO:52) were incubated for 26 days at 37° C., room temperature (+20° C.) or frozen (−20° C.). The fusion antigen exhibited a high level of stability (FIG. 2A).

Aliquots of the fusion polypeptide BCA (SEQ ID NO:49) were incubated at 37° C., room temperature (+20° C.), at +5° C. or frozen (−20° C.). Stability was evaluated after 1, 2, 3, 7 and 9 months. The fusion antigen exhibited a high level of stability (FIG. 2B).

Aliquots of the fusion polypeptide 4Zn+3 (SEQ ID NO:50) were incubated at 37° C., room temperature (+20° C.), at +5° C. or frozen (−20° C.). Stability was evaluated after 1 week, 3 weeks, 2 months, 12 months and 14 months. The fusion antigen exhibited a good stability (FIG. 2C).

Aliquots of the fusion polypeptide 5AsL (SEQ ID NO:51) were incubated at 37° C., room temperature (+20° C.), at +5° C. or frozen (−20° C.). Stability was evaluated after 1 week, 3 weeks, 2 months and 14 months. The fusion antigen exhibited a good stability (FIG. 2D).

Aliquots of the fusion polypeptide 3PSC (SEQ ID NO:54) were incubated at 37° C. room temperature (+20° C.), at +5° C. or frozen (−20° C.). Stability was evaluated after 1 week. The fusion antigen exhibited a good stability (FIG. 2E).

### Example 6

### Immunological Response in Rabbit

In this Example, BCA, 4Zn+3, 5AsL and SP274C-S, corresponding to SEQ ID NO: 49, 50, 51 and 52 and comprising SEQ ID NO: 43, 44, 45 and 47 (which in turn comprises SEQ ID NO:46) respectively, were injected into rabbits and the antibody responses thereto were investigated.

Materials and methods: Immunization of rabbits: Prior to immunization, blood was collected from rabbits and pre-immunisation serum prepared and stored for later analysis. The rabbits were immunized at Capra Science Antibodies AB three times with a mixture of four fusion proteins (10 μg of each fusion); BCA, 4Zn+3, 5AsL and SP274C-S, corresponding to SEQ ID NO: 49, 50, 51 and 52. The first immunization was done using FCA (Freund's complete adjuvant) and the second and third immunization was done with FIA (Freund's incomplete adjuvant). The second immunization was performed 4 weeks after the first immunization and the third immunization was performed 4 weeks after the second. Blood was taken for serum preparation two weeks after the last immunization.

Western Blot: Fusion polypeptides (BCA, SP274C-S, 4Zn+3 and 5AsL) and polypeptides HtpsA, HtpsB, HtpsC, SP2C, SP4C and SP7C (corresponding to SEQ ID NO:1, 2, 3, 74, 75 and 76, respectively) were loaded into wells and run on a pre-cast 8-25% gradient gel from Cytiva (PhastGel; GE17-0542-01) using SDS buffer strips (PhastGel; GE17-0516-01) on the PhastSystem. The gel was stained with Coommasie blue according to the standard protocol. After the 20 min SDS-PAGE, the proteins were transferred onto an Amersham Hybond-C extra filter (RPN303E, Cytiva) for 30 min at 65° C. To prevent unspecific binding, the filter was blocked with Ovalbumin to a final concentration of 150 μg/ml for at least 30 min at room temperature. Next, serum was added to the filter. The serum was diluted 200 times and incubation was done overnight at room temperature. The filter was washed four times with 20 mM Tris-HCl pH 7.3, 100 mM NaCl and 0.05% Tween-20 prior to addition of 5 μg horseradish peroxidase (HRP) conjugate of protein G (P21041; Invitrogen). The filter was washed two times with 20 mM Tris-HCl pH 7.3, 100 mM NaCl and 0.05% Tween-20 and two times with 20 mM Tris-HCl pH 7.3 and 100 mM NaCl. Ten microliter hydrogen peroxidase 30% (107209; MERCK) was added to 10 ml of 20 mM Tris-HCl pH 7.3 and 100 mM NaCl and for visualization of protein-peroxidase conjugates, 2 ml of the substrate 4-chloro-1-naphtol was added for visualization of protein-peroxidase conjugates following incubation and incubated at room temperature.

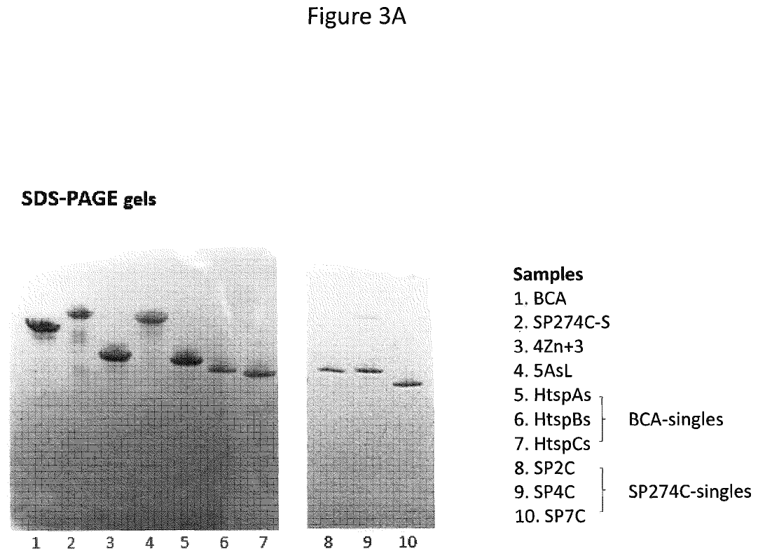

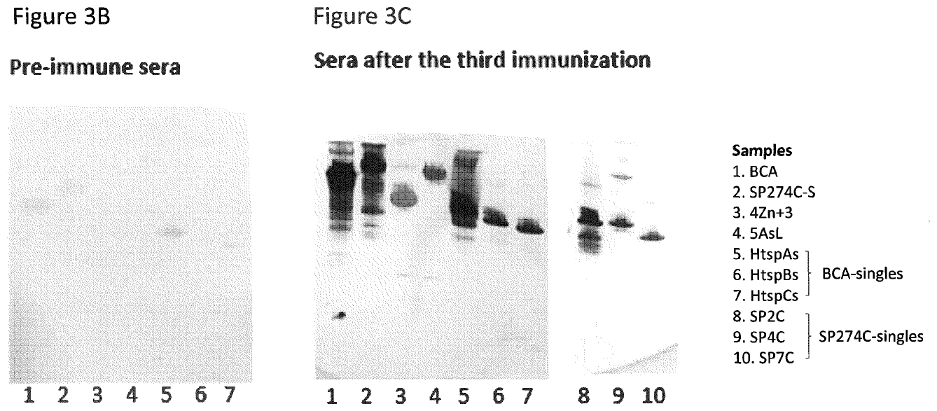

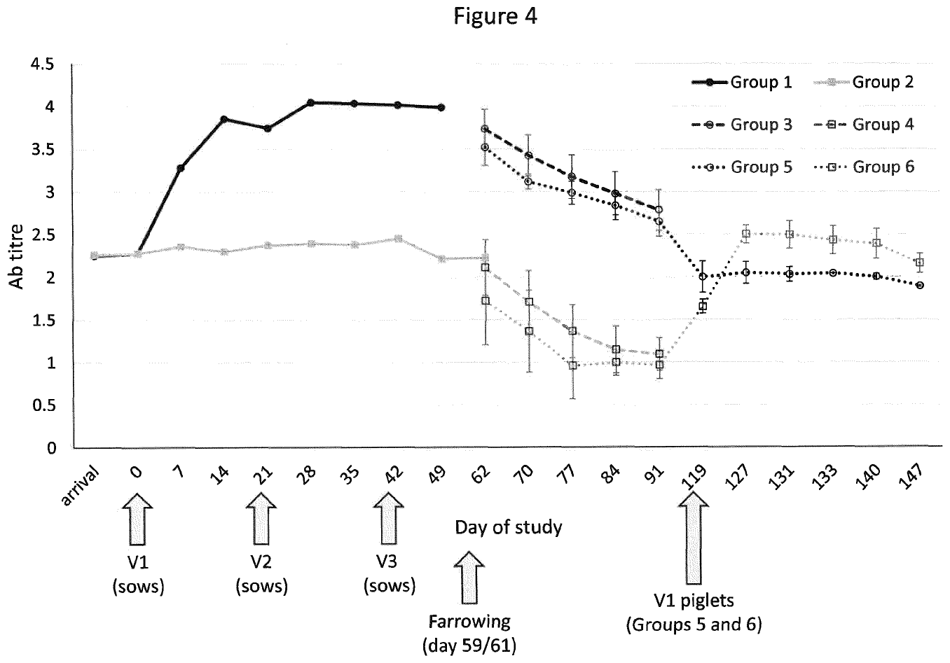

Results: The SDS gel (FIG. 3A) shows that the fusion polypeptides BCA, 4Zn+3, 5AsL and SP274C-S, corresponding to SEQ ID NO: 49, 50, 51 and 52, and polypeptides HtpsA, HtpsB, HtpsC, SP2C, SP4C and SP7C, corresponding to SEQ ID NO:1, 2, 3, 74, 75 and 76, respectively were intact. Pre-immune sera were assayed against the four fusion proteins and polypeptides HtpsA, HtpsB, HtpsC (indicated in the figures as “BCA singles”) and the results are shown in FIG. 3B. The preimmune sera was unreactive against the 4Zn+3 and 5AsL. Weak signals were visible for BCA, SP274C and the BCA singles in pre-immune sera. This may be due to cross-reaction since the zinc binding proteins in BCA and the nucleotidases in SP274 have homology with proteins in bacteria other than S. suis. It was concluded that titers or antibodies against the fusion polypeptides were low to undetectable in pre-immune sera. The strong signals observed in the positions corresponding to the predicted sizes of the fusion polypeptides BCA, 4Zn+3, 5AsL and SP274C-S and polypeptides HtpsA, HtpsB, HtpsC, SP2C, SP4C and SP7C as shown in FIG. 3C indicate the presence of fusion polypeptide specific antibodies in immunized rabbits.

Thus, this Example shows that immunisation with a mixture of the four S. suis fusion polypeptides BCA, 4Zn+3, 5AsL, and SP274C-S led to the generation of strong antibody responses to the four different fusion proteins as well as to the three polypeptides comprised in BCA and in SP274C-S, respectively, after the third immunization.

### Example 7

### Immunogenicity and Efficacy of a Streptococcus suis Vaccine Against an Experimental Streptococcus suis Challenge in Piqlets

In this Example, 1 ml of the four fusion polypeptides namely BCA, 4Zn+3, 5AsL and SP274C-S, corresponding to SEQ ID NO: 49, 50, 51 and 52 and comprising SEQ ID NO: 43, 44, 45 and 47 (which in turn comprises SEQ ID NO:46) respectively. The combination of said four fusion polypeptides is referred to as Piggivac4). Piggivac4 mixed with 100 μg of MatrixV adjuvant or an adjuvant-only control was injected into pregnant sows on days 0, 21 and 42. 100 μg of each fusion polypeptide, also referred to as fusion antigen, was used. IgG antibody responses in the sows were measured. The sow vaccinated with Piggivac4 had higher antibody levels to the components of Piggivac4 relative to the control sow from day 7 post-first vaccination.

Piglets were farrowed and weaned at approximately 4 weeks of age. IgG antibody responses in the colostrum of sows was measured. IgG antibody responses in the piglets from vaccinated and control sows were measured. The piglets from the Piggivac4-vaccinated sow had significantly higher antibody levels to the components of Piggivac4 than the piglets from the control sow from birth up to the point where the piglets in Groups 5 and 6 were vaccinated on day 119 (P<0.0001 on all days).

A group of 9 piglets from the vaccinated sow (group 3) and 10 piglets from the control sow (group 4) were challenged by intravenous injection with a dose of between 1.3×10⁹ and 1.5×10⁹ cfu of serotype 2 S. suis isolate 23/P0278/02/11, respectively. Piglets in groups 3 and 4 were then monitored for 7 days for signs of infection. Piglets were euthanized on reaching the criteria for welfare endpoint or at the end of the study on day 7 post-challenge. Piglets from the Piggivac4-vaccinated sow (group 3) had significantly lower rectal temperatures than piglets from the control sow (group 4) on day 1 and 2 post-challenge (P=0.001 and P=0.006, respectively, two-tailed student's t test). The onset of a rectal temperature of >40° C. on two consecutive days was significantly delayed in piglets in group 3 (P=0.03, log rank test). There was also a trend for the demeanour and central nervous system (CNS) signs to be lower and delayed in piglets in group 3.

Two additional piglets from the Piggivac4-vaccinated sow (Group 5) and three additional piglets from the control sow (Group 6) were vaccinated on Day 119 when they were approximately 8 weeks of age. IgG antibody responses in the piglets of groups 5 and 6 were measured. The piglets from the sow vaccinated with Piggivac4 had higher antibody levels to the components of Piggivac4 relative to the piglets from the control sow. After vaccination of the piglets in Groups 5 and 6, antibody levels to the components of Piggivac4 remained stable in group 5 piglets and increased in group 6 piglets.

In summary, vaccination of sows generated a significant antibody response, which was transferred to piglets via colostrum and reduced the effects of S. suis challenge. Vaccination of piglets at 8 weeks of age maintained antibody responses to vaccine components in piglets from the Piggivac4-vaccinated sow and generated an antibody response in piglets from the control sow.

### Materials and Methods

### Test Animals:

Details on the sows and piglets used in this study are given in Table 2 below.

TABLE 2
Detail on animals used in the study.

| Animal details | Sows | Piglets |
|---|---|---|
| Species | Porcine | Porcine |
| Breed | Yorkshire - Landrace | Yorkshire - Landrace |
| Sex | Female | Female/Male |
| Status | Pregnant (~40 day's | Age: <24 hours |
| | gestation on arrival) | |
| Body weight | | |
| Number | 2 | 19 |
| Source | High Health Status | Farrowed at Moredun |
| | Commercial pig unit without | |
| | known history of S. suis | |
| | infection | |
| Fate of | All study animals were | All study animals were |
| animals | euthanized and | euthanized and |
| | incinerated. | incinerated. |

### Test and Control Material:

The test material was prepared according to Table 3 and the control group received a control dose of adjuvant, which was selected to mimic the test material dose regime. For first vaccination of group 1 lot #1020-01 was used. For second vaccination of group 1 lot #1020-02 was used. For third vaccination of group 1 lot #1020-03 was used. For first vaccination of group 2 lot #1020-05 was used. For second vaccination of group 2 lot #1020-06 was used and for third vaccination of group 2 lot #1020-07 was used.

TABLE 3
Information on test and control material.

| Test material | Study | Control |
|---|---|---|
| Name/Code | Piggivac4 | Placebo |
| Number | | |
| Formulation | Protein solution in | Adjuvant solution in |
| | 50 mM Tris pH 7.3 | 50 mM Tris pH 7.3 |
| Concentration | 100 μg per antigen in | 100 μg/ml of Matrix |
| | 1000 μl injection volume | V (Novavax) |
| | containing 100 μg/ml of | |
| | Matrix V (Novavax) | |
| Storage Conditions | +2 to +8° C. | +2 to +8° C. |
| Required | | |
| Method of | Intramuscular injection | Intramuscular injection |
| Administration | | |
| Dose Regime | 1 ml on Day 0, 21 and 42 | 1 ml on Day 0, 21 and 42 |
| | for sow in group 1 | for sow in group 2 |
| | 0.5 ml on Day 119 to | 0.5 ml on Day 119 to |
| | piglets in group 5 | piglets in group 6 |

### Study Design and Procedure:

A summary of the study design is given in Table 4. A total of two pregnant pigs (approximately 40 days gestation) were sourced from a commercial pig farm with no history of Streptococcus suis clinical disease.

Once on site the animals were housed together on straw and were provided with drinking water and feed for pregnant sows available ad libitum. On arrival the sows were examined by a veterinarian to confirm that they were in good general health and a blood sample was collected from each. After 14 days acclimatisation (Day 0) a blood sample was collected from each sow. One sow was administered Piggivac4 (Group 1) by the intramuscular route and the second sow was injected with the Placebo (Group 2). Vaccination was repeated on Days 21 and 42. Blood samples were collected from the sows on Days 0, 7, 14, 21, 28, 35, 42 and 49. On Day 55, the animals were moved to farrowing crates. Animals farrowed on Days 59 and 61. A final blood sample was collected following completion of farrowing on day 62.

Once farrowed piglets were examined to confirm health and a record was made of the number of live/dead piglets. All piglets were encouraged to consume as much colostrum as possible. Blood samples were collected from all piglets (Groups 3 and 4) within 24 hours of farrowing then weekly to weaning. At 28 days of age all piglets were examined by a vet. Nine and 10 piglets were selected from each litter and weaned. These piglets (Groups 3 and 4) were then challenged by the intravenous route with 1×10⁹ cfu of serotype 2 S. suis isolate 23/P0278/02/11.

Post-challenge the piglets from Groups 3 and 4 were observed twice daily for signs of disease and additional welfare observations were carried out as necessary.

On the 7th day post challenge, the remaining animals in Groups 3 and 4 were euthanised and samples were collected post-mortem for further analysis.

Two additional piglets from the Piggivac4-vaccinated sow (Group 5) and three additional piglets from the control sow (Group 6) were vaccinated on Day 119 when they were approximately 8 weeks of age. Serum samples were collected to measure antibody responses to the components of Piggivac4.

TABLE 4
Summary of the study design.

| Group | No. of Animals | Test Material | Route/Dose | Study Day | Challenge | End of Study |
|---|---|---|---|---|---|---|
| 1 | 1 (pregnant | Piggivac4 | IM/1 ml | Day 0, 21 | None | Weaning |
| | sow) | | | and 42 | | (Day 90) |
| 2 | 1 (pregnant | Control | IM/1 ml | Day 0, 21 | None | Weaning |
| | sow) | Material | | and 42 | | (Day 90) |
| | | (Placebo) | | | | |
| 3 | 9 (piglets) | None | N/A | N/A | S. suis/IV/2 ml/ | 7 days post |
| | | | | | 1 × 10⁹ cfu total/4 | challenge |
| | | | | | weeks of age (Day 90) | (Day 97) |
| 4 | 10 (piglets) | None | N/A | N/A | S. suis/IV/2 ml/ | 7 days post |
| | | | | | 1 × 10⁹ cfu total/4 | challenge |
| | | | | | weeks of age (Day 90) | (Day 97) |
| 5 | 2 (piglets) | Piggivac4 | IM/0.5 ml | 119 | None | Day 147 |
| 6 | 3 (piglets) | Piggivac4 | IM/0.5 ml | 119 | None | Day 147 |

Challenge Preparation: On Day 89, S. suis isolate (23/P0278/02/11) was streaked onto 5% sheep blood agar plates (2 plates in total). The plates were incubated aerobically overnight for 20 to 24 hours at +37° C. (2° C.).

Following incubation, plates were examined and confirmed as having growth consistent with that expected for the isolate. Colonies were removed from each plate and added to 2×3 ml of pre-warmed vegetable peptone broth supplemented with 3% (v/v) horse serum (supplemented VPB) in bijoux bottles and grown at +37° C. (±2° C.) to a turbidity of 1.5 McFarland turbidity units (McF) (optical density was measured using a Densitometer, BioMerieux). Each 3 ml volume was then added to 97 ml of pre-warmed supplemented VPB to give 2×100 ml challenge cultures, which were incubated for 4 (±0.5) hours at +37° C. (±2° C.) on an orbital shaker set at 150 rpm.

The contents of one culture was used as neat for the challenge of the animals from Groups 3 and 4, with an expected concentration of 5×10⁸ cfu/ml. A 2 ml sample of the challenge material was removed for back titration. The challenge material was stored chilled prior to use (+2 to +8° C.).

Challenge Administration: On Day 91, (at 4 weeks of age) all of the animals from Groups 3 and 4 were administered 2 ml of S. suis challenge material, at a concentration of 6.5×10⁸ to 7.5×10⁸ cfu/ml, as measured by back titration, by the intravenous route (jugular vein, using a separate sterile needle and syringe for each animal).

Clinical Observations: On the day of challenge (Day 91) a clinical observation was conducted by experienced personnel prior to challenge and then 4 (±1) hours post-challenge. Clinical observations were then conducted, twice daily from Day 91 until Day 97 with only one scheduled observation on Day 98 prior to necropsy. Additional welfare observations were conducted at a frequency determined by the condition of the animals during the period between Days 91 and 98.

Clinical observations consisted of assessments of demeanour, behavioral/central nervous system changes, lameness and rectal temperature (° C.) according to a scoring system (Table 5). Additional comments relating to behavioral or neurological issues were recorded as comments.

TABLE 5
Clinical scoring system

| | Score | | | |
|---|---|---|---|---|
| Parameter | 0 | 1 | 2 | 3 |
| Rectal Temp | ≥38.0° | >39.5° | >40.0° | ≥41.0° C. |
| | C.-≤39.5° C. | C.-≤40.0° C. | C.-<41.0° C. | or <38.0° C. |
| Demeanour | Normal | Mild | Moderate | Severe |
| | demeanour | Depression: | Depression: | Depression: |
| | | Slightly dull | Unwilling but | Unable to |
| | | but active and | able to rise, | rise |
| | | mobile | staying apart | |
| | | | from others | |
| Behavioural/CNS | Normal | Minor: | Moderate: | Severe: |
| | | Tremors | Uncoordinated | Fitting, |
| | | | | involuntary |
| | | | | muscle |
| | | | | movement |
| Mobility | Normal | Mild: | Moderate: | Severe: |
| | | Lameness in | Unsteady when | Paralysis |
| | | one limb | walking or | |
| | | | walking on front | |
| | | | knees, | |
| | | | lameness in | |
| | | | more than one | |
| | | | limb | |

The individual scores for each clinical score were summed during tabulation of data to also give the total clinical score for each animal on each observation.

Piglets which were recumbent/moribund and/or showing signs of severe distress, were euthanized immediately on humane grounds by administration of a lethal dose of Pentobarbitone Sodium BP. The last observation carried forward method was utilized in cases where data was missing as a result of euthanasia prior to the end of the study.

Scheduled Euthanasia: Sows were euthanized by an approved method following weaning of piglets (Day 85). On Day 98 all remaining piglets from Groups 3 and 4 were euthanized by lethal injection (Pentobarbitone Sodium BP (Vet) 20% w/v (Pentoject or equivalent).

Necropsy: On Day 98 (or as required following early euthanasia on welfare grounds) the piglets from Groups 3 and 4 were euthanized by lethal injection. A gross pathological examination of each carcass was conducted with details recorded and samples collected for further analysis.

### Sample Collection and Processing:

Blood Sampling (sows): Blood samples (up to 10 ml) were collected from all sows on Days −14, 0, 7, 14, 21, 28, 35, 42, and 49. Blood was collected into 10 ml evacuated non-heparinized blood tubes using suitably sized sterile needles and syringes.

Blood Sampling (piqlets): Blood samples (up to 5 ml) were collected from piglets in groups 3 and 4 on Days 62, 70, 77, 84 and 91. Blood samples (up to 5 ml) were collected from piglets in groups 5 and 6 on Days 62, 70, 84, 91, 119, 127, 131, 133, 140 and 147.

Blood Sample Processing: Following collection, non-heparinized blood samples were transferred to the laboratory and allowed to clot standing at +2 to +8° C. overnight (16 to 24 hours) or at +37° C. (+2° C.) for between 30 and 45 minutes. Clotted blood samples were then centrifuged at 2000×g (approximately 3000 rpm) for 20 minutes and the sera transferred into duplicate plastic serum collection tubes. Serum aliquots were stored at −20° C. (+10° C.) prior to analysis.

Colostrum Sampling: As soon as possible post-farrowing (within 24 hours) colostrum (up to 10 ml) was collected from each sow into a sterile container.

Samples were transferred to the laboratory and a volume of rennet (approximately 20 μl rennet to 1 ml of milk) were added to the colostrum, mixed thoroughly and incubated at 37° C. (±2° C.) for 1 hour. Following incubation, each sample was centrifuged at 18,000×g for 3 minutes and the whey fraction removed into a separate sterile container. Whey samples were stored at −20° C. (+10° C.) prior to analysis.

Necropsy Samples: At necropsy, one brain sample and two tonsil samples were removed from each animal using sterile forceps and scalpels and placed in sterile containers for bacteriological analysis. The remaining tonsil sample was placed in a sterile container and stored at −20° C. (±10° C.).

At necropsy, cotton tipped cotton swabs were used to collect samples from the brain stem, pleura, peritoneum, spleen, joint (front left leg) and pericardium.

ELISA with antigen-coated plates to detect IgGs from porcine serum: 8×12 flat bottomed microtiter plates (Nunc) were coated with 100 μL of each protein antigen (4 μg/ml, dissolved in PBS pH 7.3) per well, overnight at 20° C.-25° C. After the coating, 100 μL 2% BSA (bovine serum albumin, Sigma-Aldrich, cat #A4503) in PBS were added to each well, and the plates incubated for one hour at 37° C. Wells were washed 5 times with 250 μL PBST (PBS with 0.05% Tween 20, Sigma-Aldrich, cat #P1379) with a Tecan HydroSpeed plate washing machine. 100 μL of 5% animal serum in PBST were added to the first well and subsequent 2-fold serial dilutions of serum added to the remaining wells of the row. The plates were incubated for two hours at 37° C. Wells were washed 5 times with 250 μL PBST. 100 μL anti-pig IgG antibodies conjugated with HRP (horseradish peroxidase) (Sigma-Aldrich, cat #A5670), diluted 10,000-fold in PBST, were added to the wells, and the plates were incubated for one hour at 37° C. Wells were washed 5 times with 250 μL PBST. 100 μL OPD substrate prepared according to manufacturer's recommendation (Sigma-Aldrich, cat #P9187) were added to each well. After 14 minutes of incubation, the reaction was stopped by adding 100 μL 0.5 mol/L H₂SO₄. The absorbance at 492 nm was measured with a plate spectrophotometer (Tecan Sunrise). The titre was calculated from the plot of the logarithm of the sera dilutions against the absorbance at 492 nm. The regression line of the linear points around an absorbance of 1.5 was determined in MS Excel, and the titre calculated as the log of the serum dilution corresponding to an absorbance of 1.5.

Results: Piggivac4 induced antibody responses in the vaccinated sow. Both sows had similar antibody levels to the components of Piggivac4 prior to first vaccination. Sow 2580, which was vaccinated with Piggivac4 had elevated antibody levels to the vaccine components from day 7 post-vaccination, whilst there was no change in the levels measured in sera from the control sow (Table 6).

TABLE 6
Antibody titres to the Piggivac4 antigens
in sera recovered from pregnant sows.

| Animal | | Day of study | | | | | | | |
|---|---|---|---|---|---|---|---|---|---|
| No. | Group | 0 | 7 | 14 | 21 | 28 | 35 | 42 | 49 |
| 2580 | 1 | 2.2 | 3.2 | 3.8 | 3.6 | 3.8 | 3.9 | 3.9 | 3.9 |
| 2575 | 2 | 2.3 | 2.4 | 2.3 | 2.4 | 2.4 | 2.4 | 2.4 | 2.2 |

Significant Antibody Responses were Transferred to the Piglets from the Piggivac4-Vaccinated Sow.

The piglets in group 3 from the vaccinated sow had significantly higher levels of antibodies to the components of Piggivac4 from birth and throughout the study period when compared to the levels measured in piglets in group 4 from the control sow (P<0.0001 at all timepoints, Table 7).

TABLE 7
Antibody titres to the Piggivac4 antigens in sera recovered from piglets.

| | | Day of study | | | | | | | | | | |
|---|---|---|---|---|---|---|---|---|---|---|---|---|
| Animal | Group | 62 | 70 | 77 | 84 | 91 | 119 | 127 | 131 | 133 | 140 | 147 |
| 342009 | 3 | 3.9 | 3.6 | 3.4 | 3.2 | 3.0 | | | | | | |
| 342011 | | 3.7 | 3.6 | 3.4 | 3.2 | 3.0 | | | | | | |
| 342012 | | 3.5 | 3.2 | 2.9 | 2.7 | 2.5 | | | | | | |
| 342013 | | 3.3 | 3.0 | 2.7 | 2.5 | 2.4 | | | | | | |
| 342014 | | 3.8 | 3.4 | 3.2 | 3.0 | 2.8 | | | | | | |
| 342015 | | 4.0 | 3.7 | 3.5 | 3.3 | 3.1 | | | | | | |
| 342016 | | 4.1 | 3.7 | 3.4 | 3.2 | 3.0 | | | | | | |
| 342017 | | 3.7 | 3.3 | 3.0 | 2.8 | 2.6 | | | | | | |
| 342019 | | 3.6 | 3.3 | 3.0 | 2.9 | 2.7 | | | | | | |
| 342025 | 4 | 2.2 | 1.6 | 1.4 | 1.2 | 1.1 | | | | | | |
| 342026 | | 2.4 | 2.0 | 1.6 | 1.3 | 1.1 | | | | | | |
| 342027 | | 1.7 | 1.3 | 1.2 | 1.0 | 1.0 | | | | | | |
| 342028 | | 2.0 | 1.5 | 1.2 | 1.0 | 0.8 | | | | | | |
| 342030 | | 2.2 | 2.0 | 1.7 | 1.4 | 1.3 | | | | | | |
| 342033 | | 1.4 | 0.9 | 0.8 | 0.7 | 0.7 | | | | | | |
| 342034 | | 2.2 | 1.9 | 1.5 | 1.3 | 1.2 | | | | | | |
| 342036 | | 2.4 | 2.0 | 1.4 | 1.3 | 1.2 | | | | | | |
| 342042 | | 2.4 | 2.1 | 1.8 | 1.6 | 1.3 | | | | | | |
| 342043 | | — | — | 1.6 | 1.3 | 1.2 | | | | | | |
| 342010 | 5 | 3.7 | 3.2 | 3.1 | 3.0 | 2.8 | 2.2 | 2.2 | 2.1 | 2.0 | 2.0 | 1.9 |
| 342023 | | 3.3 | 3.0 | 2.9 | 2.7 | 2.5 | 1.8 | 1.9 | 1.9 | — | 2.0 | 1.9 |
| 342029 | 6 | 1.0 | 0.7 | 0.4 | 0.8 | 0.8 | — | 2.6 | 2.5 | 2.5 | 2.5 | 2.5 |
| 342031 | | 2.0 | 1.5 | 1.2 | 1.0 | 0.9 | 1.7 | 2.3 | 2.3 | 2.2 | 2.1 | 2.0 |
| 342040 | | 2.2 | 1.8 | 1.3 | 1.2 | 1.2 | 1.6 | 2.6 | 2.7 | 2.6 | 2.6 | 2.2 |

N/A indicates that the sample was unavailable for analysis.

More piglets from the Piggivac4-vaccinated sow reached the end of the study at 7 days post-challenge.

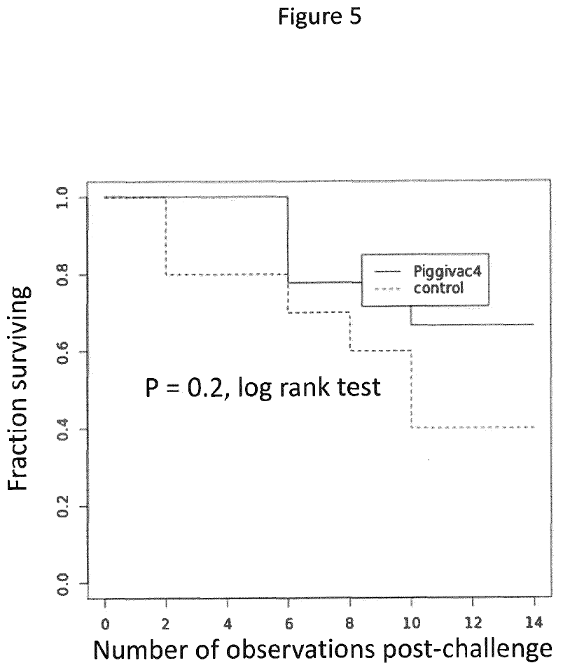

Overall, 3 of 9 piglets in group 3 and 6 of 10 piglets in group 4 were euthanized before the end of the study (P=0.4, Fisher's exact two-tailed test). The first piglets in group 4, from the control sow, were euthanized on reaching the humane endpoint on day 1 post-challenge, whilst the first piglets from group 4 were euthanized on day 3 post-challenge, but the earlier time to euthanasia did not reach statistical significance (P=0.3, two-tailed Mann-Whitney U test) and an analysis by the log rank test did not reach statistical significance (P=0.2, FIG. 5). A clear trend was however observed.

Piglets from the Piggivac4-Vaccinated Sow had Significantly Lower Rectal Temperatures on Days 1 and 2 Post-Challenge.

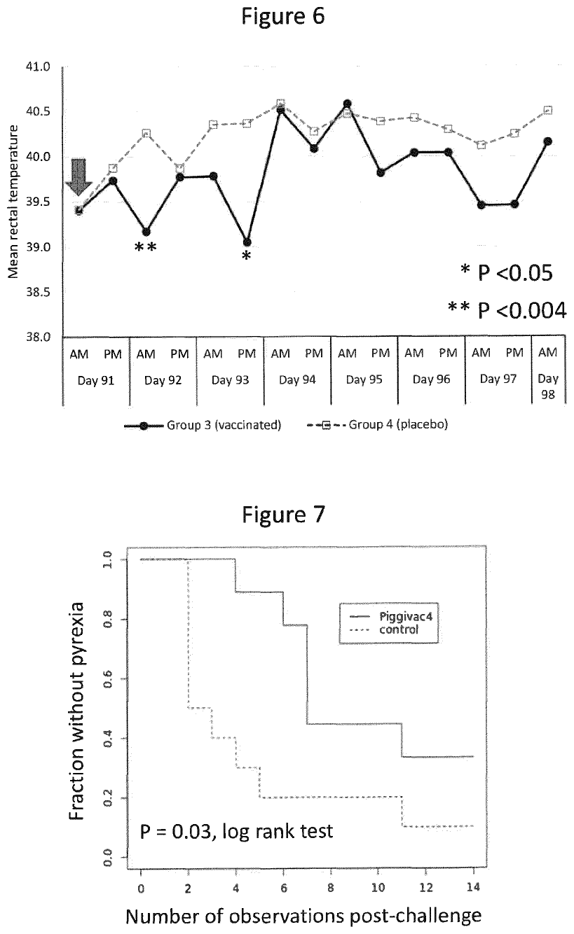

The slower onset of severe signs in piglets from the sow vaccinated with Piggivac4 was reflected by significantly lower rectal temperatures on days 1 and 2 post-challenge in the piglets in group 3 (P=0.001, P=0.006, respectively, two-tailed student's t test, FIG. 6).

The onset of rises in rectal temperature was also significantly delayed in piglets from the Piggivac4-vaccinated sow as shown by the time taken for piglets to develop a rectal temperature of 40° C. or higher on two of three consecutive observations (P=0.03, log rank test, FIG. 7).

Piglets from the Piggivac4-Vaccinated Sow had Lower Demeanour Scores Post-Challenge.

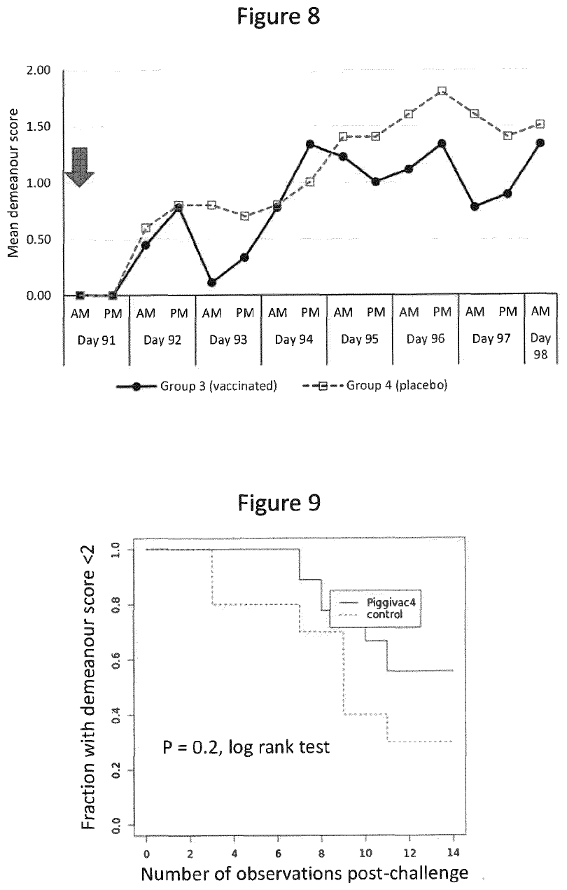

The demeanour scores of piglets in group 3 were lower than those in the control group, but these did not reach statistical significance (P≥0.1 on all days, two-tailed Mann-Whitney U test, FIG. 8).

Overall, 3 of 9 piglets in group 3 and 7 of 10 piglets in group 4 developed a demeanour score of 2 or above before the end of the study (P=0.2, Fisher's exact two-tailed test). The first piglets in both groups developed a demeanour score of 2 or higher on day 2 post-challenge, and there was no significant difference statistical significance in the onset of demeanour scores by the log rank test (P=0.2, FIG. 9). A clear trend was however observed.

Piglets from the Piggivac4-Vaccinated Sow had Lower CNS Scores Post-Challenge.

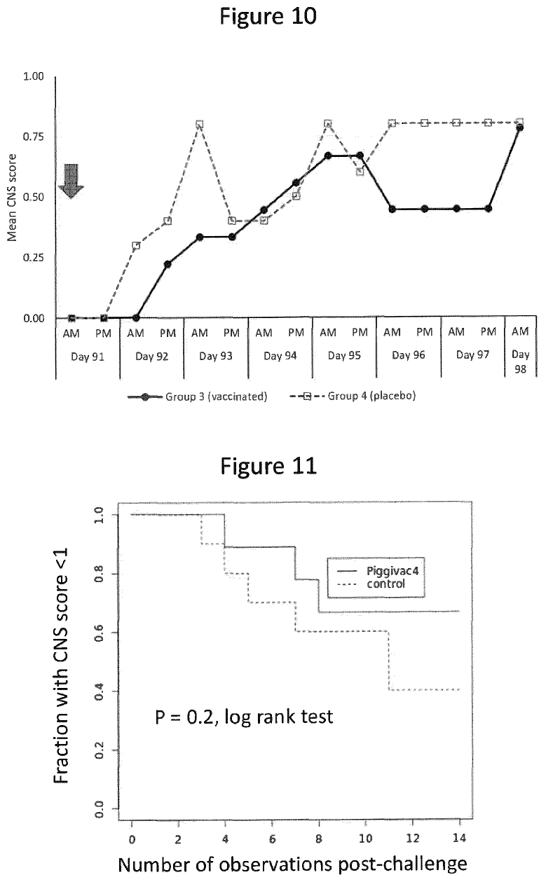

The CNS scores of piglets in group 3 were lower than those in the control group, but these did not reach statistical significance (P≥0.2 on all days, two-tailed Mann-Whitney U test, FIG. 10).

Overall, 3 of 9 piglets in group 3 and 6 of 10 piglets in group 4 developed a CNS score of 1 for two out of three consecutive days before the end of the study (P=0.4, Fisher's exact two-tailed test). The first piglets in both groups developed a CNS score of 1 or higher on day 2 post-challenge, and there was no significant difference statistical significance in the onset of CNS scores by the log rank test (P=0.3, FIG. 11). A clear trend was however observed.

Piglets from the Piggivac4-Vaccinated Sow had Lower Mobility Scores Post-Challenge.

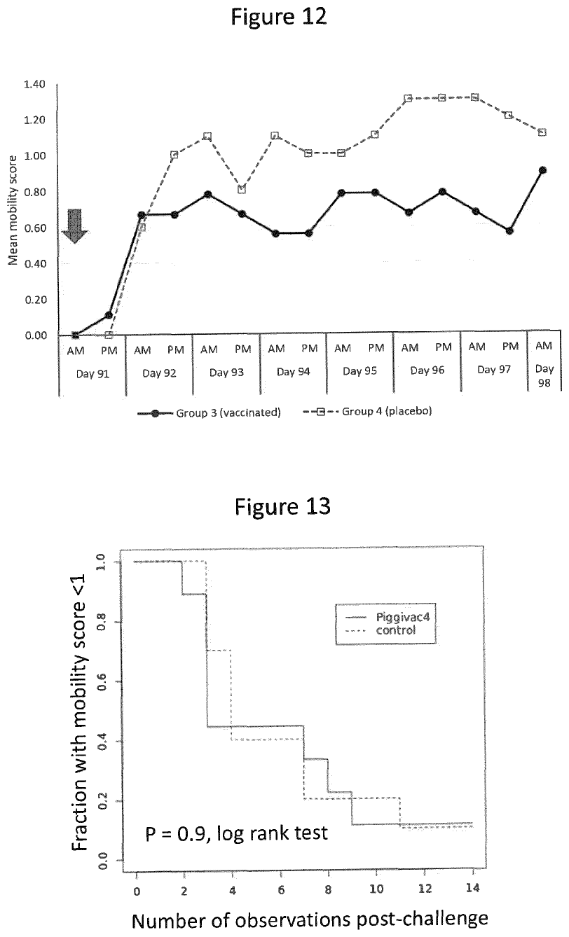

The mobility scores of piglets in group 3 were lower than those in the control group, but these did not reach statistical significance (P≥0.1 on all days, two-tailed Mann-Whitney U test, FIG. 12).

Overall, 8 of 9 piglets in group 3 and 9 of 10 piglets in group 4 developed a mobility score of 1 for two out of three consecutive days before the end of the study (P=1.0, Fisher's exact two-tailed test). The first piglets in both groups developed a mobility score of 1 or higher for two consecutive observations on day 2 post-challenge, and there was no significant difference statistical significance in the onset of mobility scores by the log rank test (P=0.9, FIG. 13). A clear trend was however observed.

Piglets from the Piggivac4-Vaccinated Sow had Lower Total Clinical Scores Post-Challenge.

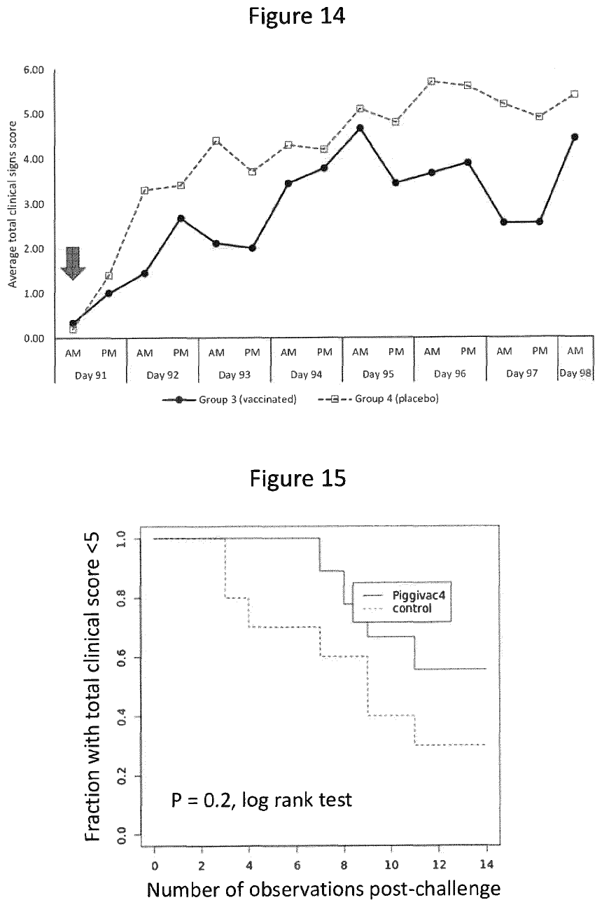

The total clinical scores of piglets in group 3 were lower than those in the control group, but these did not quite reach statistical significance (P≥0.1 on all days, two-tailed Mann-Whitney U test, FIG. 14).

Overall, 4 of 9 piglets in group 3 and 7 of 10 piglets in group 4 developed a total clinical score of >5 for two out of three consecutive days before the end of the study (P=0.4, Fisher's exact two-tailed test). The first piglets in group 3 developed a total clinical score of 5 or higher for two out of three consecutive observations on day 2 post-challenge, whilst the first piglet in group 4 reached this total clinical score for two out of three consecutive observations on day 4. However, there was no significant difference statistical significance in the onset of total clinical scores greater by the log rank test (P=0.2, FIG. 15), but a clear trend was observed.

### Example 8

### Efficacy of a Streptococcus suis Vaccine Against an Experimental Streptococcus suis Challenge in Piglets

In this Example, 1 ml of the five fusion polypeptides namely BCA, 4Zn+3, 5AsL, SP274C-ny and 3PCS, corresponding to SEQ ID NO: 49, 50, 51, 53 and 54 and comprising SEQ ID NO:43, 44, 45, 46 and 48, respectively. The combination of said five fusion polypeptides is referred to as Piggivac5. 1 ml containing 100 μg of said five fusion polypeptides, also referred to as fusion antigens, with 100 μg of MatrixV adjuvant or an adjuvant-only control were injected into two groups of four pregnant sows (Group 1 and 2, respectively) on days −92, −71 and −43.

Piglets were farrowed and weaned at approximately 4 weeks of age. Two groups of 20 piglets, comprised of approximately 5 piglets from each vaccinated (Group 3) or control (Group 4) sow were challenged by intravenous injection with a dose of between 8.3×10⁷ and 1.1×10⁸ of serotype 2 S. suis isolate 23/P0278/02/11 at four weeks of age. Piglets in Groups 3 and 4 were then monitored for 14 days for signs of infection. Piglets were euthanized on reaching the criteria for welfare endpoint or at the end of the study on day 14 post-challenge.

In a second challenge phase, two groups of 20 piglets, comprised of approximately 5 piglets from each vaccinated (Group 5) or control (Group 6) sow were challenged by intravenous injection with a dose of between 6.5×10⁷ and 7.1×10⁷ of serotype 2 S. suis isolate 23/P0278/02/11 at 7 weeks of age. Piglets in Groups 5 and 6 were then monitored for 14 days for signs of infection. Piglets were euthanized on reaching the criteria for welfare endpoint or at the end of the study on day 14 post-challenge.

Piglets from the Piggivac5-vaccinated sows (Group 3) had significantly lower rectal temperatures, demeanour scores, CNS scores, mobility scores and total clinical scores than piglets from the control sows (Group 4). The onset of rectal temperatures of >40.5° C. and all clinical scores were significantly delayed in piglets in Group 3. There was a trend for piglets in Group 3 to be euthanized on reaching the clinical endpoint later than piglets in Group 4 with 11 of 20 piglets in Group 3 vs. 8 of 20 in Group 4 remaining alive at the end of the study period. Piglets from Piggivac5-vaccinated sows in Group 3 gained significantly more weight post-challenge than those in Group 4.

In the second challenge phase, piglets from the Piggivac5-vaccinated sows (Group 5) had significantly lower demeanour scores, CNS scores, mobility scores and total clinical scores than piglets from the control sows (Group 6). The onset of all clinical scores were significantly delayed in piglets in Group 5. There was a trend for piglets in Group 5 to be euthanized on reaching the clinical endpoint later than piglets in Group 6 with 8 of 20 piglets in Group 5 vs. 4 of 20 in Group 6 remaining alive at the end of the study period. Piglets from Piggivac5-vaccinated sows in Group 5 tended to gain more weight post-challenge than those in Group 6.

In summary, vaccination of sows with Piggivac5 significantly protected their piglets from the effects of S. suis challenge at both 4 and 7 weeks of age.

### Materials and Methods:

Test animals: Details on the sows and piglets used in this study are given in Table 7 below.

TABLE 8
Detail on animals used in the study.

| Animal details | Sows | Piglets |
|---|---|---|
| Species | Porcine | Porcine |
| Breed | Yorkshire - Landrace | Yorkshire - Landrace |
| Sex | Female | Female/Male |
| Status | Pregnant (~40 day's | Age: <24 hours |
| | gestation on arrival) | |
| Number | 8 | 80 |
| Source | High Health Status | Farrowed at Moredun |
| | Commercial pig unit without | |
| | known history of S. suis | |
| | infection | |
| Fate of | All study animals were | All study animals were |
| animals | euthanized and | euthanized and |
| | incinerated. | incinerated. |

Test and control material: The test material was prepared according to Table 9 and the control Group received a control dose of adjuvant, which was selected to mimic the test material dose regime. The formulation comprising BCA, 4Zn+3, 5AsL, SP274 and 3PCS is herein also referred to as “Piggivac5”.

Study design and procedure: A summary of the study design is given in Table 10. A total of eight pregnant pigs (approximately 40 days gestation) were sourced from a commercial pig farm with no history of Streptococcus suis clinical disease.

Once on site the animals were housed together on straw and were provided with drinking water and feed for pregnant sows available ad libitum. On arrival the sows were examined by a veterinarian to confirm that they were in good general health and a blood sample was collected from each. A blood sample was also collected prior to each vaccination and every week during the vaccination period. Sows in Group 1 were administered Piggivac5 by the intramuscular route, and sows in Group 2 were injected with the Placebo vaccine on days −92, −71 and −43. On day −36, the animals were moved to farrowing crates. Animals farrowed on days −29 and −28. A final blood sample was collected following completion of farrowing.

TABLE 9
Information on test and control material.

| Test material | Study | Control |
|---|---|---|
| Name/Code | Piggivac5 | Placebo |
| Number | | |
| Formulation | Protein solution in 50 | Adjuvant solution in 50 |
| | mM Tris pH 7.3 | mM Tris pH 7.3 |
| Concentration | 100 μg per antigen in | 100 μg/ml of Matrix V |
| | 1000 μl injection volume | (Novavax) |
| | containing 100 μg/ml of | |
| | Matrix V (Novavax) | |
| Storage | +2 to +8° C. | +2 to +8° C. |
| Conditions | | |
| Required | | |
| Method of | Intramuscular injection | Intramuscular injection |
| Administration | | |
| Dose Regime | 1 ml on days −92, −71 | 1 ml on days −92, −71 |
| | and −43 | and −43 |

Once farrowed, piglets were examined to confirm they were in good health and a record was made of the number of live/dead piglets. All piglets were encouraged to consume as much colostrum as possible. Blood samples were collected from all piglets (Groups 3, 4, 5 and 6) within 24 hours of farrowing then on days 0 and 14. At 28 days of age all piglets were examined by a vet. Ten piglets were selected from each litter and weaned. The piglets from the sows vaccinated with Piggivac5 (Groups 3 and 5) and those from the placebo vaccinated sows (Groups 4 and 6) were then challenged by the intravenous route with serotype 2 S. suis isolate 23/P0278/02/11 at 4 weeks of age (Groups 3 and 4) or at 7 weeks of age (Groups 5 and 6).

Post-challenge the piglets were observed twice daily for signs of disease and additional welfare observations were carried out as necessary.

On the 14th day post challenge, the remaining animals were euthanised and samples were collected post-mortem for further analysis.

TABLE 10
Summary of the study design.

| | No. of | Test | | Study | | End of |
|---|---|---|---|---|---|---|
| Group | Animals | Material | Route/Dose | Day | Challenge | Study |
| 1 | 4 (pregnant | Piggivac5 | IM/1 ml | Day −92, −71 | None | Weaning |
| | sows) | | | and −43 | | (Day −4) |
| 2 | 4 (pregnant | Control | IM/1 ml | Day −92, −71 | None | Weaning |
| | sows) | Material | | and −43 | | (Day −4) |
| | | (Placebo) | | | | |
| 3 | 20 (piglets) | None | N/A | N/A | S. suis/IV/2 ml/of 8.3 x 10⁷ | 14 days |
| | | | | | to 1.1 x 10⁸ cfu total/4 | post |
| | | | | | weeks of age (Day 0) | challenge |
| | | | | | | (Day 14) |
| 4 | 20 (piglets) | None | N/A | N/A | S. suis/IV/2 ml/of 8.3 x 10⁷ | 14 days |
| | | | | | to 1.1 x 10⁸ cfu total/4 | post |
| | | | | | weeks of age (Day 0) | challenge |
| | | | | | | (Day 14) |
| 5 | 20 (piglets) | None | N/A | N/A | S. suis/IV/2 ml/of 6.5 x 10⁷ | 14 days |
| | | | | | to 7.1 x 10⁷ cfu total/7 | post |
| | | | | | weeks of age (Day 23) | challenge |
| | | | | | | (Day 37) |
| 6 | 20 (piglets) | None | N/A | N/A | S. suis/IV/2 ml/of 6.5 x 10⁷ | 14 days |
| | | | | | to 7.1 x 10⁷ cfu total/7 | post |
| | | | | | weeks of age (Day 23) | challenge |
| | | | | | | (Day 37) |

TABLE 11
Study schedule

| Study Day(s) | Procedure(s) |
|---|---|
| −99 | Sow arrival (~40 days gestation), blood sample and |
| | veterinary examination |
| −92 | Sow blood sample, veterinary examination and Test |
| | Material Administration |
| −85 | Sow blood sample |
| −78 | Sow blood sample |
| −71 | Sow blood sample and Test Material Administration |
| −64 | Sow blood sample |
| −57 | Sow blood sample |
| −50 | Sow blood sample |
| −43 | Sow blood sample and Test Material Administration |
| −36 | Sow Blood sample and Move to farrowing crates |
| −29 | Farrowing, colostrum sample collection, veterinary |
| | assessment, ear tagging, blood sampling (non- |
| | heparinized) of piglets and sows |
| −28 | Farrowing, colostrum sample collection, sow blood sample |
| −27/−26 | Veterinary assessment, body weight, ear tagging, piglet |
| | blood sample, iron injection |
| Approx. −4 | Veterinary Observation, allocation to groups |
| 0 | Blood sample, Body weight, Clinical observation, |
| | Challenge (Groups 3 and 4) |
| 0 (+4 | Clinical observations (Groups 3 and 4) |
| hours) | |
| 1 to 13 | Clinical observations x 2 (Groups 3 and 4) |
| 7 and 14 | Clinical observations, Body weight, Blood sample |
| | collection, Euthanasia and Necropsy (Groups 3 and 4) |
| 23 | Blood sample, Body weight, Clinical observation, |
| | Challenge (Groups 5 and 6) |
| 23 (+4 | Clinical observations (Groups 5 and 6) |
| hours) | |
| 24 to 37 | Clinical observations x 2 (Groups 5 and 6) |
| 23 and 35 | Clinical observations, Body weight, Blood sample |
| | collection, Euthanasia and Necropsy (Groups 5 and 6) |

Challenge Preparation: On Day −1 and Day 22, S. suis isolate (23/P0278/02/11) was streaked onto 5% sheep blood agar plates (2 plates in total). The plates were incubated aerobically overnight for 20 to 24 hours at +37° C. (±2° C.).

Following incubation, plates were examined and confirmed as having growth consistent with that expected for the isolate. Colonies were removed from each plate and added to 2×3 ml of pre-warmed vegetable peptone broth supplemented with 3% (v/v) horse serum (supplemented VPB) in bijoux bottles and grown at +37° C. (±2° C.) to a turbidity of 1.5 McFarland turbidity units (McF) (optical density was measured using a Densitometer, BioMerieux). Each 3 ml volume was then added to 97 ml of pre-warmed supplemented VPB to give 2×100 ml challenge cultures, which were incubated for 4 (±0.5) hours at +37° C. (±2° C.) on an orbital shaker set at 150 rpm.

The contents of one culture was diluted 1:10 in 90 ml of VPB and used for the challenge of the animals from Groups 3 and 4, with an expected concentration of 5×10⁷ cfu/ml. A 2 ml sample of the challenge material was removed for back titration. The challenge material was stored chilled prior to use (+2 to +8° C.).

Challenge Administration: On Day 0, (at 4 weeks of age) and Day 23 (at 7 weeks of age) all of the animals from Groups 3 and 4 or Groups 5 and 6 were administered 2 ml of S. suis challenge material, at a concentration of 4.15×10⁷ to 5.05×10⁷ cfu/ml, and 3.25×10⁷ to 3.55×10⁷ cfu/ml, respectively, as measured by back titration, by the intravenous route (jugular vein, using a separate sterile needle and syringe for each animal).

Clinical Observations: On the day of challenge (Day 0 and 23) a clinical observation was conducted by experienced personnel prior to challenge and then 4 (±1) hours post-challenge. Clinical observations were then conducted, twice daily from Day 1 until Day 13 in the first phase and Day 24 to 37 in the second phase with only one scheduled observation on Days 14 or 37 prior to necropsy. Additional welfare observations were conducted at a frequency determined by the condition of the animals during the period between Days 1 and 14 or Days 24 to 37.

Clinical observations consisted of assessments of demeanour, behavioural/central nervous system changes, lameness and rectal temperature (° C.) according to a scoring system (Table 12). Additional comments relating to behavioural or neurological issues were recorded as comments.

TABLE 12
Clinical scoring system

| | Score | | | |
|---|---|---|---|---|
| Parameter | 0 | 1 | 2 | 3 |
| Rectal Temp | ≥38.0° | >39.5° | >40.0° | ≥41.0° C. |
| | C.-≤39.5° C. | C.-≤40.0° C. | C.-<41.0° C. | or <38.0° C. |
| Demeanour | Normal | Mild | Moderate | Severe |
| | demeanour | Depression: | Depression: | Depression: |
| | | Slightly dull | Unwilling but | Unable to |
| | | but active and | able to rise, | rise |
| | | mobile | staying apart | |
| | | | from others | |
| Behavioural/CNS | Normal | Minor: | Moderate: | Severe: |
| | | Tremors | Uncoordinated | Fitting, |
| | | | | involuntary |
| | | | | muscle |
| | | | | movement |
| Mobility | Normal | Mild: | Moderate: | Severe: |
| | | Lameness in | Unsteady when | Paralysis |
| | | one limb | walking or | |
| | | | walking on front | |
| | | | knees, | |
| | | | lameness in | |
| | | | more than one | |
| | | | limb | |

The individual scores for each clinical score were summed during tabulation of data to also give the total clinical score for each animal on each observation. Piglets which were recumbent/moribund and/or showing signs of severe distress, were euthanized immediately on humane grounds by administration of a lethal dose of Pentobarbitone Sodium BP. The last observation carried forward method was utilized in cases where data was missing as a result of euthanasia prior to the end of the study.

Scheduled Euthanasia: Sows were euthanized by an approved method following weaning of piglets (Day −4). On Day 14 all remaining piglets from Groups 3 and 4 were euthanized by lethal injection (Pentobarbitone Sodium BP (Vet) 20% w/v (Pentoject or equivalent). On Day 37 all remaining piglets from Groups 5 and 6 were euthanized by lethal injection (Pentobarbitone Sodium BP (Vet) 20% w/v (Pentoject or equivalent).

Necropsy: On Day 14 or 37 (or as required on welfare grounds) the piglets from Groups 3 and 4 were euthanized by lethal injection. Each carcass was weighed and a gross pathological examination was conducted with details recorded and samples collected for further analysis.

### Sample Collection and Processing:

Blood Sampling (sows): Blood samples (up to 10 ml) were collected from all sows on Days −99, −92, −85, −78, −71, −64, −57, −50, −43, −36. −29 and −28. Blood was collected into 10 ml evacuated non-heparinized blood tubes using suitably sized sterile needles and syringes.

Blood Sampling (piqlets): Blood samples (up to 1 ml) were collected from all piglets within 24 hours of birth (day −27/−26). Up to 5 ml of blood was then collected on days 0 and 14 (Groups 3 and 4) or days 0, 23 and 37 (Groups 5 and 6).

Blood Sample Processing: Following collection, non-heparinized blood samples were transferred to the laboratory and allowed to clot standing at +2 to +8° C. overnight (16 to 24 hours) or at +37° C. (+2° C.) for between 30 and 45 minutes. Clotted blood samples were then centrifuged at 2000×g (approximately 3000 rpm) for 20 minutes and the sera transferred into duplicate plastic serum collection tubes. Serum aliquots were stored at −20° C. (+10° C.) prior to analysis.

Colostrum Sampling: As soon as possible post-farrowing (within 24 hours) colostrum (up to 10 ml) was collected from each sow into a sterile container. Samples were transferred to the laboratory and a volume of rennet (approximately 20 μl rennet to 1 ml of milk) were added to the colostrum, mixed thoroughly and incubated at 37° C. (±2° C.) for 1 hour. Following incubation, each sample was centrifuged at 18,000×g for 3 minutes and the whey fraction removed into a separate sterile container. Whey samples were stored at −20° C. (+10° C.) prior to analysis.

Necropsy Samples: At necropsy, one brain sample and two tonsil samples were removed from each animal using sterile forceps and scalpels and placed in sterile containers for bacteriological analysis. The remaining tonsil sample was placed in a sterile container and stored at −20° C. (±10° C.). At necropsy, cotton tipped cotton swabs were used to collect samples from the brain stem, pleura, peritoneum, spleen, joint (front left leg) and pericardium for culture analysis.

Results Challenge Phase 1 (Groups 3 and 4, aged 4 weeks at time of challenge):

More Piglets from the Piggivac5-Vaccinated Sows Reached the End of the Study at 14 Days Post-Challenge.

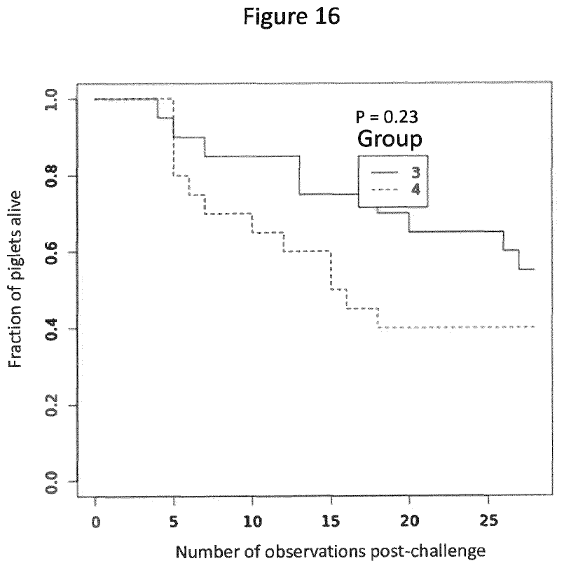

Overall, 9 of 20 piglets in Group 3 and 12 of 20 piglets in Group 4 were euthanized before the end of the study (P=0.53, Fisher's exact two-tailed test). Piglets in Group 4, from the control sows, tended to be euthanized earlier on reaching the humane endpoint, although the earlier time to euthanasia did not reach statistical significance (P=0.18, two-tailed Mann-Whitney U test) and an analysis by the log rank test did not reach statistical significance (P=0.23, FIG. 16).

Piglets from the Piggivac5-Vaccinated Sow had Significantly Lower Rectal Temperatures Post-Challenge.

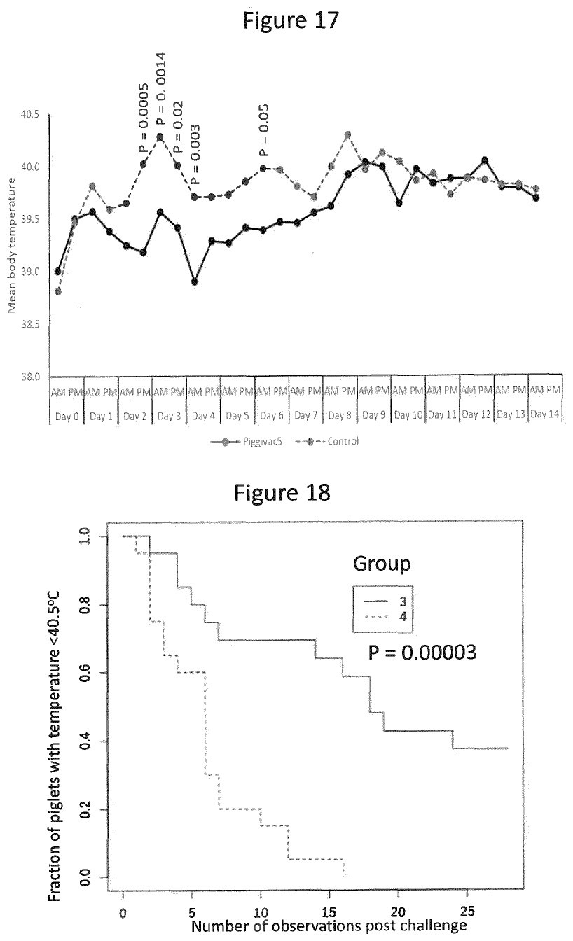

Twelve piglets from the Piggivac5-vaccinated sows and all 20 piglets from the control sows developed a body temperature of >40.5° C. (P=0.003, Fisher's exact two-tailed test). The slower onset of severe signs in piglets from the sows vaccinated with Piggivac5 was reflected by significantly lower rectal temperatures on days 2, 3, 4 and 6 post-challenge in the piglets in Group 3 (two-tailed student's t test, FIG. 17).

The onset of rises in rectal temperature was significantly delayed in piglets from the Piggivac5-vaccinated sows as shown by the time taken for piglets to develop a rectal temperature of 40.5° C. or higher (P=0.00003, log rank test, FIG. 18.

Piglets from the Piggivac5-Vaccinated Sow had Significantly Lower Demeanour Scores Post-Challenge.

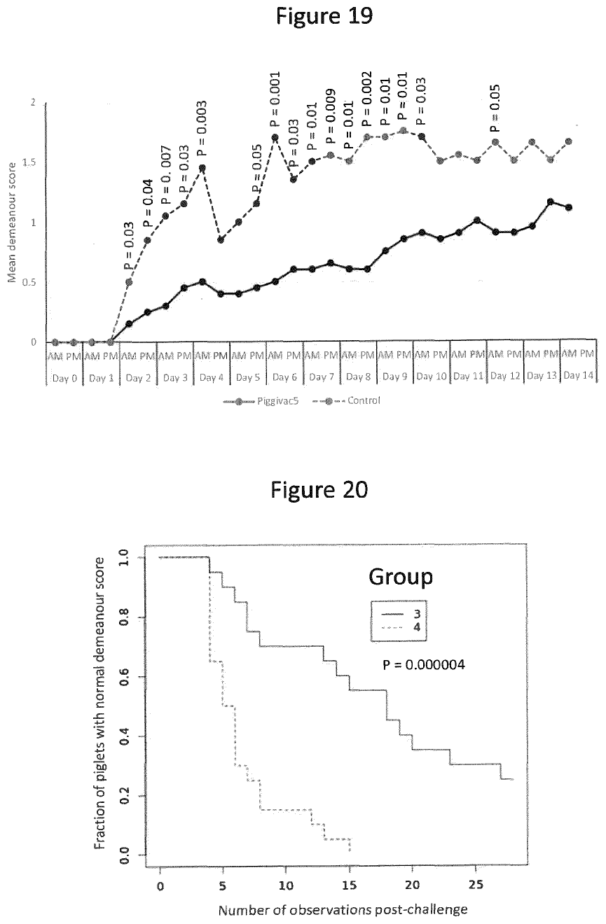

Fifteen piglets from the Piggivac5-vaccinated sows and all 20 piglets from the control sows developed a demeanour score (P=0.05, Fisher's exact two-tailed test). The demeanour scores of piglets in Group 3 were significantly lower than those in the control Group on days 2, 3, 4, 5, 6, 7, 8, 9, 10 and 12 post-challenge (Mann-Whitney U test, FIG. 19). The first piglets in both Groups developed a demeanour score of 1 or higher on day 2 post-challenge, but overall there was a highly significant statistical difference in the onset of demeanour scores by the log rank test (P=0.000004, FIG. 20).

Piglets from the Piggivac5-Vaccinated Sow had Significantly Lower CNS Scores Post-Challenge.

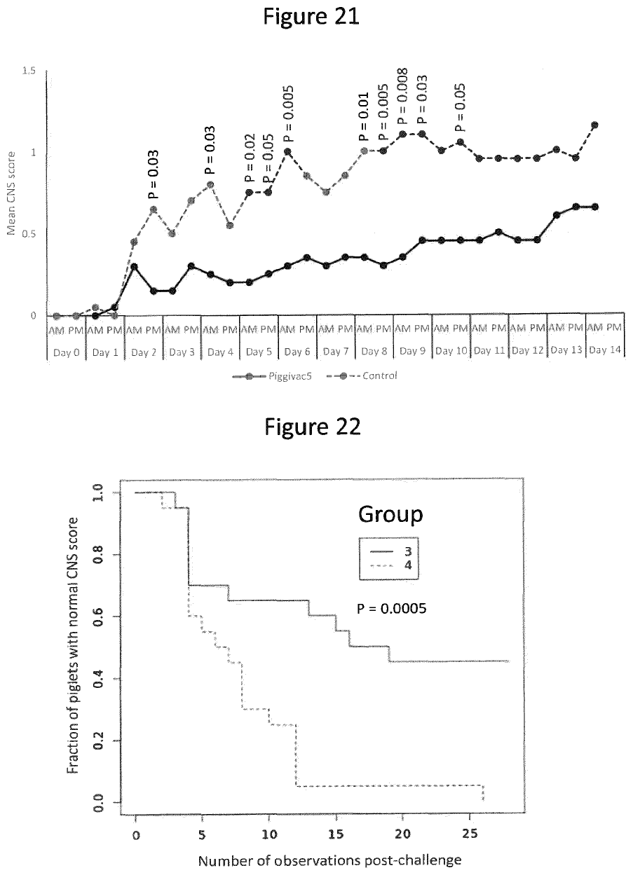

Eleven piglets from the Piggivac5-vaccinated sows and all 20 piglets from the control sows developed a CNS score (P=0.001, Fisher's exact two-tailed test). The CNS scores of piglets in Group 3 were significantly lower than those in the control Group on days 2, 4, 5, 6, 8, 9 and 10 post-challenge (two-tailed Mann-Whitney U test, FIG. 21). The first piglets in both Groups developed a CNS score of 1 or higher on day 1 post-challenge, but overall there was a highly significant difference in the onset of CNS scores by the log rank test (P=0.0005, FIG. 22).

Piglets from the Piggivac5-Vaccinated Sow had Significantly Lower Mobility scores post-challenge.

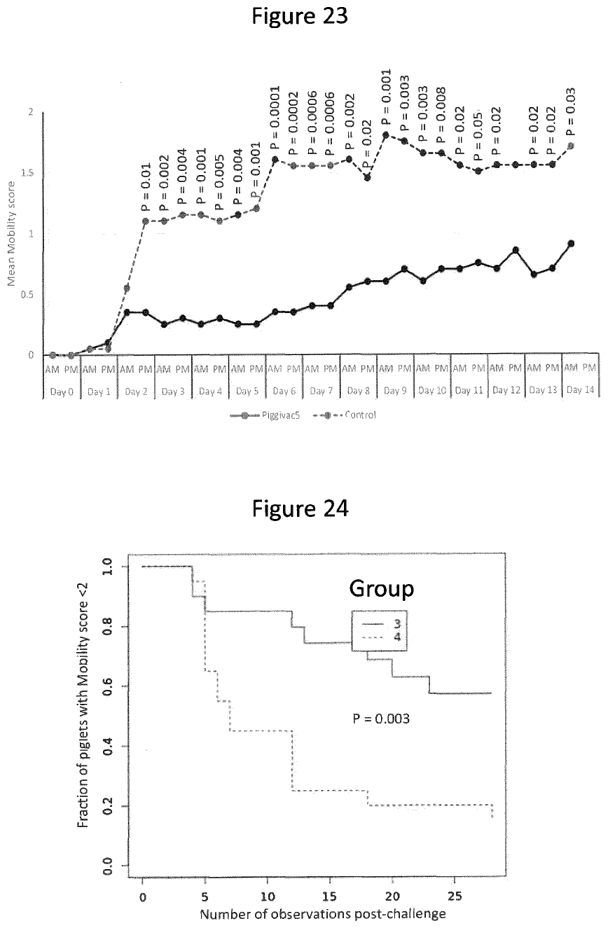

Overall, 8 of 20 piglets in Group 3 and 17 of 20 piglets in Group 4 developed a mobility score of >1 before the end of the study (P=0.008, Fisher's exact two-tailed test). The mobility scores of piglets in Group 3 were significantly lower than those in the control Group, on days 2, 3, 4, 5, 6, 7, 8, 9, 10, 11, 12, 13 and 14 post-challenge (two-tailed Mann-Whitney U test, FIG. 23). The first piglets in both Groups developed a mobility score of >1 on day 2 post-challenge, but there was a highly significant difference in the onset of mobility scores >1 by the log rank test (P=0.003, FIG. 24).

Piglets from the Piggivac5-Vaccinated Sow had Significantly Lower Total Clinical Scores Post-Challenge.

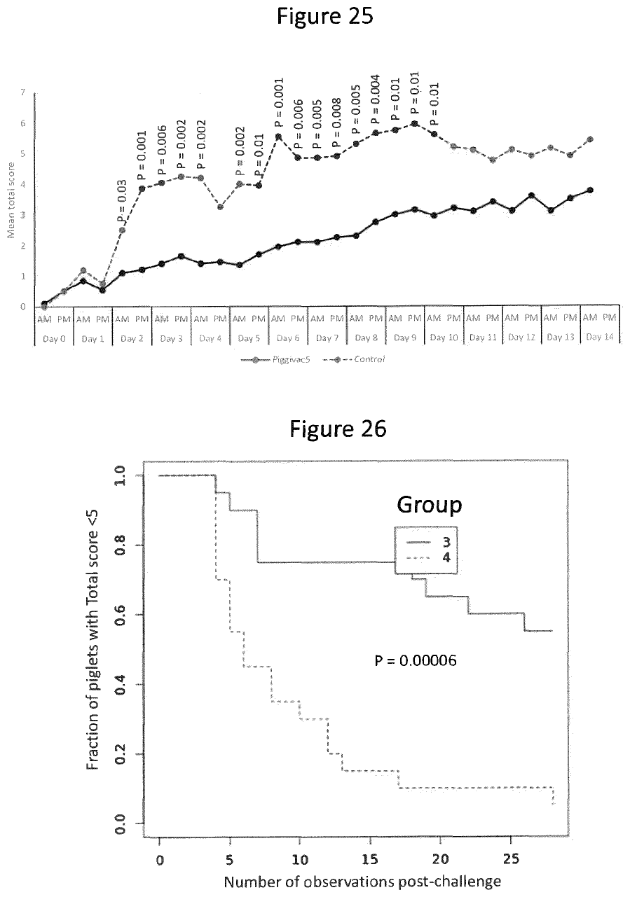

Overall, 9 of 20 piglets in Group 3 and 19 of 20 piglets in Group 4 developed a total clinical score of >5 before the end of the study (P=0.001, Fisher's exact two-tailed test). The total clinical scores of piglets in Group 3 were significantly lower than those in the control Group on days 2, 3, 4, 5, 6, 7, 8, 9 and 10 (two-tailed Mann-Whitney U test, FIG. 25). The first piglets in both Groups developed a total clinical score of 5 or higher for two out of three consecutive observations on day 2 post-challenge. However, there was a highly significant difference in the onset of total clinical scores of 5 or more by the log rank test (P=0.0006, FIG. 26).

Piglets from Vaccinated Sows Gained Significantly More Weight Post-Challenge.

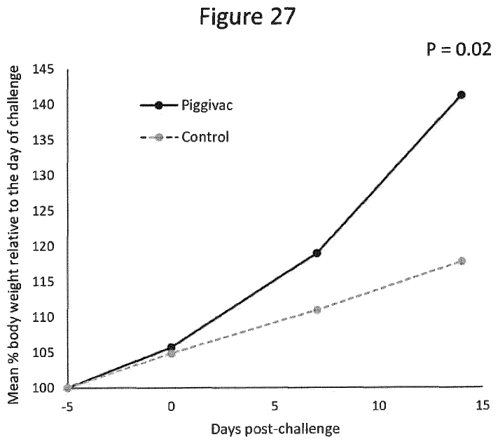

Piglets were weighed five days prior to challenge, immediately before challenge and then on days 7 and 14 post-challenge, or at the time of euthanasia. The weight of each piglet as a percentage relative to the time of challenge (day 0) was calculated. The weight of piglets from vaccinated sows did not differ significantly to those from unvaccinated sows on days −5 or 0 pre-challenge. However, piglets from sows vaccinated with Piggivac5 gained significantly more weight post-challenge than those in Group 4 by day 14 post-challenge (P=0.02, FIG. 27).

Results Challenge Phase 2 (Groups 5 and 6, aged 7 weeks at time of challenge):

More Piglets from the Piggivac5-Vaccinated Sows Reached the End of the Study at 14 Days Post-Challenge.

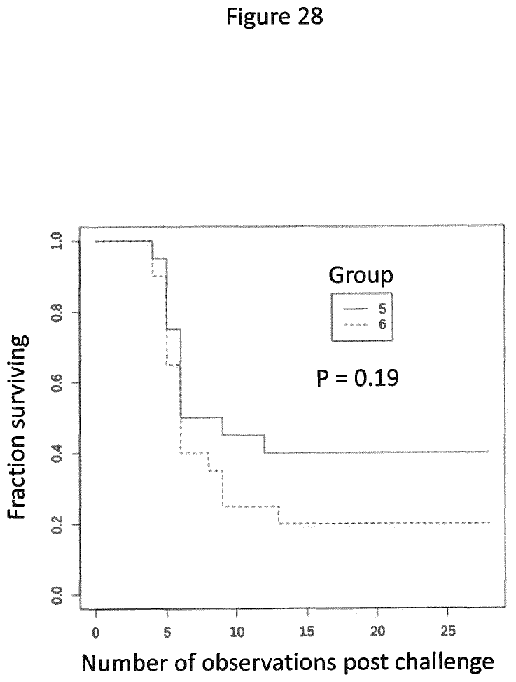

Overall, 12 of 20 piglets in Group 5 and 16 of 20 piglets in Group 4 were euthanized before the end of the study (P=0.30, Fisher's exact two-tailed test). Piglets in Group 6, from the control sows, tended to be euthanized earlier on reaching the humane endpoint, although the earlier time to euthanasia did not reach statistical significance (P=0.28, two-tailed Mann-Whitney U test) and an analysis by the log rank test did not reach statistical significance (P=0.19, FIG. 28).

Piglets from the Piggivac5-Vaccinated Sow Tended to have Lower Rectal Temperatures Post-Challenge.

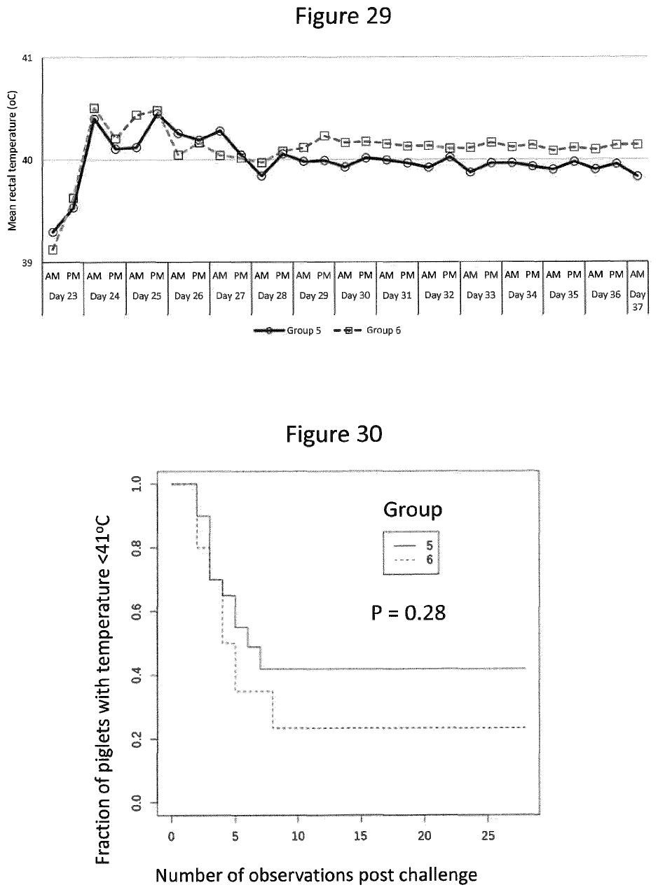

Eleven piglets from the Piggivac5-vaccinated sows and 14 piglets from the control sows developed a body temperature of >41° C. (P=0.51, Fisher's exact two-tailed test). The piglets from the sows vaccinated with Piggivac5 (Group 5) tended to have lower rectal temperatures than the piglets from control sows in Group 6 from day 6 post challenge (FIG. 29). The onset of rises in rectal temperature was delayed in piglets from the Piggivac5-vaccinated sows as shown by the time taken for piglets to develop a rectal temperature of 41° C. or higher, but this did not achieve statistical significance (P=0.28, log rank test, FIG. 30).

Piglets from the Piggivac5-Vaccinated Sow had Significantly Lower Demeanour Scores Post-Challenge.

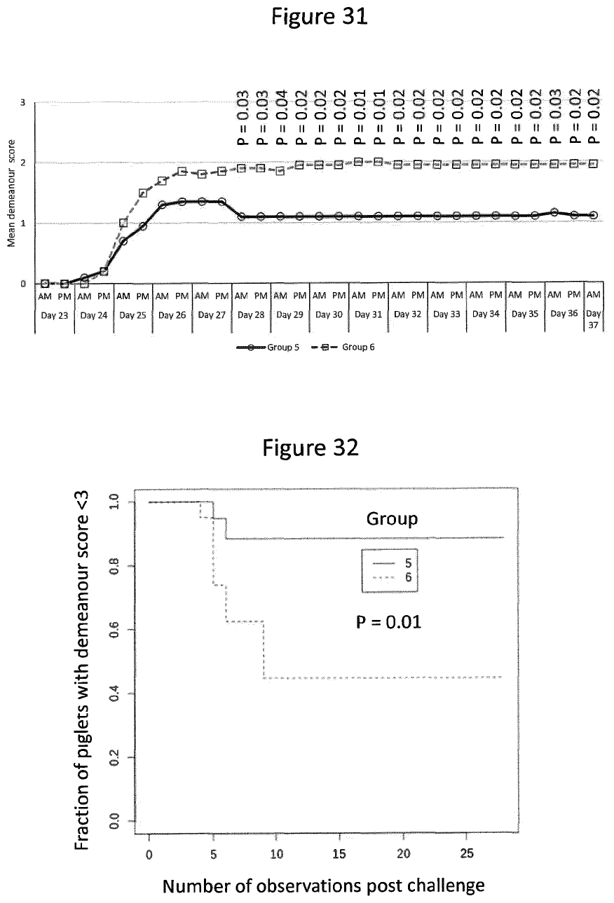

Two piglets from the Piggivac5-vaccinated sows and nine piglets from the control sows developed a demeanour score >2 (P=0.03, Fisher's exact two-tailed test). The demeanour scores of piglets in Group 5 were significantly lower than those in the control Group on days 5 to 14 post-challenge (Mann-Whitney U test, FIG. 31). There was a significant statistical difference in the onset of demeanour scores >2 by the log rank test (P=0.01, FIG. 32).

Piglets from the Piggivac5-Vaccinated Sow had Significantly Lower CNS Scores Post-Challenge.

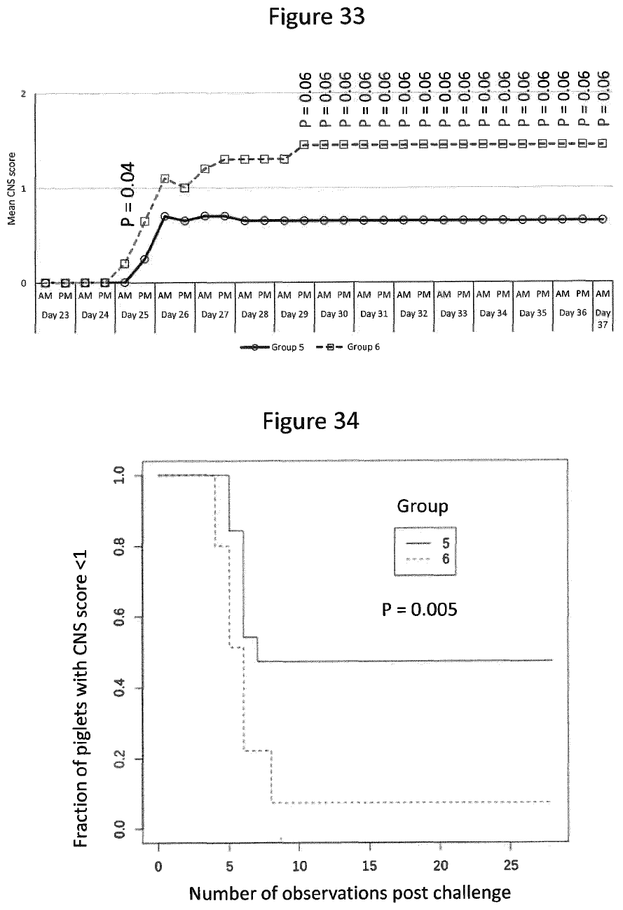

Nine piglets from the Piggivac5-vaccinated sows and 15 piglets from the control sows developed a CNS score (P=0.11, Fisher's exact two-tailed test). The CNS scores of piglets in Group 5 were significantly lower than those in the control Group on day 2 post-challenge (two-tailed Mann-Whitney U test, FIG. 33). There was a significant difference in the onset of CNS scores by the log rank test (P=0.005, FIG. 34).

Piglets from the Piggivac5-Vaccinated Sow had Significantly Lower Mobility Scores Post-Challenge.

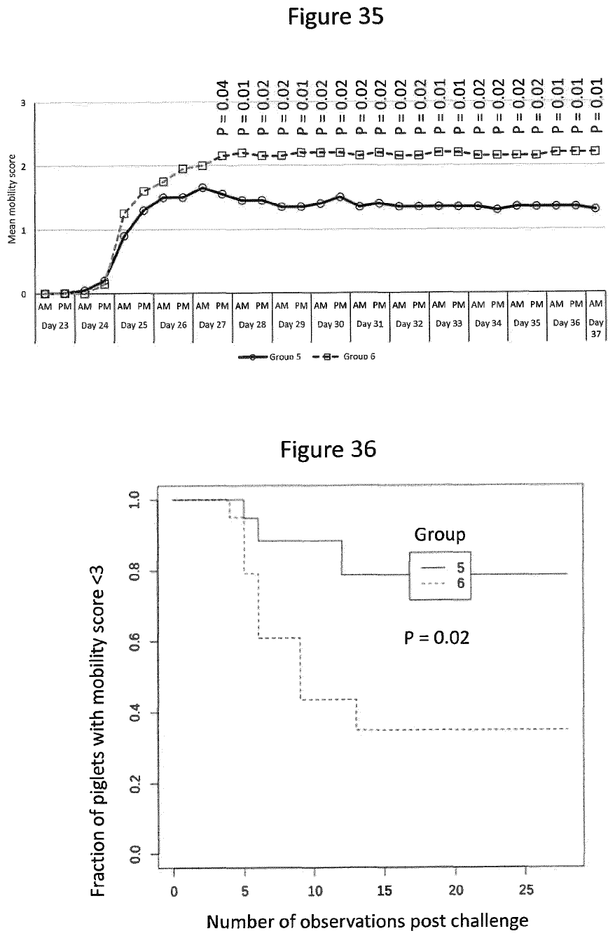

Overall, 3 of 20 piglets in Group 5 and 10 of 20 piglets in Group 6 developed a mobility score of >2 before the end of the study (P=0.04, Fisher's exact two-tailed test). The mobility scores of piglets in Group 5 were significantly lower than those in the control Group, on days 4 to 14 post-challenge (two-tailed Mann-Whitney U test, FIG. 35). There was a significant difference in the onset of mobility scores >2 by the log rank test (P=0.02, FIG. 36).

Piglets from the Piggivac5-Vaccinated Sow had Significantly Lower Total Clinical Scores Post-Challenge.

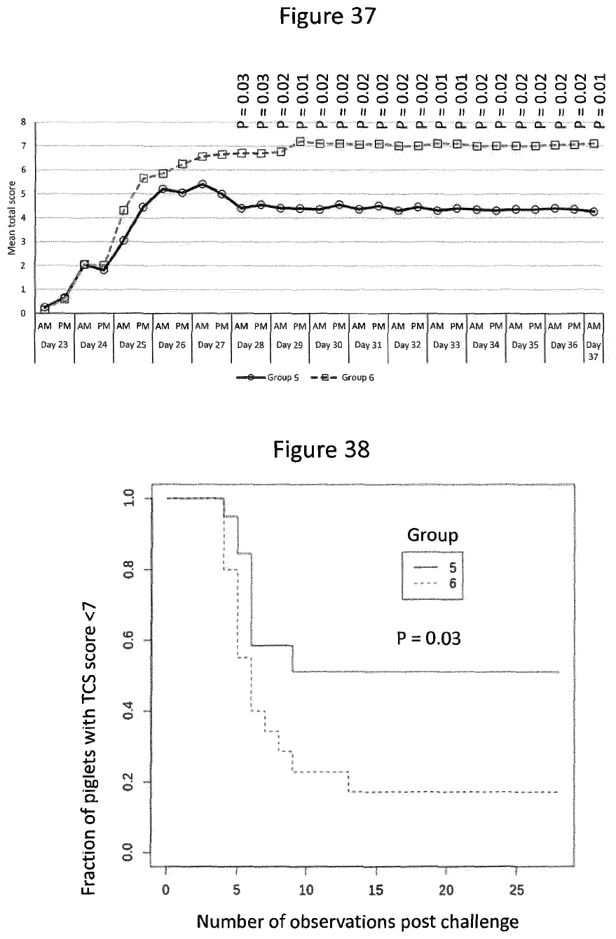

Overall, 8 of 20 piglets in Group 5 and 16 of 20 piglets in Group 6 developed a total clinical score of >7 before the end of the study (P=0.02, Fisher's exact two-tailed test). The total clinical scores of piglets in Group 5 were significantly lower than those in the control Group on days 5 to 14 post-challenge (two-tailed Mann-Whitney U test, FIG. 37). There was a significant difference in the onset of total clinical scores of 7 or more by the log rank test (P=0.03, FIG. 38).

Piglets from Vaccinated Sows Gained More Weight Post-Challenge.

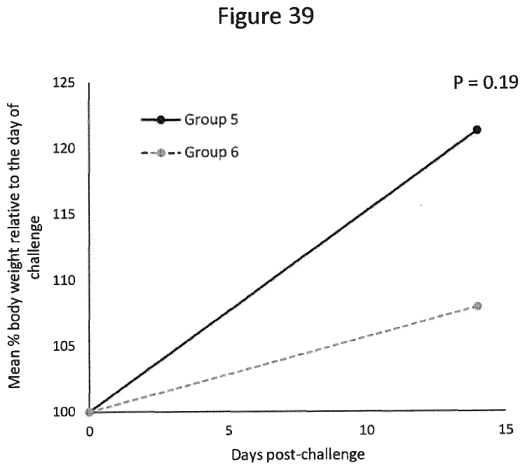

Piglets were weighed immediately before challenge and then on 14 days post-challenge, or at the time of euthanasia. The weight of each piglet as a percentage relative to the time of challenge (day 0) was calculated. The piglets from vaccinated sows gained more weight post-challenge, but this did not achieve statistical significance (P=0.19, FIG. 39). Six of the piglets from the sows vaccinated with Piggivac5 gained over 50% weight compared to only one of the piglets from control sows (P=0.09).

### Itemized List of Embodiments

1. Immunogenic composition comprising at least one first and one second fusion polypeptide, which first fusion polypeptide comprises fragments from at least three native full length polypeptides from Streptococcus suis.
2. Immunogenic composition according to items 1, wherein said second fusion polypeptide comprises fragments from at least three native full length polypeptides from S. suis.
3. Immunogenic composition according to items 1 or 2, optionally further comprising a third fusion polypeptide.
4. Immunogenic composition according to any one of items 1-3, optionally further comprising a fourth fusion polypeptide.
5. Immunogenic composition according to any one of items 1-4, optionally further comprising a fifth fusion polypeptide.
6. Immunogenic composition according to any one of items 3-5, wherein said third fusion polypeptide, fourth fusion polypeptide and/or fifth fusion polypeptide comprise/comprises fragments from at least three native full length polypeptides from S. suis.
7. Immunogenic composition according to any one of items 1-6, wherein said at least three native full length polypeptides from S. suis are independently selected or selected from the group consisting of zinc-binding proteins from S. suis; proteases from S. suis; nucleotidases from S. suis; proteins from S. suis which comprise an LPXTG-motif; Amid1Sa (SEQ ID NO:14) and any polypeptides with at least 80% identity to SEQ ID NO:14; 15BSa (SEQ ID NO:15) and polypeptides with at least 80% identity to SEQ ID NO:15; and Hom17Sa (SEQ ID NO:16) and any polypeptides with at least 80% identity to SEQ ID NO:16.
8. Immunogenic composition according to any one of items 1-7, wherein said at least three native full length polypeptides from S. suis are independently selected or selected from the group consisting of zinc-binding proteins from S. suis, proteases from S. suis, nucleotidases from S. suis and proteins from S. suis which comprise an LPXTG-motif.
9. Immunogenic composition according to any one of items 1-8, wherein said first fusion polypeptide comprises at least three fragments selected from fragments of zinc-binding proteins from S. suis; or from fragments of proteases from S. suis; or from fragments of nucleotidases from S. suis or from proteins from S. suis which comprise an LPXTG-motif.
10. Immunogenic composition according to any one of items 1-9, wherein said second fusion polypeptide comprises at least three fragments selected from fragments of zinc-binding proteins from S. suis; or from fragments of proteases from S. suis; or from fragments of nucleotidases from S. suis or from proteins from S. suis which comprise an LPXTG-motif.
11. Immunogenic composition according to any one of items 3-10, wherein said third fusion polypeptide comprises at least three fragments selected from fragments of zinc-binding proteins from S. suis; or from fragments of proteases from S. suis; or from fragments of nucleotidases from S. suis or from proteins from S. suis which comprise an LPXTG-motif.
12. Immunogenic composition according to any one of items 4-11, wherein said fourth fusion polypeptide comprises at least three fragments selected from fragments of zinc-binding proteins from S. suis; or from fragments of proteases from S. suis; or from fragments of nucleotidases from S. suis or from proteins from S. suis which comprise an LPXTG-motif.
13. Immunogenic composition according to any one of items 5-12, wherein said fifth fusion polypeptide comprises at least three fragments selected from fragments of zinc-binding proteins from S. suis; or from fragments of proteases from S. suis; or from fragments of nucleotidases from S. suis or from proteins from S. suis which comprise an LPXTG-motif.
14. Immunogenic composition according to any one of items 9-13, wherein each of said first, second, third, fourth and fifth fusion polypeptides comprises fragments, such as at least three fragments, independently selected or selected from fragments of zinc-binding proteins from S. suis; or from fragments of proteases from S. suis; or from fragments of nucleotidases from S. suis or from proteins from S. suis which comprise an LPXTG-motif.
15. Immunogenic composition according to any one of items 8-14, wherein said at least two of said first, second, third, fourth and fifth fusion polypeptides comprise fragments of zinc-binding proteins from S. suis.
16. Immunogenic composition according to any one of items 1-15, wherein said at least three native full length polypeptides from S. suis are independently selected or selected from the group consisting of polypeptides having an amino acid sequence according to any one of SEQ ID NO:1-19 and any polypeptides with at least 80% identity to any one of SEQ ID NO:1-19; such as the group consisting of polypeptides having an amino acid sequence according to any one of SEQ ID NO:1-19.
17. Immunogenic composition according to any one of items 1-16, wherein said at least three native full length polypeptides from S. suis are independently selected or selected from the group consisting of polypeptides having an amino acid sequence according to any one of SEQ ID NO:1-13 and 17-19 and polypeptides with at least 80% identity to any one of SEQ ID NO:1-13 and 17-19; such as the group consisting of polypeptides having an amino acid sequence according to any one of SEQ ID NO:1-13 and 17-19.
18. Immunogenic composition according to any one of items 1-17, wherein said first fusion polypeptide comprises at least three fragments selected from fragments of the zinc-binding proteins having an amino acid sequence according to any one of SEQ ID NO:1-5 and polypeptides with at least 80% identity to any one of SEQ ID NO:1-5; or from fragments of the proteases having an amino acid sequence according to any one of SEQ ID NO:6-10 and polypeptides with at least 80% identity to any one of SEQ ID NO:6-10; or from fragments of the nucleotidases having an amino acid sequence according to any one of SEQ ID NO:11-13 and polypeptides with at least 80% identity to any one of SEQ ID NO:11-13; or from fragments of proteins comprising an LPXTG-motif having an amino acid sequence according to any one of SEQ ID NO:17-19 and polypeptides with at least 80% identity to any one of SEQ ID NO:17-19.
19. Immunogenic composition according to any one of items 1-18, wherein said first fusion polypeptide comprises at least three fragments selected from fragments of the zinc-binding proteins having an amino acid sequence according to any one of SEQ ID NO:1-5; or from fragments of the proteases having an amino acid sequence according to any one of SEQ ID NO:6-10; or from fragments of the nucleotidases having an amino acid sequence according to any one of SEQ ID NO:11-13 or from fragments of proteins comprising an LPXTG-motif having an amino acid sequence according to any one of SEQ ID NO:17-19.
20. Immunogenic composition according to any one of items 1-19, wherein said second fusion polypeptide comprises at least three fragments selected from fragments of the zinc-binding proteins having an amino acid sequence according to any one of SEQ ID NO:1-5 and polypeptides with at least 80% identity to any one of SEQ ID NO:1-5; or from fragments of the proteases having an amino acid sequence according to any one of SEQ ID NO:6-10 and polypeptides with at least 80% identity to any one of SEQ ID NO:6-10; or from fragments of the nucleotidases having an amino acid sequence according to any one of SEQ ID NO:11-13 and polypeptides with at least 80% identity to any one of SEQ ID NO:11-13; or from fragments of proteins comprising an LPXTG-motif having an amino acid sequence according to any one of SEQ ID NO:17-19 and polypeptides with at least 80% identity to any one of SEQ ID NO:17-19.
21. Immunogenic composition according to any one of items 1-20, wherein said second fusion polypeptide comprises at least three fragments selected from fragments of the zinc-binding proteins having an amino acid sequence according to any one of SEQ ID NO:1-5; or from fragments of the proteases having an amino acid sequence according to any one of SEQ ID NO:6-10; or from fragments of the nucleotidases having an amino acid sequence according to any one of SEQ ID NO:11-13 or from fragments of proteins comprising an LPXTG-motif having an amino acid sequence according to any one of SEQ ID NO:17-19.
22. Immunogenic composition according to any one of items 3-21, wherein said third fusion polypeptide comprises at least three fragments selected from fragments of the zinc-binding proteins having an amino acid sequence according to any one of SEQ ID NO:1-5 and polypeptides with at least 80% identity to any one of SEQ ID NO:1-5; or from fragments of the proteases having an amino acid sequence according to any one of SEQ ID NO:6-10 and polypeptides with at least 80% identity to any one of SEQ ID NO:6-10; or from fragments of the nucleotidases having an amino acid sequence according to any one of SEQ ID NO:11-13 and polypeptides with at least 80% identity to any one of SEQ ID NO:11-13; or from fragments of proteins comprising an LPXTG-motif having an amino acid sequence according to any one of SEQ ID NO:17-19 and polypeptides with at least 80% identity to any one of SEQ ID NO:17-19.
23. Immunogenic composition according to any one of items 3-22, wherein said third fusion polypeptide comprises at least three fragments selected from fragments of the zinc-binding proteins having an amino acid sequence according to any one of SEQ ID NO:1-5; or from fragments of the proteases having an amino acid sequence according to any one of SEQ ID NO:6-10; or from fragments of the nucleotidases having an amino acid sequence according to any one of SEQ ID NO:11-13 or from fragments of proteins comprising an LPXTG-motif having an amino acid sequence according to any one of SEQ ID NO:17-19.
24. Immunogenic composition according to any one of items 4-23, wherein said fourth fusion polypeptide comprises at least three fragments selected from fragments of the zinc-binding proteins having an amino acid sequence according to any one of SEQ ID NO:1-5 and polypeptides with at least 80% identity to any one of SEQ ID NO:1-5; or from fragments of the proteases having an amino acid sequence according to any one of SEQ ID NO:6-10 and polypeptides with at least 80% identity to any one of SEQ ID NO:6-10; or from fragments of the nucleotidases having an amino acid sequence according to any one of SEQ ID NO:11-13 and polypeptides with at least 80% identity to any one of SEQ ID NO:11-13; or from fragments of proteins comprising an LPXTG-motif having an amino acid sequence according to any one of SEQ ID NO:17-19 and polypeptides with at least 80% identity to any one of SEQ ID NO:17-19.
25. Immunogenic composition according to any one of items 4-24, wherein said fourth fusion polypeptide comprises at least three fragments selected from fragments of the zinc-binding proteins having an amino acid sequence according to any one of SEQ ID NO:1-5; or from fragments of the proteases having an amino acid sequence according to any one of SEQ ID NO:6-10; or from fragments of the nucleotidases having an amino acid sequence according to any one of SEQ ID NO:11-13 or from fragments of proteins comprising an LPXTG-motif having an amino acid sequence according to any one of SEQ ID NO:17-19.
26. Immunogenic composition according to any one of items 5-25, wherein said fifth fusion polypeptide comprises at least three fragments selected from fragments of the zinc-binding proteins having an amino acid sequence according to any one of SEQ ID NO:1-5 and polypeptides with at least 80% identity to any one of SEQ ID NO:1-5; or from fragments of the proteases having an amino acid sequence according to any one of SEQ ID NO:6-10 and polypeptides with at least 80% identity to any one of SEQ ID NO:6-10; or from fragments of the nucleotidases having an amino acid sequence according to any one of SEQ ID NO:11-13 and polypeptides with at least 80% identity to any one of SEQ ID NO:11-13; or from fragments of proteins comprising an LPXTG-motif having an amino acid sequence according to any one of SEQ ID NO:17-19 and polypeptides with at least 80% identity to any one of SEQ ID NO:17-19.
27. Immunogenic composition according to any one of items 5-26, wherein said fifth fusion polypeptide comprises at least three fragments selected from fragments of the zinc-binding proteins having an amino acid sequence according to any one of SEQ ID NO:1-5; or from fragments of the proteases having an amino acid sequence according to any one of SEQ ID NO:6-10; or from fragments of the nucleotidases having an amino acid sequence according to any one of SEQ ID NO:11-13 or from fragments of proteins comprising an LPXTG-motif having an amino acid sequence according to any one of SEQ ID NO:17-19.
28. Immunogenic composition according to any one of the preceding items, wherein each of said first, second, third, fourth and fifth fusion polypeptides comprises fragments independently selected or selected from fragments of the zinc-binding proteins having an amino acid sequence according to any one of
SEQ ID NO:1-5 and polypeptides with at least 80% identity to any one of SEQ ID NO:1-5; or from fragments of the proteases having an amino acid sequence according to any one of SEQ ID NO:6-10 and polypeptides with at least 80% identity to any one of SEQ ID NO:6-10; or from fragments of the nucleotidases having an amino acid sequence according to any one of SEQ ID NO:11-13 and polypeptides with at least 80% identity to any one of SEQ ID NO:11-13; or from fragments of proteins comprising an LPXTG-motif having an amino acid sequence according to any one of SEQ ID NO:17-19 and polypeptides with at least 80% identity to any one of SEQ ID NO:17-19.
   29. Immunogenic composition according to any one of the preceding items, wherein said each of said first, second, third, fourth and fifth fusion polypeptides comprises fragments independently selected or selected from fragments of the zinc-binding proteins having an amino acid sequence according to any one of SEQ ID NO:1-5; or from fragments of the proteases having an amino acid sequence according to any one of SEQ ID NO:6-10; or from fragments of the nucleotidases having an amino acid sequence according to any one of SEQ ID NO:11-13 or from fragments of proteins comprising an LPXTG-motif having an amino acid sequence according to any one of SEQ ID NO:17-19.
   30. Immunogenic composition according to any one of the preceding items, wherein at least two of said first, second, third, fourth and fifth fusion polypeptides comprise fragments of zinc-binding proteins having an amino acid sequence according to any one of SEQ ID NO:1-5 and polypeptides with at least 80% identity to any one of SEQ ID NO:1-5, such as wherein said at least two of said first, second, third, fourth and fifth fusion polypeptides comprise fragments of zinc-binding proteins having an amino acid sequence according to any one of SEQ ID NO:1-5.
   31. Immunogenic composition according to any one of the preceding items, wherein at least one of said first, second, third, fourth and fifth fusion polypeptide comprises at least three or four fragments from zinc-binding proteins from S. suis, such as at least three, such as at least four, such as at least five, fragments from different zinc-binding proteins from S. suis.
   32. Immunogenic composition according to any one of the preceding items, wherein at least one of said first, second, third, fourth and fifth fusion polypeptide comprises at least three or four fragments of the zinc-binding proteins having an amino acid sequence according to any one of SEQ ID NO:1-5 and polypeptides with at least 80% identity to any one of SEQ ID NO:1-5, such as at least three or four fragments of the zinc-binding proteins having an amino acid sequence according to any one of SEQ ID NO:1-5; or immunogenic composition according to any one of the preceding claims, wherein at least one of said first, second, third, fourth and fifth fusion polypeptide comprises at least three, such as at least four, such as at least five fragments of the zinc-binding proteins having an amino acid sequence according to any one of SEQ ID NO:1-5 and polypeptides with at least 80% identity to any one of SEQ ID NO:1-5, such as at least three, such as at least four, such as at least five, fragments of the zinc-binding proteins having an amino acid sequence according to any one of SEQ ID NO:1-5.
   33. Immunogenic composition according to any one the preceding items, wherein at least one of said first, second, third, fourth and fifth fusion polypeptide comprises at least three fragments from proteases from S. suis, such as at least three fragments from different proteases from S. suis.
   34. Immunogenic composition according to any one the preceding items, wherein at least one of said first, second, third, fourth and fifth fusion polypeptide comprises at least three fragments of the proteases having an amino acid sequence according to any one of SEQ ID NO:6-10 and polypeptides with at least 80% identity to any one of said amino acid sequences, such as at least three, such as at least four, such as at least five, such as at least six, fragments of the proteases having an amino acid sequence according to any one of SEQ ID NO:6-10.
   35. Immunogenic composition according to any one of the preceding items, wherein at least one of said first, second, third, fourth and fifth fusion polypeptide comprises at least three fragments from nucleotidases from S. suis, such as at least three fragments from different nucleotidases from S. suis.
   36. Immunogenic composition according to any one the preceding items, wherein at least one of said first, second, third, fourth and fifth fusion polypeptide comprises at least three fragments of the nucleotidases having an amino acid sequence according to any one of SEQ ID NO:11-13 and polypeptides with at least 80% identity to any one of said amino acid sequences, such as at least three fragments of the nucleotidases having an amino acid sequence according to any one of SEQ ID NO:11-13.
   37. Immunogenic composition according to any one of the preceding items, wherein at least one of said first, second, third, fourth and fifth fusion polypeptide comprises at least three fragments of proteins from S. suis which comprise an LPXTG-motif, such as at least three fragments from different nucleotidases from S. suis.
   38. Immunogenic composition according to any one the preceding items, wherein at least one of said first, second, third, fourth and fifth fusion polypeptide comprises at least three fragments of proteins from S. suis which comprise an LPXTG-motif having an amino acid sequence according to any one of SEQ ID NO:17-19 and polypeptides with at least 80% identity to any one of said amino acid sequences, such as at least three fragments of proteins from S. suis which comprise an LPXTG-motif having an amino acid sequence according to any one of SEQ ID NO:17-19.
   39. Immunogenic composition according to any one of the preceding items, wherein at least one of said first, second, third, fourth and fifth fusion polypeptides further comprises at least one fragment, such as at least two fragments, such as at least three fragments, of at least one native full length polypeptide having an amino acid sequence according to any one of SEQ ID NO:1-19 or any amino acid sequences with at least 80% identity to any one of SEQ ID NO:1-19.
   40. Immunogenic composition according to any one of the preceding items, wherein at least one of said first, second, third, fourth and fifth fusion polypeptides further comprises
a fragment of at least one native full length polypeptide having an amino acid sequence according to any one of SEQ ID NO:14-16 or any amino acid sequences with at least 80% identity to any one of SEQ ID NO:14-16; such as a fragment of each of at least two native full length polypeptides having an amino acid sequence according to any one of SEQ ID NO:14-16 or any amino acid sequences with at least 80% identity to any one of SEQ ID NO:14-16; such as a fragment of each of all three native full length polypeptides having an amino acid sequence according to any one of SEQ ID NO:14-16 or any amino acid sequences with at least 80% identity to any one of SEQ ID NO:14-16.
   41. Immunogenic composition according to any one of the preceding items, wherein at least one of said first, second, third, fourth and fifth fusion polypeptides further comprises a fragment of at least one native full length polypeptide
having an amino acid sequence according to any one of SEQ ID NO:14-16, such as a fragment of each of at least two native full length polypeptides having an amino acid sequence according to any one of SEQ ID NO:14-16; such as a fragment of each of all three native full length polypeptides having an amino acid sequence according to any one of SEQ ID NO:14-16.
   42. Immunogenic composition according to any one of the preceding items, wherein said at least one of said first, second, third, fourth and fifth fusion polypeptides comprises at least two fragments from the same native full length polypeptide from S. suis and wherein the fragments are different fragments from the same native full length polypeptide from S. suis.
   43. Immunogenic composition according to item 42, wherein said native full length polypeptide from S. suis is a protease from S. suis or a zinc-binding protein of S. suis.
   44. Immunogenic composition according to any one of items 42 and 43, wherein said native full length polypeptide is SEQ ID NO:9 or any amino acid sequence having at least 80% identity to SEQ ID NO:9, such as wherein said native full length polypeptide is SEQ ID NO:9.
   45. Immunogenic composition according to any one of the preceding items, wherein said at least two of said first, second, third, fourth and fifth fusion polypeptides comprise at least one fragment each from the same native full length polypeptide from S. suis, such as from the same two native full length polypeptides from S. suis, such as from the same three native full length polypeptides from S. suis; and wherein the fragments are different fragments from the same native full length polypeptide from S. suis.
   46. Immunogenic composition according to item 45, wherein said native full length polypeptide from S. suis is a zinc-binding protein from S. suis.
   47. Immunogenic composition according to any one of the preceding items, wherein the composition comprises three, four or five fusion polypeptides, such as wherein the composition comprises four or five fusion polypeptides, such as wherein the composition comprises five fusion polypeptides.
   48. Immunogenic composition, according to any one of the preceding items, wherein said composition comprises a first fusion polypeptide, a second fusion polypeptide, a third fusion polypeptide and a fourth fusion polypeptide; or wherein said composition comprises a first fusion polypeptide, a second fusion polypeptide and a fourth fusion polypeptide and fifth fusion polypeptide; or wherein said composition comprises a first fusion polypeptide, a second fusion polypeptide, a third fusion polypeptide and a fifth fusion polypeptide; or wherein said composition comprises a first fusion polypeptide, a second fusion polypeptide, a third fusion polypeptide, a fourth fusion polypeptide and a fifth fusion polypeptide.
   49. Immunogenic composition according to any one of the preceding items, wherein said fragments of said zinc-binding polypeptides are selected from the group consisting of fragments of amino acid sequence SEQ ID NO:1, SEQ ID NO:2, SEQ ID NO:3, SEQ ID NO:4 and SEQ ID NO:5 and any amino acid sequences having at least 80% identity to said fragments, such as the group consisting of fragments of amino acid sequence SEQ ID NO:1, SEQ ID NO:2, SEQ ID NO:3 and any amino acid sequences having at least 80% identity to said fragments.
   50. Immunogenic composition according to item 49, wherein said fragments of said zinc-binding protein are selected from the group consisting of polypeptide fragments comprising an amino acid sequence selected from SEQ ID NO:20-27 and any amino acid sequences with at least 80% identity to any one of SEQ ID NO:20-27, such as the group consisting of polypeptide fragments comprising an amino acid sequence selected from SEQ ID NO:20-27.
   51. Immunogenic composition according to any one of the preceding items, wherein said fragments of said proteases are selected from the group consisting of fragments of amino acid sequence SEQ ID NO:6, SEQ ID NO:7, SEQ ID NO:8, SEQ ID NO:9, SEQ ID NO:10 and any amino acid sequences having at least 80% identity to said fragments.
   52. Immunogenic composition according to item 51, wherein said fragments of said proteases are selected from the group consisting of polypeptide fragments comprising an amino acid sequence selected from SEQ ID NO:28-33 and any amino acid sequences with at least 80% identity to any one of SEQ ID NO:28-33, such as the group consisting of polypeptide fragments comprising an amino acid sequence selected from SEQ ID NO:28-33.
   53. Immunogenic composition according to any one of the preceding items, wherein said fragments of said nucleotidases are selected from the group consisting of fragments of amino acid sequence SEQ ID NO:11, SEQ ID NO:12, SEQ ID NO:13 and any amino acid sequences having at least 80% identity to said fragments.
   54. Immunogenic composition according to item 53, wherein said fragments of said nucleotidases are selected from the group consisting of polypeptide fragments comprising an amino acid sequence selected from SEQ ID NO:34-36 and any amino acid sequences with at least 80% identity to any one of SEQ ID NO:34-36, such as the group consisting of polypeptide fragments comprising an amino acid sequence selected from SEQ ID NO:34-36.
   55. Immunogenic composition according to any one of the preceding items, wherein said fragments of said proteins comprising an LPXTG-motif are selected from the group consisting of fragments of amino acid sequence SEQ ID NO:17, SEQ ID NO:18, SEQ ID NO:19 and any amino acid sequences having at least 80% identity to said fragments.
   56. Immunogenic composition according to item 55, wherein said fragments of said proteins comprising an LPXTG-motif are selected from the group consisting of polypeptide fragments comprising an amino acid sequence selected from SEQ ID NO:40-42 and any amino acid sequences with at least 80% identity to any one of SEQ ID NO:40-42, such as the group consisting of polypeptide fragments comprising an amino acid sequence selected from SEQ ID NO:40-42.
   57. Immunogenic composition according to any one of the preceding items, wherein said fragments are selected from the group consisting of fragments of amino acid sequence SEQ ID NO:14, SEQ ID NO:15, SEQ ID NO:16 and any amino acid sequences having at least 80% identity to said fragments.
   58. Immunogenic composition according to item 57, wherein said fragments are selected from the group consisting of SEQ ID NO:37-39 and any amino acid sequences with at least 80% identity to any one of SEQ ID NO:37-39, such as the group consisting of polypeptide fragments comprising an amino acid sequence selected from SEQ ID NO:37-39.
   59. Immunogenic composition according to any one of the preceding items, wherein one of said first, second, third, fourth and fifth fusion polypeptides comprises at least one, such as at least two, such as at least three, fragment(s) selected from the group consisting of SEQ ID NO:20-22 and any amino acid sequence with at least 80% identity to any one of SEQ ID NO:20-22.
   60. Immunogenic composition according to item 59, wherein one of said first, second, third, fourth and fifth fusion polypeptides comprises SEQ ID NO:20, 21 and 22.
   61. Immunogenic composition according to item 59 or 60, wherein one of said first, second, third, fourth and fifth fusion polypeptides comprises SEQ ID NO:43 or an amino acid sequence with at least 80% identity to SEQ ID NO:43 or wherein one of said first, second, third, fourth and fifth fusion polypeptides comprises SEQ ID NO:106 or an amino acid sequence with at least 80% identity to SEQ ID NO:106.
   62. Immunogenic composition according to item 59 or 60, wherein one of said first, second, third, fourth and fifth fusion polypeptides comprises SEQ ID NO:43 wherein independently of each other the amino acid residue in position 303 may be absent or present or not G and the amino acid residue in position 304 may be absent or present or not S.
   63. Immunogenic composition according to item 61 or 62, wherein said SEQ ID NO:43 or 106 further comprises an N-terminal M or MT and optionally comprises a C-terminal His-tag.
   64. Immunogenic composition according to any one of the preceding items, wherein one of said first, second, third, fourth and fifth fusion polypeptides comprises at least one, such as at least two, such as at least three, such as at least four, such as at least five, such as at least six, such as at least seven, such as eight, fragment(s) selected from the group consisting of SEQ ID NO:23-27 and SEQ ID NO:37-39 and any amino acid sequence with at least 80% identity to any one of SEQ ID NO: 23-27 and SEQ ID NO:37-39.
   65. Immunogenic composition according to item 64, wherein one of said first, second, third, fourth and fifth fusion polypeptides comprises SEQ ID NO:23-27 and SEQ ID NO:37-39.
   66. Immunogenic composition according to item 64 or 65, wherein one of said first, second, third, fourth and fifth fusion polypeptides comprises SEQ ID NO:44 or an amino acid sequence with at least 80% identity to SEQ ID NO:44.
   67. Immunogenic composition according to item 66, wherein said SEQ ID NO:44 further comprises N-terminal M, such as N-terminal amino acid residues MT or MTG or MTGS.
   68. Immunogenic composition according to any one of the preceding items, wherein one of said first, second, third, fourth and fifth fusion polypeptides comprises at least one, such as at least two, such as at least three, such as at least four, such as at least five, such as at least six fragment(s) selected from the group consisting of SEQ ID NO:28-33 and amino acid sequences with at least 80% identity to any one of SEQ ID NO:28-33.
   69. Immunogenic composition according to item 68, wherein one of said first, second, third, fourth and fifth fusion polypeptides comprises SEQ ID NO:28-33.
   70. Immunogenic composition according to item 58 or 59, wherein one of said first, second, third, fourth and fifth fusion polypeptides comprises SEQ ID NO:45 or an amino acid sequence with at least 80% identity to SEQ ID NO:45.
   71. Immunogenic composition according to item 70, wherein said SEQ ID NO:45 further comprises N-terminal M, such as N-terminal amino acid residues MT or MTG or MTGS.
   72. Immunogenic composition according to any one of the preceding items, wherein one of said first, second, third, fourth and fifth fusion polypeptides comprises at least one, such as at least two, such as at least three of SEQ ID NO:34-36 or amino acid sequences with at least 80% identity to any one of SEQ ID NO:34-36.
   73. Immunogenic composition according to item 72, wherein one of said first, second, third, fourth and fifth fusion polypeptides comprises SEQ ID NO:34-36.
   74. Immunogenic composition according to item 72 or 73, wherein one of said first, second, third, fourth and fifth fusion polypeptides comprises SEQ ID NO:46 or an amino acid sequence with at least 80% identity to SEQ ID NO:46
or
wherein one of said first, second, third, fourth and fifth fusion polypeptides comprises SEQ ID NO:47 or an amino acid sequence with at least 80% identity to SEQ ID NO:47
or
wherein one of said first, second, third, fourth and fifth fusion polypeptides comprises SEQ ID NO:107 or an amino acid sequence with at least 80% identity to SEQ ID NO:107
or
wherein one of said first, second, third, fourth and fifth fusion polypeptides comprises SEQ ID NO:108 or an amino acid sequence with at least 80% identity to SEQ ID NO:108.
   75. Immunogenic composition according to item 73 or 74, wherein one of said first, second, third, fourth and fifth fusion polypeptides comprises SEQ ID NO:46 or 47 wherein independently of each other the amino acid residue in position 317 may be absent or present or not E; the amino acid residue in position 318 may be absent or present or not F; the amino acid residue in position 608 may be absent or present or not G; the amino acid residue in position 609 may be absent or present or not T; the amino acid residue in position 944 may be absent or present or not L; and the amino acid residue in position 945 may be absent or present or not E.
   76. Immunogenic composition according to item 74 or 75, wherein said SEQ ID NO:46, 47, 107 or 108 further comprises N-terminal M, such as N-terminal amino acid residues MT or MTG or MTGS, and optionally comprises a C-terminal SL2-tag.
   77. Immunogenic composition according to any one of the preceding items, wherein one of said first, second, third, fourth and fifth fusion polypeptides comprises at least one, such as at least two, such as at least three of SEQ ID NO:40-42 or amino acid sequences with at least 80% identity to any one of SEQ ID NO:40-42.
   78. Immunogenic composition according to item 77, wherein one of said first, second, third, fourth and fifth fusion polypeptides comprises SEQ ID NO:40-42.
   79. Immunogenic composition according to item 77 or 78, wherein one of said first, second, third, fourth and fifth fusion polypeptides comprises SEQ ID NO:48 or an amino acid sequence with at least 80% identity to SEQ ID NO:48.
   80. Immunogenic composition according to item 79, wherein said SEQ ID NO:48 further comprises N-terminal M, such as N-terminal amino acid residues MT or MTG or MTGS, or comprises N-terminal GPLGS.
   81. Immunogenic composition according to any one of the preceding items, wherein at least two, at least three, at least four or all five of said first, second, third, fourth and fifth fusion polypeptides are selected from the group consisting of:
fusion polypeptides comprising SEQ ID NO:20, 21 and 22 or amino acid sequences with at least 80% identity to any one of SEQ ID NO:20, 21 and 22;
fusion polypeptides comprising SEQ ID NO:23-27 and SEQ ID NO:37-39 or amino acid sequences with at least 80% identity to any one of SEQ ID NO:23-27 and SEQ ID NO:37-39;
fusion polypeptides comprising SEQ ID NO:28-33 or amino acid sequences with at least 80% identity to any one of SEQ ID NO:28-33;
fusion polypeptides comprising SEQ ID NO:34-36 or amino acid sequences with at least 80% identity to any one of SEQ ID NO:34-36; and
fusion polypeptides comprising SEQ ID NO:40-42 or amino acid sequences with at least 80% identity to any one of SEQ ID NO:40-42.
   82. Immunogenic composition according to item 64, wherein at least two, at least three, at least four or all five of said first, second, third, fourth and fifth
fusion polypeptides are selected from the group consisting of:
fusion polypeptides comprising SEQ ID NO:20-22;
fusion polypeptides comprising SEQ ID NO:23-27 and SEQ ID NO:37-39;
fusion polypeptides comprising SEQ ID NO:28-33;
fusion polypeptides comprising SEQ ID NO:34-36; and
fusion polypeptides comprising SEQ ID NO:40-42
   83. Immunogenic composition according to any one of the preceding items, wherein at least two, at least three, at least four or all five of said first, second, third, fourth and fifth fusion polypeptides are selected from the group consisting of:
fusion polypeptides comprising SEQ ID NO:106 or an amino acid sequence with at least 80% identity to SEQ ID NO:106;
fusion polypeptides comprising SEQ ID NO:44 or an amino acid sequence with at least 80% identity to SEQ ID NO:44;
fusion polypeptides comprising SEQ ID NO:45 or an amino acid sequence with at least 80% identity to SEQ ID NO:45;
fusion polypeptides comprising SEQ ID NO:107 or an amino acid sequence with at least 80% identity to SEQ ID NO:107, and
fusion polypeptides comprising SEQ ID NO:48 or an amino acid sequence with at least 80% identity to SEQ ID NO:48;
or from the group consisting of:
fusion polypeptides comprising SEQ ID NO:106 or an amino acid sequence with at least 80% identity to SEQ ID NO:106;
fusion polypeptides comprising SEQ ID NO:44 or an amino acid sequence with at least 80% identity to SEQ ID NO:44;
fusion polypeptides comprising SEQ ID NO:45 or an amino acid sequence with at least 80% identity to SEQ ID NO:45;
fusion polypeptides comprising SEQ ID NO:108 or an amino acid sequence with at least 80% identity to SEQ ID NO:108, and
fusion polypeptides comprising SEQ ID NO:48 or an amino acid sequence with at least 80% identity to SEQ ID NO:48,
such as wherein at least two, at least three, at least four or all five of said first, second, third, fourth and fifth fusion polypeptides are selected from the group consisting of
fusion polypeptides comprising SEQ ID NO:43 or an amino acid sequence with at least 80% identity to SEQ ID NO:43;
fusion polypeptides comprising SEQ ID NO:44 or an amino acid sequence with at least 80% identity to SEQ ID NO:44;
fusion polypeptides comprising SEQ ID NO:45 or an amino acid sequence with at least 80% identity to SEQ ID NO:45;
fusion polypeptides comprising SEQ ID NO:46 or an amino acid sequence with at least 80% identity to SEQ ID NO:46, and
fusion polypeptides comprising SEQ ID NO:48 or an amino acid sequence with at least 80% identity to SEQ ID NO:48;
or from the group consisting of:
fusion polypeptides comprising SEQ ID NO:43 or an amino acid sequence with at least 80% identity to SEQ ID NO:43;
fusion polypeptides comprising SEQ ID NO:44 or an amino acid sequence with at least 80% identity to SEQ ID NO:44;
fusion polypeptides comprising SEQ ID NO:45 or an amino acid sequence with at least 80% identity to SEQ ID NO:45;
fusion polypeptides comprising SEQ ID NO:47 or an amino acid sequence with at least 80% identity to SEQ ID NO:47, and
fusion polypeptides comprising SEQ ID NO:48 or an amino acid sequence with at least 80% identity to SEQ ID NO:48.
   84. Immunogenic composition according to item 83, wherein at least two, at least three, at least four or all five of said of said first, second, third, fourth and fifth fusion polypeptides are selected from the group consisting of: fusion
polypeptides comprising SEQ ID NO:106;
fusion polypeptides comprising SEQ ID NO:44;
fusion polypeptides comprising SEQ ID NO:45;
fusion polypeptides comprising SEQ ID NO 107; and
fusion polypeptides comprising SEQ ID NO:48;
or from the group consisting of:
fusion polypeptides comprising SEQ ID NO:106;
fusion polypeptides comprising SEQ ID NO:44;
fusion polypeptides comprising SEQ ID NO:108;
fusion polypeptides comprising SEQ ID NO:47; and
fusion polypeptides comprising SEQ ID NO:48.
such as wherein at least two, at least three, at least four or all five of said first, second, third, fourth and fifth fusion polypeptides are selected from the group consisting of
fusion polypeptides comprising SEQ ID NO:43;
fusion polypeptides comprising SEQ ID NO:44;
fusion polypeptides comprising SEQ ID NO:45;
fusion polypeptides comprising SEQ ID NO:46; and
fusion polypeptides comprising SEQ ID NO:48;
or from the group consisting of:
fusion polypeptides comprising SEQ ID NO:43;
fusion polypeptides comprising SEQ ID NO:44;
fusion polypeptides comprising SEQ ID NO:45;
fusion polypeptides comprising SEQ ID NO:47; and
fusion polypeptides comprising SEQ ID NO:48.
   85. Immunogenic composition according to item 83 or 84, wherein said fusion polypeptide comprising SEQ ID NO:48 is SEQ ID NO:54 or SEQ ID NO:55.
   86. Immunogenic composition according to any one of the preceding items, wherein at least one, such as at least two or at least three, of said fusion polypeptides is/are not denatured and/or not precipitated by heating to approximately 100° C.
   87. Fusion polypeptide comprising fragments from at least 3, such as at least 4, such as at least 5, such as at least 6, such as at least 7, native full length polypeptides from Streptococcus suis, wherein said native full length polypeptides are independently selected or selected from the group consisting of amino acid sequences according to SEQ ID NO:1-10 and SEQ ID NO:14-19 and amino acid sequences having at least 80% identity to any one of SEQ ID NO:1-10 and SEQ ID NO:14-19, such as such as the group consisting of amino acid sequences according to SEQ ID NO:1-10 and SEQ ID NO:14-19.
   88. Fusion polypeptide according to item 87, wherein said native full length polypeptides are independently selected or selected from the group consisting of amino acid sequences according to SEQ ID NO:1-3 and amino acid sequences having at least 80% identity to any one of SEQ ID NO:1-3, such as wherein said native full length polypeptides are SEQ ID NO:1-3.
   89. Fusion polypeptide according to item 87, wherein said native full length polypeptides are independently selected or selected from the group consisting of amino acid sequences according to SEQ ID NO:1-5 and 14-16 and amino acid sequences having at least 80% identity to any one of SEQ ID NO:1-5 and 14-16, such as the group consisting of amino acid sequences according to SEQ ID NO:1-5 and 14-16, such as wherein said native full length polypeptides are SEQ ID NO:1-5 and 14-16.
   90. Fusion polypeptide according to item 87, wherein said native full length polypeptides are independently selected or selected from the group consisting of amino acid sequences according to SEQ ID NO:6-10 and amino acid sequences having at least 80% identity to any one of SEQ ID NO:6-10, such as the group consisting of amino acid sequences according to SEQ ID NO:6-10, such as wherein said native full length polypeptides are SEQ ID NO:6-10.
   91. Fusion polypeptide according to item 87, wherein said native full length polypeptides are independently selected or selected from the group consisting of amino acid sequences according to SEQ ID NO:17-19 and amino acid sequences having at least 80% identity to any one of SEQ ID NO:17-19, such as the group consisting of amino acid sequences according to SEQ ID NO:17-19, such as wherein said native full length polypeptides are SEQ ID NO:17-19.
   92. Fusion polypeptide according to item 87, wherein said fragments are independently selected or selected from the group consisting of fragments having amino acid sequences according to SEQ ID NO:20-33 and 37-42 and amino acid sequences having at least 80% identity to any one of SEQ ID NO:20-33 and 37-42, such as the group consisting of amino acid sequences according to SEQ ID NO: 20-33 and 37-42.
   93. Fusion polypeptide according to item 87, 88 or 93, wherein said fragments are selected from a group consisting of SEQ ID NO:20-22 and amino acid sequences having at least 80% identity to any one of SEQ ID NO:20-22.
   94. Fusion polypeptide according to item 93, wherein said fragments have amino acid sequences according to SEQ ID NO:20-22.
   95. Fusion polypeptide according to any one of items 87-88 and 92-94, comprising or consisting of the amino acid sequence SEQ ID NO:106 or an amino acid sequence having 80% identity to SEQ ID NO:106, such as comprising or consisting of the amino acid sequence SEQ ID NO:43 or an amino acid sequence having 80% identity to SEQ ID NO:43.
   96. Fusion polypeptide according to any one of items 87-88 and 92-95, comprising or consisting of the amino acid sequence SEQ ID NO:43 wherein independently of each other the amino acid residue in position 303 may be absent or present or not G and the amino acid residue in position 304 may be absent or present or not S.
   97. Fusion polypeptide according to any one of items 87-88 and 92-96, comprising or consisting of the amino acid sequence SEQ ID NO:43.
   98. Fusion polypeptide according to item 87 and 89, wherein said fragments are selected from a group of fragments having amino acid sequences according to SEQ ID NO:23-27 and 37-39 and amino acid sequences having at least 80% identity to any one of SEQ ID NO: 23-27 and 37-39.
   99. Fusion polypeptide according to any one of items 87, 89 and 98, wherein said fragments are selected from a group of fragments having amino acid sequences according to SEQ ID NO:23-27 and 37-39.
   100. Fusion polypeptide according to any one of items 87, 98 and 99, comprising or consisting of the amino acid sequence SEQ ID NO:44 or an amino acid sequence having 80% identity to SEQ ID NO:44.
   101. Fusion polypeptide according to any one of items 87, 89 and 98-100, comprising or consisting of the amino acid sequence SEQ ID NO:44.
   102. Fusion polypeptide according to any one of items 87 and 90, wherein said fragments are selected from a group of fragments having amino acid sequences according to SEQ ID NO:28-33 and amino acid sequences having at least 80% identity to any one of SEQ ID NO:28-33.
   103. Fusion polypeptide according to any one of items 87, 90 and 102, wherein said fragments are selected from a group of fragments having amino acid sequences according to SEQ ID NO:28-33.
   104. Fusion polypeptide according to any one of items 87, 90, 102 and 103, comprising or consisting of the amino acid sequence SEQ ID NO:45 or an amino acid sequence having 80% identity to SEQ ID NO:45.
   105. Fusion polypeptide according to any one of items 87, 90, 102-104, comprising or consisting of the amino acid sequence SEQ ID NO:45.
   106. Fusion polypeptide according to any one of items 87 and 91, wherein said fragments are selected from a group of fragments having amino acid sequences according to SEQ ID NO:40-42 and amino acid sequences having at least 80% identity to any one of SEQ ID NO:40-42.
   107. Fusion polypeptide according to any one of items 87, 91 and 106, wherein said fragments are selected from a group of fragments having amino acid sequences according to SEQ ID NO:40-42.
   108. Fusion polypeptide according to any one of items 87, 91, 106 and 107, comprising or consisting of the amino acid sequence SEQ ID NO:48 or an amino acid sequence having 80% identity to SEQ ID NO:48.
   109. Fusion polypeptide according to any one of items 87, 91, 106-108, comprising or consisting of the amino acid sequence SEQ ID NO:48.
   110. Fusion polypeptide according to any one of items 69-85, which fusion polypeptide comprises one or several additional N-terminal and/or C-terminal residue(s), such as N-terminal M or MTGS or GPLGS.
   111. Fusion polypeptide according to any one of items 87-105, which fusion polypeptide is not denatured and/or not precipitated by heating to approximately 100° C.
   112. Fusion polypeptide according to any one of items 87-111, which fusion polypeptide is stable after storage for at least 1 week at ambient temperature, such as at a temperature of approximately 20° C.
   113. Fusion polypeptide according to any one of items 87-112, for the use as a medicament.
   114. Fusion polypeptide according to any one of items 87-113, for use in the prophylactic treatment of a mammalian subject susceptible to Streptococcus suis infection.
   115. Polynucleotide encoding a fusion polypeptide as defined in any one of items 87-113.
   116. Expression vector comprising a polynucleotide according to item 115.
   117. Host cell comprising an expression vector according to item 116.
   118. Method of producing a fusion polypeptide according to any one of items 87-114 comprising
culturing a host cell according to item 117 under conditions permissive of expression of said polypeptide from said expression vector, and
isolating said polypeptide.
   119. Method of producing a fusion polypeptide according to item 118, wherein said method comprises heating the fusion polypeptide to approximately 100° C.
   120. Immunogenic composition, comprising at least one fusion polypeptide according to any one of items 87-114.
   121. Immunogenic composition according to any one of items 1-86 and 120, wherein said fusion polypeptide(s) is/are isolated or purified.
   122. Immunogenic composition according to any one of items 1-86 and 120-121, wherein said fusion polypeptide(s) is/are recombinantly produced.
   123. Immunogenic composition according to any one of items 1-86 or 120-122, further comprising an agent with adjuvant effect.
   124. Vaccine composition comprising an immunogenic composition according to any one of items 1-86 or 120-123 and a pharmaceutically acceptable carrier or excipient.
   125. Vaccine composition according to item 124, further comprising an agent with adjuvant effect.
   126. Vaccine composition according to any one of items 124-125 or immunogenic composition according to item 123, wherein said agent with adjuvant effect is selected from the group consisting of Matrix V, Abisco/Matrix M, Matrix C and Matrix Q and silica.
   127. Vaccine composition or immunogenic composition according to item 126, wherein said agent with adjuvant effect is Matrix V.
   128. Vaccine composition according to any one of items 124-127, which composition is formulated for intramuscular, intradermal, subcutaneous or intranasal administration, such as for intramuscular administration.
   129. Vaccine composition according to any one of items 124-128, which composition is capable of eliciting serum antibody response and/or mucosal antibody response in a mammalian subject, such as a porcine or human subject, such as a porcine subject.
   130. Vaccine composition according to item 129, wherein said antibody response is in the form of IgG, IgA and/or IgM antibodies in the serum and/or mucosa and/or colostrum.
   131. Vaccine composition according to any one of items 124-130, for use in the prophylactic treatment of a mammalian subject susceptible to S. suis infection, such as a human subject or porcine subject, such as a porcine subject.
   132. Vaccine composition according to any one of items 124-131, for use in the prophylactic treatment of piglets susceptible to S. suis infection.
   133. Method for the production of an antiserum or colostrum comprising the step of administering an immunogenic composition according to any one of items 1-86 and 120-123, to a mammalian host to produce antibodies in said host and recovering antiserum or colostrum containing the antibodies produced in the host.
   134. Antiserum or colostrum obtainable by the method defined item 133.
   135. Antibody or fragment thereof, which is capable of binding to a fusion polypeptide as defined in any one of items 87-114, which antibody or fragment thereof is polyclonal or monoclonal.
   136. An antibody preparation comprising one antibody or several antibodies according to item 135.
   137. Antibody preparation according to item 136, for use as a medicament, such as for use in the prophylactic treatment of a mammalian subject susceptible to Streptococcus suis infection.
   138. An immunogenic composition according to any one of the items 1-86 and 120-123, a fusion polypeptide according to any one of items 87-114, a vaccine composition according to any one of items 120-132, an antiserum according to item 134, or an antibody preparation according to any one of items 135-137, for use as medicament.
   139. An immunogenic composition according to any one of the items 1-86 and 120-123, a fusion polypeptide according to any one of items 87-114, a vaccine composition according to any one of items 121-132, an antiserum or colostrum according to item 134, or an antibody preparation according to any one of items 135-137, for use in the treatment, such as prophylactic treatment, of Streptococcus suis infection in a mammalian subject susceptible to, such as a human subject or porcine subject, such as a porcine subject.
   140. Immunogenic composition, fusion polypeptide, vaccine composition, an antiserum, colostrum or an antibody preparation for use according to item 139, wherein said use comprises administering on one single occasion or on multiple separate occasions, such at least two or at least three occasions.
   141. Immunogenic composition, fusion polypeptide, vaccine composition, an antiserum, colostrum or an antibody preparation for use according to item 139 or 140, wherein said use comprises administration to piglets, gilts or sows, such as pregnant sows or gilts or non-pregnant sows or gilts.
   142. Immunogenic composition, fusion polypeptide, vaccine composition, an antiserum or an antibody preparation for use according to any one of items 139-141, wherein said administration is intramuscular, intradermal, subcutaneous or intranasal administration, such as intramuscular administration.
   143. Immunogenic composition, fusion polypeptide, vaccine composition, an antiserum, colostrum or an antibody preparation for use according to any one of items 139-142, wherein, upon administration, serum and/or mucosal antibody response is elicited in said mammalian subject.
   144. Immunogenic composition, fusion polypeptide, vaccine composition, an antiserum, colostrum or an antibody preparation for use according to any one of items 143, wherein said antibody response is in the form of IgG, IgA and/or IgM antibodies in the serum and/or mucosa and/or colostrum.
   145. Immunogenic composition according any one of the items 1-86 and 119-122, a fusion polypeptide according to any one of items 87-114, a vaccine composition according to any one of items 121-132, for use in the prophylactic treatment of Streptococcus suis infection in piglets susceptible to, wherein said use comprises administration of said immunogenic composition, fusion polypeptide or vaccine composition to pregnant gilts or sows.
   146. Immunogenic composition, fusion polypeptide or vaccine composition, for use according to item 145, wherein said administration is at least two or at least three separate occasions, such as separate occasion at least approximately two weeks, such at approximately three weeks apart.
   147. Immunogenic composition, fusion polypeptide or vaccine composition, for use according to item 145 or 146, wherein said administration is administration is at a dose in the range of approximately 4-300 μg per fusion polypeptide, such as 60-140 μg, such as in the range of approximately 70-130 μg, such as in the range of approximately 80-120 μg per fusion polypeptide, such as in the range of approximately 90-110 μg, such as approximately 100 μg.
   148. Immunogenic composition, fusion polypeptide or vaccine composition, for use according to any one of items 145-147, wherein said piglets obtain antibodies via the colostrum after birth.
   149. Method for prophylactic treatment of a Streptococcus suis infection in a mammalian subject, comprising administering to said mammalian subject in need thereof an immunologically effective amount of an immunogenic composition according to any one of items 1-86 and 120-123, a fusion polypeptide according to any one of items 87-114, a vaccine composition according to any one of items 121-132, an antiserum or colostrum according to item 134 or an antibody preparation according to any one of items 135-137.
   150. Method for prophylactic treatment according to item 149, wherein said mammalian subject is a porcine or human subject, such as a porcine subject.
   151. Method for prophylactic treatment according to any one of items 149-150, comprising administering on one single occasion or on multiple separate occasions, such at least two or at least three occasions.
   152. Method for prophylactic treatment according to any one of items 149-150, wherein said administration is to piglets, gilts or sows, such as pregnant gilts or sows.
   153. Method for prophylactic treatment according to any one of times 149-152, wherein said administration is intramuscular, intradermal, subcutaneous or intranasal administration, such as intramuscular administration.
   154. Method for prophylactic treatment according to any one of times 149-153, wherein, upon administration, serum and/or mucosal antibody response is elicited in said mammalian subject.
   155. Method for prophylactic treatment according to item 154, wherein said antibody response is in the form of IgG, IgA and/or IgM antibodies in the serum and/or mucosa or colostrum.
   156. Method for prophylactic treatment of a Streptococcus suis infection in a mammalian subject, comprising administering to said mammalian subject in need thereof an immunologically effective amount of an immunogenic composition according to any one of items 1-86 and 120-123, a fusion polypeptide according to any one of items 87-114, a vaccine composition according to any one of items 121-132 wherein said subject is a piglet and said method comprises the step of administration of said immunogenic composition, fusion polypeptide or vaccine composition to pregnant gilts or sows.
   157. Method for prophylactic treatment according to item 156, wherein said administration is at least two or at least three separate occasions, such as separate occasions at least approximately two weeks, such at approximately at least three weeks apart.
   158. Method for prophylactic treatment according to item 156 or 157, wherein said administration is administration is at a dose in the range of approximately 4-300 μg per fusion polypeptide, such as 60-140 μg, such as in the range of approximately 70-130 μg, such as in the range of approximately 80-120 μg per fusion polypeptide, such as in the range of approximately 90-110 μg, such as approximately 100 μg.
   159. Method for prophylactic treatment according to item one of items 156-158, wherein said piglets obtain antibodies via the colostrum after birth.
   160. Method for prophylactic treatment of a Streptococcus suis infection in a mammalian subject, comprising passive immunization by administering to said mammalian subject in need thereof an antibody preparation according to item 136 or 137.
   161. Use of an immunogenic composition according to any one of items 1-86 and 120-123, a fusion polypeptide according to any one of items 87-114, a vaccine composition according to any one of items 121-132, an antiserum or colostrum according to item 134, or an antibody preparation according to any one of items 135-137, for the manufacture of a medicament for use in the prophylactic treatment of a mammalian subject susceptible to Streptococcus suis infection.
   162. A method for immunizing a mammalian subject against a Streptococcus suis infection, comprising administering to said mammalian subject in need thereof an immunologically effective amount of an immunogenic composition according to any one of items 1-86 and 120-123, a fusion polypeptide according to any one of items 87-114, a vaccine composition according to any one of items 121-132, an antiserum or colostrum according to item 134 or an antibody preparation according to any one of items 135-137.
   163. Method for immunizing a mammalian subject against a Streptococcus suis infection according to item 162, wherein said mammalian subject is a porcine or human subject, such as a porcine subject.
   164. Method for immunizing a mammalian subject against a Streptococcus suis infection according to any one of items 162-163, comprising administering on one single occasion or on multiple separate occasions, such at least two or at least three occasions.
   165. Method for immunizing a mammalian subject against a Streptococcus suis infection according to any one of items 162-164, wherein said administration is to piglets, gilts or sows, such as pregnant gilts or sows.
   166. Method for immunizing a mammalian subject against a Streptococcus suis infection according to any one of times 162-165, wherein said administration is intramuscular, intradermal, subcutaneous or intranasal administration, such as intramuscular administration.
   167. Method for immunizing a mammalian subject against a Streptococcus suis infection according to any one of times 162-166, wherein, upon administration, serum and/or mucosal antibody response is elicited in said mammalian subject.
   168. Method for immunizing a mammalian subject against a Streptococcus suis infection according to item 167, wherein said antibody response is in the form of IgG, IgA and/or IgM antibodies in the serum and/or mucosa or colostrum.
   169. A method for immunizing a mammalian subject against a Streptococcus suis infection, comprising administering to said mammalian subject in need thereof an immunologically effective amount of an immunogenic composition according to any one of items 1-86 and 120-123, a fusion polypeptide according to any one of items 87-114, a vaccine composition according to any one of items 121-132 wherein said subject is a piglet and said method comprises the step of administration of said immunogenic composition, fusion polypeptide or vaccine composition to pregnant gilts or sows.
   170. Method for immunizing a mammalian subject against a Streptococcus suis infection according to item 169, wherein said administration is at least two or at least three separate occasions, such as separate occasions at least approximately two weeks, such at approximately at least three weeks apart.
   171. Method for immunizing a mammalian subject against a Streptococcus suis infection according to item 169 or 170, wherein said administration is administration is at a dose in the range of approximately 4-300 μg per fusion polypeptide, such as 60-140 μg, such as in the range of approximately 70-130 μg, such as in the range of approximately 80-120 μg per fusion polypeptide, such as in the range of approximately 90-110 μg, such as approximately 100 μg.
   172. Method for immunizing a mammalian subject against a Streptococcus suis infection according to item one of items 169-171, wherein said piglets obtain antibodies via the colostrum after birth, such as antibodies which confer protection against S. suis infection.
   173. Method for immunizing a mammalian subject against a Streptococcus suis infection, comprising passive immunization by administering to said mammalian subject in need thereof an antibody preparation according to item 136 or 137.

1. An immunogenic composition comprising at least one first and one second fusion polypeptide, wherein the first fusion polypeptide comprises fragments from at least three native full length polypeptides from Streptococcus suis and the second fusion polypeptide comprises fragments from at least three native full length polypeptides from S. suis, and wherein said at least three native full length polypeptides from S. suis are independently selected from the group consisting of zinc-binding proteins from S. suis; proteases from S. suis; nucleotidases from S. suis; proteins from S. suis which comprise an LPXTG-motif, Amid1Sa (SEQ ID NO:14) and any polypeptides with at least 80% identity to SEQ ID NO:14; 15BSa (SEQ ID NO:15) and any polypeptides with at least 80% identity to SEQ ID NO:15; and Hom17Sa (SEQ ID NO:16) and any polypeptides with at least 80% identity to SEQ ID NO:16.

2. The immunogenic composition according to claim 1, wherein said immunogenic composition further comprises one or more of a third fusion polypeptide, a fourth fusion polypeptide and a fifth fusion polypeptide.

3. (canceled)

4. (canceled)

5. The immunogenic composition according to claim 1, wherein said at least three native full length polypeptides from S. suis are in independently selected from the group consisting of polypeptides having an amino acid sequence according to any one of SEQ ID NO:1-19 and any polypeptides with at least 80% identity to any one of SEQ ID NO:1-19; such as the group consisting of polypeptides having an amino acid sequence according to any one of SEQ ID NO:1-19.

6. (canceled)

7. (canceled)

8. (canceled)

9. (canceled)

10. (canceled)

11. (canceled)

12. (canceled)

13. (canceled)

14. (canceled)

15. The immunogenic composition according to claim 1, wherein the fragments of said zinc-binding proteins are selected from the group consisting of polypeptide fragments comprising an amino acid sequence selected from SEQ ID NO:20-27 and any amino acid sequences with at least 80% identity to any one of SEQ ID NO:20-27, such as the group consisting of polypeptide fragments comprising an amino acid sequence selected from SEQ ID NO:20-27.

16. The immunogenic composition according to claim 1, wherein said fragments of said proteases are selected from the group consisting of polypeptide fragments comprising an amino acid sequence selected from SEQ ID NO:28-33 and any amino acid sequences with at least 80% identity to any one of SEQ ID NO:28-33, such as the group consisting of polypeptide fragments comprising an amino acid sequence selected from SEQ ID NO:28-33.

17. The immunogenic composition according to claim 1, wherein said fragments of said nucleotidases are selected from the group consisting of polypeptide fragments comprising an amino acid sequence selected from SEQ ID NO:34-36 and any amino acid sequences with at least 80% identity to any one of SEQ ID NO:34-36, such as the group consisting of polypeptide fragments comprising an amino acid sequence selected from SEQ ID NO:34-36.

18. The immunogenic 4 composition according to claim 1, wherein said fragments of said proteins comprising an LPXTG-motif are selected from the group consisting of polypeptide fragments comprising an amino acid sequence selected from SEQ ID NO:40-42 and any amino acid sequences with at least 80% identity to any one of SEQ ID NO:40-42, such as the group consisting of polypeptide fragments comprising an amino acid sequence selected from SEQ ID NO:40-42.

19. The immunogenic composition according to claim 1, wherein said fragments selected from the group consisting of fragments of amino acid sequence SEQ ID NO:14, SEQ ID NO:15, SEQ ID NO:16 and any amino acid sequences having at least 80% identity to said fragments are fragments selected from the group consisting of SEQ ID NO:37-39 and any amino acid sequences with at least 80% identity to any one of SEQ ID NO:37-39, such as the group consisting of polypeptide fragments comprising an amino acid sequence selected from SEQ ID NO:37-39.

20. The immunogenic composition according to claim 1, wherein one of said first, second, third, fourth and fifth fusion polypeptides comprises at least one, such as at least two, such as at least three, fragment(s) selected from the group consisting of SEQ ID NO:20-22 and any amino acid sequence with at least 80% identity to any one of SEQ ID NO:20-22.

21. (canceled)

22. (canceled)

23. (canceled)

24. (canceled)

25. The immunogenic composition according to claim 1, wherein one of said first, second, third, fourth and fifth fusion polypeptides comprises at least one, such as at least two, such as at least three, such as at least four, such as at least five, such as at least six, such as at least seven such as eight, fragment(s) selected from the group consisting of SEQ ID NO:23-27 and SEQ ID NO:37-39 and any amino acid sequence with at least 80% identity to any one of SEQ ID NO: 23-27 and SEQ ID NO:37-39.

26. (canceled)

27. (canceled)

28. (canceled)

29. The immunogenic composition according to claim 1, wherein one of said first, second, third, fourth and fifth fusion polypeptides comprises at least one, such as at least two, such as at least three, such as at least four, such as at least five, such as at least six, fragment(s) selected from the group consisting of SEQ ID NO:28-33 and amino acid sequences with at least 80% identity to any one of SEQ ID NO:28-33.

30. (canceled)

31. (canceled)

32. (canceled)

33. The immunogenic composition according to claim 1, wherein one of said first, second, third, fourth and fifth fusion polypeptides comprises at least one, such as at least two, such as at least three, fragment(s) selected from the group consisting of SEQ ID NO:34-36 or amino acid sequences with at least 80% identity to any one of SEQ ID NO:34-36.

34. (canceled)

35. (canceled)

36. (canceled)

37. (canceled)

39. (canceled)

40. (canceled)

41. (canceled)

42. The immunogenic composition according to claim 1, wherein at least two, at least three, at least four or all five of said first, second, third, fourth and fifth fusion polypeptides are selected from the group consisting of:
fusion polypeptides comprising SEQ ID NO:20, 21 and 22 or amino acid sequences with at least 80% identity to any one of SEQ ID NO:20, 21 and 22;
fusion polypeptides comprising SEQ ID NO:23-27 and SEQ ID NO:37-39 or amino acid sequences with at least 80% identity to any one of SEQ ID NO:23-27 and SEQ ID NO:37-39;
fusion polypeptides comprising SEQ ID NO:28-33 or amino acid sequences with at least 80% identity to any one of SEQ ID NO:28-33;
fusion polypeptides comprising SEQ ID NO:34-36 or amino acid sequences with at least 80% identity to any one of SEQ ID NO:34-36; and
fusion polypeptides comprising SEQ ID NO:40-42 or amino acid sequences with at least 80% identity to any one of SEQ ID NO:40-42.

43. (canceled)

44. The immunogenic composition according to claim 1, wherein at least two, at least three, at least four or all five of said first, second, third, fourth and fifth fusion polypeptides are selected from the group consisting of:
fusion polypeptides comprising SEQ ID NO:106 or an amino acid sequence with at least 80% identity to SEQ ID NO:106;
fusion polypeptides comprising SEQ ID NO:44 or an amino acid sequence with at least 80% identity to SEQ ID NO:44;
fusion polypeptides comprising SEQ ID NO:45 or an amino acid sequence with at least 80% identity to SEQ ID NO:45;
fusion polypeptides comprising SEQ ID NO:107 or an amino acid sequence with at least 80% identity to SEQ ID NO:107; and
fusion polypeptides comprising SEQ ID NO:48 or an amino acid sequence with at least 80% identity to SEQ ID NO:48.

45. (canceled)

46. (canceled)

47. (canceled)

48. (canceled)

49. A fusion polypeptide comprising fragments from at least 3, such as at least 4, such as at least 5, such as at least 6, such as at least 7, native full length polypeptides from Streptococcus suis, wherein said native full length polypeptides are independently selected from the group consisting of amino acid sequences according to SEQ ID NO:1-10 and SEQ ID NO:14-19 and amino acid sequences having at least 80% identity to any one of SEQ ID NO:1-10 and SEQ ID NO:14-19; such as the group consisting of amino acid sequences according to SEQ ID NO:1-10 and SEQ ID NO:14-19.

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. The fusion polypeptide according to claim 49, comprising or consisting of the amino acid sequence SEQ ID NO:106 or 109 or an amino acid sequence with at least 80% identity to SEQ ID NO:106 or 109; or
comprising or consisting of the amino acid sequence SEQ ID NO:44 or 110 or an amino acid sequence with at least 80% identity to SEQ ID NO:44 or 110; or
comprising or consisting the amino acid sequence SEQ ID NO:45 or 111 or an amino acid sequence with at least 80% identity to SEQ ID NO:45 or 111; or
comprising or consisting the amino acid sequence SEQ ID NO:54 or 114 or an amino acid sequence with at least 80% identity to SEQ ID NO:54 or 114; or
comprising or consisting the amino acid sequence SEQ ID NO:55 or 115 or an amino acid sequence with at least 80% identity to SEQ ID NO:55 or 115.

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. A polynucleotide encoding a fusion polypeptide as defined in claim 49.

77. (canceled)

78. (canceled)

79. (canceled)

80. (canceled)

81. (canceled)

82. A vaccine composition comprising an immunogenic composition according to claim 1, and a pharmaceutically acceptable carrier or excipient.

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. A method for prophylactic treatment of a Streptococcus suis infection in a mammalian subject, such as a porcine subject, comprising administering to said mammalian subject in need thereof an immunologically effective amount of an immunogenic composition according to claim 1.

100. The method for prophylactic treatment of a Streptococcus suis infection in a mammalian subject according to claim 99, comprising administering to said mammalian subject in need thereof the immunologically effective amount of the immunogenic composition, wherein said subject is a piglet and said method comprises the step of administration of said immunogenic composition to pregnant gilts or sows.
